(19) United States
(12) Patent Application Publication
(10) Pub. No.: US 2021/0188839 A1
(43) Pub. Date: Jun. 24, 2021

(54) COMPOUNDS AND THEIR METHODS OF USE

(71) Applicant: Praxis Precision Medicines, Inc., Cambridge, MA (US)

(72) Inventors: Kiran Reddy, Boston, MA (US); Gabriel Martinez Botella, Wayland, MA (US); Andrew Mark Griffin, L'lle Bizard (CA); Brian Edward Marron, Durham, NC (US)

(21) Appl. No.: 16/464,467

(22) PCT Filed: Nov. 28, 2017
(86) PCT No.: PCT/US2017/063534

§ 371 Date: May 28, 2019

Related U.S. Application Data
(60) Provisional application No. 62/427,050, filed on Nov. 28, 2016.
(60) Provisional application No. 62/458,308, filed on Feb. 13, 2017.

(51) Int. Cl. C07D 471/04; C07D 487/04

(52) U.S. Cl. CPC C07D 471/04; C07D 487/04

(57) ABSTRACT

The present invention is directed to, in part, fused heteroaryl compounds and compositions useful for preventing and/or treating a disease or condition relating to aberrant function of a voltage-gated, sodium ion channel, for example, abnormal late/persistent sodium current. Methods of treating a disease or condition relating to aberrant function of a sodium ion channel including Dravet syndrome or epilepsy are also provided herein.

### CROSS REFERENCE TO RELATED APPLICATIONS

This application claims priority to and the benefit of U.S. Provisional Application No. 62/427,050 filed Nov. 28, 2016 and U.S. Provisional Application No. 62/458,308 filed Feb. 13, 2017, each of which is incorporated herein by reference in its entirety.

### BACKGROUND

Sodium ion (Na+) channels primarily open in a transient manner and are quickly inactivated, thereby generating a fast Na+ current to initiate the action potential. The late or persistent sodium current (INaL) is a sustained component of the fast Na+ current of cardiac myocytes and neurons. Many common neurological and cardiac conditions are associated with abnormal INaL enhancement, which contributes to the pathogenesis of both electrical and contractile dysfunction in mammals (see, e.g., Pharmacol Ther (2008) 119:326-339). Accordingly, pharmaceutical compounds that selectively modulate sodium channel activity, e.g., abnormal INaL, are useful in treating such disease states.

### SUMMARY OF THE INVENTION

Described herein are fused heteroaryl compounds and compositions useful for preventing and/or treating a disease, disorder, or condition, e.g., a disease, disorder, or condition relating to aberrant function of a sodium ion channel, e.g., abnormal late sodium current (INaL). In one aspect, the present disclosure features compounds of Formula (I):

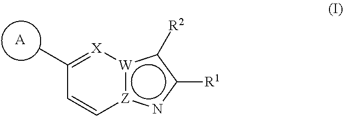

or a pharmaceutically acceptable salt thereof, wherein X is N or CR′; each of W and Z is independently N or C, wherein only one of W and Z is independently N; A is aryl or heteroaryl (e.g., a monocyclic 6-membered aryl or heteroaryl), optionally substituted by one or more R³; R′ is hydrogen or alkyl; each of R¹ and R² is independently hydrogen or alkyl, wherein alkyl is optionally substituted by one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; R^{c} is hydrogen, alkyl, aryl, or heteroaryl, wherein alkyl, aryl, and heteroaryl is optionally substituted by one or more R⁶; each R^{d} is independently hydrogen or alkyl; and each R⁶ is independently alkyl, carbocyclyl, heterocyclyl, halo, cyano, nitro, or —OH.

Described herein are fused heteroaryl compounds and compositions useful for preventing and/or treating a disease, disorder, or condition, e.g., a disease, disorder, or condition relating to aberrant function of a sodium ion channel, e.g., abnormal late sodium current (INaL). In one aspect, the present disclosure features compounds of Formula (I-2):

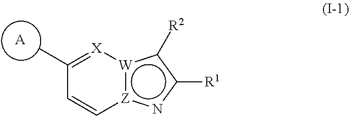

or a pharmaceutically acceptable salt thereof, wherein X is N or CR′; each of W and Z is independently N or C, wherein only one of W and Z is independently N; A is aryl or heteroaryl (e.g., a monocyclic 6-membered aryl or heteroaryl), optionally substituted by one or more R³; R′ is hydrogen, halogen, or alkyl; R¹ and R² is independently hydrogen, —OR^{c}, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴; wherein alkyl is optionally substituted by one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; R^{c} is hydrogen, alkyl, aryl, cycloalkyl, heterocyclyl or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl carbocyclyl, heterocyclyl, halo, cyano, nitro, or —OH.

In another aspect, the present disclosure features compounds of Formula (II):

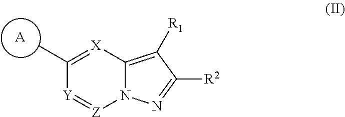

or a pharmaceutically acceptable salt thereof, wherein each of X, Y, and Z is independently N or CR′; A is aryl or heteroaryl (e.g., a monocyclic 6-membered aryl or heteroaryl), wherein aryl and heteroaryl are optionally substituted with one or more R³; R′ is hydrogen, alkyl, or —OR^{c}; each of R¹ and R² is independently hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, or heteroaryl, wherein alkyl, aryl, and heteroaryl is optionally substituted by one or more R⁶; each R^{d} is independently hydrogen or alkyl; and each R⁶ is independently alkyl, carbocyclyl, heterocyclyl, halo, cyano, nitro, or —OH.

In another aspect, the present disclosure features compounds of Formula (II-2):

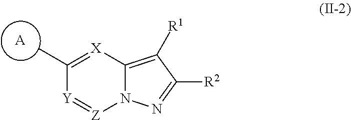

or a pharmaceutically acceptable salt thereof, wherein each of X, Y, and Z is independently N or CR′; A is aryl or heteroaryl (e.g., a monocyclic 6-membered aryl or heteroaryl), wherein aryl and heteroaryl are optionally substituted with one or more R³; R′ is hydrogen, halogen, alkyl, or —OR^{c}; each of R¹ and R² is independently hydrogen, —OR^{c}, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In another aspect, the present invention provides a compound of formula IIa:

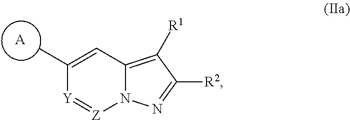

or a pharmaceutically acceptable salt thereof, wherein the variables are as defined herein.

In another aspect, the present invention provides a compound of formula IIb:

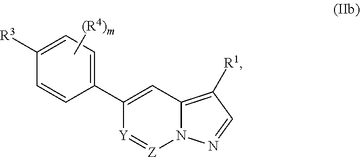

or a pharmaceutically acceptable salt thereof, wherein the variables are as defined herein.

In some embodiments, the present invention provides a compound of formula (IIb-1):

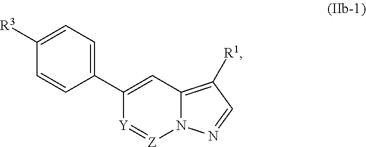

or a pharmaceutically acceptable salt thereof, wherein the variables are as defined herein.

In another aspect, the present disclosure provides a compound of formula IIc:

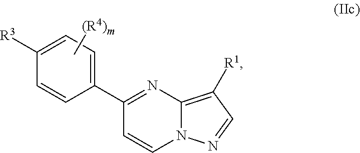

or a pharmaceutically acceptable salt thereof, wherein the variables are as defined herein.

In another aspect, the present disclosure provides a compound of formula IId:

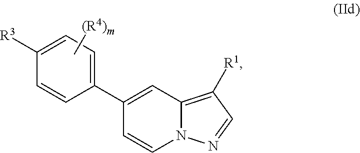

or a pharmaceutically acceptable salt thereof, wherein the variables are as defined herein.

In another aspect, the present invention provides a compound of formula (IIe):

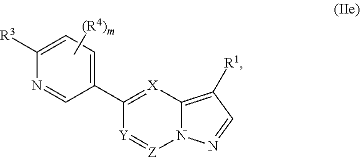

or a pharmaceutically acceptable salt thereof, wherein the variables are as defined herein.

In some embodiments, the present invention provides a compound of formula (IIe-1):

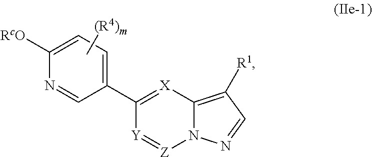

or a pharmaceutically acceptable salt thereof, wherein the variables are as defined herein.

In another aspect, the present invention provides a compound of formula IIf:

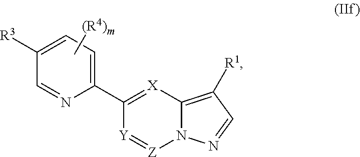

or a pharmaceutically acceptable salt thereof, wherein the variables are as defined herein.

In some embodiments, the present invention provides a compound of formula IIf-1:

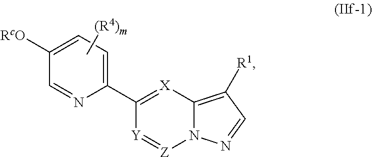

or a pharmaceutically acceptable salt thereof, wherein the variables are as defined herein.

In another aspect, the present invention features compound of Formula (III):

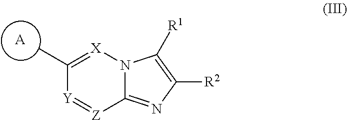

or a pharmaceutically acceptable salt thereof, wherein each of X, Y, and Z is independently N or CR′; A is aryl or heteroaryl (e.g., a monocyclic 6-membered aryl or heteroaryl), wherein aryl and heteroaryl are optionally substituted with one or more R³; R′ is hydrogen, alkyl, or —OR^{c}; each of R¹ and R² is independently hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, heterocyclyl, cycloalkyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In another aspect, the present invention provides a compound of formula IIIa:

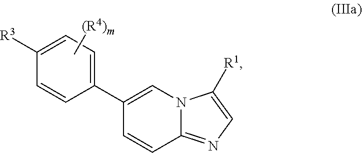

or a pharmaceutically acceptable salt thereof, wherein the variables are as defined herein.

In another aspect, the present invention provides a compound of formula (IIIb):

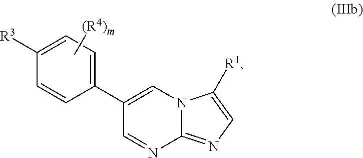

or a pharmaceutically acceptable salt thereof, wherein the variables are as defined herein.

In another aspect, the present invention provides a compound of formula (IIIc):

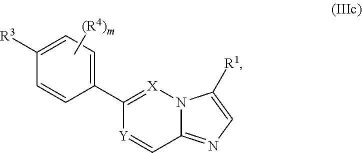

or a pharmaceutically acceptable salt thereof, wherein the variables are as defined herein.

In another aspect, the present invention provides a compound of formula (IIId):

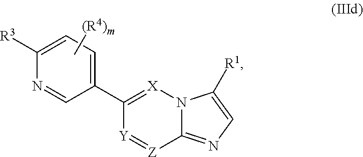

or a pharmaceutically acceptable salt thereof, wherein the variables are as defined herein.

In some embodiments, the compound is a compound of formula (IIId-1):

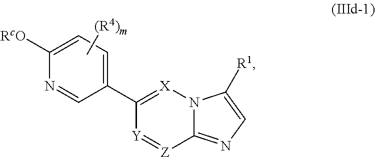

or a pharmaceutically acceptable salt thereof, wherein the variables are as defined herein.

In another aspect the present invention provides a compound of formula IIIe:

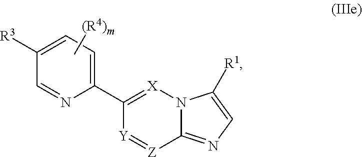

or a pharmaceutically acceptable salt thereof, wherein the variables are as defined herein.

In some embodiments, the compound is a compound of formula IIIe-1:

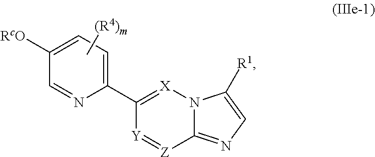

or a pharmaceutically acceptable salt thereof, wherein the variables are as defined herein.

In another aspect, the present disclosure provides a method of treating a neurological disorder or a psychiatric disorder, wherein the method comprises administering to a subject in need thereof a compound of Formula (II):

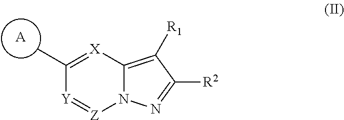

or a pharmaceutically acceptable salt thereof, wherein:

each of X, Y, and Z is independently N or CR;

A is aryl or heteroaryl (e.g., a monocyclic 6-membered aryl or heteroaryl), wherein aryl and heteroaryl are optionally substituted with one or more R³.

R is hydrogen, alkyl, or —OR^{c};

each of R¹ and R² is hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴.

each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵;

each R⁴ and R⁵ is independently alkyl, halo, cyano, nitro, or —OR^{c};

each R^{c} is independently hydrogen, alkyl, aryl, or heteroaryl, wherein alkyl, aryl, or heteroaryl is optionally substituted by one or more R⁶;

each R^{d} is independently hydrogen or alkyl; and

each R⁶ is independently alkyl, carbocyclyl, heterocyclyl, halo, cyano, nitro, or —OH.

In another aspect, the present disclosure provides a method of treating a neurological disorder or a psychiatric disorder, wherein the method comprises administering to a subject in need thereof a compound of Formula (II-2):

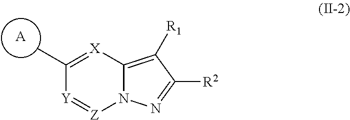

or a pharmaceutically acceptable salt thereof, wherein:

each of X, Y, and Z is independently N or CR′;

A is aryl or heteroaryl (e.g., a monocyclic 6-membered aryl or heteroaryl), wherein aryl and heteroaryl are optionally substituted with one or more R³;

R′ is hydrogen, alkyl, or —OR^{c};

each of R¹ and R² is hydrogen, —OR^{c}, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴;

each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵;

each R⁴ and R⁵ is independently alkyl, halo, cyano, nitro, or —OR^{c};

each R^{c} is independently hydrogen, alkyl, aryl, cycloalkyl, heterocyclyl, or heteroaryl, wherein alkyl, aryl, cycloalkyl, heterocyclyl, or heteroaryl is optionally substituted by one or more R⁶;

each R^{d} is independently hydrogen or alkyl; and

each R⁶ is independently alkyl, haloalkyl, carbocyclyl, heterocyclyl, halo, cyano, nitro, or —OH.

In some embodiments, the neurological disorder is epilepsy. In some embodiments, the neurological disorder is an epileptic encephalopathy. In some embodiments, the epileptic encephalopathy comprises Dravet syndrome, infantile spasms, or Lennox-Gastaut syndrome. In some embodiments, each of X, Y, and Z is independently CR′. In some embodiments, CR′ is CH. In some embodiments, one of X, Y, and Z is independently N. In some embodiments, X is N and each of Y and Z is independently CR′. In some embodiments, CR′ is CH. In some embodiments, A is aryl (e.g., phenyl). In some embodiments, A is phenyl substituted by 1-3 R³. In some embodiments, A is heteroaryl (e.g., pyridyl). In some embodiments, A is pyridyl substituted by 1-3 R³. In some embodiments, each R³ is independently alkyl, halo, cyano, carbocyclyl, or —OR^{c}. In some embodiments, R³ is alkyl or —OR^{c}. In some embodiments, each of R¹ and R² is hydrogen. In some embodiments, R is alkyl (e.g., substituted with one or more R⁴) and R² is hydrogen. In some embodiments, R¹ is —CF₃.

In some embodiments, the compound of Formula (II-2) is selected from the group consisting of:

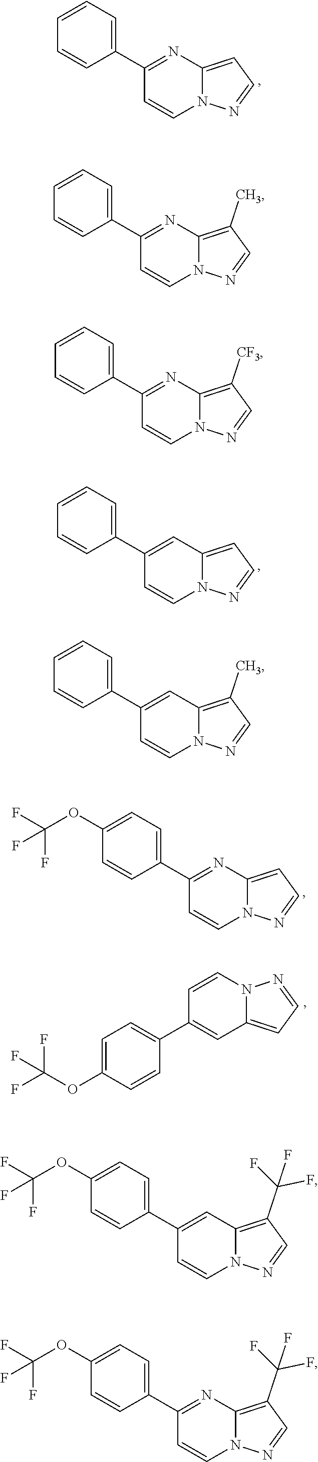

and a pharmaceutically acceptable salt thereof.

In some embodiments, the compound of Formula (II-2) is selected from the group consisting of:

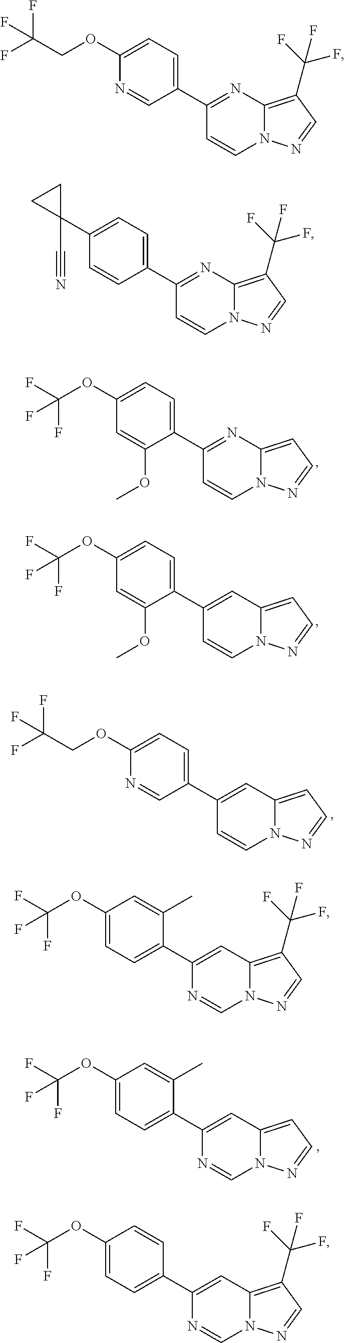

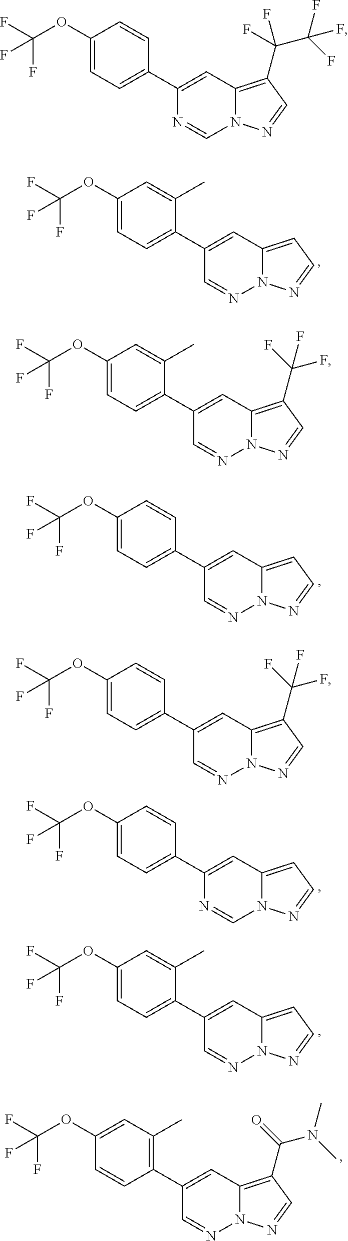

and a pharmaceutically acceptable salt thereof.

In another aspect, the present disclosure provides a method of treating a neurological disorder or a psychiatric disorder, wherein the method comprises administering to a subject in need thereof a compound of Formula (III):

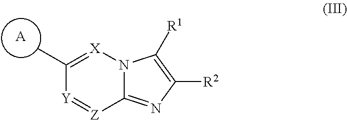

or a pharmaceutically acceptable salt thereof, wherein:

each of X, Y, and Z is independently N or CR;

A is aryl or heteroaryl (e.g., a monocyclic 6-membered aryl or heteroaryl), wherein aryl and heteroaryl are optionally substituted with one or more R³;

R′ is hydrogen, alkyl, or —OR^{c};

each of R¹ and R² is hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴;

each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵;

each R⁴ and R⁵ is independently alkyl, halo, cyano, nitro, or —OR^{c};

each R^{c} is independently hydrogen, alkyl, aryl, cycloalkyl, heterocyclyl, or heteroaryl, wherein alkyl, aryl, cycloalkyl, heterocyclyl, or heteroaryl is optionally substituted by one or more R⁶;

each R^{d} is independently hydrogen or alkyl; and

each R⁶ is independently alkyl, haloalkyl, carbocyclyl, heterocyclyl, halo, cyano, nitro, or —OH.

In some embodiments, the neurological disorder is epilepsy. In some embodiments, the neurological disorder is an epileptic encephalopathy. In some embodiments, the epileptic encephalopathy comprises Dravet syndrome, infantile spasms, or Lennox-Gastaut syndrome. In some embodiments, each of X, Y, and Z is independently CR′. In some embodiments, CR′ is CH. In some embodiments, one of X, Y, and Z is independently N. In some embodiments, X is N and each of Y and Z is independently CR′. In some embodiments, CR′ is CH. In some embodiments, A is aryl (e.g., phenyl). In some embodiments, A is phenyl substituted by 1-3 R³. In some embodiments, A is heteroaryl (e.g., pyridyl). In some embodiments, A is pyridyl substituted by 1-3 R³. In some embodiments, each R³ is independently alkyl, halo, cyano, carbocyclyl, or —OR^{c}. In some embodiments, R³ is alkyl or —OR^{c}. In some embodiments, each of R and R² is hydrogen. In some embodiments, R is alkyl (e.g., substituted with one or more R⁴) and R² is hydrogen. In some embodiments, R¹ is —CF₃. In some embodiments, the compound of Formula (III) is selected from the group consisting of:

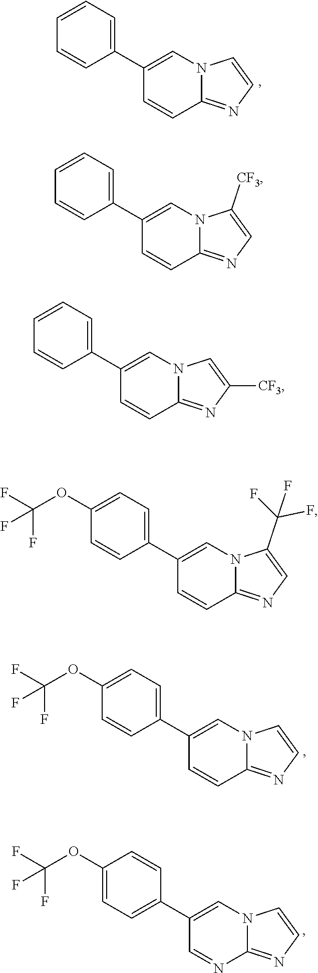 [SMILES: c1ccc(-c2ccc3nccn3c2)cc1 ; FC(F)(F)c1cnc2ccc(-c3ccccc3)cn12 ; FC(F)(F)c1cn2cc(-c3ccccc3)ccc2n1 ; FC(F)(F)Oc1ccc(-c2ccc3ncc(C(F)(F)F)n3c2)cc1 ; FC(F)(F)Oc1ccc(-c2ccc3nccn3c2)cc1 ; FC(F)(F)Oc1ccc(-c2cnc3nccn3c2)cc1]

pharmaceutically acceptable salt thereof.

In some embodiments, the compound of Formula (III) is selected from the group consisting of:

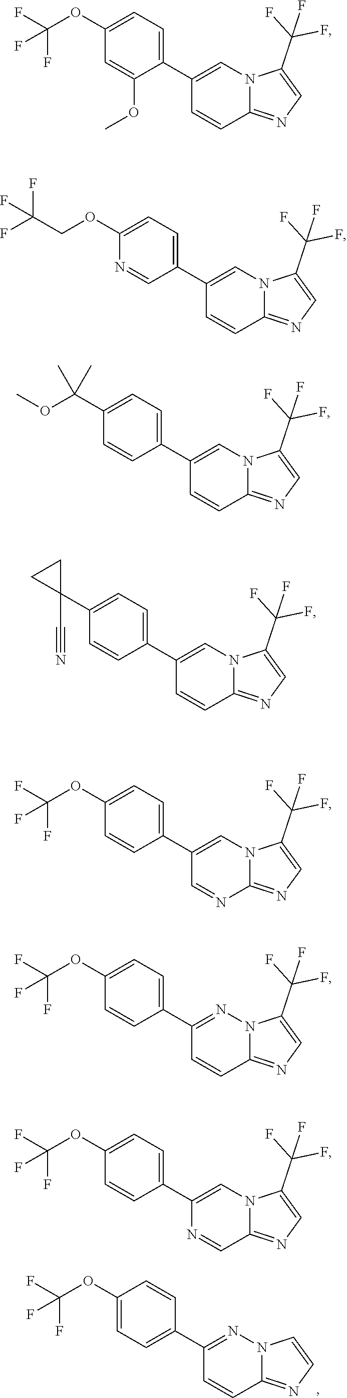

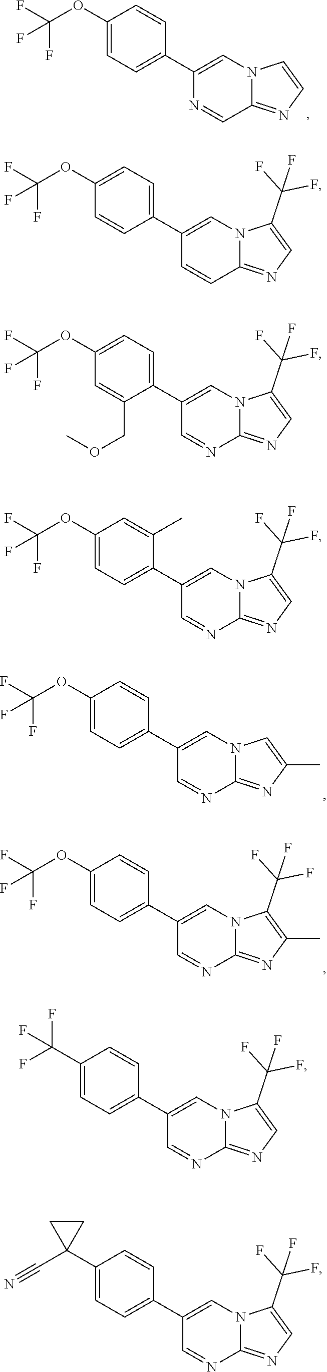

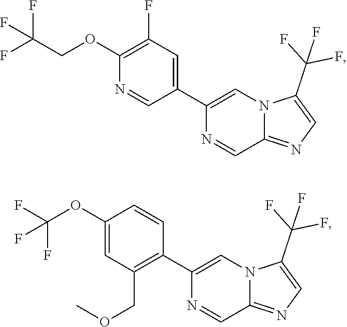 [SMILES: Fc1cc(-c2cn3c(C(F)(F)F)cnc3cn2)cnc1OCC(F)(F)F ; COCc1cc(OC(F)(F)F)ccc1-c1cn2c(C(F)(F)F)cnc2cn1]

and a pharmaceutically acceptable salt thereof.

In another aspect, the present disclosure provides a method of treating a neurological disorder or a psychiatric disorder, wherein the method comprises administering to a subject in need thereof a compound disclosed herein (a compound of Formulae (I), (I-2), (I-a), (I-b), (I-c), (I-d), (II), (II-2), (IIa), (IIb), (IIb-1), (IIc), (IId), (IIe), (IIe-1), (IIf), (IIf-1), (III), (IIIa), (IIIb), (IIIc), (IIId), (IIId-1), (IIIe), or (IIIe-1)).

Other objects and advantages will become apparent to those skilled in the art from a consideration of the ensuing Detailed Description, Examples, and Claims.

### DETAILED DESCRIPTION OF THE INVENTION

As generally described herein, the present invention provides compounds and compositions useful for preventing and/or treating a disease, disorder, or condition described herein, e.g., a disease, disorder, or condition relating to aberrant function of a sodium ion channel, such as abnormal late sodium current (INaL). Exemplary diseases, disorders, or conditions include epilepsy or an epilepsy syndrome.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including 160 and 180; and the like.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group, e.g., having 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C), and the like. Additional examples of alkenyl include heptenyl (C₇), octenyl (C), octatrienyl (C₈), and the like.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C), and the like. Additional examples of alkynyl include heptynyl (C₇), octynyl (C₈), and the like.

As used herein, “alkylene,” “alkenylene,” and “alkynylene,” refer to a divalent radical of an alkyl, alkenyl, and alkynyl group respectively. When a range or number of carbons is provided for a particular “alkylene,” “alkenylene,” or “alkynylene,” group, it is understood that the range or number refers to the range or number of carbons in the linear carbon divalent chain. “Alkylene,” “alkenylene,” and “alkynylene,” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

“Fused aryl” refers to an aryl having two of its ring carbon in common with a second aryl or heteroaryl ring or with a carbocyclyl or heterocyclyl ring.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

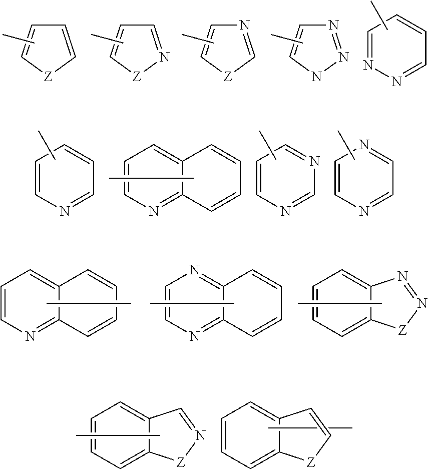

wherein each Z is selected from carbonyl, N, NR⁶⁵, O, and S; and R⁶⁵ is independently hydrogen, C₁-C₈ alkyl, C₃-C₁₀ carbocyclyl, 4-10 membered heterocyclyl, C₆-C₁₀ aryl, and 5-10 membered heteroaryl.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C), cyclohexenyl (C), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

The term “cycloalkyl,” as used herein, refers to a monocyclic saturated or partially unsaturated hydrocarbon ring system, for example, having 3-8 or 3-6 carbon atoms in its ring system, referred to herein as C₃₋₈ cycloalkyl or C₃₋₆ cycloalkyl, respectively. Exemplary cycloalkyl groups include, but are not limited to, cyclohexyl, cyclohexenyl, cyclopentyl, cyclopentenyl, cyclobutyl, and cyclopropyl.

The term “haloalkyl” as used herein refers to an alkyl group, in which one or more hydrogen atoms of the alkyl group are replaced with one or more independently selected halogens. A haloalkyl group can have 1 to 10 carbon atoms (i.e., C₁₋₁₀ haloalkyl group), for example, 1 to 6 carbon atoms (i.e., C₁₋₆ haloalkyl group). Examples of haloalkyl groups include —CF₃, —C₂F₅, —CHF₂, —CH₂F, —CCl₃, —CHC₂, —CH₂Cl, —CH₂CH₂Cl, —CHFCH₂Cl, and —C₂Cl₅.

Perhaloalkyl groups, i.e., alkyl groups where all of the hydrogen atoms are replaced with halogen atoms (e.g., —CF₃ and —C₂F₅), are included within the definition of “haloalkyl.”

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, carbocyclyl, e.g., heterocyclyl, aryl, e.g., heteroaryl, cycloalkenyl, e.g., cycloheteroalkenyl, and the like having from 1 to 5, and particularly from 1 to 3 heteroatoms.

“Cyano” refers to the radical —CN.

“Halo” or “halogen” refers to fluoro (F), chloro (Cl), bromo (Br), and iodo (I). In certain embodiments, the halo group is either fluoro or chloro.

“Haloalkyl” refers to an alkyl group substituted with one or more halogen atoms.

“Nitro” refers to the radical —NO₂.

In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, SO₄⁻² sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quaternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}—, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{d} groups, and wherein R^{aa}, R^{bb}, R^{cc} and R^{dd} are as defined above.

These and other exemplary substituents are described in more detail in the Detailed Description, Examples, and Claims. The invention is not intended to be limited in any manner by the above exemplary listing of substituents.

### Other Definitions

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

### Compounds

In one aspect, the present invention features a compound of Formula (I-a):

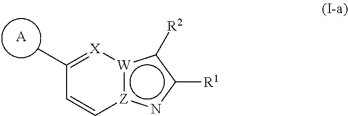

or a pharmaceutically acceptable salt thereof, wherein X is N or CR; each of W and Z is independently N or C, wherein only one of W and Z is independently N; A is aryl or heteroaryl (e.g., a monocyclic 6-membered aryl or heteroaryl), optionally substituted by one or more R³; R′ is hydrogen or alkyl; each of R¹ and R² is independently hydrogen or alkyl, wherein alkyl is optionally substituted by one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In some embodiments, X is N. In some embodiments, X is CR′. In some embodiments, R′ is hydrogen. In some embodiments, R′ is alkyl.

In some embodiments, W is N. In some embodiments, W is C.

In some embodiments, Z is N. In some embodiments, Z is C.

In some embodiments, W is N and Z is C. In some embodiments, W is C and Z is N.

In some embodiments, X is CR′ (e.g., CH), W is N, and Z is C. In some embodiments, X is CR′ (e.g., CH), W is C, and Z is N. In some embodiments, X is N, W is C, and Z is N.

In some embodiments, A is aryl (e.g., phenyl). In some embodiments, A is heteroaryl.

In some embodiments, A is a 6-membered heteroaryl. In some embodiments, A is a nitrogen-containing heteroaryl (e.g., pyridyl).

In some embodiments, A is not substituted with R³. In some embodiments, A is substituted by one or more R³. In some embodiments, A is a 6-membered aryl or heteroaryl and is substituted by R³ in the para position. In some embodiments, A is a 6-membered aryl or heteroaryl and is substituted by R³ in the meta position. In some embodiments, A is a 6-membered aryl or heteroaryl and is substituted by R³ in the ortho position.

In some embodiments, each R³ is independently alkyl, halo, cyano, carbocyclyl, heterocyclyl, or —OR^{c}. In some embodiments, R³ is alkyl (e.g., C₁-C₄ alkyl). In some embodiments, R³ is unsubstituted alkyl (e.g., unsubstituted C₁-C₄ alkyl) or substituted alkyl (e.g., substituted C₁-C₄ alkyl). In some embodiments, R³ is methyl. In some embodiments, R³ is substituted C₁ alkyl (e.g., wherein the C₁ alkyl is substituted with alkyl (e.g., methyl), halo (e.g., fluoro), cyano, carbocyclyl, or —OR′ (e.g., —OCH₃)). In some embodiments, R³ is —CF₃.

In some embodiments, R³ is halo (e.g., fluoro).

In some embodiments, R³ is —OR^{c}. In some embodiments, R³ is —OR^{c}, wherein R^{c} is alkyl (e.g., substituted alkyl or unsubstituted alkyl). In some embodiments, R³ is —OCF₃, —OCH₃, —OCH(CH₃)(CF₃), or —OCH₂CF₃.

In some embodiments, R¹ is hydrogen. In some embodiments, R¹ is alkyl. In some embodiments, R¹ is C₁₋₆ alkyl. In some embodiments, R¹ is unsubstituted C₁₋₆ alkyl (e.g., —CH₃).

In some embodiments, R¹ is substituted alkyl (e.g., substituted C₁₋₆ alkyl, e.g., CF₃).

In some embodiments, R² is hydrogen. In some embodiments, R² is alkyl. In some embodiments, R² is C₁₋₆ alkyl. In some embodiments, R² is unsubstituted C₁₋₆ alkyl (e.g., —CH₃).

In some embodiments, R² is substituted alkyl (e.g., substituted C₁₋₆ alkyl, e.g., CF₃).

In some embodiments, each of R¹ and R² is independently hydrogen. In some embodiments, each of R¹ and R² is independently alkyl. In some embodiments, R¹ is hydrogen and R² is alkyl. In some embodiments, R¹ is alkyl and R² is hydrogen.

In some embodiments, the compound of Formula (I-a) is not:

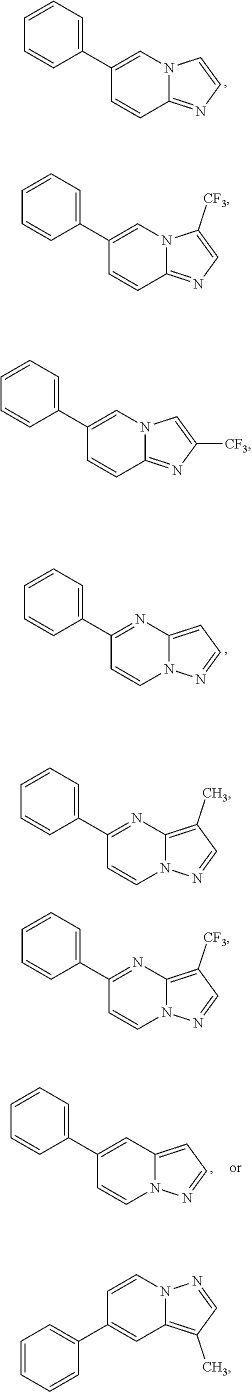

or a pharmaceutically acceptable salt thereof.

In another aspect, provided is a compound of Formula (I-b):

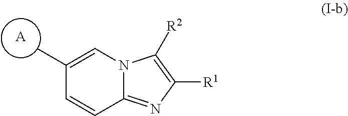

or a pharmaceutically acceptable salt thereof, A is aryl or heteroaryl (e.g., a monocyclic 6-membered aryl or heteroaryl), optionally substituted by one or more R³; each of R¹ and R² is independently hydrogen or alkyl, wherein alkyl is optionally substituted by one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In some embodiments, A is aryl (e.g., phenyl). In some embodiments, A is heteroaryl. In some embodiments, A is a 6-membered heteroaryl. In some embodiments, A is a nitrogen-containing heteroaryl (e.g., pyridyl).

In some embodiments, A is substituted by one or more R³. In some embodiments, A is a 6-membered aryl or heteroaryl and is substituted by R³ in the para position. In some embodiments, A is a 6-membered aryl or heteroaryl and is substituted by R³ in the meta position.

In some embodiments, A is a 6-membered aryl or heteroaryl and is substituted by R³ in the ortho position.

In some embodiments, each R³ is independently alkyl, halo, cyano, carbocyclyl, heterocyclyl, or —OR^{c}. In some embodiments, R³ is alkyl (e.g., C₁-C₄ alkyl). In some embodiments, R³ is unsubstituted alkyl (e.g., unsubstituted C₁-C₄ alkyl) or substituted alkyl (e.g., substituted C₁-C₄ alkyl). In some embodiments, R³ is methyl. In some embodiments, R³ is substituted C₁ alkyl (e.g., wherein the C₁ alkyl is substituted with halo (e.g., fluoro), cyano, carbocyclyl, or —OR′ (e.g., —OCH₃)). In some embodiments, R³ is —CF₃.

In some embodiments, R³ is halo (e.g., fluoro).

In some embodiments, R³ is —OR^{c}. In some embodiments, R³ is —OR^{c}, wherein R^{c} is alkyl (e.g., substituted alkyl or unsubstituted alkyl). In some embodiments, R³ is —OCF₃, —OCH₃, —OCH(CH₃)(CF₃), or —OCH₂CF₃.

In some embodiments, R¹ is hydrogen. In some embodiments, R¹ is alkyl. In some embodiments, R¹ is C₁₋₆ alkyl. In some embodiments, R¹ is unsubstituted C₁₋₆ alkyl (e.g., —CH₃).

In some embodiments, R¹ is substituted alkyl (e.g., substituted C₁₋₆ alkyl, e.g., CF₃).

In some embodiments, R² is hydrogen. In some embodiments, R² is alkyl. In some embodiments, R² is C₁₋₆ alkyl. In some embodiments, R² is unsubstituted C₁₋₆ alkyl (e.g., —CH₃). In some embodiments, R² is substituted alkyl (e.g., substituted C₁₋₆ alkyl, e.g., CF₃).

In some embodiments, each of R¹ and R² is independently hydrogen. In some embodiments, each of R¹ and R² is independently alkyl. In some embodiments, R¹ is hydrogen and R² is alkyl. In some embodiments, R¹ is alkyl and R² is hydrogen.

In some embodiments, the compound of Formula (I-b) is not:

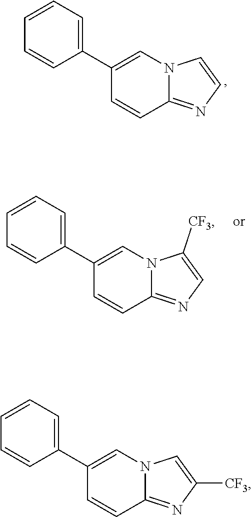 [SMILES: c1ccc(-c2ccc3nccn3c2)cc1 ; FC(F)(F)c1cnc2ccc(-c3ccccc3)cn12 ; FC(F)(F)c1cn2cc(-c3ccccc3)ccc2n1]

or a pharmaceutically acceptable salt thereof.

In another aspect, provided is a compound of Formula (I-c):

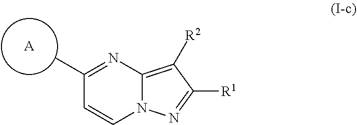

or a pharmaceutically acceptable salt thereof, A is aryl or heteroaryl (e.g., a monocyclic 6-membered aryl or heteroaryl), optionally substituted by one or more R³; each of R¹ and R² is independently hydrogen or alkyl, wherein alkyl is optionally substituted by one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In some embodiments, A is aryl (e.g., phenyl). In some embodiments, A is heteroaryl. In some embodiments, A is a 6-membered heteroaryl. In some embodiments, A is a nitrogen-containing heteroaryl (e.g., pyridyl).

In some embodiments, A is substituted by one or more R³. In some embodiments, A is a 6-membered aryl or heteroaryl and is substituted by R³ in the para position. In some embodiments, A is a 6-membered aryl or heteroaryl and is substituted by R³ in the meta position. In some embodiments, A is a 6-membered aryl or heteroaryl and is substituted by R³ in the ortho position.

In some embodiments, each R³ is independently alkyl, halo, cyano, carbocyclyl, heterocyclyl, or —OR^{c}. In some embodiments, R³ is alkyl (e.g., C₁-C₄ alkyl). In some embodiments, R³ is unsubstituted alkyl (e.g., unsubstituted C₁-C₄ alkyl) or substituted alkyl (e.g., substituted C₁-C₄ alkyl). In some embodiments, R³ is methyl. In some embodiments, R³ is substituted C₁ alkyl (e.g., wherein the C₁ alkyl is substituted with halo (e.g., fluoro), cyano, carbocyclyl, or —OR′ (e.g., —OCH₃)). In some embodiments, R³ is —CF₃.

In some embodiments, R³ is halo (e.g., fluoro).

In some embodiments, R³ is —OR^{c}. In some embodiments, R³ is —OR^{c}, wherein R^{c} is alkyl (e.g., substituted alkyl or unsubstituted alkyl). In some embodiments, R³ is —OCF₃, —OCH₃, —OCH(CH₃)(CF₃), or —OCH₂CF₃.

In some embodiments, R¹ is hydrogen. In some embodiments, R¹ is alkyl. In some embodiments, R¹ is C₁₋₆ alkyl. In some embodiments, R¹ is unsubstituted C₁₋₆ alkyl (e.g., —CH₃). In some embodiments, R¹ is substituted alkyl (e.g., substituted C₁₋₆ alkyl, e.g., CF₃).

In some embodiments, R² is hydrogen. In some embodiments, R² is alkyl. In some embodiments, R² is C₁₋₆ alkyl. In some embodiments, R² is unsubstituted C₁₋₆ alkyl (e.g., —CH₃). In some embodiments, R² is substituted alkyl (e.g., substituted C₁₋₆ alkyl, e.g., CF₃).

In some embodiments, each of R¹ and R² is independently hydrogen. In some embodiments, each of R¹ and R² is independently alkyl. In some embodiments, R¹ is hydrogen and R² is alkyl. In some embodiments, R¹ is alkyl and R² is hydrogen.

In some embodiments, the compound of Formula (I-c) is not:

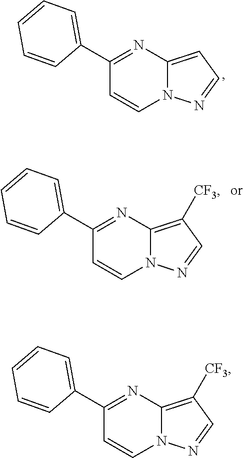 [SMILES: c1ccc(-c2ccn3nccc3n2)cc1 ; FC(F)(F)c1cnn2ccc(-c3ccccc3)nc12 ; FC(F)(F)c1cnn2ccc(-c3ccccc3)nc12]

or a pharmaceutically acceptable salt thereof.

In another aspect, provided is a compound of Formula (I-d):

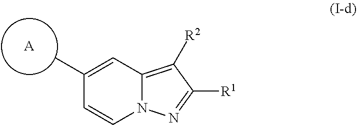

or a pharmaceutically acceptable salt thereof, A is aryl or heteroaryl (e.g., a monocyclic 6-membered aryl or heteroaryl), optionally substituted by one or more R³; each of R¹ and R² is independently hydrogen or alkyl, wherein alkyl is optionally substituted by one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In some embodiments, A is aryl (e.g., phenyl). In some embodiments, A is heteroaryl. In some embodiments, A is a 6-membered heteroaryl. In some embodiments, A is a nitrogen-containing heteroaryl (e.g., pyridyl).

In some embodiments, A is substituted by one or more R³. In some embodiments, A is a 6-membered aryl or heteroaryl and is substituted by R³ in the para position. In some embodiments, A is a 6-membered aryl or heteroaryl and is substituted by R³ in the meta position. In some embodiments, A is a 6-membered aryl or heteroaryl and is substituted by R³ in the ortho position.

In some embodiments, each R³ is independently alkyl, halo, cyano, carbocyclyl, heterocyclyl, or —OR^{c}. In some embodiments, R³ is alkyl (e.g., C₁-C₄ alkyl). In some embodiments, R³ is unsubstituted alkyl (e.g., unsubstituted C₁-C₄ alkyl) or substituted alkyl (e.g., substituted C₁-C₄ alkyl). In some embodiments, R³ is methyl. In some embodiments, R³ is substituted C₁ alkyl (e.g., wherein the C₁ alkyl is substituted with alkyl (e.g., methyl), halo (e.g., fluoro), cyano, carbocyclyl, or —OR^{c} (e.g., —OCH₃)). In some embodiments, R³ is —CF₃.

In some embodiments, R³ is halo (e.g., fluoro).

In some embodiments, R³ is —OR^{c}. In some embodiments, R³ is —OR^{c}, wherein R^{c} is alkyl (e.g., substituted alkyl or unsubstituted alkyl). In some embodiments, R³ is —OCF₃, —OCH₃, —OCH(CH₃)(CF₃), or —OCH₂CF₃.

In some embodiments, R¹ is hydrogen. In some embodiments, R¹ is alkyl. In some embodiments, R¹ is C₁₋₆ alkyl. In some embodiments, R¹ is unsubstituted C₁₋₆ alkyl (e.g., —CH₃). In some embodiments, R¹ is substituted alkyl (e.g., substituted C₁₋₆ alkyl, e.g., CF₃).

In some embodiments, R² is hydrogen. In some embodiments, R² is alkyl. In some embodiments, R² is C₁₋₆ alkyl. In some embodiments, R² is unsubstituted C₁₋₆ alkyl (e.g., —CH₃). In some embodiments, R² is substituted alkyl (e.g., substituted C₁₋₆ alkyl, e.g., CF₃).

In some embodiments, each of R¹ and R² is independently hydrogen. In some embodiments, each of R¹ and R² is independently alkyl. In some embodiments, R¹ is hydrogen and R² is alkyl. In some embodiments, R¹ is alkyl and R² is hydrogen.

In some embodiments, the compound of Formula (I-d) is not:

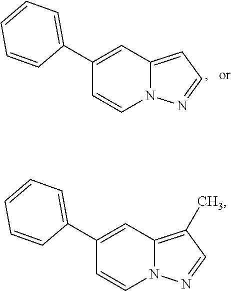 [SMILES: c1ccc(-c2ccn3nccc3c2)cc1 ; Cc1cnn2ccc(-c3ccccc3)cc12]

or a pharmaceutically acceptable salt thereof.

In another aspect, the present invention features a compound of Formula (II):

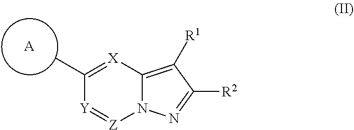

or a pharmaceutically acceptable salt thereof, wherein each of X, Y, and Z is independently N or CR′; A is aryl or heteroaryl (e.g., a monocyclic 6-membered aryl or heteroaryl), wherein aryl and heteroaryl are optionally substituted with one or more R³; R′ is hydrogen, alkyl, or —OR^{c}; each of R¹ and R² is independently hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In some embodiments, X is CR′. In some embodiments, Y is CR′. In some embodiments, Z is CR′. In some embodiments, each of X, Y, and Z is independently CR′. In some embodiments, R′ is hydrogen.

In some embodiments, one of X, Y, and Z is independently N. In some embodiments, X is N. In some embodiments, X is N and Y is CR′. In some embodiments, X is N and Z is CR′. In some embodiments, X is N and each of Y and Z is independently CR′. In some embodiments, R′ is hydrogen.

In some embodiments, A is aryl. In some embodiments, A is 6-membered aryl (e.g., phenyl). In some embodiments, A is phenyl is substituted by 1-3 R³. In some embodiments, A is phenyl is substituted by 1 R³ in the para position.

In some embodiments, A is heteroaryl. In some embodiments, A is a 6-membered heteroaryl. In some embodiments, A is a nitrogen-containing heteroaryl (e.g., pyridyl). In some embodiments, A is pyridyl substituted by 1-3 R³. In some embodiments, A is pyridyl substituted by 1 R³ in the para position.

In some embodiments, R¹ is hydrogen.

In some embodiments, R¹ is alkyl. In some embodiments, R¹ is C₁₋₆ alkyl. In some embodiments, R¹ is alkyl (e.g., C₁₋₆ alkyl) substituted with 1-4 R⁴. In some embodiments, R⁴ is halo (e.g., fluoro). In some embodiments, R is —CF₃.

In some embodiments, R² is hydrogen.

In some embodiments, each R³ is independently alkyl, carbocyclyl or —OR^{c}. In some embodiments, R³ is alkyl (e.g., C₁-C₄ alkyl). In some embodiments, R³ is C₁-C₄ alkyl. In some embodiments, R³ is isopropyl. In some embodiments, R³ is substituted C₁-C₄ alkyl (e.g., wherein the C₁-C₄ alkyl is substituted with one or more R⁵, e.g., halo (e.g., fluoro), cyano, carbocyclyl, or —OR′ (e.g., —OCH₃)). In some embodiments, R³ is —C(CH₃)₂OCH₃.

In some embodiments, R³ is carbocyclyl. In some embodiments, R³ is unsubstituted carbocyclyl (e.g., unsubstituted cyclopropyl) or substituted carbocyclyl (e.g., substituted with cyano).

In some embodiments, R³ is —OR^{c}. In some embodiments, R³ is —OR^{c}, wherein R^{c} is alkyl, e.g., substituted by one or more R⁶. In some embodiments, R⁶ is alkyl or halo. In some embodiments, R³ is —OCF₃, —OCH₃, or —OCH₂CF₃.

In one aspect, the present invention provides a compound of Formula (IIa):

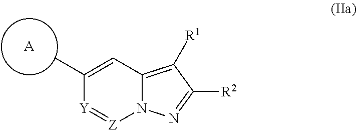

or a pharmaceutically acceptable salt thereof, wherein:

each of Y and Z is independently N or CR′, wherein at least one of Y and Z is N;

A is aryl or heteroaryl (e.g., a monocyclic 6-membered aryl or heteroaryl), wherein aryl and heteroaryl are substituted with one or more R³;

R′ is hydrogen, alkyl, or —OR^{c};

each of R¹ and R² is hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴;

each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵;

each R⁴ and R⁵ is independently alkyl, halo, cyano, nitro, or —OR^{c};

each R^{c} is independently hydrogen, alkyl, aryl, cycloalkyl, heterocyclyl, or heteroaryl, wherein alkyl, aryl, cycloalkyl, heterocyclyl, or heteroaryl is optionally substituted by one or more R⁶;

each R^{d} is independently hydrogen or alkyl; and

each R⁶ is independently alkyl, haloalkyl, carbocyclyl, heterocyclyl, halo, cyano, nitro, or —OH.

In some embodiments, Y is N and Z is CR′ (e.g., CH).

In other embodiments, Z is N and Y is CR′ (e.g., CH).

In other embodiments, A is aryl (e.g., phenyl) substituted by 1-3 R³.

In certain embodiments, A is heteroaryl (e.g., pyridyl) substituted by 1-3 R³.

In some embodiments, each R³ is independently alkyl, halo, cyano, carbocyclyl, or —OR^{c}.

In certain embodiments, at least one R³ is alkyl or —OR^{c}.

In other embodiments, R¹ is hydrogen or alkyl (e.g., substituted with one or more R⁴) and R² is hydrogen.

In some embodiments, R¹ is —CF₃.

In another aspect, the present invention provides a compound of Formula (IIb):

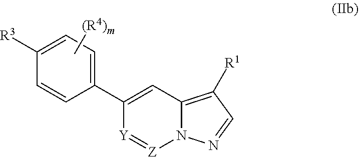

or a pharmaceutically acceptable salt thereof, wherein:

each of Y and Z is independently N or CH, wherein one of Y and Z is N and the other is CH;

R¹ is hydrogen or C₁₋₆alkyl, —C(O)N(R^{d})₂, —C(O)R^{c}, wherein C₁₋₆ alkyl is optionally substituted with one or more halo, cyano, nitro, C₃₋₁₀ carbocyclyl, C₃₋₁₀ heterocyclyl, —OR^{c}, —N(R^{d})₂, —C(O)R^{c}, —C(O)OR^{c}, or —C(O)N(R^{d})₂, wherein C₃₋₁₀ carbocyclyl and C₃₋₁₀ heterocyclyl are each optionally substituted with one or more R⁵;

each R³ is independently C₁₋₆alkyl, halo, cyano, nitro, C₃₋₁₀ carbocyclyl, C₃₋₁₀ heterocyclyl, —OR^{c}, —N(R^{d})₂, —C(O)R^{c}, —C(O)OR^{c}, or —C(O)N(R^{d})₂, wherein C₁₋₆ alkyl, C₃₋₁₀ carbocyclyl, C₃₋₁₀ heterocyclyl are optionally substituted with one or more R⁵;

R⁴ is C₁₋₆alkyl, halo, or OR^{c}, wherein C₁₋₆ alkyl is optionally substituted with one or more R⁵;

m is 0, 1, or 2;

R⁵ is independently C₁₋₆ alkyl, halo, cyano, nitro, or —OR^{c};

each R^{c} is independently hydrogen, C₁₋₆ alkyl, phenyl, C₃₋₈carbocyclyl, 5-8 membered heterocyclyl or 5-8 membered heteroaryl, wherein C₁₋₆ alkyl, phenyl, C₃₋₈carbocyclyl, 5-8 membered heterocyclyl, or 5-8 membered heteroaryl is optionally substituted by one or more R⁶;

each R^{d} is independently hydrogen or C₁₋₆ alkyl; and

each R⁶ is independently C₁₋₆ alkyl, C₁₋₆haloalkyl, C₃₋₁₀ carbocyclyl, C₃₋₁₀ heterocyclyl, halo, cyano, nitro, or —OH.

In some embodiments, the compound is a compound of formula IIb-1:

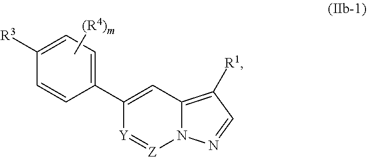

or a pharmaceutically acceptable salt thereof, wherein the variables are as defined herein.

In certain embodiments, Y is N and Z is CH.

In other embodiments, Y is CH and Z is N.

In some embodiments, R¹ is selected from the group consisting of hydrogen or C₁₋₆alkyl, and —C(O)N(R^{d})₂, wherein C₁₋₆alkyl is optionally substituted with one or more halogen.

In certain embodiments, R¹ is selected from the group consisting of hydrogen, —CF₃, —CF₂—CF₃, or —C(O)N(CH₃)₂.

In other embodiments, R³ is independently selected from C₁₋₆alkyl or —OR^{c}, wherein R^{c} is C₁₋₆alkyl optionally substituted with one or more halogen or C₃₋₈carbocyclyl optionally substituted with C₁₋₆haloalkyl or cyano.

In some embodiments, R³ is independently selected from the group consisting of methyl, —OCF₃ and O—CH₂—CF₃.

In other embodiments, m is 0.

In some embodiments, R⁴ is selected from the group consisting of methyl, F, —OMe, and —CH₂—OMe.

In certain embodiments, the compound is selected from the group consisting of:

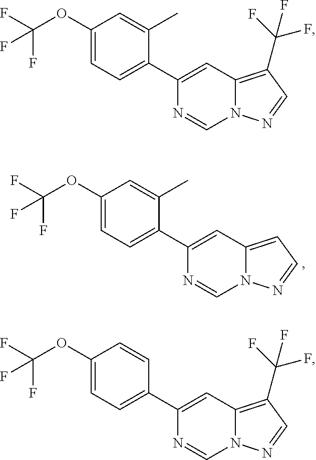

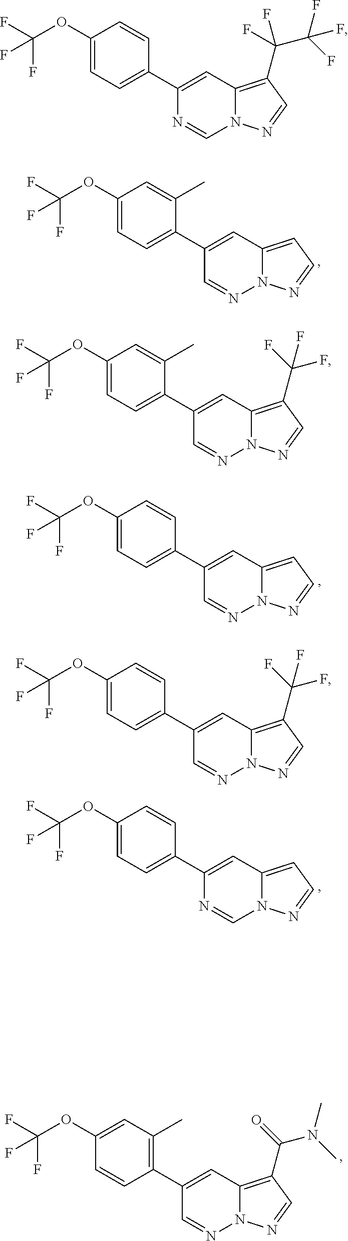

and a pharmaceutically acceptable salt thereof.

In another aspect, the present disclosure provides a compound of Formula (IIc):

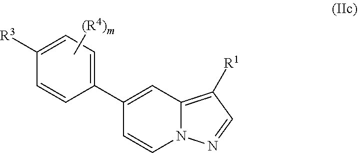

or a pharmaceutically acceptable salt thereof, wherein:

R¹ is hydrogen or C₁₋₆alkyl, —C(O)N(R^{d})₂, —C(O)R^{c}, wherein C₁₋₆ alkyl is optionally substituted with one or more halo, cyano, nitro, C₃₋₁₀ carbocyclyl, C₃₋₁₀ heterocyclyl, —OR^{c}, —N(R^{d})₂, —C(O)R^{c}, —C(O)OR^{c}, or —C(O)N(R^{d})₂, wherein C₃₋₁₀ carbocyclyl and C₃₋₁₀ heterocyclyl are each optionally substituted with one or more R⁵;

each R³ is independently C₁₋₆alkyl, halo, cyano, nitro, C₃₋₁₀ carbocyclyl, C₃₋₁₀ heterocyclyl, —OR^{c}, —N(R^{d})₂, —C(O)R^{c}, —C(O)OR^{c}, or —C(O)N(R^{d})₂, wherein C₁₋₆ alkyl, C₃₋₁₀ carbocyclyl, C₃₋₁₀ heterocyclyl are optionally substituted with one or more R⁵;

R⁴ is C₁₋₆alkyl, halo, or OR^{c}, wherein C₁₋₆ alkyl is optionally substituted with one or more R⁵;

m is 0, 1, or 2;

R⁵ is independently C₁₋₆ alkyl, halo, cyano, nitro, or —OR^{c};

each R^{c} is independently hydrogen, C₁₋₆ alkyl, phenyl, C₃₋₈carbocyclyl, 5-8 membered heterocyclyl, or 5-8 membered heteroaryl, wherein C₁₋₆ alkyl, phenyl, C₃₋₈carbocyclyl, 5-8 membered heterocyclyl, or 5-8 membered heteroaryl is optionally substituted by one or more R⁶;

each R^{d} is independently hydrogen or C₁₋₆ alkyl; and

each R⁶ is independently C₁₋₆ alkyl, C₁₋₆haloalkyl, C₃₋₁₀ carbocyclyl, C₃₋₁₀ heterocyclyl, halo, cyano, nitro, or —OH, wherein the compound is not:

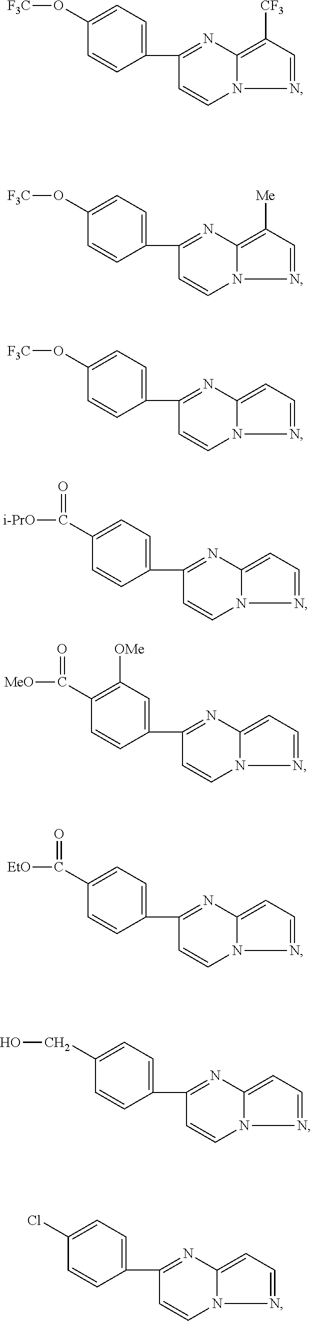

or a pharmaceutically acceptable salt thereof.

In some embodiments, R¹ is selected from the group consisting of hydrogen or C₁₋₆alkyl, and —C(O)N(R^{d})₂, wherein C₁₋₆alkyl is optionally substituted with one or more halogen.

In other embodiments, R¹ is selected from the group consisting of hydrogen, —CF₃, —CF₂—CF₃, or —C(O)N(CH₃)₂.

In certain embodiments, R³ is independently selected from C₁₋₆alkyl or —OR^{c}, wherein R^{c} is C₁₋₆alkyl optionally substituted with one or more halogen or C₃₋₈carbocyclyl optionally substituted with C₁₋₆haloalkyl or cyano.

In some embodiments, R³ is independently selected from the group consisting of methyl, —OCF₃ and O—CH₂—CF₃.

In certain embodiments, m is 0.

In other embodiments, R⁴ is selected from the group consisting of methyl, F, —OMe, and —CH₂—OMe.

In some embodiments, the compound is selected from the group consisting of:

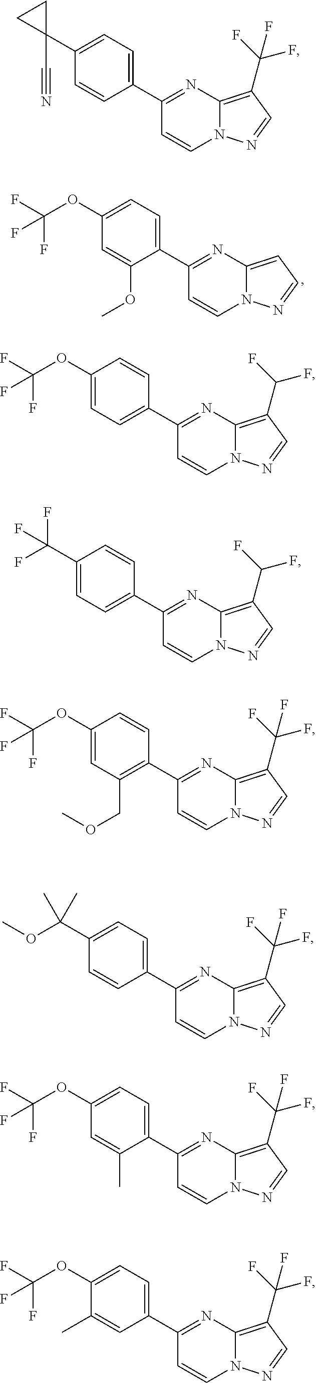

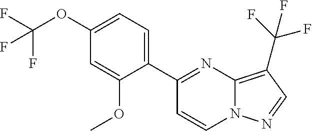 [SMILES: COc1cc(OC(F)(F)F)ccc1-c1ccn2ncc(C(F)(F)F)c2n1]

and a pharmaceutically acceptable salt thereof.

In another aspect, the present disclosure provides a compound of Formula (IId):

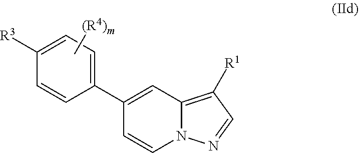

or a pharmaceutically acceptable salt thereof, wherein:

R¹ is hydrogen or C₁₋₆alkyl, —C(O)N(R^{d})₂, —C(O)R^{c}, wherein C₁₋₆ alkyl is optionally substituted with one or more halo, cyano, nitro, C₃₋₁₀ carbocyclyl, C₃₋₁₀ heterocyclyl, —OR^{c}, —N(R^{d})₂, —C(O)R^{c}, —C(O)OR^{c}, or —C(O)N(R^{d})₂, wherein C₃₋₁₀ carbocyclyl and C₃₋₁₀ heterocyclyl are each optionally substituted with one or more R⁵;

each R³ is independently C₁₋₆alkyl, halo, cyano, nitro, C₃₋₁₀ carbocyclyl, C₃₋₁₀ heterocyclyl, —OR^{c}, —N(R^{d})₂, —C(O)R^{c}, —C(O)OR^{c}, or —C(O)N(R^{d})₂, wherein C₁₋₆ alkyl, C₃₋₁₀ carbocyclyl, C₃₋₁₀ heterocyclyl are optionally substituted with one or more R⁵;

R⁴ is C₁₋₆alkyl, halo, or OR^{c}, wherein C₁₋₆ alkyl is optionally substituted with one or more R⁵;

m is 0, 1, or 2;

R⁵ is independently C₁₋₆ alkyl, halo, cyano, nitro, or —OR^{c};

each R^{c} is independently hydrogen, C₁₋₆ alkyl, phenyl, C₃₋₈carbocyclyl, 5-8 membered heterocyclyl, or 5-8 membered heteroaryl, wherein C₁₋₆ alkyl, phenyl, C₃₋₈carbocyclyl, 5-8 membered heterocyclyl, or 5-8 membered heteroaryl is optionally substituted by one or more R⁶;

each R^{d} is independently hydrogen or C₁₋₆ alkyl; and

each R⁶ is independently C₁₋₆ alkyl, C₁₋₆haloalkyl, C₃₋₁₀ carbocyclyl, C₃₋₁₀ heterocyclyl, halo, cyano, nitro, or —OH, wherein the compound is not:

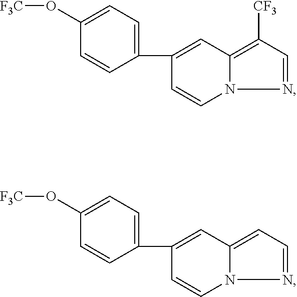 [SMILES: FC(F)(F)Oc1ccc(-c2ccn3ncc(C(F)(F)F)c3c2)cc1 ; FC(F)(F)Oc1ccc(-c2ccn3nccc3c2)cc1]

or a pharmaceutically acceptable salt thereof.

In some embodiments, R¹ is selected from the group consisting of hydrogen or C₁₋₆alkyl, and —C(O)N(R^{d})₂, wherein C₁₋₆alkyl is optionally substituted with one or more halogen.

In other embodiments, R¹ is selected from the group consisting of hydrogen, —CF₃, —CF₂—CF₃, or —C(O)N(CH₃)₂.

In certain embodiments, R³ is independently selected from C₁₋₆alkyl or —OR^{c}, wherein R^{c} is C₁₋₆alkyl optionally substituted with one or more halogen or C₃₋₈carbocyclyl optionally substituted with C₁₋₆haloalkyl or cyano.

In some embodiments, R³ is independently selected from the group consisting of methyl, —OCF₃ and O—CH₂—CF₃.

In other embodiments, m is 0.

In certain embodiments, R⁴ is selected from the group consisting of methyl, F, —OMe, and —CH₂—OMe.

In some embodiments, the compound is selected from the group consisting of:

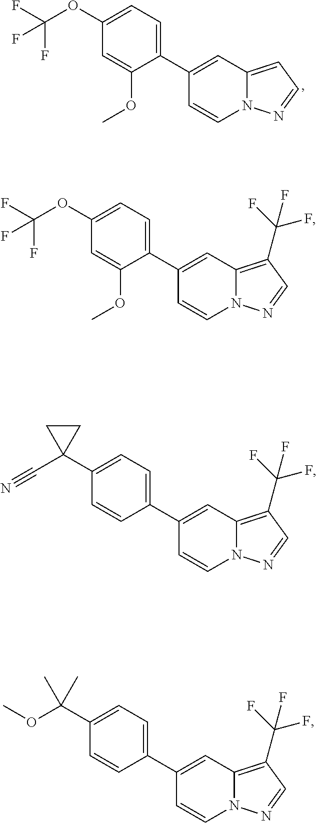 [SMILES: COc1cc(OC(F)(F)F)ccc1-c1ccn2nccc2c1 ; COc1cc(OC(F)(F)F)ccc1-c1ccn2ncc(C(F)(F)F)c2c1 ; N#CC1(c2ccc(-c3ccn4ncc(C(F)(F)F)c4c3)cc2)CC1 ; COC(C)(C)c1ccc(-c2ccn3ncc(C(F)(F)F)c3c2)cc1]

and a pharmaceutically acceptable salt thereof.

In another aspect, the present invention provides a compound of formula (IIe):

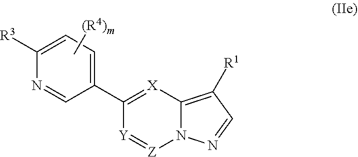

or a pharmaceutically acceptable salt thereof, wherein:

each of X, Y, and Z is independently N or CH;

R¹ is selected from the group consisting of: C₁₋₆ alkyl, C₁₋₆haloalkyl, and C₃₋₈carbocyclyl;

R³ is selected from the group consisting of: C₁₋₆alkyl, cyano, C₃₋₁₀ carbocyclyl, —OR^{c}, —C(O)R^{c}, —C(O)OR^{c}, or —C(O)N(R^{d})₂, wherein C₁₋₆ alkyl or C₃₋₁₀ carbocyclyl is optionally substituted with one or more R⁵;

R⁴ is C₁₋₆alkyl, halo, or OR^{c}, wherein C₁₋₆ alkyl is optionally substituted with one or more R⁵;

m is 0, 1, or 2;

R⁵ is independently C₁₋₆ alkyl, halo, cyano, nitro, or —OR^{c};

each R^{c} is independently hydrogen, C₁₋₆ alkyl, phenyl, C₃₋₈carbocyclyl, 5-8 membered heterocyclyl, or 5-8 membered heteroaryl, wherein C₁₋₆ alkyl, phenyl, C₃₋₈carbocyclyl, 5-8 membered heterocyclyl, or 5-8 membered heteroaryl is optionally substituted by one or more R⁶;

each R^{d} is independently hydrogen or C₁₋₆ alkyl; and

each R⁶ is independently C₁₋₆ alkyl, C₁₋₆haloalkyl, C₃₋₁₀ carbocyclyl, C₃₋₁₀ heterocyclyl, halo, cyano, nitro, or —OH.

In some embodiments, the compound is a compound of formula (IIe-1):

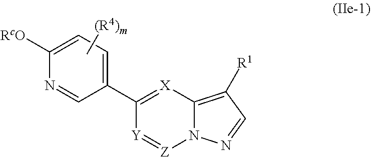

or a pharmaceutically acceptable salt thereof, wherein the variables are as defined herein.

In some embodiments, X is N, Y and Z are CH.

In certain embodiments, Y is N, Y and Z are CH.

In other embodiments, Z is N, X and Y are CH.

In some embodiments, X, Y, and Z are CH.

In other embodiments, R¹ is selected from the group consisting of: —CH₃, CF₃, CHF₂, and cyclopropyl.

In certain embodiments, R³ is —OR^{c}, wherein R^{c} is selected from the group consisting of C₁₋₆alkyl substituted with 1, 2, or 3 halogens or C₃₋₈carbocyclyl optionally substituted with cyano or CF₃.

In other embodiments, R^{c} is selected from the group consisting of: —CF₃, —CH₂CF₃, or

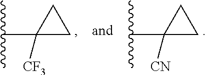 [SMILES: *C1(C(F)(F)F)CC1 ; *C1(C#N)CC1]

In some embodiments, R^{c} is —CH₂CF₃.

In certain embodiments, m is 0.

In other embodiments, R⁴ is selected from the group consisting of methyl, F, —OMe, and —CH₂—OMe.

In some embodiments, the compound is selected from the group consisting of:

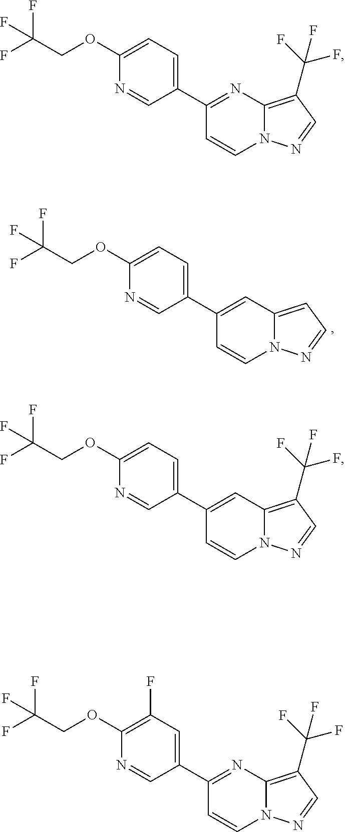 [SMILES: FC(F)(F)COc1ccc(-c2ccn3ncc(C(F)(F)F)c3n2)cn1 ; FC(F)(F)COc1ccc(-c2ccn3nccc3c2)cn1 ; FC(F)(F)COc1ccc(-c2ccn3ncc(C(F)(F)F)c3c2)cn1 ; Fc1cc(-c2ccn3ncc(C(F)(F)F)c3n2)cnc1OCC(F)(F)F]

and a pharmaceutically acceptable salt thereof.

In another aspect, the present invention provides a compound of formula IId:

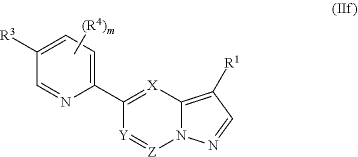

or a pharmaceutically acceptable salt thereof, wherein:

each of X, Y, and Z is independently N or CH;

R¹ is selected from the group consisting of: C₁₋₆ alkyl, C₁₋₆ haloalkyl, and C₃₋₈ carbocyclyl;

R³ is selected from the group consisting of: C₁₋₆alkyl, cyano, C₃₋₁₀ carbocyclyl, —OR^{c}, —C(O)R^{c}, —C(O)OR^{c}, and —C(O)N(R^{d})₂, wherein C₁₋₆ alkyl or C₃₋₁₀ carbocyclyl is optionally substituted with one or more R⁵;

R⁴ is C₁₋₆alkyl, halo, or OR^{c}, wherein C₁₋₆ alkyl is optionally substituted with one or more R⁵;

m is 0, 1, or 2;

R⁵ is independently C₁₋₆ alkyl, halo, cyano, nitro, or —OR^{c}—;

each R^{c} is independently hydrogen, C₁₋₆ alkyl, phenyl, C₃₋₈carbocyclyl, 5-8 membered heterocyclyl, or 5-8 membered heteroaryl, wherein C₁₋₆ alkyl, phenyl, C₃₋₈carbocyclyl, 5-8 membered heterocyclyl, or 5-8 membered heteroaryl is optionally substituted by one or more R⁶;

each R^{d} is independently hydrogen or C₁₋₆ alkyl; and

each R⁶ is independently C₁₋₆ alkyl, C₁₋₆haloalkyl, C₃₋₁₀ carbocyclyl, C₃₋₁₀ heterocyclyl, halo, cyano, nitro, or —OH.

In some embodiments, the compound is a compound of formula IId-1:

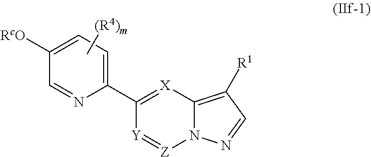

or a pharmaceutically acceptable salt thereof, wherein the variables are as defined herein.

In some embodiments, X is N, Y and Z are CH.

In certain embodiments, Y is N, Y and Z are CH.

In other embodiments, Z is N, X and Y are CH.

In some embodiments, X, Y, and Z are CH.

In certain embodiments, R¹ is selected from the group consisting of: —CH₃, CF₃, CHF₂, and cyclopropyl.

In other embodiments, R³ is —OR^{c}, wherein R^{c} is selected from the group consisting of C₁₋₆alkyl substituted with 1, 2, or 3 halogens or C₃₋₈carbocyclyl optionally substituted with cyano or CF₃.

In some embodiments, R³ is selected from the group consisting of: —CF₃, —CH₂CF₃,

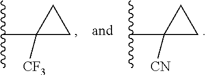 [SMILES: *C1(C(F)(F)F)CC1 ; *C1(C#N)CC1]

In other embodiments, m is 0.

In certain embodiments, R⁴ is selected from the group consisting of methyl, F, —OMe, and —CH₂—OMe.

In another aspect, the present invention features compound of Formula (III):

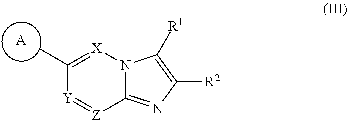

or a pharmaceutically acceptable salt thereof, wherein each of X, Y, and Z is independently N or CR′; A is aryl or heteroaryl (e.g., a monocyclic 6-membered aryl or heteroaryl), wherein aryl and heteroaryl are optionally substituted with one or more R³; R′ is hydrogen, alkyl, or —OR^{c}; each of R¹ and R² is independently hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In some embodiments, X is CR′. In some embodiments, Y is CR′. In some embodiments, Z is CR′. In some embodiments, each of X, Y, and Z is independently CR′. In some embodiments, R′ is hydrogen.

In some embodiments, one of X, Y, and Z is independently N. In some embodiments, X is N. In some embodiments, X is N and Y is CR. In some embodiments, X is N and Z is CR′. In some embodiments, X is N and each of Y and Z is independently CR′. In some embodiments, R′ is hydrogen.

In some embodiments, Y is N. In some embodiments, Y is N and X is CR. In some embodiments, Y is N and Z is CR′. In some embodiments, Y is N and each of X and Z is independently CR′. In some embodiments, R′ is hydrogen.

In some embodiments, Z is N. In some embodiments, Z is N and X is CR. In some embodiments, Z is N and Y is CR′. In some embodiments, Z is N and each of X and Y is independently CR′. In some embodiments, R′ is hydrogen.

In some embodiments, A is aryl. In some embodiments, A is 6-membered aryl (e.g., phenyl). In some embodiments, A is phenyl is substituted by 1-3 R³. In some embodiments, A is phenyl is substituted by 1 R³ in the para position.

In some embodiments, A is heteroaryl. In some embodiments, A is a 6-membered heteroaryl. In some embodiments, A is a nitrogen-containing heteroaryl (e.g., pyridyl). In some embodiments, A is pyridyl substituted by 1-3 R³. In some embodiments, A is pyridyl substituted by 1 R³ in the para position.

In some embodiments, R is hydrogen.

In some embodiments, R¹ is alkyl. In some embodiments, R¹ is C₁₋₆ alkyl. In some embodiments, R¹ is alkyl (e.g., C₁₋₆ alkyl) substituted with 1-4 R⁴. In some embodiments, R⁴ is halo (e.g., fluoro). In some embodiments, R is —CF₃.

In some embodiments, R² is hydrogen.

In some embodiments, each R³ is independently alkyl, carbocyclyl or —OR^{c}. In some embodiments, R³ is alkyl (e.g., C₁-C₄ alkyl). In some embodiments, R³ is C₁-C₄ alkyl. In some embodiments, R³ is isopropyl. In some embodiments, R³ is substituted C₁-C₄ alkyl (e.g., wherein the C₁-C₄ alkyl is substituted with one or more R⁵, e.g., halo (e.g., fluoro), cyano, carbocyclyl, or —OR′ (e.g., —OCH₃)). In some embodiments, R³ is —C(CH₃)₂OCH₃.

In some embodiments, R³ is carbocyclyl. In some embodiments, R³ is unsubstituted carbocyclyl (e.g., unsubstituted cyclopropyl) or substituted carbocyclyl (e.g., substituted with cyano).

In some embodiments, R³ is —OR^{c}. In some embodiments, R³ is —OR^{c}, wherein R^{c} is alkyl, e.g., substituted by one or more R⁶. In some embodiments, R⁶ is alkyl or halo. In some embodiments, R³ is —OCF₃, —OCH₃, or —OCH₂CF₃.

In another aspect, the present invention provides a compound of Formula (IIIa):

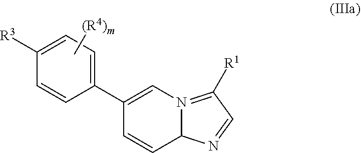

or a pharmaceutically acceptable salt thereof, wherein:

R¹ is hydrogen or C₁₋₆ haloalkyl;

R³ is —(C₁₋₆alkylene)-O—(C₁₋₆alkyl) or C₃₋₈ cycloalkyl optionally substituted with —CF₃ or —CN;

R⁴ is selected from the group consisting of methyl, F, —OMe, and —CH₂—OMe; and

m is 0, 1, or 2.

In some embodiments, R¹ is hydrogen or CF₃.

In other embodiments, R³ is —(C₁₋₆alkylene)-O—(C₁₋₆alkyl).

In certain embodiments, R³ is

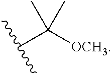 [SMILES: *C(C)(C)OC]

In some embodiments, R³ is C₃₋₈ cycloalkyl optionally substituted with —CF₃ or —CN.

In certain embodiments, R³ is

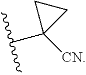 [SMILES: *C1(C#N)CC1]

In other embodiments, m is 0.

In some embodiments, the compound is selected from the group consisting of:

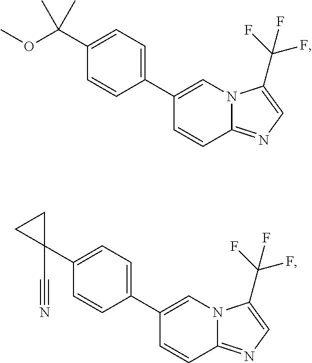 [SMILES: COC(C)(C)c1ccc(-c2ccc3ncc(C(F)(F)F)n3c2)cc1 ; N#CC1(c2ccc(-c3ccc4ncc(C(F)(F)F)n4c3)cc2)CC1]

and a pharmaceutically acceptable salt thereof.

In another aspect, the present invention provides a compound represented by:

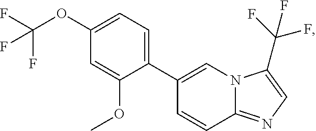 [SMILES: COc1cc(OC(F)(F)F)ccc1-c1ccc2ncc(C(F)(F)F)n2c1]

or a pharmaceutically acceptable salt thereof.

In another aspect, the present invention provides a compound of formula (IIIb):

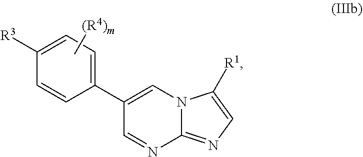

or a pharmaceutically acceptable alt thereof, wherein:

R is C₁₋₆haloalkyl,

R³ is selected from the group consisting of —O—C₁₋₄haloalkyl, —(C₁₋₆alkylene)-O—(C₁₋₆alkyl), and C₃₋₇ cycloalkyl optionally substituted with —CF₃ or —CN;

R⁴ is selected from the group consisting of methyl, F, —OMe, and —CH₂—OMe; and

m is 0, 1, or 2.

In some embodiments, R¹ is CF₃ or CHF₂.

In certain embodiments, R¹ is CF₃.

In other embodiments, R³ is —O—C₁₋₄haloalkyl.

In some embodiments, R³ is —O—CH₂—CF₃ or —O—CF₃.

In certain embodiments, R³ is —O—CF₃.

In other embodiments, R³ is —(C₁₋₆alkylene)-O—(C₁₋₆alkyl).

In some embodiments, R is

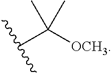 [SMILES: *C(C)(C)OC]

In certain embodiments, R³ is C₃₋₇ cycloalkyl optionally substituted with —CF₃ or —CN.

In other embodiments, R³ is

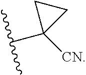 [SMILES: *C1(C#N)CC1]

In some embodiments, m is 0.

In certain embodiments, the compound is selected from the group consisting of:

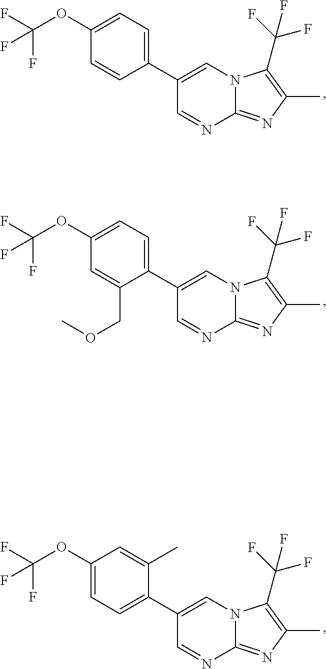 [SMILES: Cc1nc2ncc(-c3ccc(OC(F)(F)F)cc3)cn2c1C(F)(F)F ; COCc1cc(OC(F)(F)F)ccc1-c1cnc2nc(C)c(C(F)(F)F)n2c1 ; Cc1cc(OC(F)(F)F)ccc1-c1cnc2nc(C)c(C(F)(F)F)n2c1]

and a pharmaceutically acceptable salt thereof.

In another aspect, the present invention provides a compound selected from the group consisting of:

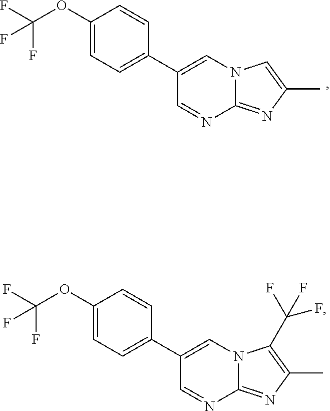 [SMILES: Cc1cn2cc(-c3ccc(OC(F)(F)F)cc3)cnc2n1 ; Cc1nc2ncc(-c3ccc(OC(F)(F)F)cc3)cn2c1C(F)(F)F]

and a pharmaceutically acceptable salt thereof.

In another aspect, the present invention provides a compound of formula (IIIc):

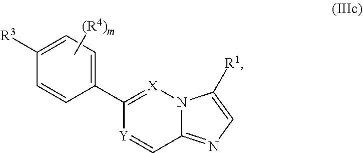

or a pharmaceutically acceptable salt thereof, wherein:

X is N and Y is CH, or X is CH and Y is N;

R¹ is hydrogen or C₁₋₄haloalkyl;

R³ is selected from the group consisting of —O—C₁₋₆haloalkyl, —(C₁₋₆alkylene)-O—(C₁₋₆alkyl), and C₃₋₇ cycloalkyl optionally substituted with —CF₃ or —CN;

R⁴ is selected from the group consisting of methyl, F, —OMe, and —CH₂—OMe; and

m is 0, 1, or 2.

In some embodiments, X is N and Y is CH.

In certain embodiments, X is CH and Y is N.

In other embodiments, R¹ is hydrogen.

In some embodiments, R¹ is CF₃ or CHF₂.

In other embodiments, R¹ is CF₃.

In some embodiments, R³ is —O—C₁₋₄haloalkyl.

In other embodiments, R³ is —CH₂—CF³ or —O—CF₃.

In certain embodiments, R³ is —O—CF₃.

In some embodiments, R³ is —(C₁₋₆alkylene)-O—(C₁₋₆alkyl).

In certain embodiments, R³ is

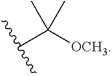 [SMILES: *C(C)(C)OC]

In other embodiments, R³ is C₃₋₇ cycloalkyl optionally substituted with —CF₃ or —CN.

In some embodiments, R³ is C₃₋₇ cycloalkyl substituted with —CN.

In other embodiments, R³ is

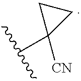 [SMILES: *C1(C#N)CC1]

In certain embodiments, m is 0.

In some embodiments, the compound is selected from the group consisting of:

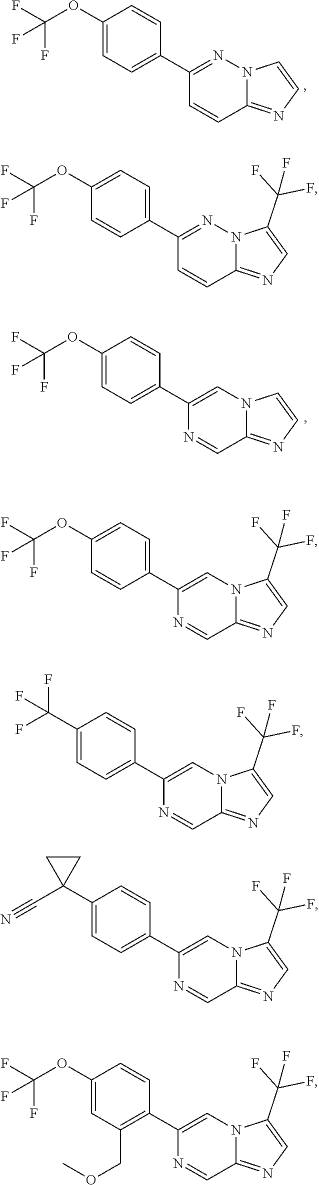

and a pharmaceutically acceptable salt thereof.

In another aspect, the present invention provides a compound of formula (IIId):

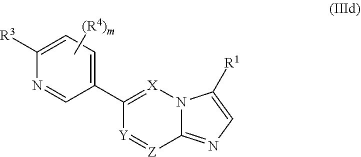

or a pharmaceutically acceptable salt thereof, wherein:

each of X, Y, and Z is independently N or CH;

R¹ is selected from the group consisting of: unsubstituted C₁₋₆ alkyl, C₁₋₆haloalkyl, and unsubstituted C₃₋₈cycloalkyl;

R³ is selected from the group consisting of: C₁₋₆alkyl, cyano, C₃₋₁₀ carbocyclyl, —OR^{c}, —C(O)R^{c}, —C(O)OR^{c}, and —C(O)N(R^{d})₂, wherein C₁₋₆ alkyl or C₃₋₁₀ carbocyclyl is optionally substituted with one or more R⁵;

R⁴ is C₁₋₆alkyl, halo, or OR^{c}, wherein C₁₋₆ alkyl is optionally substituted with one or more R⁵;

m is 0, 1, or 2;

R⁵ is independently C₁₋₆ alkyl, halo, cyano, nitro, or —OR^{c};

each R^{c} is independently hydrogen, C₁₋₆ alkyl, phenyl, C₃₋₈carbocyclyl, 5-8 membered heterocyclyl, or 5-8 membered heteroaryl, wherein C₁₋₆ alkyl, phenyl, C₃₋₈carbocyclyl, 5-8 membered heterocyclyl, or 5-8 membered heteroaryl is optionally substituted by one or more R⁶;

each R^{d} is independently hydrogen or C₁₋₆ alkyl; and

each R⁶ is independently C₁₋₆ alkyl, C₁₋₆haloalkyl, C₃₋₁₀ carbocyclyl, C₃₋₁₀ heterocyclyl, halo, cyano, nitro, or —OH.

In some embodiments, the compound is a compound of formula (IIId-1):

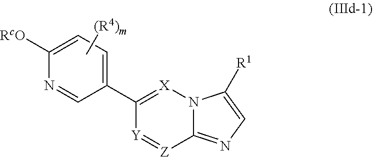

or a pharmaceutically acceptable salt thereof, wherein the variables are as defined herein.

In certain embodiments, X is N, Y and Z are CH.

In other embodiments, Y is N, Y and Z are CH.

In certain embodiments, Z is N, X and Y are CH.

In some embodiments, X, Y, and Z are CH.

In certain embodiments, R¹ is selected from the group consisting of: —CH₃, CF₃, CHF₂, and cyclopropyl.

In other embodiments, R³ is —OR^{c}, wherein R³ is selected from the group consisting of C₁₋₆alkyl substituted with 1, 2, or 3 halogens or C₃₋₈carbocyclyl optionally substituted with cyano or CF₃.

In some embodiments, R³ is selected from the group consisting of: —CF₃, CF₃,

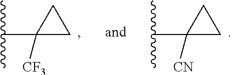 [SMILES: *C1(C(F)(F)F)CC1 ; *C1(C#N)CC1]

In certain embodiments, m is 0.

In other embodiments, R⁴ is selected from the group consisting of methyl, F, —OMe, and —CH₂—OMe.

In some embodiments, the compound is selected from the group consisting of:

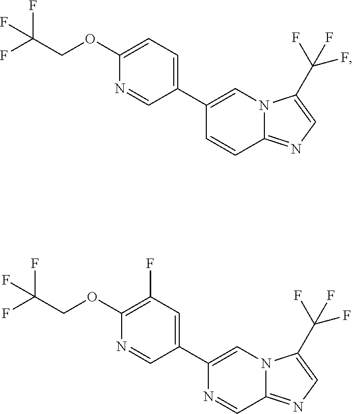 [SMILES: FC(F)(F)COc1ccc(-c2ccc3ncc(C(F)(F)F)n3c2)cn1 ; Fc1cc(-c2cn3c(C(F)(F)F)cnc3cn2)cnc1OCC(F)(F)F]

or a pharmaceutically acceptable salt thereof.

In another aspect, the present invention provides a compound of formula IIIe:

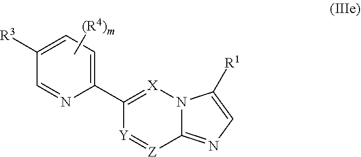

or a pharmaceutically acceptable salt thereof, wherein:

each of X, Y, and Z is independently N or CH;

R¹ is selected from the group consisting of: unsubstituted C₁₋₆ alkyl, C₁₋₆haloalkyl, and C₃₋₈carbocyclyl;

R³ is selected from the group consisting of: C₁₋₆alkyl, cyano, C₃₋₁₀ carbocyclyl, —OR^{c}, —C(O)R^{c}, —C(O)OR^{c}, or —C(O)N(R^{d})₂, wherein C₁₋₆ alkyl, C₃₋₁₀ carbocyclyl, C₃₋₁₀ heterocyclyl are optionally substituted with one or more R⁵;

R⁴ is C₁₋₆alkyl, halo, or OR^{c}, wherein C₁₋₆ alkyl is optionally substituted with one or more R⁵;

m is 0, 1, or 2;

R⁵ is independently C₁₋₆ alkyl, halo, cyano, nitro, or —OR^{c};

each R^{c} is independently hydrogen, C₁₋₆ alkyl, phenyl, C₃₋₈carbocyclyl, 5-8 membered heterocyclyl, or 5-8 membered heteroaryl, wherein C₁₋₆ alkyl, phenyl, C₃₋₈carbocyclyl, 5-8 membered heterocyclyl, or 5-8 membered heteroaryl is optionally substituted by one or more R⁶;

each R^{d} is independently hydrogen or C₁₋₆ alkyl; and

each R⁶ is independently C₁₋₆ alkyl, C₁₋₆haloalkyl, C₃₋₁₀ carbocyclyl, C₃₋₁₀ heterocyclyl, halo, cyano, nitro, or —OH.

In some embodiments, the compound is a compound of formula IIIe-1:

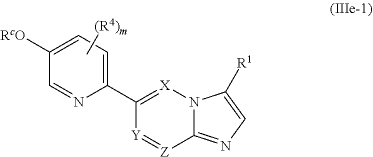

or a pharmaceutically acceptable salt thereof, wherein the variables are as defined herein.

In certain embodiments, X is N, Y and Z are CH.

In other embodiments, Y is N, Y and Z are CH.

In some embodiments, Z is N, X and Y are CH.

In certain embodiments, X, Y, and Z are CH.

In other embodiments, R¹ is selected from the group consisting of: —CH₃, CF₃, CHF₂, and cyclopropyl.

In some embodiments, R³ is —OR^{c}, wherein R³ is selected from the group consisting of C₁₋₆alkyl substituted with 1, 2, or 3 halogens or C₃₋₈carbocyclyl optionally substituted with cyano or CF₃.

In other embodiments, R³ is selected from the group consisting of: —CF₃, CF₃, and

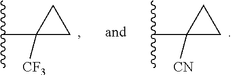 [SMILES: *C1(C(F)(F)F)CC1 ; *C1(C#N)CC1]

In some embodiments, m is 0.

In other embodiments, R⁴ is selected from the group consisting of methyl, F, —OMe, and —CH₂—OMe.

### Methods of Treatment

Described herein are compounds and compositions thereof and their use to treat a disease, disorder, or condition relating to aberrant function of a sodium channel ion channel, e.g., abnormal late or persistent sodium (INaL) current. In some embodiments, a compound provided by the present invention is effective in the treatment of epilepsy or an epilepsy syndrome, a neurodevelopmental disorder, pain, or a neuromuscular disorder. Compounds of the invention may also modulate all sodium ion channels, or may be specific to only one or a plurality of sodium ion channels, e.g., Nav 1.1, 1.2, 1.5, 1.6, 1.7, 1.8, or 1.9.

In typical embodiments, the present invention is intended to encompass the compounds disclosed herein, and the pharmaceutically acceptable salts, pharmaceutically acceptable esters, tautomeric forms, polymorphs, and prodrugs of such compounds. In some embodiments, the present invention includes a pharmaceutically acceptable addition salt, a pharmaceutically acceptable ester, a hydrate of an addition salt, a tautomeric form, a polymorph, an enantiomer, a mixture of enantiomers, a stereoisomer or mixture of stereoisomers (pure or as a racemic or non-racemic mixture) of a compound described herein, e.g. a compound of Formula (I), (I-2), (I-a), (I-b), (I-c), (I-d), (II), (II-2), (IIa), (IIb), (IIb-1), (IIc), (IIc-1), (IId), (IId-1), (III), (IIIa), (IIIb), (IIIc), (IIId), (IIId-1), (IIIe), or (IIIe-1).

### Epilepsy and Epilepsy Syndromes

The compounds described herein are useful in the treatment of epilepsy and epilepsy syndromes. Epilepsy is a CNS disorder in which nerve cell activity in the brain becomes disrupted, causing seizures or periods of unusual behavior, sensations and sometimes loss of consciousness. Seizure symptoms will vary widely, from a simple blank stare for a few seconds to repeated twitching of their arms or legs during a seizure.

Epilepsy may involve a generalized seizure or a partial or focal seizure. All areas of the brain are involved in a generalized seizure. A person experiencing a generalized seizure may cry out or make some sound, stiffen for several seconds to a minute a then have rhythmic movements of the arms and legs. The eyes are generally open, the person may appear not to be breathing and actually turn blue. The return to consciousness is gradual and the person may be confused from minutes to hours. There are six main types of generalized seizures: tonic-clonic, tonic, clonic, myoclonic, absence, and atonic seizures. In a partial or focal seizure, only part of the brain is involved, so only part of the body is affected. Depending on the part of the brain having abnormal electrical activity, symptoms may vary.

Epilepsy, as described herein, includes a generalized, partial, complex partial, tonic clonic, clonic, tonic, refractory seizures, status epilepticus, absence seizures, febrile seizures, or temporal lobe epilepsy.

The compounds described herein may also be useful in the treatment of epilepsy syndromes. Severe syndromes with diffuse brain dysfunction caused, at least partly, by some aspect of epilepsy, are also referred to as epileptic encephalopathies. These are associated with frequent seizures that are resistant to treatment and severe cognitive dysfunction, for instance West syndrome.

In some embodiments, the epilepsy syndrome comprises an epileptic encephalopathy, such as Dravet syndrome, Angelman syndrome, CDKL5 disorder, frontal lobe epilepsy, infantile spasms, West's syndrome, Juvenile Myoclonic Epilepsy, Landau-Kleffner syndrome, Lennox-Gastaut syndrome, Ohtahara syndrome, PCDH19 epilepsy, or Glut1 deficiency.

In some embodiments, the epilepsy or epilepsy syndrome is a genetic epilepsy or a genetic epilepsy syndrome. In some embodiments, epilepsy or an epilepsy syndrome comprises epileptic encephalopathy, epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, or KCNT1 epileptic encephalopathy.

In some embodiments, the methods described herein further comprise identifying a subject having epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, or KCNT1 epileptic encephalopathy) prior to administration of a compound described herein (e.g., a compound of Formulae (I), (I-2), (I-a), (I-b), (I-c), (I-d), (II), (II-2), (IIa), (IIb), (IIb-1), (IIc), (IId), (IIe), (IIe-1), (IIf), (IIf-1), (III), (IIIa), (IIIb), (IIIc), (IIId), (IIId-1), (IIIe), or (IIIe-1)).

In one aspect, the present invention features a method of treating epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, or KCNT1 epileptic encephalopathy) comprising administering to a subject in need thereof a compound of Formula (I):

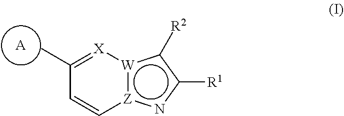

or a pharmaceutically acceptable salt thereof, wherein X is N or CR′; each of W and Z is independently N or C, wherein only one of W and Z is N; A is aryl or heteroaryl (e.g., a monocyclic 6-membered aryl or heteroaryl), optionally substituted by one or more R³; R′ is hydrogen or alkyl; each of R¹ and R² is independently hydrogen or alkyl, wherein alkyl is optionally substituted by one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In some embodiments, the compound is a compound of formula (I-2), (I-a), (I-b), (I-c), or (I-d), or a pharmaceutically acceptable salt thereof.

In another aspect, the present invention features a method of treating epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, or KCNT1 epileptic encephalopathy) comprising administering to a subject in need thereof a compound of Formula (II):

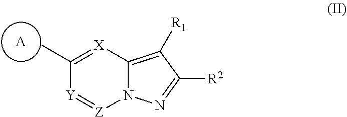

or a pharmaceutically acceptable salt thereof, wherein each of X, Y, and Z is independently N or CR′; A is aryl or heteroaryl (e.g., a monocyclic 6-membered aryl or heteroaryl), wherein aryl and heteroaryl are optionally substituted with one or more R³; R′ is hydrogen, alkyl, or —OR^{c}; each of R¹ and R² is independently hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In some embodiments, the compound is a compound of formula (II-2), (IIa), (IIb), (IIb-1), (IIc), (IId), (IIe), (IIe-1), (IIf), or (IIf-1), or a pharmaceutically acceptable salt thereof.

In another aspect, the present invention features a method of treating epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, or KCNT1 epileptic encephalopathy) comprising administering to a subject in need thereof a compound of Formula (III):

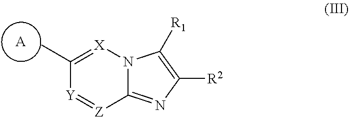

or a pharmaceutically acceptable salt thereof, wherein each of X, Y, and Z is independently N or CR′; A is aryl or heteroaryl (e.g., a monocyclic 6-membered aryl or heteroaryl), wherein aryl and heteroaryl are optionally substituted with one or more R³; R′ is hydrogen, alkyl, or —OR^{c}; each of R¹ and R² is independently hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In some embodiments, the compound is a compound of formula (IIIa), (IIIb), (IIIc), (IIId), (IIId-1), (IIIe), or (IIIe-1), or a pharmaceutically acceptable salt thereof.

A compound of the present invention (e.g., a compound of Formulae (I), (I-2), (I-a), (I-b), (I-c), (I-d), (II), (II-2), (IIa), (IIb), (IIb-1), (IIc), (IId), (IIe), (IIe-1), (IIf), (IIf-1), (III), (IIIa), (IIIb), (IIIc), (IIId), (IIId-1), (IIIe), or (IIIe-1)) may also be used to treat an epileptic encephalopathy, wherein the subject has a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIKI, SLC13A5, SLC25A22, SLC2A1, SLC35A2, SLC6A1, SNIP1, SPTAN1, SRPX2, ST3GAL3, STRADA, STX1B, STXBP1, SYN1, SYNGAPI, SZT2, TBC1D24, and WWOX.

In some embodiments, the methods described herein further comprise identifying a subject having a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIKI, SLC13A5, SLC25A22, SLC2A1, SLC35A2, SLC6A1, SNIP1, SPTAN1, SRPX2, ST3GAL3, STRADA, STX1B, STXBP1, SYN1, SYNGAPI, SZT2, TBC1D24, and WWOX prior to administration of a compound described herein (e.g., a compound of Formulae (I), (I-2), (I-a), (I-b), (I-c), (I-d), (II), (II-2), (IIa), (IIb), (IIb-1), (IIc), (IId), (IIe), (IIe-1), (IIf), (IIf-1), (III), (IIIa), (IIIb), (IIIc), (IIId), (IIId-1), (IIIe), or (IIIe-1)).

### Neurodevelopmental Disorders

The compounds described herein may be useful in the treatment of a neurodevelopmental disorder. In some embodiments, the neurodevelopmental disorder comprises autism, autism with epilepsy, tuberous sclerosis, Fragile X syndrome, Rett syndrome, Angelman syndrome, Dup15q syndrome, 22q13.3 Deletion syndrome, Prader-Willi syndrome, velocardiofacial syndrome, Smith-Lemli-Opitz syndrome, or a neurodevelopmental disorder with epilepsy. In some embodiments, the methods described herein further comprise identifying a subject having a neurodevelopmental disorder (e.g., autism, autism with epilepsy, tuberous sclerosis, Fragile X syndrome, Rett syndrome, Angelman syndrome, Dup15q syndrome, 22q13.3 Deletion syndrome, Prader-Willi syndrome, velocardiofacial syndrome, Smith-Lemli-Opitz syndrome, or a neurodevelopmental disorder with epilepsy) prior to administration of a compound described herein (e.g., a compound of Formulae (I), (I-2), (I-a), (I-b), (I-c), (I-d), (II), (11-2), (IIa), (IIb), (IIb-1), (IIc), (IId), (IIe), (IIe-1), (IIf), (IIf-1), (III), (IIIa), (IIIb), (IIIc), (IIId), (IIId-1), (IIIe), or (IIIe-1)).

In one aspect, the present invention features a method of treating a neurodevelopmental disorder (e.g., autism, autism with epilepsy, tuberous sclerosis, Fragile X syndrome, Rett syndrome, Angelman syndrome, Dup15q syndrome, 2213.3 Deletion syndrome, Prader-Willi syndrome, velocardiofacial syndrome, Smith-Lemli-Opitz syndrome, or a neurodevelopmental disorder with epilepsy) comprising administering to a subject in need thereof a compound of Formula (I):

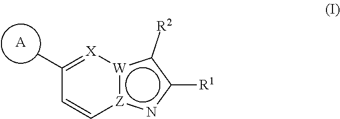

or a pharmaceutically acceptable salt thereof, wherein X is N or CR′; each of W and Z is independently N or C, wherein only one of W and Z is N; A is aryl or heteroaryl (e.g., a monocyclic 6-membered aryl or heteroaryl), optionally substituted by one or more R³; R′ is hydrogen or alkyl; each of R¹ and R² is independently hydrogen or alkyl, wherein alkyl is optionally substituted by one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In another aspect, the present invention features a method of treating a neurodevelopmental disorder (e.g., autism, autism with epilepsy, tuberous sclerosis, Fragile X syndrome, Rett syndrome, Angelman syndrome, Dup15q syndrome, 22q13.3 Deletion syndrome, Prader-Willi syndrome, velocardiofacial syndrome, Smith-Lemli-Opitz syndrome, or a neurodevelopmental disorder with epilepsy) comprising administering to a subject in need thereof a compound of Formula (II):

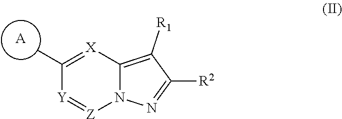

or a pharmaceutically acceptable salt thereof, wherein each of X, Y, and Z is independently N or CR′; A is aryl or heteroaryl (e.g., a monocyclic 6-membered aryl or heteroaryl), wherein aryl and heteroaryl are optionally substituted with one or more R³; R′ is hydrogen, alkyl, or —OR^{c}; each of R¹ and R² is independently hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In some embodiments, the compound is a compound of Formulae (II-2), (IIa), (IIb), (IIb-1), (IIc), (IId), (IIe), (IIe-1), (IIf), (IIf-1), or a pharmaceutically acceptable salt thereof.

In another aspect, the present invention features a method of treating a neurodevelopmental disorder (e.g., autism, autism with epilepsy, tuberous sclerosis, Fragile X syndrome, Rett syndrome, Angelman syndrome, Dup15q syndrome, 22q13.3 Deletion syndrome, Prader-Willi syndrome, velocardiofacial syndrome, Smith-Lemli-Opitz syndrome, or a neurodevelopmental disorder with epilepsy) comprising administering to a subject in need thereof a compound of Formula (III):

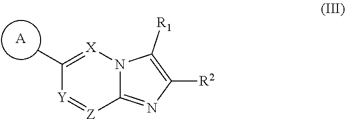

or a pharmaceutically acceptable salt thereof, wherein each of X, Y, and Z is independently N or CR′; A is aryl or heteroaryl (e.g., a monocyclic 6-membered aryl or heteroaryl), wherein aryl and heteroaryl are optionally substituted with one or more R³; R′ is hydrogen, alkyl, or —OR^{c}; each of R¹ and R² is independently hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In some embodiments, the compound is a compound of formula (IIIa), (IIIb), (IIIc), (IIId), (IIId-1), (IIIe), or (IIIe-1), or a pharmaceutically acceptable salt thereof.

### Pain

The compounds described herein may be useful in the treatment of pain. In some embodiments, the pain comprises neuropathic pain, trigeminal neuralgia, migraine, hemiplegic migraine, familial hemiplegic migraine, familial hemiplegic migraine type 3, cluster headache, trigeminal neuralgia, cerebellar ataxia, or a related headache disorder. In some embodiments, the methods described herein further comprise identifying a subject having pain (e.g., neuropathic pain, trigeminal neuralgia, migraine, hemiplegic migraine, familial hemiplegic migraine, familial hemiplegic migraine type 3, cluster headache, trigeminal neuralgia, cerebellar ataxia, or a related headache disorder) prior to administration of a compound described herein (e.g., a compound of Formulae (I), (I-2), (I-a), (I-b), (I-c), (I-d), (II), (11-2), (IIa), (IIb), (IIb-1), (IIc), (IId), (IIe), (IIe-1), (IIf), (IIf-1), (III), (IIIa), (IIIb), (IIIc), (IIId), (IIId-1), (IIIe), or (IIIe-1)).

In one aspect, the present invention features a method of treating pain (e.g., neuropathic pain, trigeminal neuralgia, migraine, hemiplegic migraine, familial hemiplegic migraine, familial hemiplegic migraine type 3, cluster headache, trigeminal neuralgia, cerebellar ataxia, or a related headache disorder) comprising administering to a subject in need thereof a compound of Formula (I):

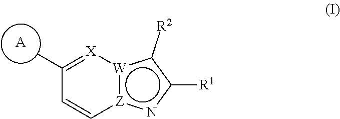

or a pharmaceutically acceptable salt thereof, wherein X is N or CR; each of W and Z is independently N or C, wherein only one of W and Z is N; A is aryl or heteroaryl (e.g., a monocyclic 6-membered aryl or heteroaryl), optionally substituted by one or more R³; R′ is hydrogen or alkyl; each of R¹ and R² is independently hydrogen or alkyl, wherein alkyl is optionally substituted by one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, carbocyclyl, heterocyclyl, halo, cyano, nitro, or —OH.

In some embodiments, the compound is a compound of formula (I-2), (I-a), (I-b), (I-c), or (I-d), or a pharmaceutically acceptable salt thereof.

In another aspect, the present invention features a method of treating pain (e.g., neuropathic pain, trigeminal neuralgia, migraine, hemiplegic migraine, familial hemiplegic migraine, familial hemiplegic migraine type 3, cluster headache, trigeminal neuralgia, cerebellar ataxia, or a related headache disorder) comprising administering to a subject in need thereof a compound of Formula (II):

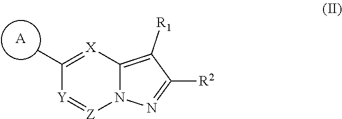

or a pharmaceutically acceptable salt thereof, wherein each of X, Y, and Z is independently N or CR′; A is aryl or heteroaryl (e.g., a monocyclic 6-membered aryl or heteroaryl), wherein aryl and heteroaryl are optionally substituted with one or more R³; R′ is hydrogen, alkyl, or —OR^{c}; each of R¹ and R² is independently hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In some embodiments, the compound is a compound of Formulae (II-2), (IIa), (IIb), (IIb-1), (IIc), (IId), (IIe), (IIe-1), (IIf), (IIf-1), or a pharmaceutically acceptable salt thereof.

In another aspect, the present invention features a method of treating pain (e.g., neuropathic pain, trigeminal neuralgia, migraine, hemiplegic migraine, familial hemiplegic migraine, familial hemiplegic migraine type 3, cluster headache, trigeminal neuralgia, cerebellar ataxia, or a related headache disorder) comprising administering to a subject in need thereof a compound of Formula (III):

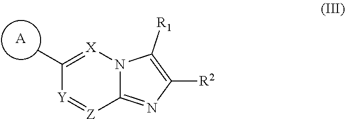

or a pharmaceutically acceptable salt thereof, wherein each of X, Y, and Z is independently N or CR′; A is aryl or heteroaryl (e.g., a monocyclic 6-membered aryl or heteroaryl), wherein aryl and heteroaryl are optionally substituted with one or more R³; R′ is hydrogen, alkyl, or —OR^{c}; each of R¹ and R² is independently hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In some embodiments, the compound is a compound of formula (IIIa), (IIIb), (IIIc), (IIId), (IIId-1), (IIIe), or (IIIe-1), or a pharmaceutically acceptable salt thereof.

### Neuromuscular Disorders

The compounds described herein may be useful in the treatment of a neuromuscular disorder. In some embodiments, the neuromuscular disorder comprises amyotrophic lateral sclerosis, multiple sclerosism, myotonia, paramyotonia congenita, potassium-aggravated myotonia, periodic paralysis, hyperkalemic periodic paralysis, hypokalemic periodic paralysis, and laryngospasm with SCN4A mutation. In some embodiments, the methods described herein further comprise identifying a subject having a neuromuscular disorder (e.g., amyotrophic lateral sclerosis, multiple sclerosism, myotonia, paramyotonia congenita, potassium-aggravated myotonia, periodic paralysis, hyperkalemic periodic paralysis, hypokalemic periodic paralysis, or laryngospasm with SCN4A mutation) prior to administration of a compound described herein (e.g., a compound of Formulae (I), (I-2), (I-a), (I-b), (I-c), (I-d), (II), (11-2), (IIa), (IIb), (IIb-1), (IIc), (IId), (IIe), (IIe-1), (IIf), (IIf-1), (III), (IIIa), (IIIb), (IIIc), (IIId), (IIId-1), (IIIe), or (IIIe-1)).

In one aspect, the present invention features a method of treating a neuromuscular disorder (e.g., amyotrophic lateral sclerosis, multiple sclerosism, myotonia, paramyotonia congenita, potassium-aggravated myotonia, periodic paralysis, hyperkalemic periodic paralysis, hypokalemic periodic paralysis, or laryngospasm with SCN4A mutation) comprising administering to a subject in need thereof a compound of Formula (I):

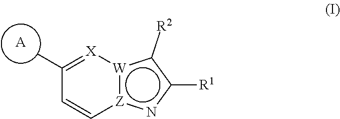

or a pharmaceutically acceptable salt thereof, wherein X is N or CR; each of W and Z is independently N or C, wherein only one of W and Z is N; A is aryl or heteroaryl (e.g., a monocyclic 6-membered aryl or heteroaryl), optionally substituted by one or more R³; R′ is hydrogen or alkyl; each of R¹ and R² is independently hydrogen or alkyl, wherein alkyl is optionally substituted by one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In another aspect, the present invention features a method of treating a neuromuscular disorder (e.g., amyotrophic lateral sclerosis, multiple sclerosism, myotonia, paramyotonia congenita, potassium-aggravated myotonia, periodic paralysis, hyperkalemic periodic paralysis, hypokalemic periodic paralysis, or laryngospasm with SCN4A mutation) comprising administering to a subject in need thereof a compound of Formula (II):

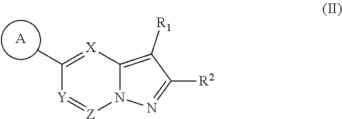

or a pharmaceutically acceptable salt thereof, wherein each of X, Y, and Z is independently N or CR′; A is aryl or heteroaryl (e.g., a monocyclic 6-membered aryl or heteroaryl), wherein aryl and heteroaryl are optionally substituted with one or more R³; R′ is hydrogen, alkyl, or —OR^{c}; each of R¹ and R² is independently hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In some embodiments, the compound is a compound of Formulae (II), (II-2), (IIa), (IIb), (IIb-1), (IIc), (IId), (IIe), (IIe-1), (IIf), (IIf-1), or a pharmaceutically acceptable salt thereof.

In another aspect, the present invention features a method of treating a neuromuscular disorder (e.g., amyotrophic lateral sclerosis, multiple sclerosism, myotonia, paramyotonia congenita, potassium-aggravated myotonia, periodic paralysis, hyperkalemic periodic paralysis, hypokalemic periodic paralysis, or laryngospasm with SCN4A mutation) comprising administering to a subject in need thereof a compound of Formula (III):

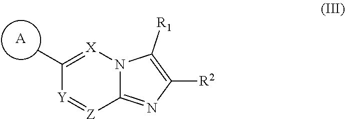

or a pharmaceutically acceptable salt thereof, wherein each of X, Y, and Z is independently N or CR′; A is aryl or heteroaryl (e.g., a monocyclic 6-membered aryl or heteroaryl), wherein aryl and heteroaryl are optionally substituted with one or more R³; R′ is hydrogen, alkyl, or —OR^{c}; each of R¹ and R² is independently hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴; each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵; each R⁴ and R⁵ is independently alkyl, halo, cyano, nitro, or —OR^{c}; each R^{c} is independently hydrogen, alkyl, aryl, cycloalkyl, heterocyclyl, or heteroaryl, wherein alkyl, aryl, cycloalkyl, heterocyclyl, and heteroaryl is optionally substituted by one or more R⁶; each R^{d} is independently hydrogen or alkyl; and each R⁶ is independently alkyl, haloalkyl, carbocyclyl, heterocyclyl, halo, cyano, nitro, or —OH.

In some embodiments, the compound is a compound of formula (IIIa), (IIIb), (IIIc), (IIId), (IIId-1), (IIIe), or (IIIe-1), or a pharmaceutically acceptable salt thereof.

### Other Disorders

In some embodiments, a compound of the present invention (e.g., a compound of Formulae (I), (I-2), (I-a), (I-b), (I-c), (I-d), (II), (IIa), (IIb), (IIb-1), (IIc), (IId), (IIe), (IIe-1), (IIf), (IIf-1), (III), (III-2), (IIIa), (IIIb), (IIIc), (IIId), (IIId-1), (IIIe), or (IIIe-1) may have appropriate pharmacokinetic properties such that they may active with regard to the central and/or peripheral nervous system. In some embodiments, the compounds provided herein are used to treat a cardiovascular disease such as atrial and ventricular arrhythmias, including atrial fibrillation, Prinzmetal's (variant) angina, stable angina, unstable angina, ischemia and reperfusion injury in cardiac, kidney, liver and the brain, exercise induced angina, pulmonary hypertension, congestive heart disease including diastolic and systolic heart failure, and myocardial infarction. In some embodiments, the compounds provided herein may be used in the treatment of diseases affecting the neuromuscular system resulting in itching, seizures, or paralysis, or in the treatment of diabetes or reduced insulin sensitivity, and disease states related to diabetes, such as diabetic peripheral neuropathy.

In any and all aspects, in some embodiments, the compound of Formula Formulae (I), (I-2), (I-a), (I-b), (I-c), (I-d), (II), (II-2), (IIa), (IIb), (IIb-1), (IIc), (IId), (IIe), (IIe-1), (IIf), (IIf-1), (III), (IIIa), (IIIb), (IIIc), (IIId), (IIId-1), (IIIe), or (IIIe-1) is selected from the group consisting of:

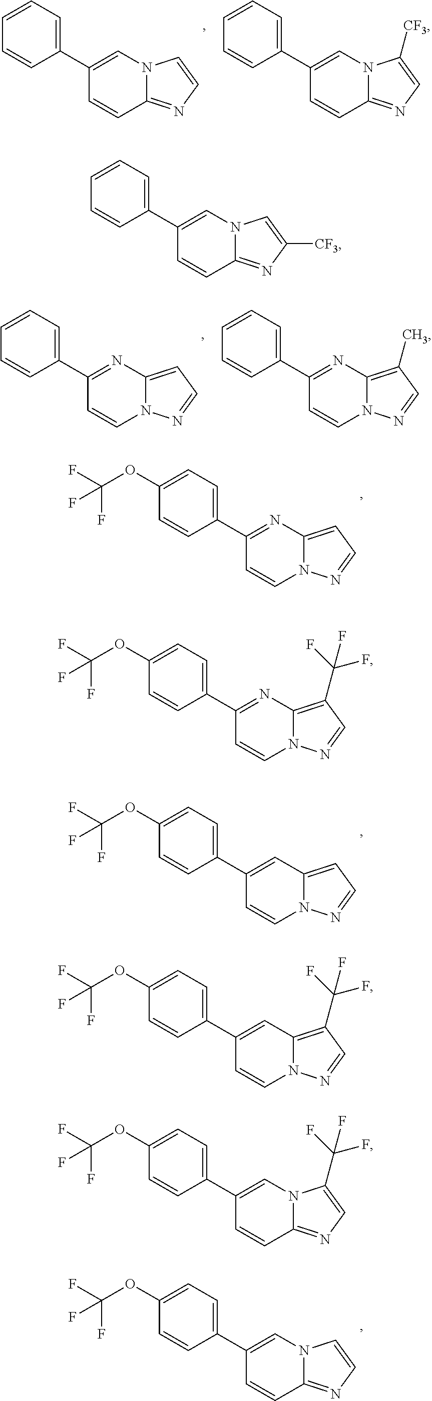

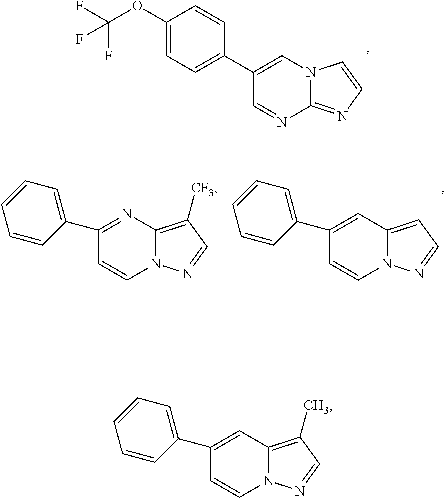

and a pharmaceutically acceptable salt thereof.

In any and all aspects, in some embodiments, the compound of Formulae (I), (I-2), (I-a), (I-b), (I-c), (I-d), (II), (II-2), (IIa), (IIb), (IIb-1), (IIc), (IId), (IIe), (IIe-1), (IIf), (IIf-1), (III), (IIIa), (IIIb), (IIIc), (IIId), (IIId-1), (IIIe), or (IIIe-1) is selected from the group consisting of:

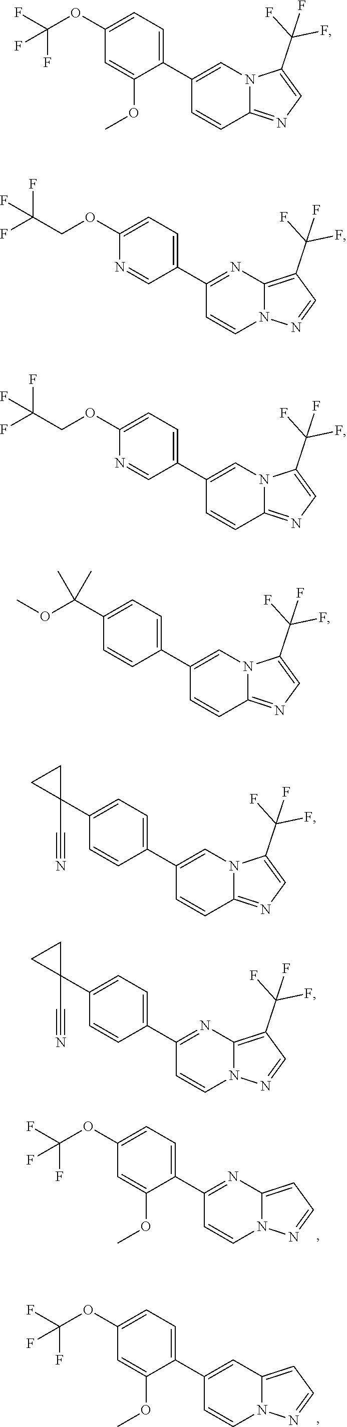

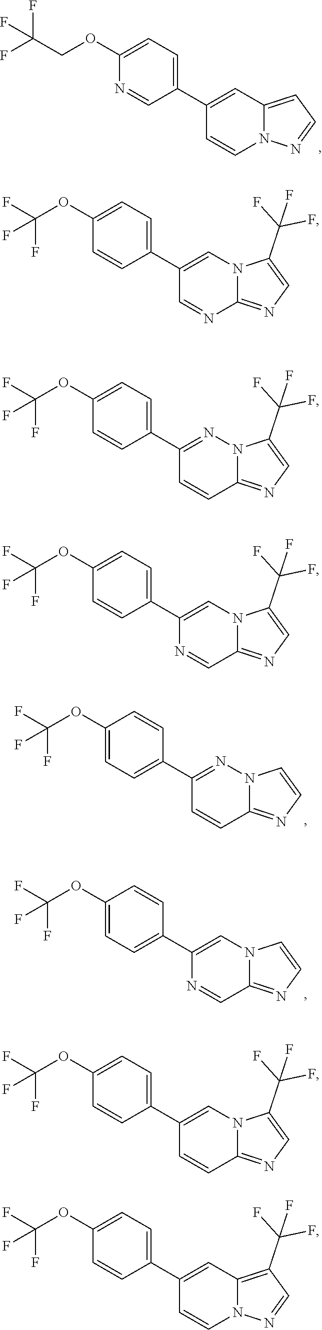

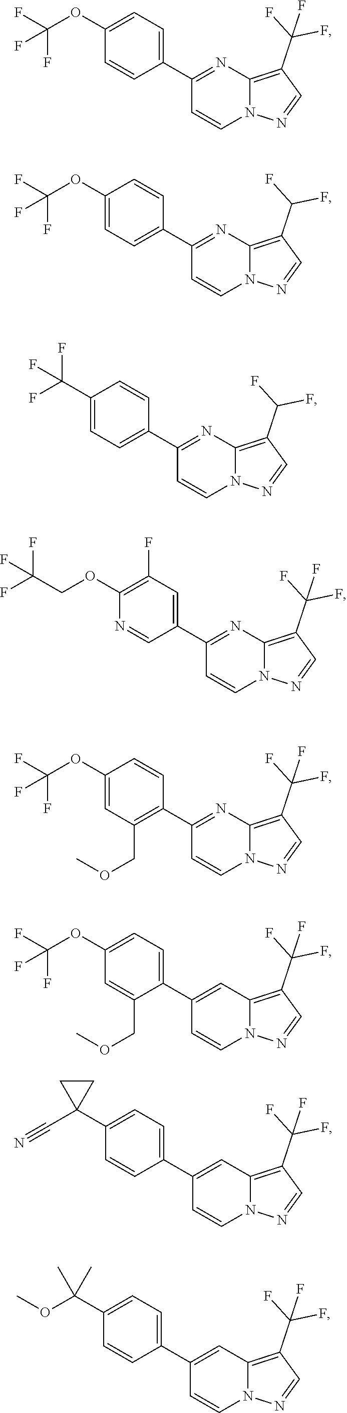

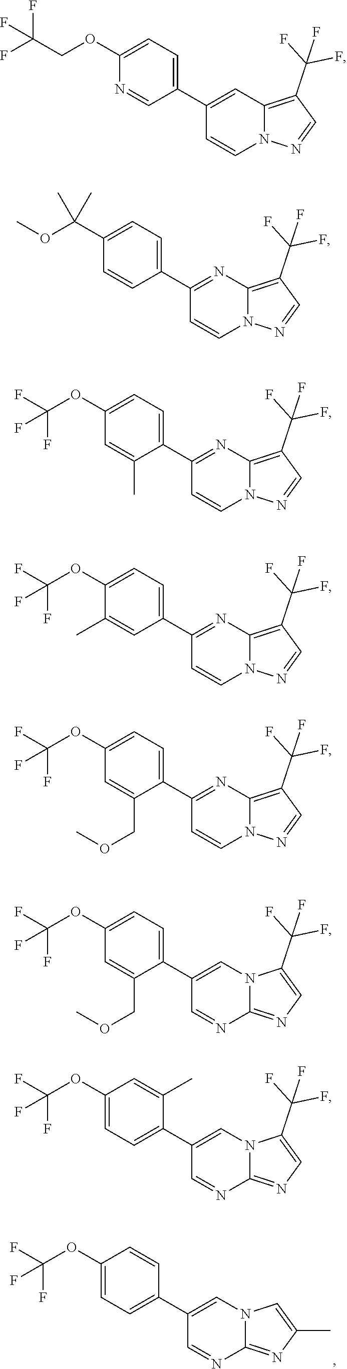

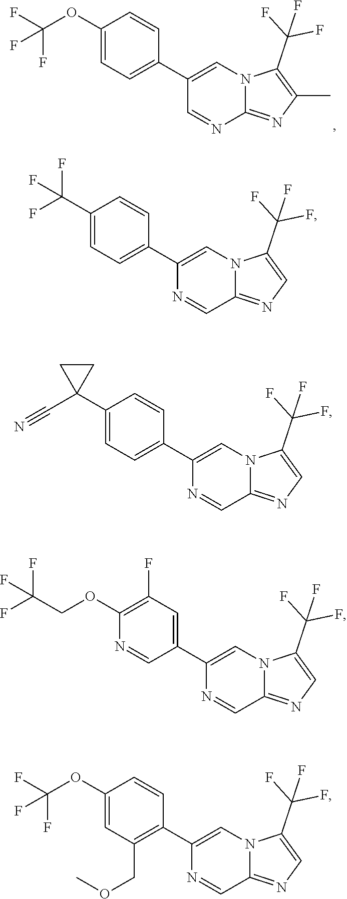 [SMILES: Cc1nc2ncc(-c3ccc(OC(F)(F)F)cc3)cn2c1C(F)(F)F ; FC(F)(F)c1ccc(-c2cn3c(C(F)(F)F)cnc3cn2)cc1 ; N#CC1(c2ccc(-c3cn4c(C(F)(F)F)cnc4cn3)cc2)CC1 ; Fc1cc(-c2cn3c(C(F)(F)F)cnc3cn2)cnc1OCC(F)(F)F ; COCc1cc(OC(F)(F)F)ccc1-c1cn2c(C(F)(F)F)cnc2cn1]

and a pharmaceutically acceptable salt thereof.

### Pharmaceutical Compositions and Routes of Administration

Compounds provided in accordance with the present invention are usually administered in the form of pharmaceutical compositions. This invention therefore provides pharmaceutical compositions that contain, as the active ingredient, one or more of the compounds described, or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.)

In one aspect, the present disclosure provides a pharmaceutical composition comprising a compound disclosed herein and a pharmaceutically acceptable salt thereof.

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compounds in accordance with the invention. Administration may be via capsule or enteric coated tablets, or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from 1 mg to 2 g of a compound described herein, and for parenteral administration, preferably from 0.1 to 700 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### Combination Therapy

A compound or composition described herein (e.g., for use in modulating a sodium ion channel, e.g., the late sodium (INaL) current) may be administered in combination with another agent or therapy. A subject to be administered a compound disclosed herein may have a disease, disorder, or condition, or a symptom thereof, that would benefit from treatment with another agent or therapy. These diseases or conditions can relate to epilepsy or an epilepsy syndrome, a neurodevelopmental disorder, pain, or a neuromuscular disorder.

### Antiepilepsy Agents

Anti-epilepsy agents include brivaracetam, carbamazepine, clobazam, clonazepam, diazepam, divalproex, eslicarbazepine, ethosuximide, ezogabine, felbamate, gabapentin, lacosamide, lamotrigine, levetiracetam, lorazepam, oxcarbezepine, permpanel, phenobarbital, phenytoin, pregabalin, primidone, rufinamide, tigabine, topiramate, valproic acid, vigabatrin, zonisamide.

### Cardiovascular Agent Combination Therapy

Cardiovascular related diseases or conditions that can benefit from a combination treatment of the sodium channel blockers of the invention with other therapeutic agents include, without limitation, angina including stable angina, unstable angina (UA), exercised-induced angina, variant angina, arrhythmias, intermittent claudication, myocardial infarction including non-STE myocardial infarction (NSTEMI), pulmonary hypertension including pulmonary arterial hypertension, heart failure including congestive (or chronic) heart failure and diastolic heart failure and heart failure with preserved ejection fraction (diastolic dysfunction), acute heart failure, or recurrent ischemia.

Therapeutic agents suitable for treating cardiovascular related diseases or conditions include anti-anginals, heart failure agents, antithrombotic agents, antiarrhythmic agents, antihypertensive agents, and lipid lowering agents.

The co-administration of the sodium channel blockers of the invention with therapeutic agents suitable for treating cardiovascular related conditions allows enhancement in the standard of care therapy the patient is currently receiving.

### Anti-Anginals

Anti-anginals include beta-blockers, calcium channel blockers, and nitrates. Beta blockers reduce the heart's need for oxygen by reducing its workload resulting in a decreased heart rate and less vigorous heart contraction. Examples of beta-blockers include acebutolol (Sectral), atenolol (Tenormin), betaxolol (Kerlone), bisoprolol/hydrochlorothiazide (Ziac), bisoprolol (Zebeta), carteolol (Cartrol), esmolol (Brevibloc), labetalol (Normodyne, Trandate), metoprolol (Lopressor, Toprol XL), nadolol (Corgard), propranolol (Inderal), sotalol (Betapace), and timolol (Blocadren).

Nitrates dilate the arteries and veins thereby increasing coronary blood flow and decreasing blood pressure. Examples of nitrates include nitroglycerin, nitrate patches, isosorbide dinitrate, and isosorbide-5-mononitrate.

Calcium channel blockers prevent the normal flow of calcium into the cells of the heart and blood vessels causing the blood vessels to relax thereby increasing the supply of blood and oxygen to the heart. Examples of calcium channel blockers include amlodipine (Norvasc, Lotrel), bepridil (Vascor), diltiazem (Cardizem, Tiazac), felodipine (Plendil), nifedipine (Adalat, Procardia), nimodipine (Nimotop), nisoldipine (Sular), verapamil (Calan, Isoptin, Verelan), and nicardipine.

### Heart Failure Agents

Agents used to treat heart failure include diuretics, ACE inhibitors, vasodilators, and cardiac glycosides. Diuretics eliminate excess fluids in the tissues and circulation thereby relieving many of the symptoms of heart failure. Examples of diuretics include hydrochlorothiazide, metolazone (Zaroxolyn), furosemide (Lasix), bumetanide (Bumex), spironolactone (Aldactone), and eplerenone (nspra).

Angiotensin converting enzyme (ACE) inhibitors reduce the workload on the heart by expanding the blood vessels and decreasing resistance to blood flow. Examples of ACE inhibitors include benazepril (Lotensin), captopril (Capoten), enalapril (Vasotec), fosinopril (Monopril), lisinopril (Prinivil, Zestril), moexipril (Univasc), perindopril (Aceon), quinapril (Accupril), ramipril (Altace), and trandolapril (Mavik).

Vasodilators reduce pressure on the blood vessels by making them relax and expand. Examples of vasodilators include hydralazine, diazoxide, prazosin, clonidine, and methyldopa. ACE inhibitors, nitrates, potassium channel activators, and calcium channel blockers also act as vasodilators.

Cardiac glycosides are compounds that increase the force of the heart's contractions. These compounds strengthen the pumping capacity of the heart and improve irregular heartbeat activity. Examples of cardiac glycosides include digitalis, digoxin, and digitoxin.

### Antithrombotic Agents

Antithrombotics inhibit the clotting ability of the blood. There are three main types of antithrombotics—platelet inhibitors, anticoagulants, and thrombolytic agents.

Platelet inhibitors inhibit the clotting activity of platelets, thereby reducing clotting in the arteries. Examples of platelet inhibitors include acetylsalicylic acid (aspirin), ticlopidine, clopidogrel (plavix), dipyridamole, cilostazol, persantine sulfinpyrazone, dipyridamole, indomethacin, and glycoprotein IIb/IIIa inhibitors, such as abciximab, tirofiban, and eptifibatide (Integrelin). Beta blockers and calcium channel blockers also have a platelet-inhibiting effect.

Anticoagulants prevent blood clots from growing larger and prevent the formation of new clots. Examples of anticoagulants include bivalirudin (Angiomax), warfarin (Coumadin), unfractionated heparin, low molecular weight heparin, danaparoid, lepirudin, and argatroban.

Thrombolytic agents act to break down an existing blood clot. Examples of thrombolytic agents include streptokinase, urokinase, and tenecteplase (TNK), and tissue plasminogen activator (t-PA).

### Antiarrhythmic Agents

Antiarrhythmic agents are used to treat disorders of the heart rate and rhythm. Examples of antiarrhythmic agents include amiodarone, dronedarone, quinidine, procainamide, lidocaine, and propafenone. Cardiac glycosides and beta blockers are also used as antiarrhythmic agents.

Combinations with amiodarone and dronedarone are of particular interest given the recently discovered synergistic effects of the sodium channel blocker ranolazine and amioarone and dronedarone.

### Antihypertensive Agents

Antihypertensive agents are used to treat hypertension, a condition in which the blood pressure is consistently higher than normal. Hypertension is associated with many aspects of cardiovascular disease, including congestive heart failure, atherosclerosis, and clot for illation. Examples of antihypertensive agents include alpha-1-adrenergic antagonists, such as prazosin (Minipress), doxazosin mesylate (Cardura), prazosin hydrochloride (Minipress), prazosin, polythiazide (Minizide), and terazosin hydrochloride (Hytrin); beta-adrenergic antagonists, such as propranolol (Inderal), nadolol (Corgard), timolol (Blocadren), metoprolol (Lopressor), and pindolol (Visken); central alpha-adrenoceptor agonists, such as clonidine hydrochloride (Catapres), clonidine hydrochloride and chlorthalidone (Clorpres, Combipres), guanabenz Acetate (Wytensin), guanfacine hydrochloride (Tenex), methyldopa (Aldomet), methyldopa and chlorothiazide (Aldoclor), methyldopa and hydrochlorothiazide (Aldoril); combined alpha/beta-adrenergic antagonists, such as labetalol (Normodyne, Trandate), Carvedilol (Coreg); adrenergic neuron blocking agents, such as guanethidine (ismelin), reserpine (Serpasil); central nervous system-acting antihypertensives, such as clonidine (Catapres), methyldopa (Aldomet), guanabenz (Wytensin); anti-angiotensin II agents; ACE inhibitors, such as perindopril (Aceon) captopril (Capoten), enalapril (Vasotec), lisinopril (Prinivil, Zestril); angiotensin-II receptor antagonists, such as Candesartan (Atacand), Eprosartan (Teveten), Irbesartan (Avapro), Losartan (Cozaar), Telmisartan (Micardis), Valsartan (Diovan); calcium channel blockers, such as verapamil (Calan, Isoptin), diltiazem (Cardizem), nifedipine (Adalat, Procardia); diuretics; direct vasodilators, such as nitroprusside (Nipride), diazoxide (Hyperstat IV), hydralazine (Apresoline), minoxidil (Loniten), verapamil; and potassium channel activators, such as aprikalim, bimakalim, cromakalim, emakalim, nicorandil, and pinacidil.

### Lipid Lowering Agents

Lipid lowering agents are used to lower the amounts of cholesterol or fatty sugars present in the blood. Examples of lipid lowering agents include bezafibrate (Bezalip), ciprofibrate (Modalim), and statins, such as atorvastatin (Lipitor), fluvastatin (Lescol), lovastatin (Mevacor, Altocor), mevastatin, pitavastatin (Livalo, Pitava) pravastatin (Lipostat), rosuvastatin (Crestor), and simvastatin (Zocor).

In this invention, the patient presenting with an acute coronary disease event often suffers from secondary medical conditions such as one or more of a metabolic disorder, a pulmonary disorder, a peripheral vascular disorder, or a gastrointestinal disorder. Such patients can benefit from treatment of a combination therapy comprising administering to the patient ranolazine in combination with at least one therapeutic agent.

### Pulmonary Disorders Combination Therapy

Pulmonary disorder refers to any disease or condition related to the lungs. Examples of pulmonary disorders include, without limitation, asthma, chronic obstructive pulmonary disease (COPD), bronchitis, and emphysema.

Examples of therapeutics agents used to treat pulmonary disorders include bronchodilators including beta2 agonists and anticholinergics, corticosteroids, and electrolyte supplements. Specific examples of therapeutic agents used to treat pulmonary disorders include epinephrine, terbutaline (Brethaire, Bricanyl), albuterol (Proventil), salmeterol (Serevent, Serevent Diskus), theophylline, ipratropium bromide (Atrovent), tiotropium (Spiriva), methylprednisolone (Solu-Medrol, Medrol), magnesium, and potassium.

### Metabolic Disorders Combination Therapy

Examples of metabolic disorders include, without limitation, diabetes, including type I and type II diabetes, metabolic syndrome, dyslipidemia, obesity, glucose intolerance, hypertension, elevated serum cholesterol, and elevated triglycerides.

Examples of therapeutic agents used to treat metabolic disorders include antihypertensive agents and lipid lowering agents, as described in the section “Cardiovascular Agent Combination Therapy” above. Additional therapeutic agents used to treat metabolic disorders include insulin, sulfonylureas, biguanides, alpha-glucosidase inhibitors, and incretin mimetics.

### Peripheral Vascular Disorders Combination Therapy

Peripheral vascular disorders are disorders related to the blood vessels (arteries and veins) located outside the heart and brain, including, for example peripheral arterial disease (PAD), a condition that develops when the arteries that supply blood to the internal organs, arms, and legs become completely or partially blocked as a result of atherosclerosis.

### Gastrointestinal Disorders Combination Therapy

Gastrointestinal disorders refer to diseases and conditions associated with the gastrointestinal tract. Examples of gastrointestinal disorders include gastroesophageal reflux disease (GERD), inflammatory bowel disease (IBD), gastroenteritis, gastritis and peptic ulcer disease, and pancreatitis.

Examples of therapeutic agents used to treat gastrointestinal disorders include proton pump inhibitors, such as pantoprazole (Protonix), lansoprazole (Prevacid), esomeprazole (Nexium), omeprazole (Prilosec), rabeprazole; H2 blockers, such as cimetidine (Tagamet), ranitidine (Zantac), famotidine (Pepcid), nizatidine (Axid); prostaglandins, such as misoprostoL (Cytotec); sucralfate; and antacids.

### Antibiotics, Analgesics, Antidepressants and Anti-Anxiety Agents Combination Therapy

Patients presenting with an acute coronary disease event may exhibit conditions that benefit from administration of therapeutic agent or agents that are antibiotics, analgesics, antidepressant and anti-anxiety agents in combination with ranolazine.

### Antibiotics

Antibiotics are therapeutic agents that kill, or stop the growth of, microorganisms, including both bacteria and fungi. Example of antibiotic agents include. beta.-Lactam antibiotics, including penicillins (amoxicillin), cephalosporins, such as cefazolin, cefuroxime, cefadroxil (Duricef), cephalexin (Keflex), cephradine (Velosef), cefaclor (Ceclor), cefuroxime axtel (Ceftin), cefprozil (Cefzil), loracarbef (Lorabid), cefixime (Suprax), cefpodoxime proxetil (Vantin), ceftibuten (Cedax), cefdinir (Omnicef), ceftriaxone (Rocephin), carbapenems, and monobactams; tetracyclines, such as tetracycline; macrolide antibiotics, such as erythromycin; aminoglycosides, such as gentamicin, tobramycin, amikacin; quinolones such as ciprofloxacin; cyclic peptides, such as vancomycin, streptogramins, polymyxins; lincosamides, such as clindamycin; oxazolidinoes, such as linezolid; and sulfa antibiotics, such as sulfisoxazole.

### Analgesics

Analgesics are therapeutic agents that are used to relieve pain. Examples of analgesics include opiates and morphinomimetics, such as fentanyl and morphine; paracetamol; NSAIDs, and COX-2 inhibitors. Given the ability of the sodium channel blockers of the invention to treat neuropathic pain via inhibition of the Nav 1.7 and 1.8 sodium channels, combination with analgesics are particularly envisioned. See U.S. Patent Application Publication 20090203707.

### Antidepressant and Anti-anxiety Agents

Antidepressant and anti-anxiety agents include those agents used to treat anxiety disorders, depression, and those used as sedatives and tranquillizers. Examples of antidepressant and anti-anxiety agents include benzodiazepines, such as diazepam, lorazepam, and midazolam; benzodiazepines; barbiturates; glutethimide; chloral hydrate; meprobamate; sertraline (Zoloft, Lustral, Apo-Sertral, Asentra, Gladem, Serlift, Stimuloton); escitalopram (Lexapro, Cipralex); fluoxetine (Prozac, Sarafem, Fluctin, Fontex, Prodep, Fludep, Lovan); venlafaxine (Effexor XR, Efexor); citalopram (Celexa, Cipramil, Talohexane); paroxetine (Paxil, Seroxat, Aropax); trazodone (Desyrel); amitriptyline (Elavil); and bupropion (Wellbutrin, Zyban).

Accordingly, one aspect of the invention provides for a composition comprising the sodium channel blockers of the invention and at least one therapeutic agent. In an alternative embodiment, the composition comprises the sodium channel blockers of the invention and at least two therapeutic agents. In further alternative embodiments, the composition comprises the sodium channel blockers of the invention and at least three therapeutic agents, the sodium channel blockers of the invention and at least four therapeutic agents, or the sodium channel blockers of the invention and at least five therapeutic agents.

The methods of combination therapy include co-administration of a single formulation containing the sodium channel blockers of the invention and therapeutic agent or agents, essentially contemporaneous administration of more than one formulation comprising the sodium channel blocker of the invention and therapeutic agent or agents, and consecutive administration of a sodium channel blocker of the invention and therapeutic agent or agents, in any order, wherein preferably there is a time period where the sodium channel blocker of the invention and therapeutic agent or agents simultaneously exert their therapeutic effect.

### Examples

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimal reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include recrystallization, filtration, flash chromatography, trituration, high pressure liquid chromatography (HPLC), or supercritical fluid chromatography (SFC). Note that flash chromatography may either be performed manually or via an automated system. The compounds provided herein may be characterized by known standard procedures, such as nuclear magnetic resonance spectroscopy (NMR) or liquid chromatography mass spectrometry (LCMS). NMR chemical shifts are reported in part per million (ppm) and are generated using methods well known to those of skill in the art.

Exemplary general methods for analytical LCMS include Method A (Xtimate C₁₈ (2.1 mm×30 mm, 3 μm); A=H₂O (0.04% TFA) and B═CH₃CN (0.02% TFA); 50° C.; 1.2 mL/min; 10-80% B over 0.9 minutes, then 80% B for 0.6 minutes); Method B (Chromolith Flash RP-18 endcapped C₁₈ (2 mm×25 mm); A=H₂O (0.04% TFA) and B═CH₃CN (0.02% TFA); 50° C.; 1.5 mL/min; 5-95% B over 0.7 minutes, then 95% B for 0.4 minutes); and Method C (Xtimate C₁₈ (2.1 mm×30 mm, 3 μm); A=H₂O (0.04% TFA) and B═CH₃CN (0.02% TFA); 50° C.; 0.8 mL/min; 10-80% B over 6 minutes, then 80% B for 0.5 minutes).

### List of Abbreviations

NIS N-iodosuccinimide
DMF N,N-dimethylformamide
THF tetrahydrofuran
MeOH methanol
DCM dichloromethane
LiHMDS lithium bis(trimethylsilyl)amide
EtOH ethanol
Et₃N trimethylamine
Pd(dppf)Cl₂ [1,1′-bis(diphenylphosphino)ferrocene]palladium(II)dichloride
Mel methyliodide
Et₃SiH triethylsilane
DBU 1,8-diazabicyclo(5.4.0)undec-7-ene
AcN acetonitrile
TMSCF₃ trifluoromethyltrimethylsilane
TBAB tetrabutylammonium bromide
Pd(t-Bu₃P)₂ bis(tri-tert-butylphosphine)palladium(0)
DAST diethylaminosulfur trifluoride
DIPEA N,N-diisopropylethylamine
HATU 1-[bis(dimethylamino)methylene]-1H-1,2,3-triazolo[4,5-b]pyridinium 3-oxide hexafluorophosphate
Pd(dba)₃ tris(dibenzylideneacetone)dipalladium(0)
XPhos 2-dicyclohexylphosphino-2′,4′,6′-triisopropylbiphenyl

### Example 1: Synthesis of Compound 1

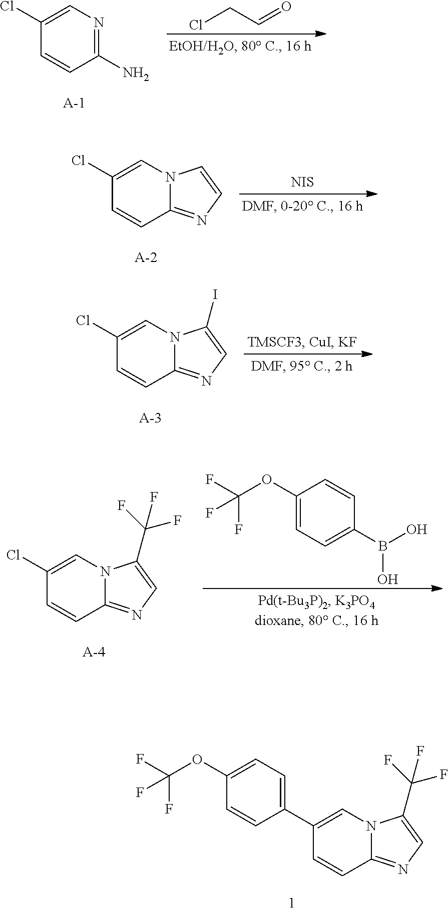

Synthesis of A-2: A mixture of A-1 (4.50 g, 35.00 mmol) and 2-chloroacetaldehyde (10.30 g, 52.50 mmol, 8.45 mL) in EtOH (50.00 mL) and H₂O (10 mL) was stirred at 80° C. for 16 hours The mixture was concentrated to a residue that was diluted with H₂O (100 mL) and extracted with EtOAc (150 mL×2). The combined organic phase was washed with water (50 mL×2) and brine (20 m), dried over Na₂SO₄, filtered and concentrated to afford A-2 (5.10 g, 33.43 mmol) as a solid. ¹H NMR (400 MHz, CDCl₃) δ_{H} 8.18 (d, 1H), 7.65 (s, 1H), 7.59-7.50 (m, 2H), 7.12 (dd, 1H).

Synthesis of A-3: To a mixture of A-2 (500.00 mg, 3.28 mmol) in DMF (10 mL) was added NIS (884.70 mg, 3.94 mmol) at 0° C. The mixture was allowed to warm to 20° C. and stir for 16 hours. Sat.NaHCO₃ (20 mL) was then added, and the mixture was stirred for 1 hour. The solid was collected by filtration and dried in oven to afford A-3 (900.00 mg, 3.23 mmol) as a solid. ¹H NMR (400 MHz, CDCl₃) δ_{H} 8.20 (s, 1H), 7.72 (s, 1H), 7.57 (d, 1H), 7.21 (dd, 1H). LCMS Rₜ=0.373 min in 1.5 min chromatography, MS ESI calcd. for C₇H₅ClIN₂ [M+H]⁺ 278.9, found 278.8.

Synthesis of A-4: A mixture of A-3 (1.00 g, 3.59 mmol), CuI (3.42 g, 17.95 mmol), TMSCF₃ (1.53 g, 10.77 mmol) and KF (625.90 mg, 10.77 mmol, 252.38 μL) in DMF (40.00 mL) was stirred at 95° C. for 2 hours under N₂, at which point the desired product was observed by LCMS. The mixture was then quenched with H₂O (100 mL) and extracted with EtOAc (200 mL×2). The combined organic layers were washed with H₂O (100 mL×2), brine (100 m), dried over Na₂SO₄, filtered and concentrated to give the crude product, which was purified by silica gel (PE:EtOAc=15:1 to EtOAc) to afford A-4 (140.00 mg, 634.69 umol) as a solid. ¹H NMR (CDCl₃ 400 MHz) δ=8.28 (s, 1H), 7.98 (d, 1H), 7.69 (d, 1H), 7.37 (dd, 1H). Synthesis of Compound 1: A mixture of A-4 (100.0 mg, 453.35 μmol), [4-(trifluoromethoxy)phenyl]boronic acid (140.04 mg, 680.03 μmol), Pd(t-Bu₃P)₂ (23.17 mg, 45.34 μmol) and K₃PO₄ (192.47 mg, 906.70 μmol) in dioxane (5.00 mL) and H₂O (1.00 mL) was stirred at 80° C. for 16 hours under N₂. The mixture was then concentrated to give the crude product that was purified by Prep-HPLC to afford Compound 1 (87.14 mg) as a solid. ¹H NMR (400 MHz, CDCl₃) δ_{H} 8.35 (s, 1H), 8.01 (d, 1H), 7.82 (d, 1H), 7.65-7.55 (m, 3H), 7.37 (d, 2H). LCMS Rₜ=1.341 min in 2.0 min chromatography, MS ESI calcd. for C₁₅H₉F₆N₂O [M+H]⁺ 347.1, found 346.9.

### Example 2: Synthesis of Compound 2

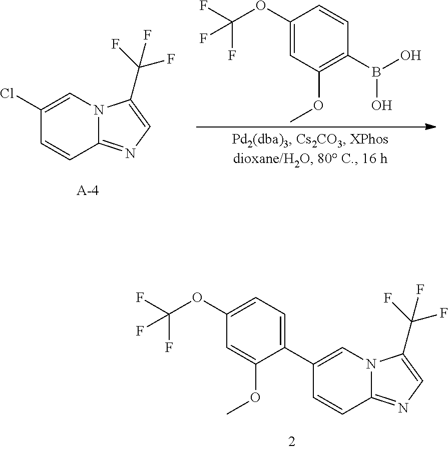

A mixture of A-4 (60.00 mg, 272.01 μmol), [2-methoxy-4-(trifluoromethoxy)-phenyl]boronic acid (77.02 mg, 326.41 μmol), Cs₂CO₃ (177.25 mg, 544.02 μmol), Pd₂(dba)₃ (37.36 mg, 40.80 μmol) and XPhos (45.39 mg, 95.20 μmol) in dioxane (5 mL) and H₂O (1 mL) was stirred at 80° C. for 16 hours under N₂. The mixture was then concentrated to give the crude product that was purified by Prep-HPLC to afford Compound 2 (82.87 mg) as a solid. ¹H NMR (400 MHz, CDCl₃) δ_{H} 8.36 (s, 1H), 7.99 (d, 1H), 7.75 (d, 1H), 7.54 (dd, 1H), 7.38 (d, 1H), 6.97 (td, 1H), 6.88 (s, 1H), 3.88 (s, 3H). LCMS Rₜ=1.195 min in 2.0 min chromatography, MS ESI calcd. for C₁₆H₁₁F₆N₂O₂ [M+H]⁺ 377.1, found 376.9.

### Example 3: Synthesis of Compound 3

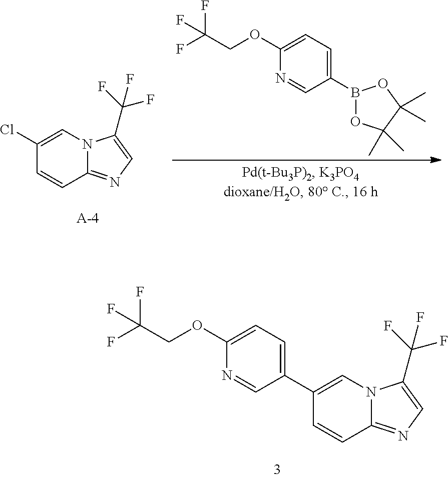

A mixture of A-4 (60.00 mg, 272.01 μmol), 5-(4,4,5,5-tetramethyl-1,3,2-dioxaborolan-2-yl)-2-(2,2,2-trifluoroethoxy)pyridine (123.66 mg, 408.02 μmol), Pd(t-Bu₃P)₂ (13.90 mg, 27.20 μmol) and K₃PO₄ (115.48 mg, 544.02 μmol) in dioxane (5 mL) and H₂O (1 mL) was stirred at 80° C. for 16 hours under N₂. The mixture was then concentrated to give the crude product, which was purified by Prep-HPLC to afford Compound 3 (29.66 mg) as a solid. ¹H NMR (400 MHz, CDCl₃) δ_{H} 8.40-8.29 (m, 2H), 8.02 (d, 1H), 7.90-7.80 (m, 2H), 7.56 (dd, 1H), 7.03 (d, 1H), 4.84 (q, 2H). LCMS Rₜ=1.153 min in 2.0 min chromatography, MS ESI calcd. for C₁₅H₁₀F₆N₃O [M+H]⁺ 362.1, found 362.0.

### Example 4: Synthesis of Compound 4

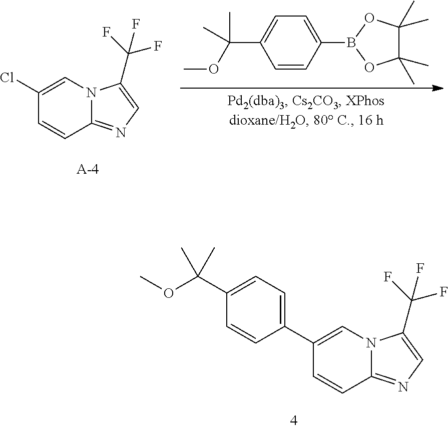

A mixture of A-4 (60.00 mg, 272.01 μmol), 2-[4-(1-methoxy-1-methyl-ethyl)phenyl]-4,4,5,5-tetramethyl-1,3,2-dioxaborolane (112.69 mg, 408.02 μmol), Cs₂CO₃ (177.25 mg, 544.02 mol), XPhos (45.39 mg, 95.20 μmol) and Pd₂(dba)₃ (37.36 mg, 40.80 μmol) in dioxane (5 mL) and H₂O (1 mL) was stirred at 80° C. for 16 hours under N₂. The mixture was concentrated to give the crude product, which was purified by Prep-TLC (PE:EtOAc=3:1) to afford Compound 4 (59.50 mg) as a solid. ¹H NMR (400 MHz, CDCl₃) δ_{H} 8.37 (s, 1H), 8.00 (d, 1H), 7.80 (d, 1H), 7.65 (dd, 1H), 7.56 (s, 4H), 3.14 (s, 3H), 1.59 (s, 6H). LCMS Rₜ=0.846 min in 1.5 min chromatography, MS ESI calcd. for C₁₈H₁₈F₃N₂O [M+H]⁺ 335.1, found 334.9.

### Example 5: Synthesis of Compound 5

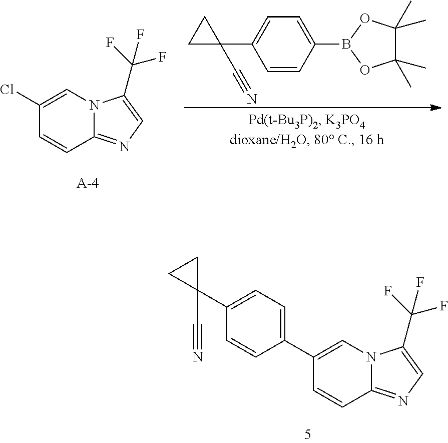

A mixture of A-4 (70.00 mg, 317.35 μmol), 1-[4-(4,4,5,5-tetramethyl-1,3,2-dioxaborolan-2-yl)phenyl]cyclopropanecarbonitrile (128.12 mg, 476.03 μmol), Pd(t-Bu₃P)₂ (32.44 mg, 63.47 μmol) and K₃PO₄ (134.73 mg, 634.70 μmol) in dioxane (5 mL) and H₂O (1 mL) was stirred at 80° C. for 16 hours under N₂. The mixture was then concentrated to give the crude product, which was purified by Prep-TLC (PE:EtOAc=5:1) and Prep-HPLC to afford Compound 5 (7.74 mg) as a solid. H NMR (400 MHz, CDCl₃) δ_{H} 8.35 (s, 1H), 8.00 (d, 1H), 7.81 (d, 1H), 7.63-7.55 (m, 3H), 7.48-7.41 (m, 2H), 1.86-1.78 (m, 2H), 1.51-1.45 (m, 2H). LCMS Rₜ=1.077 min in 2.0 min chromatography, MS ESI calcd. for C₈H₁₃F₃N₃ [M+H]⁺ 328.1, found 327.9.

### Example 6: Synthesis of Compound 6

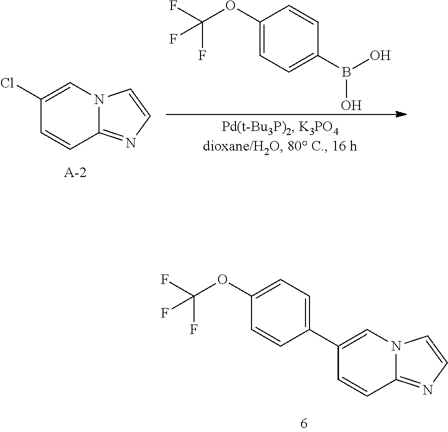

A mixture of [4-(trifluoromethoxy)phenyl]boronic acid (202.45 mg, 983.08 μmol), A-2 (100.0 mg, 655.39 μmol), Pd(t-Bu₃P)₂ (33.49 mg, 65.54 μmol) and K₃PO₄ (278.24 mg, 1.31 mmol) in dioxane (10 mL) and H₂O (1 mL) was stirred at 80° C. for 16 hours, at which point the desired product was observed by LCMS. The mixture was then concentrated to give the crude product, which was purified by Prep-HPLC to afford Compound 6 (38.55 mg) as a solid. ¹H NMR (400 MHz, CDCl₃) δ_{H} 8.30 (s, 1H), 7.74-7.63 (m, 3H), 7.58 (d, 2H), 7.39 (br d, 1H), 7.33 (br d, 2H). LCMS Rₜ=0.981 min in 2.0 min chromatography, MS ESI calcd. for C₁₄H₁₀F₃N₂O [M+H]⁺ 279.1, found 278.9.

### Example 7: Synthesis of Compound 7

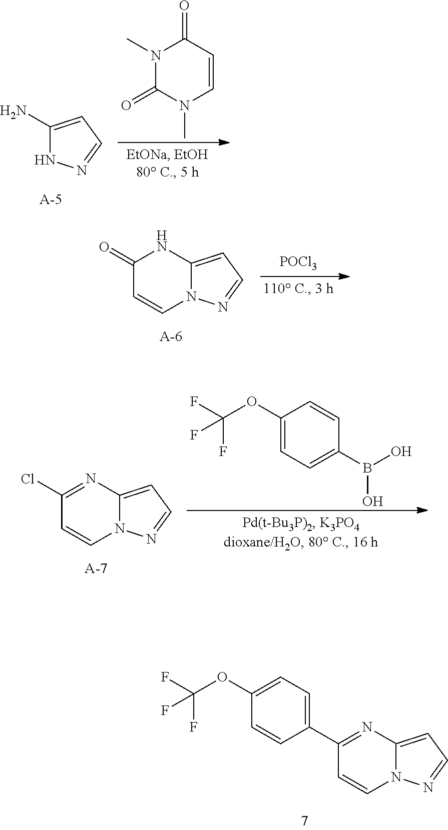

Synthesis of A-6: A mixture of A-5 (8.00 g, 96.28 mmol), 1,3-dimethylpyrimidine-2,4-dione (13.49 g, 96.28 mmol) and EtONa (32.76 g, 481.40 mmol) in EtOH (150 mL) was stirred at 80° C. for 5 hours. The solid was collected by filtration, washed with EtOH (50 mL), and dried in an oven to afford A-6 (12.50 g, 92.51 mmol) as a solid. ¹H NMR (400 MHz, DMSO-d₆) δ_{H} 7.97 (d, 1H), 7.43 (d, 1H), 5.62 (d, 1H), 5.35 (d, 1H).

Synthesis of A-7: A mixture of A-7 (10.00 g, 74.01 mmol) in POCl₃ (100 mL) was stirred at 110° C. for 3 hours. The mixture was then concentrated, and the residue was diluted with a mixture of ice and H₂O (200 m), basified with NaHCO₃ (solid) to pH-7-8, and extracted with EtOAc (150 mL×2). The combined organic phase was washed with water (50 mL×2) and brine (50 mL), dried over Na₂SO₄, filtered and concentrated to give the crude product, which was purified by silica gel (EtOAc in DCM=1% to 5%) to afford A-7 (5.40 g, 34.97 mmol) as a solid. ¹H NMR (400 MHz, CDCl₃) δ_{H} 8.59 (d, 1H), 8.14 (d, 1H), 6.82 (d, 1H), 6.64 (d, 1H). LCMS Rₜ=0.537 min in 1.5 min chromatography, MS ESI calcd. for C₆H₅CN₃ [M+H]⁺ 154.0, found 153.8.

Synthesis of Compound 7: A mixture of A-7 (1.00 g, 6.51 mmol), [4-(trifluoromethoxy)phenyl]boronic acid (1.61 g, 7.81 mmol), Pd(t-Bu₃P)₂ (665.39 mg, 1.30 mmol) and K₃PO₄ (2.76 g, 13.02 mmol) in dioxane (20 mL) and H₂O (2 mL) was stirred at 80° C. for 16 hours under N₂, at which point the desired product was observed by LCMS. The mixture was concentrated to give the crude product, which was diluted with MeOH (20 mL) and purified by silica gel (EtOAc in PE=10% to 20% to 60%) to give Compound 7 (1.42 g) as a solid. ¹H NMR (400 MHz, DMSO-d₆) δ_{H} 9.22 (d, 1H), 8.34 (d, 2H), 8.26 (d, 1H), 7.67 (d, 1H), 7.53 (d, 2H), 6.78 (d, 1H).

### Example 8: Synthesis of Compound 8

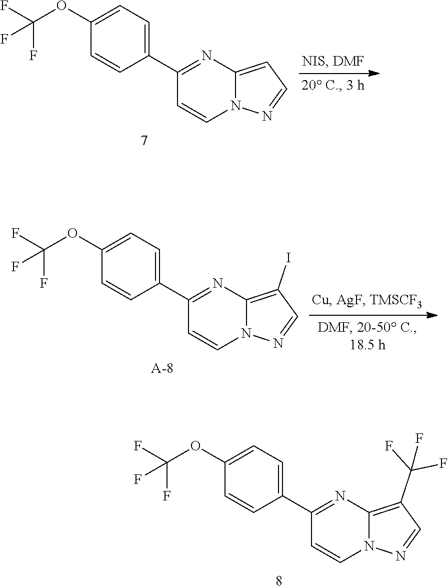 [SMILES: FC(F)(F)Oc1ccc(-c2ccn3nccc3n2)cc1 ; FC(F)(F)Oc1ccc(-c2ccn3ncc(I)c3n2)cc1 ; "A-8" ; FC(F)(F)Oc1ccc(-c2ccn3ncc(C(F)(F)F)c3n2)cc1 ; "8"]

Synthesis of A-8: To a solution of Compound 7 (300.00 mg, 1.07 mmol) in DMF (6 mL) was added NIS (288.87 mg, 1.28 mmol), and the mixture was stirred at 25° C. for 3 hours. The mixture was diluted with H₂O (30 mL). The solid was collected by filtration, washed with H₂O (5 mL×2) and dried in an oven to afford A-8 (377.00 mg) as a solid. ¹H NMR (400 MHz, CDCl₃) δ_{H} 8.70 (d, 1H), 8.26-8.20 (m, 2H), 8.17 (s, 1H), 7.38 (d, 2H), 7.30 (d, 1H). LCMS Rₜ=0.926 min in 1.5 min chromatography, MS ESI calcd. for C₁₃H₈F₃IN₃O [M+H]⁺ 406.0, found 405.9.

Synthesis of Compound 8: To a mixture of AgF (20.36 mg, 160.45 μmol, 3.48 uL) in DMF (1 mL) was added TMSCF₃ (28.08 mg, 197.47 μmol), and the mixture was stirred at 20° C. for 0.5 hour. Cu (15.69 mg, 246.84 umol, 1.75 μL) was then added, and the mixture was stirred at 20° C. for 4 hours under N₂. 3-iodo-5-[4-(trifluoromethoxy)-phenyl]pyrazolo[1,5-a]pyrimidine (50.00 mg, 123.42 μmol) was then added, then the mixture was stirred at 20° C. for 2 hours and at 50° C. for 12 hours. The mixture was then diluted with H₂O (10 mL) and extracted with EtOAc (20 mL×2). The combined organic phase was washed with water (10 mL×2) and brine (10 mL), dried over Na₂SO₄, filtered and concentrated to give the crude product, which was purified by Prep-TLC (EtOAc/PET=¼) to afford Compound 8 (12.30 mg, 34.71 μmol) as a solid. ¹H NMR (400 MHz, CDCl₃) δ_{H} 8.78 (d, 1H), 8.34 (s, 1H), 8.24 (d, 2H), 7.45 (d, 1H), 7.40 (d, 2H). LCMS Rₜ=1.075 min in 2.0 min chromatography, MS ESI calcd. for C₁₄H₈F₆N₃O [M+H]⁺ 348.0, found 348.1.

### Example 9: Synthesis of Compound 9

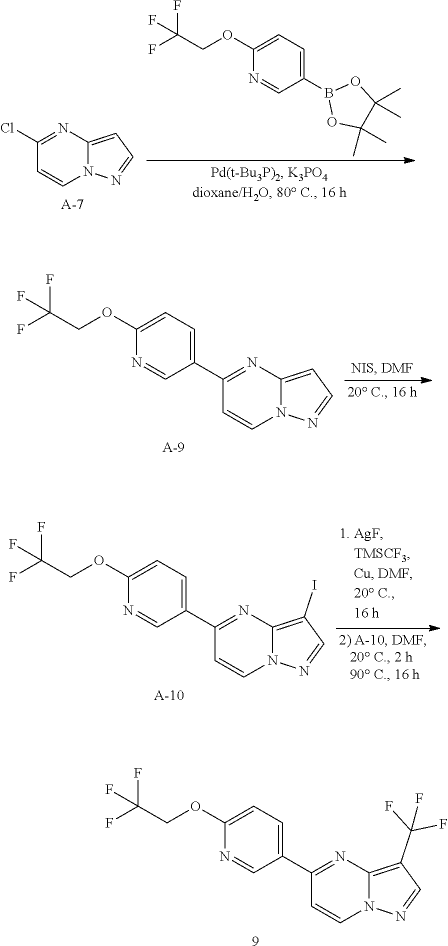

Synthesis of A-9: A mixture of A-7 (300.00 mg, 1.95 mmol), 5-(4,4,5,5-tetramethyl-1,3,2-dioxaborolan-2-yl)-2-(2,2,2-trifluoroethoxy)pyridine (709.21 mg, 2.34 mmol), Pd(t-Bu₃P)₂ (149.48 mg, 292.50 μmol) and K₃PO₄ (827.85 mg, 3.90 mmol) in dioxane (10 mL) and H₂O (3.9 mL) was stirred at 80° C. for 16 hours. The mixture was then concentrated to give the crude product, which was purified by silica gel (EtOAc in PE=20% to 40% to 100%) to afford A-9 (600.00 mg, 1.92 mmol) as a solid. H NMR (400 MHz, CDCl₃) δ_{H} 8.83 (d, 1H), 8.74 (br d, ¹H), 8.46 (dd, 1H), 8.15 (d, 1H), 7.23 (d, 1H), 7.02 (d, 1H), 6.72 (dd, 1H), 4.86 (q, 2H). LCMS Rₜ=0.805 min in 1.5 mins chromatography, MS ESI calcd. for C₁₃H₁₀F₃N₄O [M+H]⁺295.1, found 294.9.

Synthesis of A-10: To a solution of A-9 (600 mg, 2.04 mmol) in DMF (10 mL) was added NIS (550.54 mg, 2.45 mmol), and the mixture was stirred at 20° C. for 16 hours. The mixture was diluted with H₂O (8 mL), and the solid formed was collected by filtration, washed with water (5 mL×3) and dried in and oven to afford A-10 (650.00 mg, 1.50 mmol) as a solid. ¹H NMR (400 MHz, DMSO-d₆) δ_{H} 9.24 (d, 1H), 9.08 (d, 1H), 8.65 (dd, 1H), 8.34 (s, 1H), 7.76 (d, 1H), 7.21 (d, 1H), 5.11 (q, 2H). LCMS Rₜ=0.917 min in 1.5 mins chromatography, MS ESI calcd. for C₁₃H₉F₃IN₄O [M+H]⁺ 421.0, found 420.9.

Synthesis of Compound 9: To a mixture of AgF (58.89 mg, 464.14 μmol, 10.07 uL) in DMF (5.0 mL) was added TMSCF₃ (81.23 mg, 571.25 μmol), then the mixture was stirred at 20° C. for 1 hour. Cu (45.38 mg, 714.06 μmol, 5.06 μL) was added, then the mixture was stirred at 20° C. for 15 hours under N₂. 3-iodo-5-[6-(2,2,2-trifluoroethoxy)-3-pyridyl]pyrazolo[1,5-a]pyrimidine (150.00 mg, 357.03 μmol) was then added, and the mixture was stirred at 20° C. for 2 hours and at 90° C. for 16 hours. The mixture was then poured into water (10 mL) and extracted with EtOAc (10 mL×2). The combined organic phase was washed with brine (10 mL), dried over Na₂SO₄, filtered and concentrated to give the crude product, which was purified by Prep-HPLC to afford Compound 9 (17.93 mg) as a solid. ¹H NMR (400 MHz, CDCl₃) δ_{H} 8.91 (d, 1H), 8.77 (d, 1H), 8.56 (dd, 1H), 8.33 (s, 1H), 7.43 (d, 1H), 7.05 (d, 1H), 4.87 (q, 2H). LCMS Rₜ=1.253 min in 2.0 mins chromatography, MS ESI calcd. for C₁₄H₉F₆N₄O [M+H]⁺ 363.1, found 362.9.

### Example 10: Synthesis of Compound 10

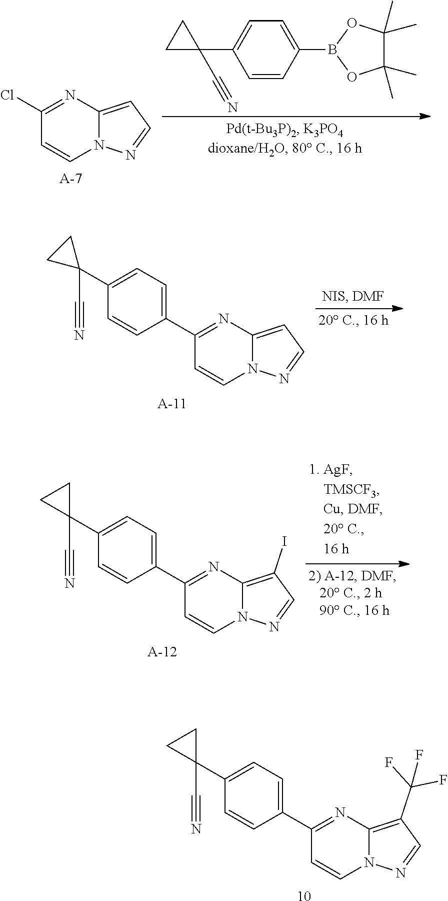

Synthesis of A-11: A mixture of A-7 (300.0 mg, 1.95 mmol), 1-[4-(4,4,5,5-tetramethyl-1,3,2-dioxaborolan-2-yl)phenyl]cyclopropanecarbonitrile (629.81 mg, 2.34 mmol), Pd(t-Bu₃P)₂ (149.48 mg, 292.50 μmol) and K₃PO₄ (827.85 mg, 3.90 mmol) in dioxane (10 mL) and H₂O (3.90 mL) was stirred at 80° C. for 16 hours. The mixture was concentrated to give the crude product, which was purified by silica gel (EtOAc in PE=10% to 20% to 100%) to afford A-11 (600.00 mg, 1.86 mmol) as a solid. ¹H NMR (400 MHz, DMSO-d₆) δ_{H} 9.19 (dd, 1H), 8.30-8.17 (m, 3H), 7.66 (d, 1H), 7.49 (dd, 2H), 6.76 (d, 1H), 1.91-1.80 (m, 2H), 1.68-1.58 (m, 2H). LCMS Rₜ=0.762 min in 1.5 mins chromatography, MS ESI calcd. for C₁₆H₁₃N₄ [M+H]⁺ 261.1, found 260.9.

Synthesis of A-12: To a solution of A-11 (600.00 mg, 2.31 mmol) in DMF (8.0 mL) was added NIS (623.64 mg, 2.77 mmol), and the mixture was stirred at 20° C. for 16 hours. The mixture was then diluted with H₂O (8 mL), and the solid formed was collected by filtration, washed with water (5 mL×3) and dried in an oven to afford A-12 (590.00 mg, 1.53 mmol) as a solid. ¹H NMR (400 MHz, CDCl₃) δ_{H} 8.68 (d, 1H), 8.21-8.13 (m, 3H), 7.45 (d, 2H), 7.31 (d, 1H), 1.87-1.82 (m, 2H), 1.55-1.48 (m, 2H). LCMS Rₜ=0.862 min in 1.5 mins chromatography, MS ESI calcd. for C₁₆H₁₂IN₄ [M+H]⁺ 387.0, found 386.9.

Synthesis of Compound 10: To a mixture of AgF (64.06 mg, 504.93 μmol in DMF (5.0 mL) was added TMSCF₃ (88.37 mg, 621.46 μmol), and the mixture was stirred at 20° C. for 1 hour. Cu (49.37 mg, 776.82 μmol) was added, and the mixture was stirred at 20° C. for 15 hours under N₂. 1-[4-(3-iodopyrazolo[1,5-a]pyrimidin-5-yl)phenyl]cyclopropanecarbonitrile (150.00 mg, 388.41 μmol) was added, then the mixture was stirred at 20° C. for 2 hours and at 90° C. for 16 hours. The mixture was then poured into water (10 mL) and extracted with EtOAc (10 mL×2). The combined organic phase was washed with brine (10 mL), dried over Na₂SO₄, filtered and concentrated to give the crude product, which was purified by Prep-HPLC to afford Compound 10 (31.04 mg) as a solid. ¹H NMR (400 MHz, CDCl₃) δ_{H} 8.77 (d, 1H), 8.33 (s, 1H), 8.18 (d, 2H), 7.49-7.43 (m, 3H), 1.88-1.83 (m, 2H), 1.54-1.50 (m, 2H). LCMS Rₜ=1.173 mins in 2.0 mins chromatography, MS ESI calcd. for C₁₇H₁₂F₃N₄ [M+H]⁺ 329.1, found 328.9.

### Example 11: Synthesis of Compound 11

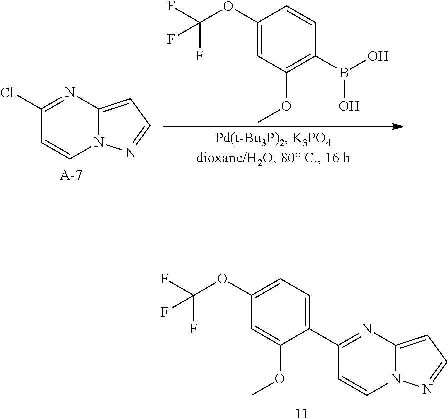

A mixture of A-7 (50.00 mg, 325.58 μmol), [2-methoxy-4-(trifluoromethoxy)phenyl]boronic acid (76.82 mg, 325.58 μmol), Pd(t-Bu₃P)₂ (33.28 mg, 65.12 mol) and K₃PO₄ (138.22 mg, 651.17 μmol) in dioxane (2.0 mL) and H₂O (0.2 mL) was stirred at 80° C. for 16 hours. The mixture was then concentrated to give the crude product, which was purified by Prep-HPLC afford Compound 11 (56.69 mg) as a solid. ¹H NMR (400 MHz, CDCl₃) δ_{H} 8.66 (d, 1H), 8.14 (d, 1H), 7.96 (d, 1H), 7.41 (d, 1H), 7.00 (d, 1H), 6.88 (s, 1H), 6.72 (d, 1H), 3.93 (s, 3H). LCMS Rₜ=1.295 min in 2.0 min chromatography, MS ESI calcd. for C₁₄H₁₁F₃N₃O₂ [M+H]⁺ 310.1, found 309.9.

### Example 12: Synthesis of Compound 12

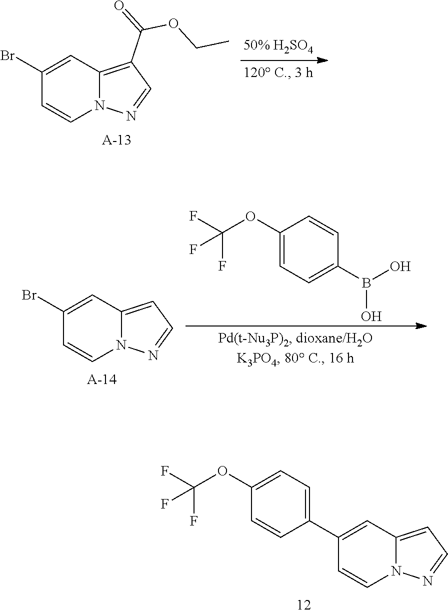

Synthesis of A-14: A colorless mixture of A-13 (900.0 mg, 3.34 mmol) in H₂SO₄ (50%, 10.0 mL) was stirred at 120° C. for 3 hours. The mixture was diluted with ice-water, neutralized with solid Na₂CO₃ to pH=7, and extracted with DCM (50 mL×2). The combined organic layers were washed with brine (10 mL), dried over Na₂SO₄, filtered and concentrated to afford A-14 (550.00 mg, 2.79 mmol) as an oil. ¹H NMR (400 MHz, CDCl₃) δ_{H} 8.38-8.32 (m, 1H), 7.95 (d, 1H), 7.72 (d, 1H), 6.83 (dd, 1H), 6.47 (d, 1H).

Synthesis of Compound 12: A mixture of A-14 (300.00 mg, 1.52 mmol), [4-(trifluoromethoxy)phenyl]boronic acid (344.31 mg, 1.67 mmol), K₃PO₄ (645.30 mg, 3.04 mmol) and Pd(t-Bu₃P)₂ (155.36 mg, 304.00 μmol) in dioxane (20.00 mL) and H₂O (2.0 mL) was stirred under N₂ at 80° C. for 16 hours. The mixture was diluted with EtOAc (20 m), filtered through silica gel, and eluted with EtOAc (10 mL), and the filtrate was concentrated to give the crude product which was purified by Prep-HPLC to give Compound 12 as a solid. ¹H NMR (400 MHz, CDCl₃) δ_{H} 8.54 (d, 1H), 8.08-7.90 (m, 1H), 7.78-7.62 (m, 3H), 7.34 (d, 2H), 6.99 (dd, 1H), 6.69-6.50 (m, 1H). LCMS R, =1.209 min in 2.0 min chromatography, MS ESI calcd. for C₁₄H₁₀F₃N₂O [M+H]⁺ 279.1, found 278.9.

### Example 13: Synthesis of Compound 13

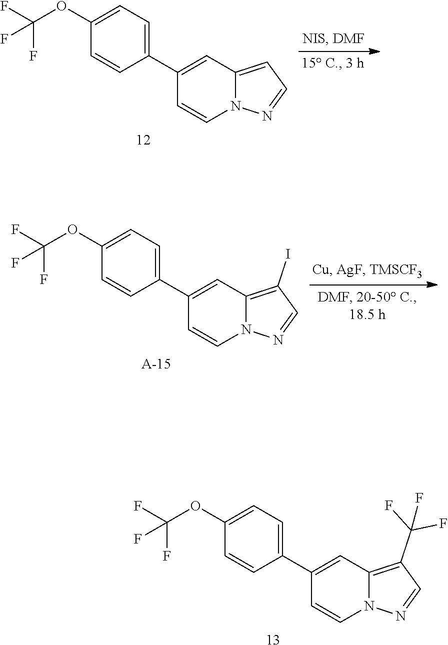 [SMILES: FC(F)(F)Oc1ccc(-c2ccn3nccc3c2)cc1 ; "12" ; FC(F)(F)Oc1ccc(-c2ccn3ncc(I)c3c2)cc1 ; "A-15" ; FC(F)(F)Oc1ccc(-c2ccn3ncc(C(F)(F)F)c3c2)cc1 ; "13"]

Synthesis of A-15: To a solution of Compound 12 (350.00 mg, 1.26 mmol) in DMF (10.0 mL) was added NIS (340.17 mg, 1.51 mmol), and the mixture was stirred at 15° C. for 3 hours. The mixture was diluted with NH₄Cl (30 mL) and extracted with EtOAc (20 mL×2). The combined organic phases were washed with brine (10 mL), dried over Na₂SO₄ and concentrated to give the crude product, which was purified by silica gel (PE:EtOAc=50:1 to 10:1) to afford A-15 (250.00 mg, 618.61 μmol) as a solid. ¹H NMR (400 MHz, CDCl₃) δ_{H} 8.52 (d, 1H), 8.00 (s, 1H), 7.74-7.67 (m, 2H), 7.61 (d, 1H), 7.36 (d, 2H), 7.03 (dd, 1H).

Synthesis of Compound 13: To a mixture of AgF (28.57 mg, 225.17 μmol) in DMF (1.5 mL) was added TMSCF₃ (39.41 mg, 277.14 μmol), then the mixture was stirred at 20° C. for 0.5 hour. Cu (22.02 mg, 346.42 μmol) was added, then the mixture was stirred at 20° C. for 4 hours under N₂. To the mixture was added 3-iodo-5-[4-(trifluoromethoxy)phenyl]pyrazolo[1,5-a]pyridine (70.00 mg, 173.21 μmol), then the mixture was stirred at 20° C. for 2 hours and at 50° C. for 12 hours. The mixture was then diluted with H₂O (10 mL) and extracted with EtOAc (30 mL×2). The combined organic phase was washed with water (10 mL×2) and brine (10 mL), dried over Na₂SO₄, filtered and concentrated to give the crude product, which was purified by Prep-TLC (EtOAc/PET=⅕) to afford Compound 13 (18.53 mg) as a solid. H NMR (400 MHz, CDCl₃) δ_{H} 8.59 (d, 1H), 8.19 (s, 1H), 7.83 (s, 1H), 7.70 (d, 2H), 7.37 (d, 2H), 7.17 (dd, 1H). LCMS Rₜ=1.333 min in 2.0 min chromatography, MS ESI calcd. for C₁₅H₉F₆N₂O [M+H]⁺ 347.1, found 347.1.

### Example 14: Synthesis of Compound 14

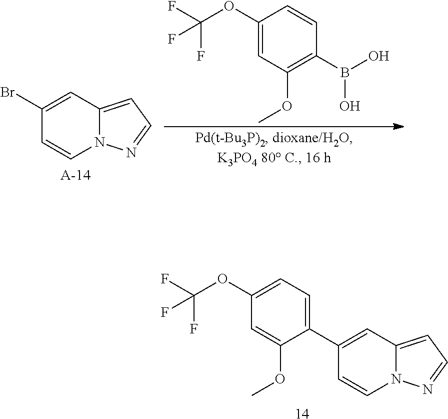

A mixture of A-14 (50.00 mg, 253.77 μmol), [2-methoxy-4-(trifluoromethoxy)phenyl]boronic acid (65.86 mg, 279.15 μmol), K₃PO₄ (107.73 mg, 507.54 μmol) and Pd(t-Bu₃P)₂ (25.94 mg, 50.75 μmol) in dioxane (2.0 mL) and H₂O (0.2 mL) was stirred under N₂ at 80° C. for 16 hours. The mixture was diluted with EtOAc (5 mL), filtered through silica gel, eluted with EtOAc (5 mL) and concentrated to give the crude product, which was purified by Prep-HPLC to afford Compound 14 (19.13 mg) as a solid. ¹H NMR (400 MHz, CDCl₃) δ_{H} 8.48 (d, 1H), 7.97 (d, 1H), 7.64 (d, 1H), 7.39 (d, 1H), 6.98-6.92 (m, 2H), 6.86 (s, 1H), 6.55 (d, 1H), 3.87 (s, 3H). LCMS Rₜ=1.221 min in 2.0 min chromatography, MS ESI calcd. for C₁₅H₁₂F₃N₂O₂ [M+H]⁺ 309.1, found 308.9.

### Example 15: Synthesis of Compound 15

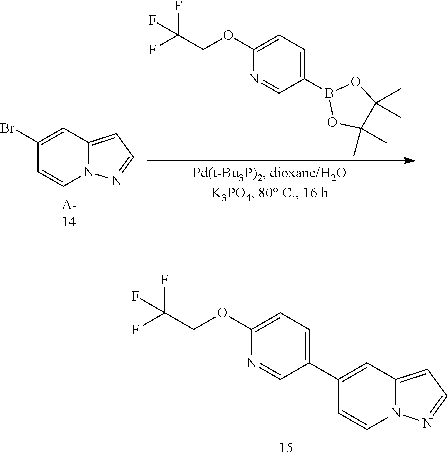

A mixture of A-14 (50.00 mg, 253.77 mol), 5-(4,4,5,5-tetramethyl-1,3,2-dioxaborolan-2-yl)-2-(2,2,2-trifluoroethoxy)pyridine (84.60 mg, 279.15 μmol), K₃PO₄ (107.73 mg, 507.54 mol) and Pd(t-Bu₃P)₂ (25.94 mg, 50.75 μmol) in dioxane (2.0 mL) and H₂O (0.2 mL) was stirred under N₂ at 80° C. for 16 hours. The mixture was diluted with EtOAc (5 mL), filtered through silica gel, and eluted with EtOAc (5 mL). The filtrate was concentrated to give the crude product, which was purified by Prep-HPLC to give Compound 15 (14.79 mg) as a solid. ¹H NMR (400 MHz, CDCl₃) δ_{H} 8.55 (d, 1H), 8.45-8.41 (m, 1H), 8.00 (d, 1H), 7.92 (dd, 1H), 7.68 (s, 1H), 6.99 (d, 1H), 6.95 (dd, 1H), 6.62-6.57 (m, 1H), 4.84 (q, 2H). LCMS Rₜ=1.295 min in 2.0 min chromatography, MS ESI calcd. for C₁₄H₁₁F₃N₃O [M+H]⁺ 294.1, found 293.9.

### Example 16: Synthesis of Compound 16

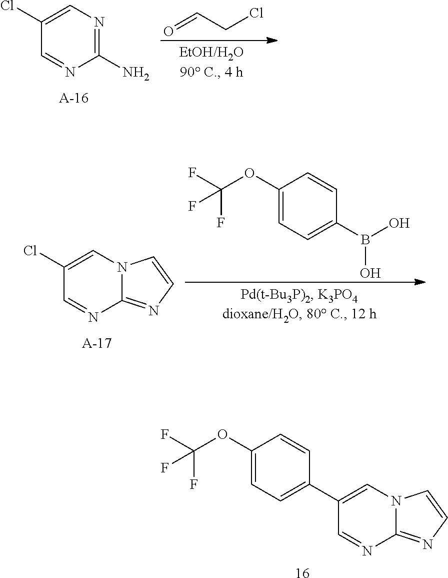

Synthesis of A-17: A mixture of A-16 (2.00 g, 15.44 mmol) and 2-chloroacetaldehyde (15.15 g, 77.20 mmol, 12.42 mL, 40% purity) in EtOH (100.0 mL) was stirred at 90° C. for 4 hours. The mixture was concentrated to a residue that was diluted with H₂O (50 mL), basified with Na₂CO₃ (solid) to pH ˜8, and extracted with DCM (100 mL×3). The combined organic phase was washed with brine (50 mL), dried over Na₂SO₄, filtered and concentrated to give the crude product, which was triturated from PET (15 mL) and dried to afford A-17 (2.05 g, 13.35 mmol) as a solid. ¹H NMR (400 MHz, CDCl₃) δ_{H} 8.59-8.44 (m, 2H), 7.87 (d, 1H), 7.56 (d, 1H).

Synthesis of Compound 16: A mixture of A-17 (100.0 mg, 651.17 μmol), [4-(trifluoromethoxy)phenyl]boronic acid (201.14 mg, 976.75 μmol), K₃PO₄ (276.45 mg, 1.30 mmol) and Pd(t-Bu₃P)₂ (33.28 mg, 65.12 μmol) in dioxane (5.0 mL) and H₂O (1.3 mL) was stirred at 80° C. for 12 hours. The mixture was then concentrated to give the crude product, which was purified by Prep-HPLC to afford Compound 16 (16.09 mg) as a solid. ¹H NMR (400 MHz, CDCl₃) δ_{H} 8.79 (d, 1H), 8.58 (d, 1H), 7.89 (d, 1H), 7.66-7.57 (m, 3H), 7.39 (d, 2H). LCMS Rₜ=0.917 min in 2.0 mins chromatography, MS ESI calcd. for C₁₃H₉F₃N₃O [M+H]⁺ 280.1, found 279.9.

### Example 17: Synthesis of Compound 17

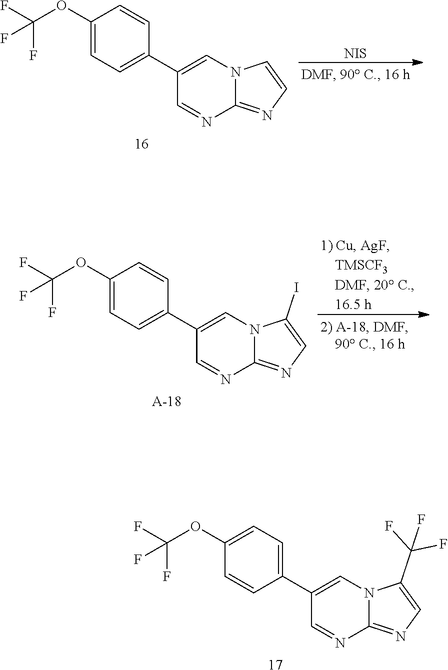

Synthesis of A-18: To a solution of Compound 16 (500.00 mg, 1.79 mmol) in DMF (7.0 mL) was added NIS (604.07 mg, 2.69 mmol), and the mixture was stirred at 90° C. for 16 hours. To the mixture was added H₂O (20 mL) and the solid formed was collected by filtration, washed with water (10 mL×3) and dried an in oven to give A-18 (700.0 mg) as a solid. ¹H NMR (400 MHz, CDCl₃) δ_{H} 8.75 (d, 1H), 8.51 (d, 1H), 7.97 (s, 1H), 7.67 (d, 2H), 7.42 (d, 2H). LCMS Rₜ=0.795 min in 1.5 mins chromatography, MS ESI calcd. for C₁₃H₈F₃IN₃O [M+H]⁺ 406.0, found 405.8.

Synthesis of Compound 17: To a mixture of AgF (28.50 mg, 224.63 μmol) in DMF (3.0 mL) was added TMSCF₃ (39.31 mg, 276.47 μmol), then the mixture was stirred at 20° C. for 0.5 hours. To the mixture was added Cu (21.96 mg, 345.59 umol), then the mixture was stirred at 20° C. for 16 hours under N₂. A-18 (70.00 mg, 172.79 μmol) was then added, then the mixture was stirred at 90° C. for 16 hours. The mixture was then diluted with H₂O (10 mL) and extracted with EtOAc (20 mL×2). The combined organic phase was washed with brine (10 mL), dried over Na₂SO₄, filtered and concentrated to give the crude product, which was purified by Prep-HPLC to give Compound 17 (10.39 mg) as a solid. ¹H NMR (400 MHz, CDCl₃) δ_{H} 8.96 (d, 1H), 8.61 (d, 1H), 8.21 (s, 1H), 7.64 (m, 2H), 7.43 (d, 2H). LCMS Rₜ=1.158 min in 2.0 mins chromatography, MS ESI calcd. for C₁₄H₈F₆N₃O [M+H]⁺ 348.0, found 347.9.

### Example 18: Synthesis of Compound 18

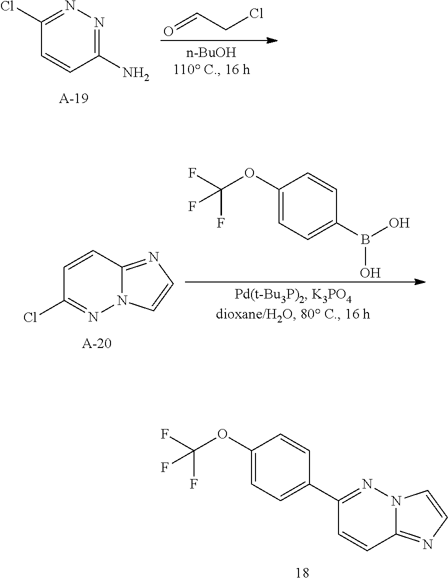

Synthesis of A-20: A mixture of A-19 (4.00 g, 30.88 mmol) and 2-chloroacetaldehyde (6.67 g, 33.97 mmol, 5.46 mL, 40% purity) in n-BuOH (100.0 mL) was stirred at 110° C. for 16 hours. The mixture was concentrated to a residue, which was purified by silica gel (PE:EtOAc=1:1 to EtOAc) to afford A-20 (2.00 g, 13.02 mmol) as a solid. ¹H NMR (400 MHz, CDCl₃) δ_{H} 7.98-7.86 (m, 2H), 7.79 (d, 1H), 7.05 (d, 1H).

Synthesis of Compound 18: A mixture of A-20 (100.00 mg, 651.17 mol), [4-(trifluoromethoxy)phenyl]boronic acid (201.14 mg, 976.75 μmol), Pd(t-Bu₃P)₂ (33.28 mg, 65.12 mol) and K₃PO₄ (276.45 mg, 1.30 mmol) in dioxane (8.0 mL) and H₂O (1.5 mL) was stirred at 80° C. for 16 hours. The mixture was concentrated to give the crude product, which was purified by Prep-HPLC to afford Compound 18 (104.47 mg) as a solid. ¹H NMR (400 MHz, CDCl₃) δ_{H} 8.08-7.98 (m, 4H), 7.82 (d, 1H), 7.47 (d, 1H), 7.38 (d, 2H). LCMS Rₜ=0.919 min in 2.0 min chromatography, MS ESI calcd. for C₁₃H₉F₃N₃O [M+H]⁺ 280.1, found 279.9.

### Example 19: Synthesis of Compound 19

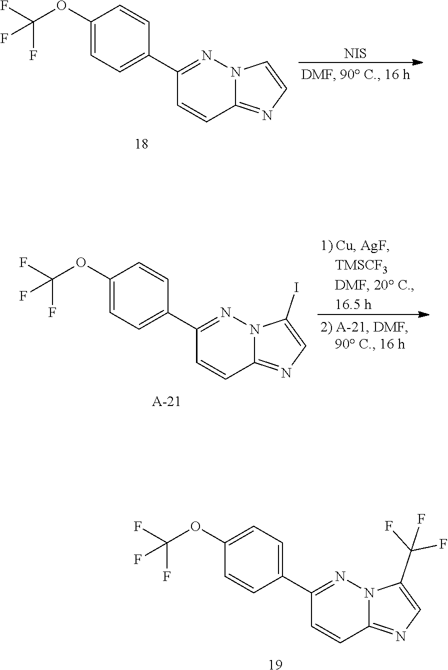

Synthesis of A-21: A mixture of Compound 18 (340.0 mg, 1.22 mmol), NIS (328.74 mg, 1.46 mmol) in DMF (10.0 mL) was stirred at 90° C. for 16 hours. To the mixture was added sat.NaHCO₃ (20 mL), and the mixture was stirred for 1 hour. The solid was collected by filtration and dried in an oven to afford A-21 (430.00 mg) as a solid. ¹H NMR (400 MHz, CDCl₃) δ_{H} 8.15-8.07 (m, 2H), 8.00 (d, 1H), 7.89 (s, 1H), 7.53 (d, 1H), 7.40 (d, 2H).

Synthesis of Compound 19: To a mixture of AgF (61.07 mg, 481.35 μmol) in DMF (3.0 mL) was added TMSCF₃ (84.24 mg, 592.43 μmol), then the mixture was stirred at 20° C. for 0.5 hours. Cu (47.06 mg, 740.54 μmol) was added, then the mixture was stirred at 20° C. for 16 hours under N₂. To the mixture was added A-21 (150.00 mg, 370.27 μmol, 1.00 eq), then the mixture was stirred at 90° C. for 16 hours. The mixture was diluted with H₂O (10 mL) and extracted with EtOAc (20 mL×2). The combined organic phase was washed with water (10 mL×2) and brine (10 mL), dried over Na₂SO₄, filtered and concentrated to give the crude product, which was purified by Prep-HPLC to afford Compound 19 (71.22 mg) as a solid. ¹H NMR (400 MHz, CDCl₃) δ_{H} 8.17-8.02 (m, 4H), 7.68 (d, 1H), 7.40 (d, 2H). LCMS Rₜ=1.258 min in 2.0 min chromatography, MS ESI calcd. for C₁₄H₈F₆N₃O [M+H]⁺ 348.0, found 347.9.

### Example 20: Synthesis of Compound 20

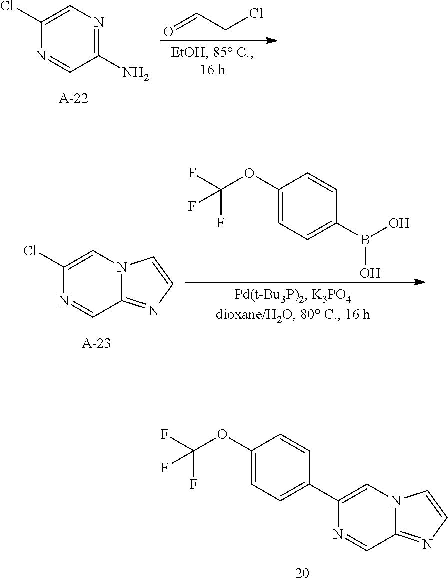

Synthesis of A-23: A mixture of A-22 (2.50 g, 19.30 mmol) and 2-chloroacetaldehyde (18.94 g, 96.50 mmol, 15.52 mL, 40% purity) in EtOH (15.0 mL) was stirred at 85° C. for 16 hours. The mixture was concentrated to a residue, which was diluted with H₂O (100 m), basified with Na₂CO₃ (solid) to pH ˜8, and extracted with EtOAc (300 mL×2). The combined organic phase was washed with brine (100 mL), dried over Na₂SO₄, filtered and concentrated to give the crude product, which was triturated from PET/EtOAc (1/1, 15 mL) and dried in an oven to afford A-23 (1.22 g, 41% yield) as a solid. ¹H NMR (400 MHz, CDCl₃) δ_{H} 8.94 (s, 1H), 8.20 (d, 1H), 7.88 (s, 1H), 7.73 (s, 1H).

Synthesis of Compound 20: A mixture of [4-(trifluoromethoxy)phenyl]boronic acid (201.14 mg, 976.75 μmol), A-23 (100.00 mg, 651.17 μmol), Pd(t-Bu₃P)₂ (33.28 mg, 65.12 mol) and K₃PO₄ (276.45 mg, 1.30 mmol) in dioxane (8.0 mL) and H₂O (1.5 mL) was stirred at 80° C. for 16 hours. The mixture was concentrated to give the crude product, which was purified by Prep-HPLC to afford Compound 20 (46.70 mg) as a solid. ¹H NMR (400 MHz, CDCl₃) δ_{H} 9.20 (d, 1H), 8.47 (d, 1H), 8.03-7.92 (m, 2H), 7.86 (d, 1H), 7.77 (s, 1H), 7.35 (d, 2H). LCMS Rₜ=0.956 min in 2.0 min chromatography, MS ESI calcd. for C₁₃H₉F₃N₃O [M+H]⁺ 280.1, found 279.9.

### Example 21: Synthesis of Compound 21

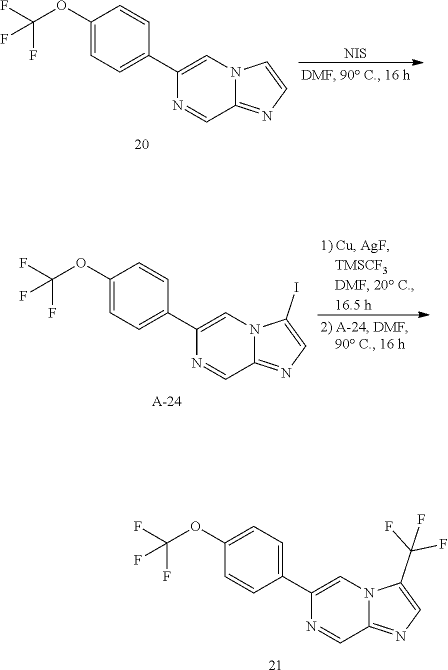

Synthesis of A-24: To a mixture of Compound 20 (350.0 mg, 1.25 mmol) in DMF (10.0 mL) was added NIS (337.47 mg, 1.50 mmol), and the mixture was heated to 90° C. and stirred for 16 hours. To the mixture was added water (30 mL), and the solid was collected by filtration and dried in an oven to afford A-23 (370.0 mg) as a solid. ¹H NMR (400 MHz, CDCl₃) δ_{H} 9.08 (d, 1H), 8.39 (d, 1H), 8.04 (d, 2H), 7.91 (s, 1H), 7.38 (d, 2H)

Synthesis of Compound 21: To a mixture of AgF (40.71 mg, 320.90 μmol) in DMF (3.0 mL) was added TMSCF₃ (56.16 mg, 394.96 μmol), then the mixture was stirred at 20° C. for 0.5 hour. Cu (31.37 mg, 493.70 μmol) was added. To the mixture was added A-23 (100.00 mg, 246.85 μmol), then the mixture was stirred at 90° C. for 16 hours. The mixture was then diluted with H₂O (10 mL) and extracted with EtOAc (20 mL×2). The combined organic phase was washed with water (10 mL×2) and brine (10 mL), dried over Na₂SO₄, filtered and concentrated to give the crude product, which was purified by Prep-HPLC to afford Compound 21 (25.17 mg) as a solid. ¹H NMR (400 MHz, CDCl₃) δ_{H} 9.32 (d, 1H), 8.47 (s, 1H), 8.14 (s, 1H), 8.01 (d, 2H), 7.39 (d, 2H). LCMS Rₜ=1.227 min in 2.0 min chromatography, MS ESI calcd. for C₁₄H₈F₆N₃O [M+H]⁺ 348.0, found 347.9.

### Example 22: Synthesis of Compound 22

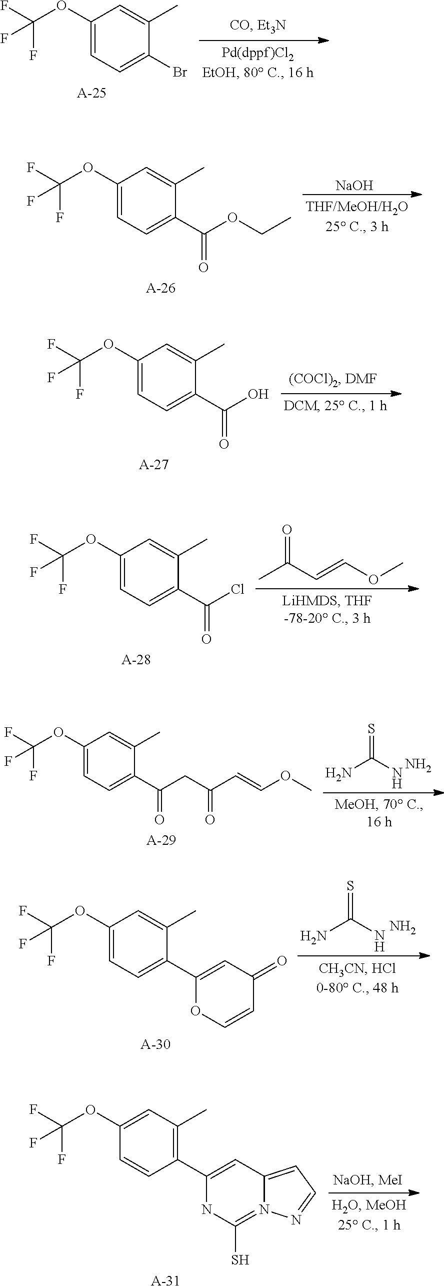

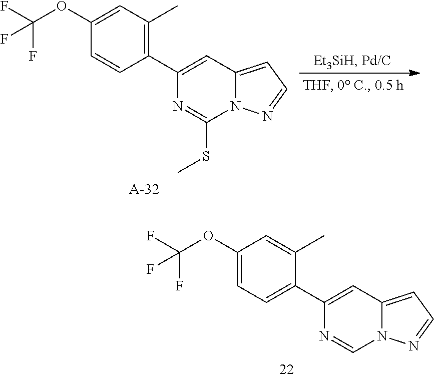

A mixture of 1-bromo-2-methyl-4-(trifluoromethoxy)benzene (3 g, 11.76 mmol), Et₃N (4.88 mL, 35.29 mmol) and Pd(dppf)Cl₂ (860.72 mg, 1.18 mmol) in ethanol (50 mL) was stirred at 80° C. under CO (50 psi), then the mixture was stirred at 80° C. for 16 hours. After cooled to r.t., the mixture was concentrated to give the crude product. The crude product was purified by flash chromatography on silica gel (EtOAc in PE=0% to 1% to 2%) to give the product of ethyl 2-methyl-4-(trifluoromethoxy)benzoate (2500 mg, 86% yield) as oil ¹H NMR (400 MHz, CDCl₃) δ_{H} 8.02-7.92 (m, 1H), 7.13-7.03 (m, 2H), 4.37 (q, 2H), 2.63 (s, 3H), 1.40 (t, 3H).

To a mixture of ethyl 2-methyl-4-(trifluoromethoxy)benzoate (2.5 g, 10.07 mmol) in THF (20 mL), water (20 mL) and methanol (10 mL) was added NaOH (2 g, 50 mmol), then the mixture was stirred at 25° C. for 3 hours. The mixture was evaporated to remove most of the THF and MeOH, the residue was acidified with 6 N HCl to pH ˜2. The solid formed was collected by filtration, washed with water (5 mL×2) and dried in an oven to give the product of 2-methyl-4-(trifluoromethoxy)benzoic acid (2200 mg, 99% yield) as a solid. ¹H NMR (400 MHz, DMSO-d₆) δ_{H} 7.93 (d, 1H), 7.31 (s, 1H), 7.27 (d, 1H), 2.55 (s, 3H).

To a mixture of 2-methyl-4-(trifluoromethoxy)benzoic acid (1.3 g, 5.91 mmol) in DCM (30 mL) was added (COCl)₂ (899.44 mg, 7.09 mmol) and DMF (86.32 mg, 1.18 mmol) at 0° C., then the mixture was stirred at 25° C. for 1 hour to give a solution. The mixture was concentrated to give the crude product of 2-methyl-4-(trifluoromethoxy)benzoyl chloride (1400 mg, 99% yield) as oil, which was used directly without any further purification.

To a mixture of LiHMDS (20.54 mL, 20.54 mmol) in THF (40 mL) was added 4-methoxybut-3-en-2-one (1.29 g, 12.91 mmol) at −78° C. under N₂, then the mixture was stirred at −78° C. for 1 hour. To the mixture was added a solution of 2-methyl-4-(trifluoromethoxy)benzoyl chloride (1.4 g, 5.87 mmol) in THF (30 mL) at −78° C., then the mixture was allowed to warmed to 20° C. slowly and stirred for 2 hours. The mixture was quenched with sat. NH₄Cl (60 mL), then the mixture was extracted with EtOAc (150 mL×2). The combined organic phase was washed with water (30 mL) and brine (30 mL), dried over anhydrous Na₂SO₄, filtered and concentrated to give the crude product of 5-methoxy-1-[2-methyl-4-(trifluoromethoxy)phenyl]pent-4-ene-1,3-dione (1700 mg, 32% purity) as oil. The crude product was used next step without further purification. LCMS Rₜ=0.82 min in 1.5 min chromatography, MS ESI calcd. for C₁₄H₁₄F₃O₄ [M+H]⁺ 303.1, found 303.0.

A mixture of 5-methoxy-1-[2-methyl-4-(trifluoromethoxy)phenyl]pent-4-ene-1,3-dione (1.7 g, 5.62 mmol) and aminothiourea (1.28 g, 14.06 mmol) in Methanol (50 mL) was stirred at 70° C. for 16 hours. After cooling to room temperature, the mixture was concentrated to give the crude product. The crude product was purified by flash chromatography on silica gel (EtOAc in PE=0% to 30% to 60%) to give the product of 2-[2-methyl-4-(trifluoromethoxy)phenyl]pyran-4-one (620 mg, 39% yield) as a solid. ¹H NMR (400 MHz, CDCl₃) δ_{H} 7.85 (d, 1H), 7.46 (d, 1H), 7.16 (s, 2H), 6.48 (d, 1H), 6.44 (dd, 1H), 2.44 (s, 3H).

To a mixture of 2-[2-methyl-4-(trifluoromethoxy)phenyl]pyran-4-one (620 mg, 2.29 mmol) and aminothiourea (313.69 mg, 3.44 mmol) in MeCN (60 mL) was added conc. HCl (418.76 mg, 11.47 mmol) at 0° C. under N₂, then the mixture was stirred at 0° C. for 15 minutes, 20° C. for 15 minutes and 80° C. for 48 hours to give a suspension. After cooling to room temperature, the mixture was concentrated to give the crude product of 5-[2-methyl-4-(trifluoromethoxy)phenyl]pyrazolo[1,5-c]pyrimidine-7-thiol (746 mg, 2.29 mmol, crude) as oil which was used directly without any further purification. LCMS Rₜ=0.78 min in 1.5 min chromatography, MS ESI calcd. for C₁₄H₁₁F₃N₃OS [M+H]⁺ 326.1, found 325.9.

To a mixture of 5-[2-methyl-4-(trifluoromethoxy)phenyl]pyrazolo[1,5-c]pyrimidine-7-thiol (746 mg, 2.29 mmol) and NaOH (458.64 mg, 11.47 mmol) in Methanol (30 mL) and Water (15 mL) was added iodomethane (1627.48 mg, 11.47 mmol) at 0° C., then the mixture was stirred at 25° C. for 1 hour to give a mixture. The mixture was acidified with 1 N HCl to pH ˜6, then the mixture was extracted with EtOAc (30 mL×2). The combined organic phase was washed with brine (15 mL), dried over Na₂SO₄, filtered and concentrated to give the crude product. The crude product was purified by flash chromatography on silica gel (EtOAc in PE=0% to 5% to 15%) to give the product of 7-methylsulfanyl-5-[2-methyl-4-(trifluoromethoxy)phenyl]pyrazolo[1,5-c]pyrimidine (120 mg, 15% yield) as oil. ¹H NMR (400 MHz, CDCl₃) δ_{H} 8.12 (d, 1H), 7.53 (d, 1H), 7.31 (s, 1H), 7.18-7.13 (m, 2H), 6.59 (d, 1H), 2.50 (s, 3H), 2.48 (s, 3H).

7-methylsulfanyl-5-[2-methyl-4-(trifluoromethoxy)phenyl]pyrazolo[1,5-c]pyrimidine (30 mg, 0.09 mmol) in THF (3 mL) was added Pd/C (20 mg, 0.19 mmol) and Et₃SiH (300 mg, 2.58 mmol) at 0° C., then the mixture was stirred at 0° C. for 30 minutes to give a black suspension. The mixture was filtered through Celite, eluted with THF (10 mL×2), the filtrate was concentrated to give the crude product. The crude product was purified by Prep-TLC (silica gel, PE:EtOAc=10:1) to give the product of 5-[2-methyl-4-(trifluoromethoxy)phenyl]pyrazolo[1,5-c]pyrimidine (13.07 mg, 50% yield) as a solid. ¹H NMR (400 MHz, CDCl₃) δ_{H} 9.33 (s, 1H), 8.14 (d, 1H), 7.52-7.48 (m, 2H), 7.18-7.14 (m, 2H), 6.60 (d, 1H), 2.47 (s, 3H). LCMS Rₜ=1.21 min in 2.0 min chromatography, MS ESI calcd. for C₁₄H₁₁F₃N₃O [M+H]⁺ 294.1, found 293.9.

### Example 23: Synthesis of Compound 23

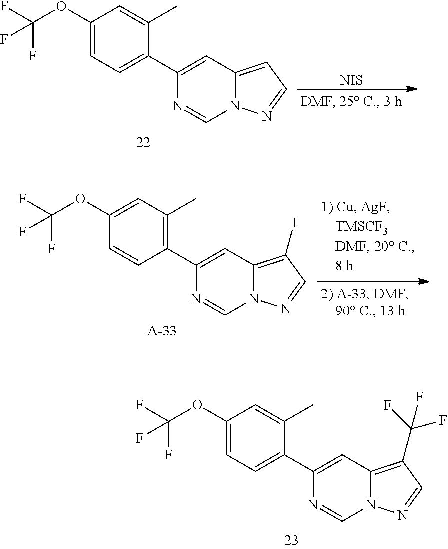

To a solution of 5-[2-methyl-4-(trifluoromethoxy)phenyl]pyrazolo[1,5-c]pyrimidine (50 mg, 0.17 mmol) in DMF (2 mL) was added NIS (57.54 mg, 0.26 mmol). The resulting mixture was stirred at 25° C. for 3 hours to give a solution. Na₂S₂O₃ (30 mg), water (15 mL) and EtOAc (20 mL) were added to the reaction mixture and stirred for 5 minutes. The mixture turned to colorless. After separated, the combined organic phase was washed with brine (10 mL×2), dried over anhydrous Na₂SO₄, filtered and concentrated to give the crude product. The crude product was purified by Prep-TLC (silica gel, PE:EtOAc=10:1) to give the product of 3-iodo-5-[2-methyl-4-(trifluoromethoxy)phenyl]pyrazolo[1,5-c]pyrimidine (50 mg, 70% yield) as a solid. ¹H NMR (400 MHz, CDCl₃) δ_{H} 9.24 (d, 1H), 8.13 (s, 1H), 7.52 (d, 1H), 7.42 (d, 1H), 7.19-7.15 (m, 2H), 2.48 (s, 3H). LCMS Rₜ=1.35 min in 2.0 min chromatography, MS ESI calcd. for C₁₄H₁₀F₃IN₃O [M+H]⁺ 420.0, found 419.9.

To a mixture of AgF (110.18 mg, 0.87 mmol) in DMF (6 mL) was added trimethyl(trifluoromethyl)silane (168.18 mg, 1.18 mmol), then the mixture was stirred at 20° C. for 3 hours. To the mixture was added Cu (88.92 mg, 1.4 mmol), then the mixture was stirred at 20° C. for 5 hours. To the mixture was added 3-iodo-5-[2-methyl-4-(trifluoromethoxy)phenyl]pyrazolo[1,5-c]pyrimidine (50 mg, 0.12 mmol), then the mixture was stirred at 20° C. for 1 hour and 90° C. for 12 hours. The green mixture turned to suspension. After cooling to r.t., the mixture was diluted with H₂O (20 mL), and the mixture was extracted with EtOAc (50 mL×2). The combined organic phase was washed with water (20 mL×2) and brine (20 mL), dried over anhydrous Na₂SO₄, filtered and concentrated to give the crude product. The crude product was purified by Prep-TLC (silica gel, PE:DCM=10:1) to give the product 5-[2-methyl-4-(trifluoromethoxy)phenyl]-3-(trifluoromethyl)pyrazolo[1,5-c]pyrimidine (11.5 mg, 27% yield) as a solid. ¹H NMR (400 MHz, CDCl₃) δ_{H} 9.38 (d, 1H), 8.31 (s, 1H), 7.67 (s, 1H), 7.53 (d, 1H), 7.21-7.16 (m, 2H), 2.48 (s, 3H). LCMS Rₜ=1.49 min in 2.0 min chromatography, MS ESI calcd. for C₁₅H₁₀F₆N₃O [M+H]⁺ 362.1, found 362.0.

### Example 24: Synthesis of Compound 24

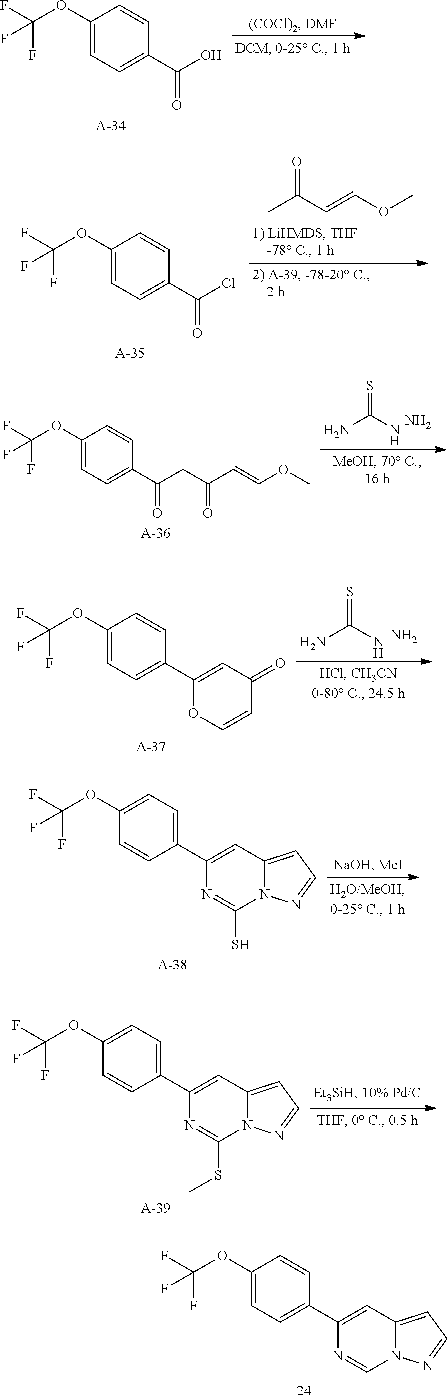

To a mixture of 4-(trifluoromethoxy)benzoic acid (1.5 g, 7.28 mmol) in DCM (30 mL) was added (COCl)₂ (1.11 g, 8.73 mmol) and DMF (106.38 mg, 1.46 mmol) at 0° C., then the mixture was stirred at 25° C. for 1 hour. The mixture was concentrated to give the crude product of 4-(trifluoromethoxy)benzoyl chloride (1600 mg, 98% yield) as oil, which was used directly without any further purification.

To a mixture of LiHMDS (23.38 mL, 23.38 mmol) in THF (40 mL) was added 4-methoxybut-3-en-2-one (1.5 g, 14.98 mmol) at −78° C. under N₂, then the mixture was stirred at −78° C. for 1 hour. To the mixture was added a solution of 4-(trifluoromethoxy)benzoyl chloride (1.5 g, 6.68 mmol) in THF (30 mL) at −78° C., then the mixture was stirred at −78° C. to 20° C. for 2 hours. The mixture was quenched with sat. NH₄Cl (60 mL), then the mixture was extracted with EtOAc (150 mL×2). The combined organic phase was washed with water (30 mL) and brine (30 mL), dried over Na₂SO₄, filtered and concentrated to give the crude product of 5-methoxy-1-[4-(trifluoromethoxy)phenyl]pent-4-ene-1,3-dione (1900 mg, 58% purity) as oil. The crude product was used in next step without further purification. LCMS Rₜ=0.90 min in 1.5 min chromatography, MS ESI calcd. for C₁₃H₁₂F₃O₄ [M]⁺ 289.1, found 288.9.

A mixture of 5-methoxy-1-[4-(trifluoromethoxy)phenyl]pent-4-ene-1,3-dione (1.9 g, 6.59 mmol) and aminothiourea (1.5 g, 16.48 mmol) in Methanol (150 mL) was stirred at 70° C. for 16 hours. After cooling to r.t., the mixture was concentrated to give a crude product which was purified by flash chromatography on silica gel (EtOAc in PE=0% to 30% to 60%) to give the product of 2-[4-(trifluoromethoxy)phenyl]pyran-4-one (920 mg, 52% yield) as a solid. ¹H NMR (400 MHz, CDCl₃) δ_{H} 7.86 (d, 1H), 7.83 (d, 2H), 7.35 (d, 2H), 6.79 (d, 1H), 6.42 (dd, 1H). LCMS Rt=0.76 min in 1.5 min chromatography, MS ESI calcd. for C₁₂H₈F₃O₃ [M+H]⁺ 257.0, found 256.9.

To a mixture of 2-[4-(trifluoromethoxy)phenyl]pyran-4-one (940 mg, 3.67 mmol) and aminothiourea (501.63 mg, 5.5 mmol) in MeCN (80 mL) was added conc. HCl (1.53 mL, 18.35 mmol) at 0° C. under N₂, then the mixture was stirred at 0° C. for 15 minutes, 20° C. for 15 minutes and 80° C. for 24 hours. After cooling to r.t., the mixture was concentrated to give the crude product (1142 mg) as oil, which was used directly without any further purification. LCMS Rt=0.67 min in 1.5 min chromatography, MS ESI calcd. for C₁₃H₉F₃N₃OS [M+H]⁺ 312.0, found 311.9.

To a mixture of 5-[4-(trifluoromethoxy)phenyl]pyrazolo[1,5-c]pyrimidine-7-thiol (1.14 g, 3.67 mmol) and NaOH (733.74 mg, 18.34 mmol) in Methanol (40 mL) and Water (20 mL) was added iodomethane (2.6 g, 18.34 mmol) at 0° C., then the mixture was stirred at 25° C. for 1 hour. The mixture was acidified with 1 N HCl to pH ˜6, then the mixture was extracted with EtOAc (30 mL×2). The combined organic phase was washed with brine (15 mL), dried over Na₂SO₄, filtered and concentrated to give the crude product. The crude product was purified by flash chromatography on silica gel (EtOAc in PE=0% to 5% to 15%) to give the product of 7-methylsulfanyl-5-[4-(trifluoromethoxy)phenyl]pyrazolo[1,5-c]pyrimidine (340 mg, 28% yield) as oil. ¹H NMR (400 MHz, CDCl₃) δ_{H} 8.13 (d, 2H), 8.10 (d, 1H), 7.65 (s, 1H), 7.34 (d, 2H), 6.60 (d, 1H), 2.84 (s, 3H).

To a mixture of 7-methylsulfanyl-5-[4-(trifluoromethoxy)phenyl]pyrazolo[1,5-c]pyrimidine (60 mg, 0.18 mmol) in THF (3 mL) was added 10% Pd/C (40 mg, 0.18 mmol) and Et₃SiH (500 mg, 4.3 mmol) at 0° C., then the mixture was stirred at 0° C. for 30 minutes. The mixture was filtered through Celite, eluted with THF (10 mL×2), the filtrate was concentrated to give the crude product. The crude product was purified by flash chromatography on silica gel (EtOAc in PE=0% to 10% to 20%) to give the product of 5-[4-(trifluoromethoxy)phenyl]pyrazolo[1,5-c]pyrimidine (21.8 mg, 42.% yield) as a solid ¹H NMR (400 MHz, CDCl₃) δ_{H} 9.33 (s, 1H), 8.11 (d, 1H), 8.08 (d, 2H), 7.83 (d, 1H), 7.34 (d, 2H), 6.61 (d, 1H). LCMS Rₜ=1.14 min in 2.0 min chromatography, MS ESI calcd. for C₁₃H₉F₃N₃O [M+H]⁺ 280.1, found 279.9.

### Example 25: Synthesis of Compounds 25a and 25b

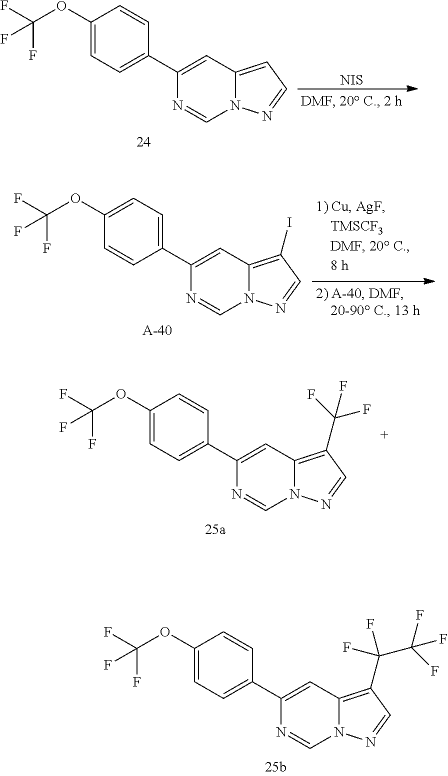

To a mixture of 5-[4-(trifluoromethoxy)phenyl]pyrazolo[1,5-c]pyrimidine (150 mg, 0.54 mmol) in DMF (5 mL) was added NIS (241.72 mg, 1.07 mmol), then the mixture was stirred at 20° C. for 2 hours. The mixture was diluted with H₂O (20 mL), and the mixture was extracted with EtOAc (30 mL×2). The combined organic phase was washed with water (15 mL) and brine (20 mL), dried over Na₂SO₄, filtered and concentrated to give the crude product. The crude product was purified by flash chromatography on silica gel (EtOAc in PE=0% to 5% to 10%) to give 3-iodo-5-[4-(trifluoromethoxy)phenyl]pyrazolo[1,5-c]pyrimidine (170 mg, 0.41 mmol, 77% yield) as a solid. ¹H NMR (400 MHz, DMSO-d₆) δ_{H} 9.62 (d, 1H), 8.38-8.30 (m, 3H), 8.04 (d, 1H), 7.50 (d, 2H). LCMS Rt=0.93 min in 1.5 min chromatography, MS ESI calcd. for C₁₃H₈F₃IN₃O [M+H]⁺ 406.0, found 405.9.

To a mixture of AgF (228 mg, 1.8 mmol) in DMF (10 mL) was added trimethyl(trifluoromethyl)silane (348 mg, 2.45 mmol), then the mixture was stirred at 20° C. for 3 hours. To the mixture was added Cu (184 mg, 2.9 mmol), then the mixture was stirred at 20° C. for 5 hours. To the mixture was added 3-iodo-5-[4-(trifluoromethoxy)phenyl]pyrazolo[1,5-c]pyrimidine (100 mg, 0.25 mmol), then the mixture was stirred at 20° C. for 1 hour and 90° C. for 12 hours. After cooling to r.t., the mixture was diluted with H₂O (20 mL), and the mixture was extracted with EtOAc (50 mL×2). The combined organic phase was washed with water (20 mL×2) and brine (20 m), dried over Na₂SO₄, filtered and concentrated to give the crude product. The crude product was purified by Prep-TLC (silica gel, PE:EtOAc=10:1) to give 5-[4-(trifluoromethoxy)phenyl]-3-(trifluoromethyl)pyrazolo[1,5-c]pyrimidine (33.91 mg, 0.10 mmol, 40% yield) as a solid and 3-(1,1,2,2,2-pentafluoroethyl)-5-[4-(trifluoromethoxy)phenyl]pyrazolo[1,5-c]pyrimidine (13.78 mg, 13.% yield) as a solid. Compound 25a: ¹H NMR (400 MHz, CDCl₃) δ_{H} 9.37 (d, 1H), 8.28 (s, 1H), 8.14 (d, 2H), 7.94 (s, 1H), 7.37 (d, 2H). LCMS Rₜ=1.01 min in 2.0 min chromatography, MS ESI calcd. for C₁₄H₈F₆N₃O [M+H]⁺ 348.1, found 348.0. Compound 25b: ¹H NMR (400 MHz, CDCl₃) δ_{H} 9.38 (d, 1H), 8.25 (s, 1H), 8.12 (d, 2H), 7.91 (s, 1H), 7.37 (d, 2H). LCMS Rₜ=1.09 min in 2.0 min chromatography, MS ESI calcd. for C₁₅H₈F₈N₃O [M+H]⁺ 398.1, found 398.0.

Mobile Phase: 1.5 ML/4 LTFA in water (solvent A) and 0.75 ML/4 LTFA in acetonitrile (solvent B), using the elution gradient 30%-90% (solvent B) over 0.9 minutes and holding at 90% for 0.6 minutes at a flow rate of 1.2 ml/min; Column: Xtimate C18 2.1*30 mm, 3 um; Wavelength: UV 220 nm&254 nm; Column temperature: 50° C.; MS ionization: ESI.

### Example 26: Synthesis of Compound 26

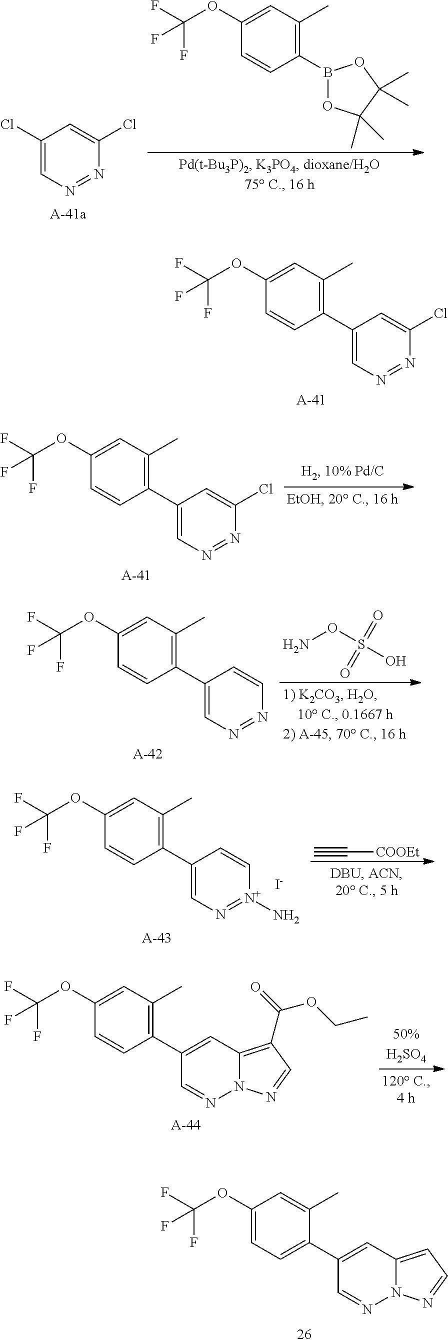

A mixture of 3,5-dichloropyridazine (500 mg, 3.36 mmol), 4,4,5,5-tetramethyl-2-[2-methyl-4-(trifluoromethoxy)phenyl]-1,3,2-dioxaborolane (861.81 mg, 2.85 mmol), Pd(t-Bu₃P)₂ (171.52 mg, 0.34 mmol) and K₃PO₄ (1.43 g, 6.71 mmol) in 1,4-dioxane (30 mL) and water (6 mL) was stirred at 75° C. for 16 hours under N₂. After cooling to r.t., the mixture was concentrated. The residue was diluted with H₂O (30 mL), and the mixture was extracted with EtOAc (50 mL×2). The combined organic phase was washed with water (20 mL) and brine (20 mL), dried over Na₂SO₄, filtered and concentrated to give the crude product. The crude product was purified by silica gel column (EtOAc in PE=0% to 10% to 20%) to give the (510 mg, 1.7066 mmol) as a solid. ¹H NMR (400 MHz, DMSO-d₆) δ_{H} 9.37 (d, 1H), 8.08 (d, 1H), 7.54 (d, 1H), 7.45 (s, 1H), 7.38 (d, 1H), 2.35 (s, 3H). LCMS Rₜ=0.832 min in 1.5 min chromatography, 5-95AB, purity 96.59%, MS ESI calcd. for Cl₂H₉CF₃N₂O [M+H]⁺ 289.0, found 288.9.

To a mixture of 3-chloro-5-[2-methyl-4-(trifluoromethoxy)phenyl]pyridazine (700 mg, 2.06 mmol) in Ethanol (15 mL) was added 10% Pd/C (300 mg), then the mixture was stirred under H₂ (15 psi) at 20° C. for 16 hours. The mixture was filtered through Celite, eluted with MeOH (30 mL×2), and the filtrate was concentrated to aresidue. The residue was diluted with H₂O (20 mL) and sat.NaHCO₃ (20 mL), and the mixture was extracted with EtOAc (50 mL×2). The combined organic phase was washed with water (20 mL) and brine (20 mL), dried over Na₂SO₄, filtered and concentrated to give the crude product. The crude product was purified by flash chromatography on silica gel (EtOAc in PE=20% to 50% to 70%) to give 4-[2-methyl-4-(trifluoromethoxy)phenyl]pyridazine (440 mg, 83% yield) as a solid. ¹H NMR (400 MHz, CDCl₃) δ_{H} 9.28 (d, 1H), 9.22 (d, 1H), 7.45 (dd, 1H), 7.29-7.25 (m, 1H), 7.23-7.17 (m, 2H), 2.34 (s, 3H). LCMS Rt=0.77 min in 1.5 min chromatography, MS ESI calcd. for C₁₂H₁₀F₃N₂O [M+H]⁺ 255.1, found 255.0.

To a mixture of amino hydrogen sulfate (1.07 g, 9.44 mmol) in Water (10 mL) was added K₂CO₃ (652.42 mg, 4.72 mmol) at 10° C., then the mixture was stirred at 10° C. for 10 minutes. To the mixture was added 4-[2-methyl-4-(trifluoromethoxy)phenyl]pyridazine (800 mg, 3.15 mmol), then the mixture was stirred at 70° C. for 16 hours. After cooling to r.t., to the mixture was added KI (783.6 mg, 4.72 mmol). The mixture was concentrated to give crude 4-[2-methyl-4-(trifluoromethoxy)phenyl]pyridazin-1-ium-1-amine iodide (1200 mg, 96% yield) as a solid, which was used next step without further purification. LCMS Rₜ=0.65 min in 1.5 min chromatography, MS ESI calcd. for C₁₂H₁₁F₃N₃O [M-I]⁺270.1, found 269.9.

To a mixture of 4-[2-methyl-4-(trifluoromethoxy)phenyl]pyridazin-1-ium-1-amine iodide (1.2 g, 3.02 mmol) and ethyl prop-2-ynoate (1.19 g, 12.09 mmol) in MeCN (20 mL) was added DBU (1.84 g, 12.09 mmol), then the mixture was stirred at 20° C. for 5 hours. The mixture was diluted with H₂O (30 mL), and the mixture was extracted with EtOAc (50 mL×2). The combined organic phase was washed with water (20 mL×2) and brine (20 mL), dried over Na₂SO₄, filtered and concentrated to give the crude product. The crude product was purified by flash chromatography on silica gel (EtOAc in PE=0% to 20% to 30%) to give ethyl 5-[2-methyl-4-(trifluoromethoxy)phenyl]pyrazolo[1,5-b]pyridazine-3-carboxylate (260 mg, 23% yield) as a solid. ¹H NMR (400 MHz, CDCl₃) δ_{H} 8.79 (d, 1H), 8.61 (s, 1H), 8.43 (d, 1H), 7.59 (d, 1H), 7.45 (s, 1H), 7.38 (d, 1H), 4.34 (q, 2H), 2.38 (s, 3H), 1.33 (t, 3H). LCMS Rₜ=0.894 min in 1.5 min chromatography, MS ESI calcd. for C₁₇H₁₅F₃N₃O₃ [M+H]⁺ 366.1, found 366.0.

A mixture of ethyl 5-[2-methyl-4-(trifluoromethoxy)phenyl]pyrazolo[1,5-b]pyridazine-3-carboxylate (200 mg, 0.55 mmol) in H₂SO₄ (1.63 mL, 30.59 mmol) and water (3 mL) was stirred at 120° C. for 4 hours. After cooling to r.t., the mixture was poured into ice water (20 mL), basified with Na₂CO₃ (solid) to pH ˜9, extracted with EtOAc (30 mL×2). The combined organic phase was washed with brine (10 mL), dried over Na₂SO₄, filtered and concentrated to give crude 5-[2-methyl-4-(trifluoromethoxy)phenyl]pyrazolo[1,5-b]pyridazine (160 mg, 0.37 mmol, 67% yield) as a solid. LCMS Rₜ=0.84 min in 1.5 min chromatography, MS ESI calcd. for C₁₄H₁₁F₃N₃O [M+H]⁺ 294.1, found 293.9.

A part of crude 5-[2-methyl-4-(trifluoromethoxy)phenyl]pyrazolo[1,5-b]pyridazine (40 mg, 0.14 mmol) was purified by Prep-TLC (silica gel, PE:EtOAc=3:1) to give 5-[2-methyl-4-(trifluoromethoxy)phenyl]pyrazolo[1,5-b]pyridazine (10.7 mg, 27% yield) as a solid. H NMR (400 MHz, CDCl₃) δ_{H} 8.26 (d, 1H), 8.10 (d, 1H), 7.86 (d, 1H), 7.31 (d, 1H), 7.23-7.15 (m, 2H), 6.68 (d, 1H), 2.37 (s, 3H). LCMS Rₜ=1.20 min in 2.0 min chromatography, MS ESI calcd. for C₁₄H₁₁F₃N₃O [M+H]⁺ 294.1, found 293.9.

### Example 27: Synthesis of Compound 27

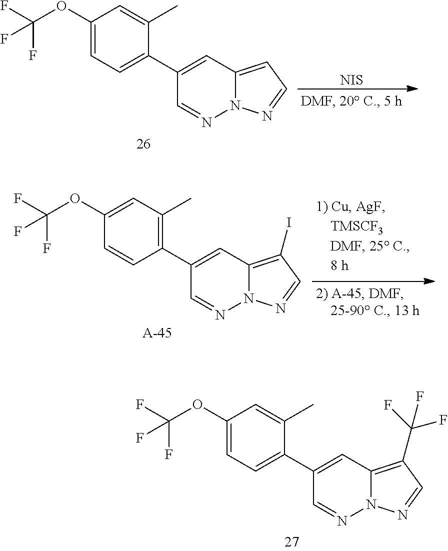

To a mixture of 5-[2-methyl-4-(trifluoromethoxy)phenyl]pyrazolo[1,5-b]pyridazine (110 mg, 0.38 mmol) in DMF (4 mL) was added NIS (168.79 mg, 0.75 mmol), then the mixture was stirred at 20° C. for 5 hours. The mixture was diluted with H₂O (20 m), and the mixture was extracted with EtOAc (30 mL×2). The combined organic phase was washed with water (20 mL) and brine (20 mL), dried over Na₂SO₄, filtered and concentrated to give the crude product. The crude product was purified by flash chromatography on silica gel (EtOAc in PE=0% to 10% to 20%) to give the product of 3-iodo-5-[2-methyl-4-(trifluoromethoxy)phenyl]pyrazolo[1,5-b]pyridazine (110 mg, 66% yield) as a solid. ¹H NMR (400 MHz, DMSO-d₆) δ_{H} 8.57 (d, 1H), 8.29 (s, 1H), 8.04 (d, 1H), 7.56 (d, 1H), 7.42 (s, 1H), 7.35 (d, 1H), 2.36 (s, 3H). LCMS Rₜ=0.92 min in 1.5 min chromatography, MS ESI calcd. for C₁₄H₁₀F₃IN₃O [M+H]⁺ 420.0, found 419.9.

To a mixture of AgF (228.65 mg, 1.8 mmol) in DMF (10 mL) was added trimethyl(trifluoromethyl)silane (347.95 mg, 2.45 mmol), then the mixture was stirred at 25° C. for 3 hours under N₂ in seal-tube. To the mixture was added Cu (183.92 mg, 2.89 mmol), then the mixture was stirred at 25° C. for 5 hours. To the mixture was added 3-iodo-5-[2-methyl-4-(trifluoromethoxy)phenyl]pyrazolo[1,5-b]pyridazine (90 mg, 0.21 mmol), then the mixture was stirred at 25° C. for 1 hours and 90° C. for 12 hours in a 20 mL sealed tube under N₂. After cooling to r.t., the mixture was diluted with H₂O (30 mL), and the mixture was extracted with EtOAc (50 mL×2). The combined organic phase was washed with water (20 mL×2) and brine (20 mL), dried over Na₂SO₄, filtered and concentrated to give the crude product. The crude product was purified by Prep-TLC (silica gel, PE:EtOAc=5:1) to give the product of 5-[2-methyl-4-(trifluoromethoxy)phenyl]-3-(trifluoromethyl)pyrazolo[1,5-b]pyridazine (27.11 mg, 35% yield) as a solid. ¹H NMR (400 MHz, CDCl₃) δ_{H} 8.42 (d, 1H), 8.32 (s, 1H), 8.02 (d, 1H), 7.34 (d, 1H), 7.25-7.19 (m, 2H), 2.38 (s, 3H). LCMS Rₜ=1.34 min in 2.0 min chromatography, MS ESI calcd. for C₁₅H₁₀F₆N₃O [M+H]⁺ 362.1, found 362.0.

### Example 28: Synthesis of Compound 28

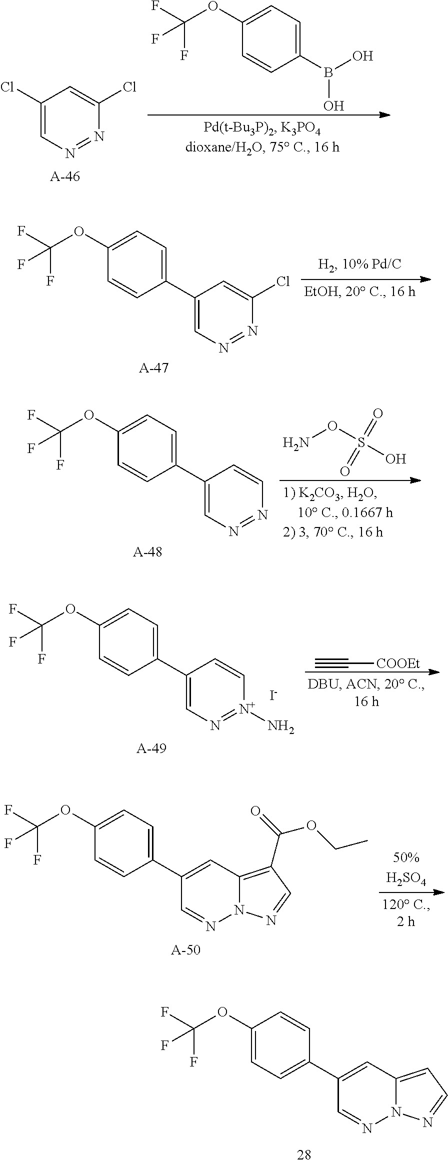

A mixture of 3,5-dichloropyridazine (1.38 g, 9.26 mmol), [4-(trifluoromethoxy)phenyl]boronic acid (1.53 g, 7.41 mmol), Pd(t-Bu₃P)₂ (378.71 mg, 0.74 mmol) and K₃PO₄ (3.93 g, 18.53 mmol) in 1,4-Dioxane (80 mL) and water (20 mL) was stirred at 75° C. for 16 hours under N₂. After cooling to r.t., the mixture was concentrated. The residue was diluted with H₂O (100 mL), and the mixture was extracted with EtOAc (150 mL×2). The combined organic phase was washed with water (50 mL) and brine (50 mL), dried over Na₂SO₄, filtered and concentrated to give the crude product. The crude product was purified by silica gel column (EtOAc in PE=0% to 10% to 20%) to give 3-chloro-5-[4-(trifluoromethoxy)phenyl]pyridazine (1800 mg, 5.3116 mmol, 57% yield) as a solid. LCMS Rₜ=0.800 min in 1.5 min chromatography, MS ESI calcd. for C₁₁H₇CF₃N₂O [M+H]⁺ 275.0, found 274.9.

To a mixture of 3-chloro-5-[4-(trifluoromethoxy)phenyl]pyridazine (1800 mg, 5.31 mmol) in Ethanol (25 mL) was added 10% Pd/C (380 mg), then the mixture was stirred under H₂ (15 psi) at 20° C. for 16 hours. The mixture was filtered through Celite and eluted with MeOH (50 mL×2), and the filtrate was concentrated to the residue. The residue was diluted with H₂O (30 mL) and sat.Na₂CO₃ (30 mL), and the mixture was extracted with EtOAc (50 mL×2). The combined organic phase was washed with water (20 mL) and brine (20 mL), dried over Na₂SO₄, filtered and concentrated to give the crude product. The crude product was purified by silica gel column (EtOAc in PE=20% to 50% to 100%) to give 4-[4-(trifluoromethoxy)phenyl]pyridazine (650 mg, 50% yield) as a solid, which was confirmed by H NMR, F NMR, LCMS. ¹H NMR (400 MHz, CDCl₃) δ_{H} 9.46 (d, 1H), 9.27 (dd, 1H), 7.72 (d, 2H), 7.64 (dd, 1H), 7.41 (d, 2H). LCMS Rt=0.741 min in 1.5 min chromatography, MS ESI calcd. for C₁₁H₈F₃N₂O [M+H]⁺ 241.1, found 241.2.

To a mixture of amino hydrogen sulfate (692.79 mg, 6.13 mmol) in Water (6 mL) was added K₂CO₃ (423.34 mg, 3.06 mmol) at 10° C., then the mixture was stirred at 10° C. for 10 minutes. To the mixture was added 4-[4-(trifluoromethoxy)phenyl]pyridazine (500 mg, 2.04 mmol), then the mixture was stirred at 70° C. for 16 hours. From LCMS, desired MS was observed and some of starting material was remained. After cooled to r.t., to the mixture was added KI (508.46 mg, 3.06 mmol). The mixture was concentrated to give the crude product of 4-[4-(trifluoromethoxy)phenyl]pyridazin-1-ium-1-amine iodide (700 mg, 1.8272 mmol, 89.478% yield) as a solid, which was used next step without further purification. LCMS Rₜ=0.607 min in 1.5 min chromatography, MS ESI calcd. for C₁₁H₉F₃₁N₃O [M-I]⁺256.1, found 256.0.

To a mixture of 4-[4-(trifluoromethoxy)phenyl]pyridazin-1-ium-1-amine iodide (700 mg, 1.83 mmol) and ethyl prop-2-ynoate (358.49 mg, 3.65 mmol) in MeCN (15 mL) was added DBU (556.33 mg, 3.65 mmol), then the mixture was stirred at 20° C. for 16 hours. From LCMS, desired MS was observed and no starting material was remained. The mixture was diluted with H₂O (30 mL), and the mixture was extracted with EtOAc (50 mL×2). The combined organic phase was washed with water (20 mL×2) and brine (20 m), dried over Na₂SO₄, filtered and concentrated to give the crude product. The crude product was purified by silica gel column (EtOAc in PE=0% to 20% to 40%) to give the product of ethyl 5-[4-(trifluoromethoxy)phenyl]pyrazolo[1,5-b]pyridazine-3-carboxylate (150 mg, 0.3905 mmol, 21.372% yield, 91.45% purity) as a solid. ¹H NMR CDCl₃ 400 MHz 6=8.67 (s, 2H), 8.51 (s, 1H), 7.75 (d, 2H), 7.42 (d, 2H), 4.44 (q, 2H), 1.45 (t, 3H). LCMS Rₜ=0.888 min in 1.5 min chromatography, MS ESI calcd. for C₆H₁₃F₃N₃O₃ [M+H]⁺ 352.1, found 351.9.

A mixture of ethyl 5-[4-(trifluoromethoxy)phenyl]pyrazolo[1,5-b]pyridazine-3-carboxylate (150 mg, 0.43 mmol) in H₂SO₄ (2.17 mL, 40.78 mmol) and Water (4 mL) was stirred at 120° C. for 2 hours. From LCMS, desired MS was observed and no starting material was remained. After cooled to r.t., the mixture was poured into ice water (20 m), basified with Na₂CO₃ (solid) to pH ˜9, and extracted with EtOAc (30 mL×2). The combined organic phase was washed with brine (10 mL), dried over Na₂SO₄, filtered and concentrated to give the crude product of 5-[4-(trifluoromethoxy)phenyl]pyrazolo[1,5-b]pyridazine (115 mg, 0.3774 mmol, 88.389% yield, 91.64% purity) as a solid.

The crude product of 5-[4-(trifluoromethoxy)phenyl]pyrazolo[1,5-b]pyridazine (20 mg, 0.07 mmol) was purified by Prep-TLC (PET/EtOAc=3/1) to give the product of 5-[4-(trifluoromethoxy)phenyl]pyrazolo[1,5-b]pyridazine (7.73 mg, 0.0277 mmol, 38.65% yield, 100% purity) as a solid. ¹H NMR CDCl₃ 400 MHz 6=8.53 (d, 1H), 8.13-8.02 (m, 2H), 7.68 (d, 2H), 7.39 (d, 2H), 6.71 (d, 1H). LCMS Rₜ=1.166 min in 2.0 min chromatography, MS ESI calcd. for C₁₃H₉F₃N₃O [M+H]⁺ 280.1, found 279.9.

### Example 29: Synthesis of Compound 29

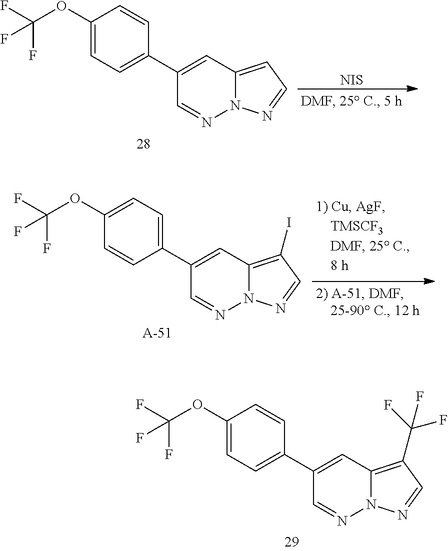

A mixture of 5-[4-(trifluoromethoxy)phenyl]pyrazolo[1,5-b]pyridazine (80 mg, 0.29 mmol) and NIS (128.92 mg, 0.57 mmol) in DMF (4 mL) was stirred at 25° C. for 5 hours. The mixture was diluted with H₂O (20 mL), and the mixture was extracted with EtOAc (30 mL×2). The combined organic phase was washed with water (15 mL×2) and brine (15 m), dried over Na₂SO₄, filtered and concentrated to give the crude product. The crude product was purified by flash chromatography on silica gel (EtOAc in PE=0% to 10% to 20%) to give 3-iodo-5-[4-(trifluoromethoxy)phenyl]pyrazolo[1,5-b]pyridazine (90 mg, 75% yield) as a solid. ¹H NMR (400 MHz, CDCl₃) δ_{H} 80.53 (d, 1H), 8.12 (s, 1H), 7.96 (d, 1H), 7.72 (d, 2H), 7.41 (d, 2H). LCMS Rₜ=0.899 min in 1.5 min chromatography, MS ESI calcd. for C₁₃H₈F₃IN₃O [M+H]⁺ 406.0, found 405.9.

To a mixture of AgF (184 mg, 1.45 mmol) in DMF (10 mL) was added trimethyl(trifluoromethyl)silane (280 mg, 1.97 mmol), then the mixture was stirred at 25° C. for 3 hours under N₂ in a sealed tube. To the mixture was added Cu (148 mg, 2.33 mmol), then the mixture was stirred at 25° C. for 5 hours. To the mixture was added 3-iodo-5-[4-(trifluoromethoxy)phenyl]pyrazolo[1,5-b]pyridazine (70 mg, 0.17 mmol), then the mixture was stirred at 25° C. for 2 hours and 90° C. for 10 hours in 20 mL sealed tube under N₂. After cooling to r.t., the mixture was diluted with H₂O (30 mL), and the mixture was extracted with EtOAc (50 mL×2). The combined organic phase was washed with water (20 mL×2) and brine (20 mL), dried over Na₂SO₄, filtered and concentrated to give the crude product. The crude product was purified by Prep-TLC (PE:EtOAc=5:1) to give 5-[4-(trifluoromethoxy)phenyl]-3-(trifluoromethyl)pyrazolo[1,5-b]pyridazine (17.36 mg, 29% yield) as a solid. ¹H NMR (400 MHz, CDCl₃) δ_{H} 8.68 (d, 1H), 8.31 (s, 1H), 8.20 (d, 1H), 7.72 (d, 2H), 7.43 (d, 2H). LCMS Rₜ=1.285 min in 2.0 min chromatography, MS ESI calcd. for C₁₄H₈F₆N₃O [M+H]⁺ 348.0, found 347.9.

### Example 30: Synthesis of Compound 30

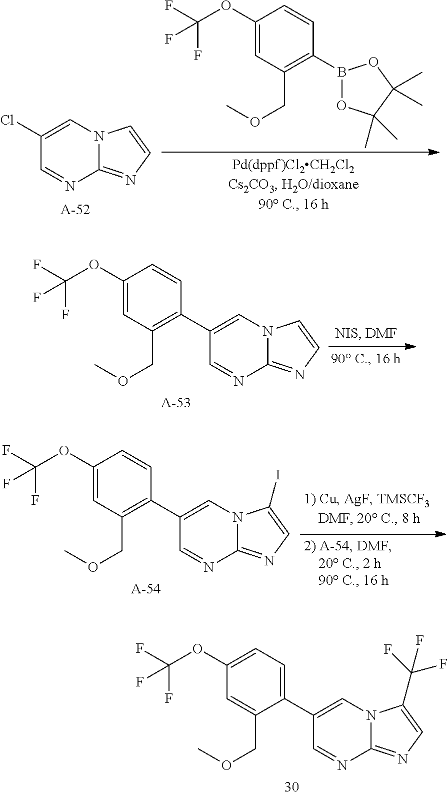

A mixture of 6-chloroimidazo[1,2-a]pyrimidine (300 mg, 1.95 mmol), 2-[2-(methoxymethyl)-4-(trifluoromethoxy)phenyl]-4,4,5,5-tetramethyl-1,3,2-dioxaborolane (843.44 mg, 2.54 mmol), Pd(dppf)Cl₂.CH₂Cl₂ (319.06 mg, 0.39 mmol) and Cs₂CO₃ (1272.9 mg, 3.91 mmol) in 1,4-dioxane (18 mL) and water (3 mL) was stirred at 90° C. for 16 hours under N₂. After cooling to r.t., the mixture was filtered through silica gel and eluted with EtOAc (20 mL×2). The filtrate was concentrated and diluted with EtOAc (30 mL), washed with water (10 mL×2) and brine (10 mL), dried over Na₂SO₄, filtered and concentrated to give the crude product. The crude product was purified by silica gel column (EtOAc in PE=0 to 50% to 100%) to give 6-[2-(methoxymethyl)-4-(trifluoromethoxy)phenyl]imidazo[1,2-a]pyrimidine (150 mg, 15% yield) as a solid. LCMS Rₜ=0.696 min in 1.5 min chromatography, MS ESI calcd. for C₁₅H₁₃F₃N₃O₂ [M+H]⁺ 324.1, found 323.9.

To a mixture of 6-[2-(methoxymethyl)-4-(trifluoromethoxy)phenyl]imidazo[1,2-a]pyrimidine (150 mg, 0.46 mmol) and DMF (3 mL) was added NIS (135.71 mg, 0.60 mmol) at 0° C. The mixture stirred at 90° C. for 16 hours. After cooling to r.t., to the mixture was added water (10 mL). The mixture was stirred for 15 mins. The solid was collected by filtration and dried in an oven to give 3-iodo-6-[2-(methoxymethyl)-4-(trifluoromethoxy)phenyl]imidazo[1,2-a]pyrimidine (110 mg, 50% yield) as a solid. ¹H NMR (400 MHz, CDCl₃) δ_{H} 8.65 (d, 1H), 8.57 (d, 1H), 7.98 (s, 1H), 7.45 (d, 2H), 7.38-7.33 (m, 1H), 4.30 (s, 2H), 3.46 (s, 3H). LCMS Rₜ=0.795 min in 1.5 min chromatography, MS ESI calcd. for C₁₅H₁₂F₃IN₃O₂ [M+H]⁺ 450.0, found 449.9.

To a mixture of fluorosilver (920 mg, 7.25 mmol) in DMF (50 mL) was added trimethyl(trifluoromethyl)silane (1.34 g, 9.43 mmol) under N₂ at 20° C., then the mixture was stirred at 20° C. for 2 hours. To the mixture was added copper (737.33 mg, 11.6 mmol), then the mixture was stirred at 20° C. for 6 hours under N₂. Trifluoromethylcopper (1.46 g) was obtained as a solution in DMF (0.145 M), which was used next step directly.

A mixture of 3-iodo-6-[2-(methoxymethyl)-4-(trifluoromethoxy)phenyl]imidazo[1,2-a]pyrimidine (110 mg, 0.24 mmol) in trifluoromethylcopper in DMF (10 mL, 1.45 mmol) was stirred at 20° C. for 2 hours in a sealed tube under N₂. Then the mixture was heated to 90° C. and stirred for 16 hours. After cooling to r.t., the mixture was filtered through Celite, and eluted with EtOAc (20 mL×2). The filtrate was concentrated and diluted with EtOAc (30 mL), washed with water (10 mL×2), brine (10 mL), dried over Na₂SO₄, filtered and concentrated to give the crude product. The crude product was purified by Prep-HPLC (column: Phenomenex Gemini C18 250*50 10u; mobile phase: [water (0.05% ammonia hydroxide v/v)-ACN]; B %: 58%-68%, 8 min) to give 6-[2-(methoxymethyl)-4-(trifluoromethoxy)phenyl]-3-(trifluoromethyl)imidazo[1,2-a]pyrimidine (22.71 mg, 23% yield) as a solid. ¹H NMR (400 MHz, CDCl₃) δ_{H} 8.80 (br d, 2H), 8.22 (br s, 1H), 7.47-7.43 (m, 2H), 7.40-7.34 (m, 1H), 4.28 (s, 2H), 3.43 (s, 3H). LCMS Rₜ=1.209 min in 2.0 min chromatography, MS ESI calcd. for C₁₆H₁₂F₆N₃O₂ [M+H]⁺ 392.1, found 392.0.

### Example 31: Synthesis of Compound 31

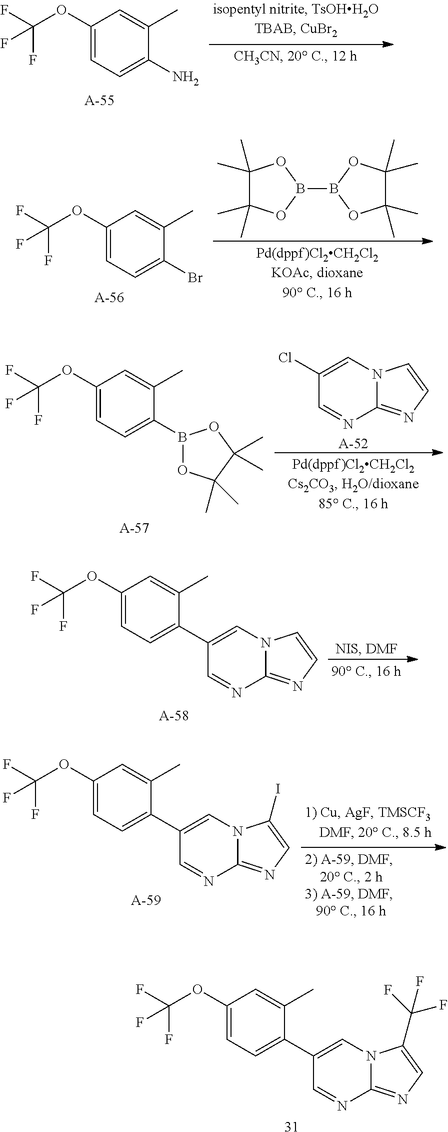

To a mixture of 2-methyl-4-(trifluoromethoxy)aniline (4 g, 20.93 mmol), TBAB (16.86 g, 52.31 mmol) and TsOH.H₂O (5.17 g, 27.2 mmol) in MeCN (40 mL) was added isopentyl nitrite (2.94 g, 25.11 mmol) and CuBr₂ (467.38 mg, 2.09 mmol), then the mixture was stirred at 20° C. for 12 hours. The mixture was diluted with H₂O (100 mL), and the mixture was extracted with DCM (100 mL×2). The combined organic phase was washed with water (40 mL×2) and brine (50 mL), dried over Na₂SO₄, filtered and concentrated to give the crude product. The crude product was purified by silica gel column (EtOAc in PE=0% to 2%) to give 1-bromo-2-methyl-4-(trifluoromethoxy)benzene (4 g, 75% yield) as an oil. ¹H NMR (400 MHz, CDCl₃) δ_{H} 7.54 (d, 1H), 7.11 (s, 1H), 6.94 (dd, 1H), 2.42 (s, 3H).

A mixture of 1-bromo-2-methyl-4-(trifluoromethoxy)benzene (4 g, 15.68 mmol), 4,4,5,5-tetramethyl-2-(4,4,5,5-tetramethyl-1,3,2-dioxaborolan-2-yl)-1,3,2-dioxaborolane (11.95 g, 47.05 mmol), Pd(dppf)Cl₂.CH₂Cl₂ (1.28 g, 1.57 mmol) and KOAc (3.08 g, 31.37 mmol) in 1,4-dioxane (100 mL) was stirred at 90° C. for 16 hours under N₂. After cooling to r.t., the mixture was concentrated. The residue was diluted with H₂O (50 mL), and the mixture was extracted with EtOAc (150 mL×2). The combined organic phase was washed with water (50 mL×2) and brine (50 mL), dried over Na₂SO₄, filtered and concentrated to give the crude product. The crude product was purified by silica gel column (EtOAc in PE=0% to 1%) to give 4,4,5,5-tetramethyl-2-[2-methyl-4-(trifluoromethoxy)phenyl]-1,3,2-dioxaborolane (4.4 g, 93% yield) as an oil. ¹H NMR (400 MHz, CDCl₃) δ_{H} 7.79 (d, 1H), 7.04-6.98 (m, 2H), 2.56 (s, 3H), 1.35 (s, 12H).

A mixture of 6-chloroimidazo[1,2-a]pyrimidine (305 mg, 1.99 mmol), 4,4,5,5-tetramethyl-2-[2-methyl-4-(trifluoromethoxy)phenyl]-1,3,2-dioxaborolane (300 mg, 0.99 mmol), Cs₂CO₃ (647.07 mg, 1.99 mmol) and Pd(dppf)Cl₂.CH₂Cl₂ (162.19 mg, 0.20 mmol) in 1,4-dioxane (8 mL) and water (2 mL) was stirred at 85° C. for 16 hours under N₂. After cooling to r.t., the mixture was filtered through silica gel and eluted with EtOAc (20 mL×2). The filtrate was concentrated and diluted with EtOAc (30 mL), washed with water (10 mL×2) and brine (10 mL), dried over Na₂SO₄, filtered and concentrated to give the crude product. The crude product was purified by silica gel column (EtOAc in PE=0 to 30% to 50%) to give 6-[2-methyl-4-(trifluoromethoxy)phenyl]imidazo[1,2-a]pyrimidine (250 mg, 24% yield) as a solid. ¹H NMR (400 MHz, CDCl₃) δ_{H} 8.54 (d, 1H), 8.38 (d, 1H), 7.90 (d, 1H), 7.61 (d, 1H), 7.30 (d, 1H), 7.23-7.16 (m, 2H), 2.35 (s, 3H). LCMS Rₜ=0.706 min in 1.5 min chromatography, MS ESI calcd. for C₁₄H₁₁F₃N₃O [M+H]⁺ 294.1, found 293.9.

To a mixture of 6-[2-methyl-4-(trifluoromethoxy)phenyl]imidazo[1,2-a]pyrimidine (250 mg, 0.85 mmol) and DMF (3 mL) was added NIS (249.35 mg, 1.11 mmol). The mixture stirred at 90° C. for 16 hours After cooling to r.t, to the mixture was added water (10 mL). The solid was collected by filtration and dried in oven to give 3-iodo-6-[2-methyl-4-(trifluoromethoxy)phenyl]imidazo[1,2-a]pyrimidine (280 mg, 29% yield) as a solid. LCMS Rₜ=0.805 min in 1.5 min chromatography, MS ESI calcd. for C₁₄H₁₀F₃IN₃O [M+H]⁺ 420.0, found 419.9.

To a mixture of fluorosilver (920 mg, 7.25 mmol) in DMF (50 mL) was added trimethyl(trifluoromethyl)silane (1.34 g, 9.43 mmol) under N₂ at 20° C., then the mixture was stirred at 20° C. for 2 hours. To the mixture was added copper (737.33 mg, 11.6 mmol), then the mixture was stirred at 20° C. for 6 hours under N₂. Trifluoromethylcopper (1.46 g) was obtained as a solution in DMF (0.145 M), which was used next step directly.

A mixture of 3-iodo-6-[2-methyl-4-(trifluoromethoxy)phenyl]imidazo[1,2-a]pyrimidine (280 mg, 0.67 mmol) in trifluoromethylcopper in DMF (10 mL, 1.45 mmol) was stirred at 20° C. for 2 hours in a sealed tube under N₂. Then the mixture was heated to 90° C. and stirred for 16 hours. After cooling to r.t., the mixture was filtered through Celite, and eluted with EtOAc (20 mL×2). The filtrate was concentrated and diluted with EtOAc (30 mL), washed with water (10 mL×2), brine (10 mL), dried over Na₂SO₄, filtered and concentrated to give the crude product. The crude product was purified by Prep-HPLC (column: Phenomenex Gemini C18 250*50 10u; mobile phase: [water (0.05% ammonia hydroxide v/v)-ACN]; B %: 58%-68%, 8 min) to give 6-[2-methyl-4-(trifluoromethoxy)phenyl]-3-(trifluoromethyl)imidazo[1,2-a]pyrimidine (11.13 mg, 5% yield) as a solid. ¹H NMR (400 MHz, CDCl₃) δ_{H} 8.73 (d, 1H), 8.45 (d, 1H), 8.22 (s, 1H), 7.33 (d, 1H), 7.26-7.20 (m, 2H), 2.37 (s, 3H). LCMS Rₜ=1.218 min in 2.0 min chromatography, MS ESI calcd. for C₁₅H₁₀F₆N₃O [M+H]⁺ 362.1, found 362.0.

### Example 32: Synthesis of Compound 32

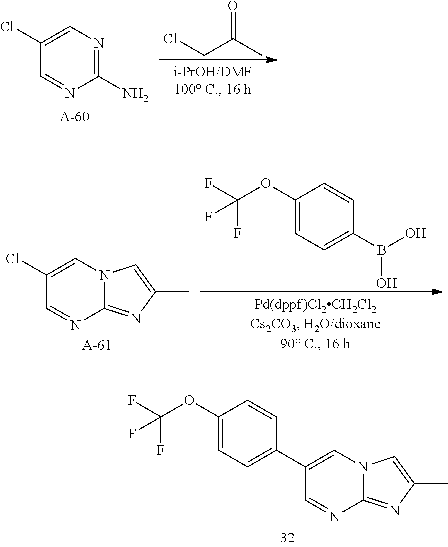

To a mixture of 5-chloropyrimidin-2-amine (1 g, 7.72 mmol) in DMF (5 mL) and IPA (20 mL) was added 1-chloropropan-2-one (5 g, 54.03 mmol) at 100° C. for 16 hours. After cooling to r.t., the reaction was quenched with sat.NH₄Cl (30 mL), and the mixture was extracted with EtOAc (50 mL×2). The combined organic phase was washed with brine (50 mL), dried over Na₂SO₄, filtered and concentrated to give the crude product. The crude product was purified by silica gel column (EtOAc in PE=0 to 50% to 100%) to give 6-chloro-2-methyl-imidazo[1,2-a]pyrimidine (260 mg, 20% yield) as a solid. ¹H NMR (400 MHz, CDCl₃) δ_{H} 8.43-8.38 (m, 2H), 7.31 (s, 1H), 2.51 (s, 3H).

A mixture of 6-chloro-2-methyl-imidazo[1,2-a]pyrimidine (260 mg, 1.55 mmol), [4-(trifluoromethoxy)phenyl]boronic acid (415.3 mg, 2.02 mmol), Pd(dppf)Cl₂.CH₂Cl₂ (253.37 mg, 0.31 mmol) and Cs₂CO₃ (1.01 g, 3.1 mmol) in 1,4-dioxane (15 mL) and water (3 mL) was stirred at 90° C. for 16 hours. After cooling to r.t., the mixture was filtered through silica gel and eluted with EtOAc (20 mL×2). The filtrate was concentrated and diluted with EtOAc (30 mL), washed with water (10 mL×2) and brine (10 mL), dried over Na₂SO₄, filtered and concentrated to give the crude product. The crude product was purified by Prep-HPLC (column: Phenomenex Gemini C18 250*50 10u; mobile phase: [water (0.05% ammonia hydroxide v/v)-ACN]; B %: 38%-48%, 8 min) to give (2-methyl-6-[4-(trifluoromethoxy)phenyl]imidazo[1,2-a]pyrimidine (28.78 mg, 6% yield) as a solid. ¹H NMR (400 MHz, CDCl₃) δ_{H} 8.71 (d, 1H), 8.47 (d, 1H), 7.59 (d, 2H), 7.39-7.35 (m, 3H), 2.55 (s, 3H). LCMS Rₜ=0.889 min in 2.0 min chromatography, MS ESI calcd. for C₁₄H₁₁F₃N₃O [M+H]⁺ 294.1, found 293.9.

### Example 33: Synthesis of Compound 33

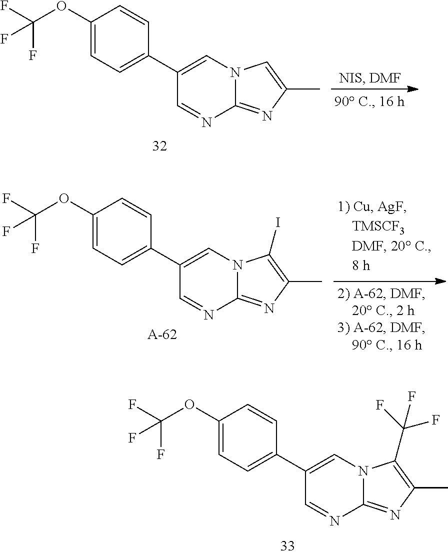

To a mixture of 2-methyl-6-[4-(trifluoromethoxy)phenyl]imidazo[1,2-a]pyrimidine (270 mg, 0.92 mmol) in DMF (10 mL) was added NIS (310.72 mg, 1.38 mmol) at 0° C. Then the mixture was heated to 90° C. and stirred for 16 hours. After cooling to r.t., to the mixture was added water (30 mL). The solid was collected by filtration and dried in oven to give 3-iodo-2-methyl-6-[4-(trifluoromethoxy)phenyl]imidazo[1,2-a]pyrimidine (250 mg, 47% yield) as a solid. LCMS Rₜ=0.788 min in 1.5 min chromatography, MS ESI calcd. for C₁₄H₁₀F₃IN₃O [M+H]⁺ 420.0, found 419.9.

To a mixture of fluorosilver (920 mg, 7.25 mmol) in DMF (50 mL) was added trimethyl(trifluoromethyl)silane (1.34 g, 9.43 mmol) under N₂ at 20° C., then the mixture was stirred at 20° C. for 2 hours. To the mixture was added copper (737.33 mg, 11.6 mmol), then the mixture was stirred at 20° C. for 6 hours under N₂. Trifluoromethylcopper (1.46 g) was obtained as a solution in DMF (0.145 M), which was used next step directly.

A mixture of 3-iodo-2-methyl-6-[4-(trifluoromethoxy)phenyl]imidazo[1,2-a]pyrimidine (250 mg, 0.60 mmol) in trifluoromethylcopper in DMF (10 mL, 1.45 mmol) was stirred at 20° C. for 2 hours in a sealed tube under N₂. Then the mixture was heated to 90° C. and stirred for 16 hours. After cooling to r.t., the mixture was filtered through Celite, and eluted with EtOAc (20 mL×2). The filtrate was concentrated and diluted with EtOAc (30 mL), washed with water (10 mL×2), brine (10 mL), dried over Na₂SO₄, filtered and concentrated to give the crude product. The crude product was purified by Prep-HPLC (column: Phenomenex Gemini C18 250*50 10u; mobile phase: [water (0.05% ammonia hydroxide v/v)-ACN]; B %: 58%-68%, 8 min) to give 2-methyl-6-[4-(trifluoromethoxy)phenyl]-3-(trifluoromethyl)imidazo[1,2-a]pyrimidine (18.42 mg, 8% yield) as a solid. ¹H NMR (400 MHz, CDCl₃) δ_{H} 8.87 (d, 1H), 8.56 (d, 1H), 7.62 (d, 2H), 7.41 (d, 2H), 2.67 (d, 3H). LCMS Rₜ=1.233 min in 2.0 min chromatography, MS ESI calcd. for C₁₅H₁₀F₆N₃O [M+H]⁺ 362.1, found 362.0.

### Example 34: Synthesis of Compound 34

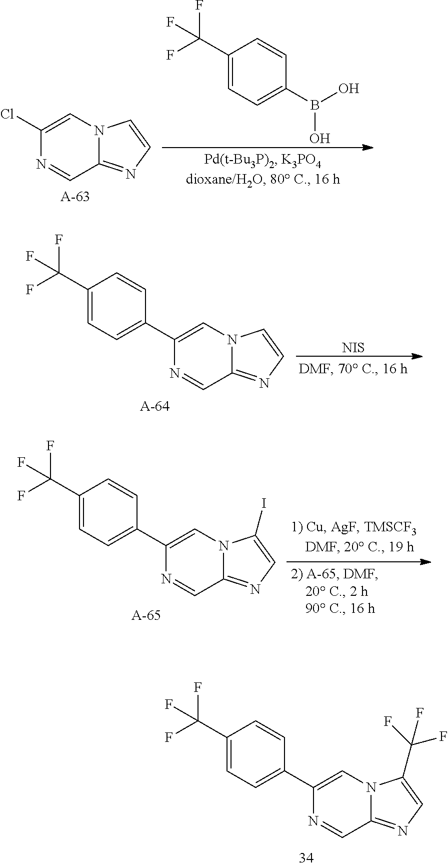

To a solution of 6-chloroimidazo[1,2-a]pyrazine (200 mg, 1.3 mmol) in 1,4-dioxane (4 mL) and water (0.40 mL) was added [4-(trifluoromethyl)phenyl]boronic acid (296.82 mg, 1.56 mmol), Pd(t-Bu₃P)₂ (99.84 mg, 0.20 mmol) and K₃PO₄ (552.97 mg, 2.6 mmol). The resulting mixture was stirred at 80° C. under N₂ for 16 hours to give a suspension. The reaction mixture was cooled to room temperature and filtered through Celite. The filtrate was concentrated to give the crude product. The crude product was purified by silica gel column with EtOAc in PE (10% to 30% to 50% to 80%) to give 6-[4-(trifluoromethyl)phenyl]imidazo[1,2-a]pyrazine (235 mg, 069% yield) as a solid. ¹H NMR (400 MHz, CDCl₃) δ_{H} 9.22 (s, 1H), 8.55 (s, 1H), 8.09 (d, 2H), 7.88 (s, 1H), 7.82-7.74 (m, 3H).

To a solution of 6-[4-(trifluoromethyl)phenyl]imidazo[1,2-a]pyrazine (235 mg, 0.89 mmol) in DMF (5 mL) was added NIS (301.29 mg, 1.34 mmol). The resulting mixture was stirred at 70° C. for 16 hours to give a solution. The reaction mixture was cooled to room temperature. EtOAc (20 mL) and H₂O (20 mL) were added to the mixture. After separation, the organic layer was washed with brine (15 mL×2), dried over anhydrous Na₂SO₄, filtered and concentrated to give the crude product. The crude product was purified by silica gel column with EtOAc in PE (10% to 30% to 50%) to give 3-iodo-6-[4-(trifluoromethyl)phenyl]imidazo[1,2-a]pyrazine (240 mg, 53% yield) as a solid. ¹H NMR (400 MHz, CDCl₃) δ_{H} 9.10 (d, 1H), 8.46 (d, 1H), 8.14 (d, 2H), 7.92 (s, 1H), 7.79 (d, 2H). LCMS Rₜ=1.235 min in 2.0 min chromatography, MS ESI calcd. for C₁₃H₈F₃IN₃ [M+H]⁺ 390.0, found 389.9.

To a mixture of fluorosilver (1.4 g, 11.03 mmol) in DMF (75 mL) was added trimethyl(trifluoromethyl)silane (2.04 g, 14.35 mmol) under N₂ at 20° C., then the mixture was stirred at 20° C. for 3 hours. To the mixture was added copper (1.12 g, 17.66 mmol), then the mixture was stirred at 20° C. for 16 hours under N₂. The mixture turned to green. trifluoromethylcopper (1460 mg) was obtained as a solution in DMF (˜0.145 M), which was used next step directly.

A mixture of 3-iodo-6-[4-(trifluoromethyl)phenyl]imidazo[1,2-a]pyrazine (100 mg, 0.26 mmol) in CuCF₃/DMF (12 mL, 1.74 mmol) in a sealed tube was stirred at 20° C. for 2 hours and 90° C. for 16 hours to give a suspension. The reaction mixture was cooled to room temperature. EtOAc (50 mL) and saturated NH₄Cl aqueous (50 mL) were added to the mixture and filtered through Celite. After separation, the organic layer was washed with brine (30 mL×2), dried over anhydrous Na₂SO₄, filtered and concentrated to give the crude product. The crude product was purified by Prep-HPLC (water (0.05% ammonia hydroxide v/v)-ACN) to give 3-(trifluoromethyl)-6-[4-(trifluoromethyl)phenyl]imidazo[1,2-a]pyrazine (27.77 mg, 33% yield) as a solid. ¹H NMR (400 MHz, CDCl₃) δ_{H} 9.34 (s, 1H), 8.54 (s, 1H), 8.16 (s, 1H), 8.11 (d, 2H), 7.80 (d, 2H). LCMS Rₜ=1.269 min in 2.0 min chromatography, MS ESI calcd. for C₁₄H₈F₆N₃ [M+H]⁺ 332.1, found 331.9.

### Example 35: Synthesis of Compound 35

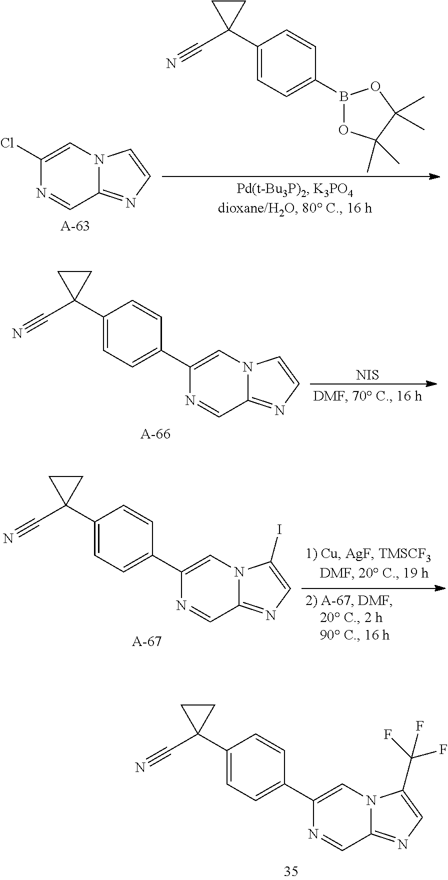

To a solution of 6-chloroimidazo[1,2-a]pyrazine (200 mg, 1.3 mmol) in 1,4-dioxane (4 mL) and water (0.40 mL) was added 1-[4-(4,4,5,5-tetramethyl-1,3,2-dioxaborolan-2-yl)phenyl]cyclopropanecarbonitrile (420.63 mg, 1.56 mmol), Pd(t-Bu₃P)₂ (99.84 mg, 0.20 mmol) and K₃PO₄ (552.97 mg, 2.6 mmol). The resulting mixture was stirred at 80° C. under N₂ for 16 hours to give a suspension. The reaction mixture was cooled to room temperature and filtered through Celite. The filtrate was concentrated to give the crude product. The crude product was purified by silica gel column with EtOAc in PE (10% to 30% to 50% to 80%) to give 1-(4-imidazo[1,2-a]pyrazin-6-ylphenyl)cyclopropanecarbonitrile (240 mg, 71% yield) as a solid. ¹H NMR (400 MHz, CDCl₃) δ_{H} 9.20 (s, 1H), 8.48 (s, 1H), 7.94 (d, 2H), 7.85 (s, 1H), 7.77 (s, 1H), 7.42 (d, 2H), 1.83-1.79 (m, 2H), 1.51-1.46 (m, 2H).

To a solution of 1-(4-imidazo[1,2-a]pyrazin-6-ylphenyl)cyclopropanecarbonitrile (240 mg, 0.92 mmol) in DMF (5 mL) was added NIS (311.16 mg, 1.38 mmol). The resulting mixture was stirred at 70° C. for 16 hours to give a solution. The reaction mixture was cooled to room temperature. EtOAc (20 mL) and H₂O (20 mL) was added to the mixture. After separation, the organic layer was washed with brine (15 mL×2), dried over anhydrous Na₂SO₄, filtered and concentrated to give the crude product. The crude product was purified by silica gel column with EtOAc in PE (10% to 30% to 50%) to give 1-[4-(3-iodoimidazo[1,2-a]pyrazin-6-yl)phenyl]cyclopropanecarbonitrile (130 mg, 35% yield) as solid. ¹H NMR (400 MHz, CDCl₃) δ_{H} 9.08 (s, 1H), 8.39 (s, 1H), 8.00 (d, 2H), 7.90 (s, 1H), 7.45 (d, 2H), 1.84-1.80 (m, 2H), 1.52-1.48 (m, 2H). LCMS Rₜ=1.118 min in 2.0 min chromatography, MS ESI calcd. for C₁₆H₁₂IN₄ [M+H]⁺ 387.0, found 386.9.

To a mixture of fluorosilver (1.4 g, 11.03 mmol) in DMF (75 mL) was added trimethyl(trifluoromethyl)silane (2.04 g, 14.35 mmol) under N₂ at 20° C., then the mixture was stirred at 20° C. for 3 hours. To the mixture was added copper (1.12 g, 17.66 mmol), then the mixture was stirred at 20° C. for 16 hours under N₂. The mixture turned to green. trifluoromethylcopper (1460 mg) was obtained as a solution in DMF (˜0.145 M), which was used next step directly.

A mixture of 1-[4-(3-iodoimidazo[1,2-a]pyrazin-6-yl)phenyl]cyclopropanecarbonitrile (130 mg, 0.32 mmol) in CuCF₃/DMF (12 mL, 1.74 mmol) was stirred in a sealed tube at 20° C. for 2 hours and 90° C. for 16 hours to give a suspension. The reaction mixture was cooled to room temperature and filtered through Celite. EtOAc (30 mL) and saturated NH₄Cl (30 mL) was added to the filtrate, after separated, the organic layer was washed with brine (20 mL×2), dried over anhydrous Na₂SO₄, filtered and concentrated to give the crude product. The crude product was purified by Prep-HPLC (water (0.05% ammonia hydroxide v/v)-ACN) to give 1-[4-[3-(trifluoromethyl)imidazo[1,2-a]pyrazin-6-yl]phenyl]cyclopropanecarbonitrile (25.15 mg, 24% yield) as a solid. ¹H NMR (400 MHz, CDCl₃) δ_{H} 9.31 (d, 1H), 8.47 (s, 1H), 8.13 (s, 1H), 7.96 (d, 2H), 7.45 (d, 2H), 1.86-1.80 (m, 2H), 1.53-1.47 (m, 2H). LCMS Rₜ=1.159 min in 2.0 min chromatography, MS ESI calcd. for C₁₇H₁₂F₃N₄ [M+H]⁺ 329.1, found 328.9.

### Example 36: Synthesis of Compound 36

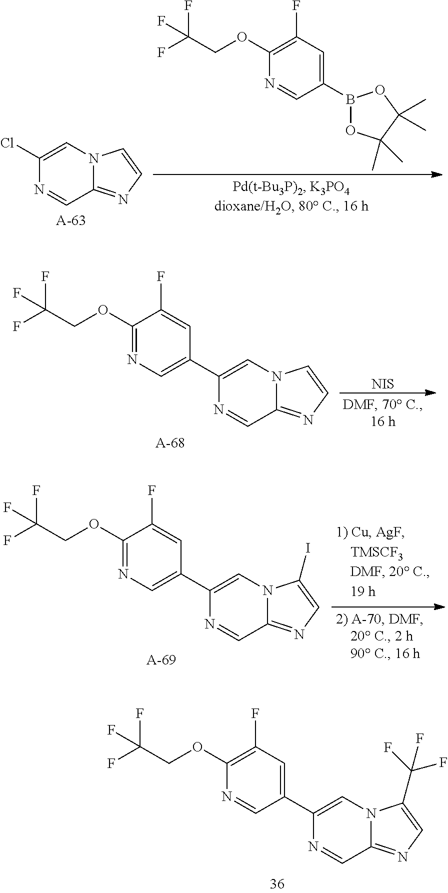

A mixture of 6-chloroimidazo[1,2-a]pyrazine (150 mg, 0.98 mmol), 3-fluoro-5-(4,4,5,5-tetramethyl-1,3,2-dioxaborolan-2-yl)-2-(2,2,2-trifluoroethoxy)pyridine (376.34 mg, 1.17 mmol), K₃PO₄ (414.67 mg, 1.95 mmol) and Pd(t-Bu₃P)₂ (74.88 mg, 0.15 mmol) in 1,4-dioxane (4 mL) and water (0.40 mL) was stirred at 80° C. under N₂ for 16 hours to give a suspension. The reaction mixture was cooled to room temperature and filtered through Celite. The filtrate was concentrated to give the crude product. The crude product was purified by silica gel column with EtOAc in PE (20% to 30% to 50%) to give 6-[5-fluoro-6-(2,2,2-trifluoroethoxy)-3-pyridyl]imidazo[1,2-a]pyrazine (170 mg, 56% yield) as a solid. ¹H NMR (400 MHz, CDCl₃) δ_{H} 9.19 (s, 1H), 8.48 (d, 1H), 8.46 (d, 1H), 8.08 (dd, 1H), 7.89 (s, 1H), 7.80 (s, 1H), 4.92 (q, 2H). LCMS Rₜ=1.021 min in 2.0 min chromatography, MS ESI calcd. for C₁₃H₉F₄N₄O [M+H]⁺ 313.1, found 312.9.

To a solution of 6-[5-fluoro-6-(2,2,2-trifluoroethoxy)-3-pyridyl]imidazo[1,2-a]pyrazine (170 mg, 0.54 mmol) in DMF (3 mL) was added NIS (183.75 mg, 0.82 mmol). The resulting solution was stirred at 70° C. for 16 hours to give a solution. The reaction solution was cooled to room temperature. Saturated NH₄Cl aqueous (20 mL) and EtOAc (20 mL) were added to the reaction mixture. After separation, the organic layer was washed with brine (20 mL×2), dried over anhydrous Na₂SO₄, filtered and concentrated to give the crude product. The crude product was purified by silica gel column with EtOAc in PE (0% to 10% to 30%) to give 6-[5-fluoro-6-(2,2,2-trifluoroethoxy)-3-pyridyl]-3-iodo-imidazo[1,2-a]pyrazine (140 mg, 59% yield) as a solid. ¹H NMR (400 MHz, CDCl₃) δ_{H} 9.07 (d, 1H), 8.53 (d, 1H), 8.37 (d, 1H), 8.11 (dd, 1H), 7.92 (s, 1H), 4.93 (q, 2H). LCMS Rₜ=0.844 min in 1.5 min chromatography, MS ESI calcd. for C₁₃H₈F₄IN₄ [M+H]⁺ 439.0, found 398.9.

To a mixture of fluorosilver (1.4 g, 11.03 mmol) in DMF (75 mL) was added trimethyl(trifluoromethyl)silane (2.04 g, 14.35 mmol) under N₂ at 20° C., then the mixture was stirred at 20° C. for 3 hours. To the mixture was added copper (1.12 g, 17.66 mmol), then the mixture was stirred at 20° C. for 16 hours under N₂. The mixture turned to green. trifluoromethylcopper (1460 mg) was obtained as a solution in DMF (˜0.145 M), which was used next step directly.

A mixture of 6-[5-fluoro-6-(2,2,2-trifluoroethoxy)-3-pyridyl]-3-iodo-imidazo[1,2-a]pyrazine (140 mg, 0.32 mmol) in CuCF₃/DMF (12 mL, 1.74 mmol) was stirred in a sealed tube at 20° C. for 2 hours and at 90° C. for 16 hours to give a suspension. The reaction mixture was cooled to room temperature and filtered through Celite. EtOAc (30 mL) and saturated NH₄Cl (30 mL) was added to the filtrate, after separated, the organic layer was washed with brine (20 mL×2), dried over anhydrous Na₂SO₄, filtered and concentrated to give the crude product. The crude product was purified by Prep-HPLC (water (0.05% ammonia hydroxide v/v)-ACN) to give 6-[5-fluoro-6-(2,2,2-trifluoroethoxy)-3-pyridyl]-3-(trifluoromethyl)imidazo[1,2-a]pyrazine (43.41 mg, 36% yield) as a solid. ¹H NMR (400 MHz, CDCl₃) δ_{H} 9.31 (d, 1H), 8.50 (d, 1H), 8.46 (s, 1H), 8.15 (s, 1H), 8.08 (dd, 1H), 4.92 (q, 2H). LCMS Rₜ=1.219 min in 2.0 min chromatography, MS ESI calcd. for C₁₄H₈F₇N₄O [M+H]⁺ 381.1, found 381.0.

### Example 37: Synthesis of Compound 37

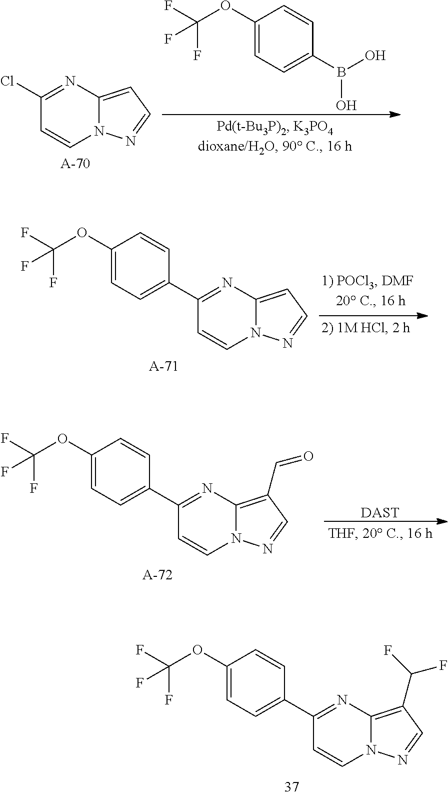

A mixture of 5-chloropyrazolo[1,5-a]pyrimidine (300 mg, 1.95 mmol), [4-(trifluoromethoxy)phenyl]boronic acid (442.51 mg, 2.15 mmol), K₃PO₄ (829.46 mg, 3.91 mmol) and Pd(t-Bu₃P)₂ (99.83 mg, 0.2 mmol) in 1,4-dioxane (8 mL) and water (0.8 mL) was stirred under N₂ at 90° C. for 16 hours. The mixture was cooled to r.t., diluted with EtOAc (10 m), filtered through silica gel, eluted with EtOAc (20 mL) and concentrated to give the crude product. The crude product was purified by silica gel column (PE:EtOAc=5:1 to 2:1) to give 5-[4-(trifluoromethoxy)phenyl]pyrazolo[1,5-a]pyrimidine (420 mg, 77% yield) as a solid. ¹H NMR (400 MHz, CDCl₃) δ_{H} 8.75 (d, 1H), 8.17-8.14 (m, 3H), 7.37 (d, 2H), 7.25 (s, 1H), 6.74 (d, 1H).

To a solution of the 5-[4-(trifluoromethoxy)phenyl]pyrazolo[1,5-a]pyrimidine (200 mg, 0.72 mmol) in DMF (2 mL) was added POCl₃ (0.2 mL, 2.15 mmol) and the mixture was stirred at 20° C. for 16 hours. The mixture was pour into water (0° C.), then 1N HCl (5 ml) was added, and the mixture was stirred for 2 hours. The mixture was extracted with DCM (20 mL×2). The combined organic phase was washed with brine (10 mL), dried over Na₂SO₄, filtered and concentrated to give the crude product. The crude product was purified by silica gel column (PE:EtOAc=5:1 to 1:1) to give 5-[4-(trifluoromethoxy)phenyl]pyrazolo[1,5-a]pyrimidine-3-carbaldehyde (110 mg, 50% yield) as a solid. ¹H NMR (400 MHz, DMSO-d₆) δ_{H} 10.24 (s, 1H), 9.46 (d, 1H), 8.75 (s, 1H), 8.48 (d, 2H), 8.01 (d, 1H), 7.61 (d, 2H).

To the mixture of 5-[4-(trifluoromethoxy)phenyl]pyrazolo[1,5-a]pyrimidine-3-carbaldehyde (100 mg, 0.33 mmol) in THF (1 mL) was added DAST (1311.64 mg, 8.14 mmol) and the mixture was stirred at 20° C. for 16 hours. The mixture was pour into water (0° C.), neutralized with sat.NaHCO₃ to pH=8-10, then extracted with EtOAc (30 mL×2). The combined phase was washed with brine (10 mL), dried over Na₂CO₃, filtered and concentrated to give the crude product. The crude product was purified by Prep-HPLC (water (10 mM NH₄HCO₃)-ACN) to give 3-(difluoromethyl)-5-[4-(trifluoromethoxy)phenyl]pyrazolo[1,5-a]pyrimidine (75 mg, 68% yield) as a solid. ¹H NMR (400 MHz, DMSO-d₆) δ_{H} 9.34 (d, 1H), 8.53 (s, 1H), 8.41 (d, 2H), 7.86 (d, 1H), 7.58 (d, 2H), 7.39 (t, 1H). LCMS Rₜ=1.247 min in 2.0 min chromatography, MS ESI calcd. for C₁₄H₉F₅N₃O [M+H]⁺ 330.1, found 329.9.

### Example 38: Synthesis of Compound 38

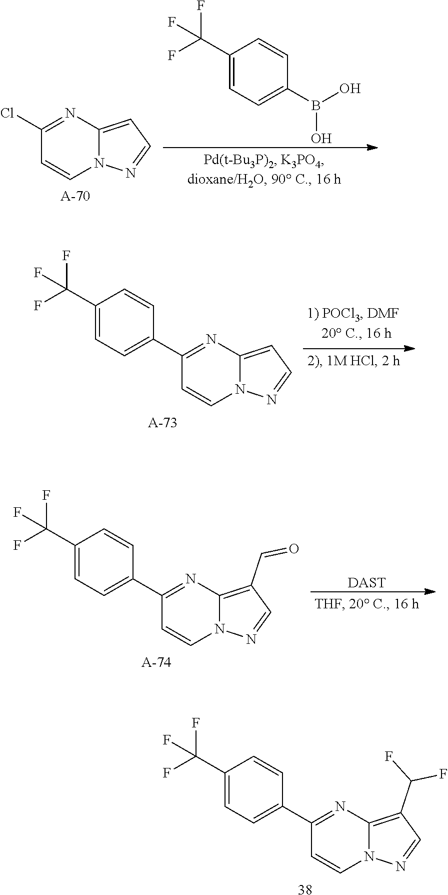

A mixture of 5-chloropyrazolo[1,5-a]pyrimidine (300 mg, 1.95 mmol) [4-(trifluoromethyl)phenyl]boronic acid (408.13 mg, 2.15 mmol) K₃PO₄ (829.46 mg, 3.91 mmol) and Pd(t-Bu₃P)₂ (99.83 mg, 0.2 mmol) in 1,4-dioxane (8 mL) and water (0.8 mL) was stirred under N₂ at 90° C. for 16 hours. The mixture was cooled to r.t., diluted with EtOAc (5 mL), filtered through silica gel, eluted with EtOAc (5 mL) and concentrated to give the crude product. The crude product was purified by silica gel column (PE:EtOAc=5:1 to 2:1) to give 5-[4-(trifluoromethyl)phenyl]pyrazolo[1,5-a]pyrimidine (470 mg, 91% yield) as a solid. ¹H NMR (400 MHz, CDCl₃) δ_{H} 8.78 (d, 1H), 8.23 (d, 2H), 8.19 (d, 1H), 7.79 (d, 2H), 7.31 (d, 1H), 6.78 (dd, 1H).

To a solution of the 5-[4-(trifluoromethyl)phenyl]pyrazolo[1,5-a]pyrimidine (200 mg, 0.76 mmol) in DMF (2 mL) was added POCl₃ (0.21 mL, 2.28 mmol), and the mixture was stirred at 20° C. for 16 hours. The mixture was pour into water (0° C.), then 1N HCl (5 ml) was added, and the mixture was stirred for 2 hours. The mixture was extracted with DCM (20 mL×2). The combined organic phase was washed with brine (10 mL), dried over Na₂SO₄, filtered and concentrated to give the crude product. The crude product was purified by silica gel column (PE:EtOAc=5:1 to 1:1) to give 5-[4-(trifluoromethyl)phenyl]pyrazolo[1,5-a]pyrimidine-3-carbaldehyde (150 mg, 68% yield) as a solid. ¹H NMR (400 MHz, DMSO-d₆) δ_{H} 10.26 (s, 1H), 9.50 (d, 1H), 8.78 (s, 1H), 8.56 (d, 2H), 8.08 (d, 1H), 7.98 (d, 2H).

To the mixture of 5-[4-(trifluoromethyl)phenyl]pyrazolo[1,5-a]pyrimidine-3-carbaldehyde (100, mg, 0.34 mmol) in THF (1 mL) was added DAST (1383.7 mg, 8.58 mmol) and the mixture was stirred at 20° C. for 16 hours. The mixture was pour into water (0° C.), neutralized with sat. NaHCO₃ to pH=8-10, and extracted with EtOAc (30 mL×2). The combined organic phase was washed with brine (10 mL), dried over Na₂CO₃, filtered and concentrated to give the crude product. The crude product was purified by Prep-HPLC (water (10 mM NH₄HCO₃)-ACN) to give 3-(difluoromethyl)-5-[4-(trifluoromethyl)phenyl]pyrazolo[1,5-a]pyrimidine (69.37 mg, 64% yield) as a solid. ¹H NMR (400 MHz, DMSO-d₆) δ_{H} 9.34 (d, 1H), 8.53 (s, 1H), 8.40 (d, 2H), 7.86 (d, 2H), 7.58 (d, 1H), 7.42 (t, 1H). LCMS Rₜ=1.225 min in 2.0 min chromatography, MS ESI calcd. for C₁₄H₉F₅N₃ [M+H]⁺ 314.1, found 313.9.

### Example 39: Synthesis of Compound 39

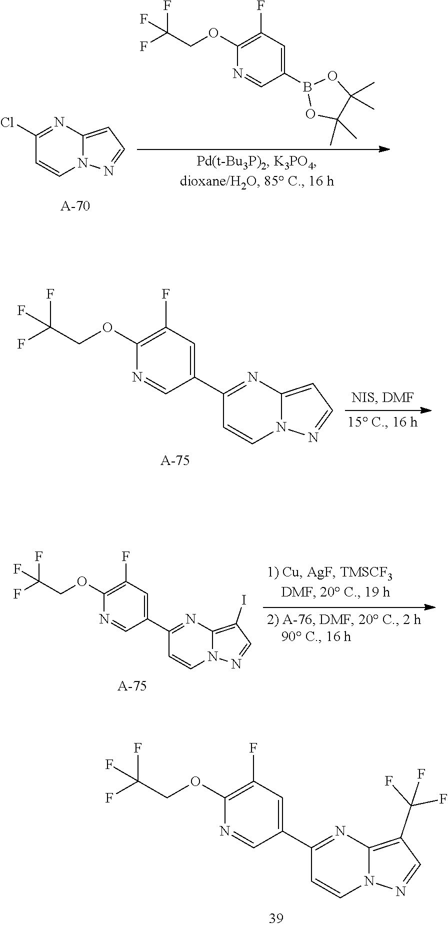

A mixture of 5-chloropyrazolo[1,5-a]pyrimidine (150 mg, 0.98 mmol) 3-fluoro-5-(4,4,5,5-tetramethyl-1,3,2-dioxaborolan-2-yl)-2-(2,2,2-trifluoroethoxy)pyridine (313.62 mg, 0.98 mmol), K₃PO₄ (414.73 mg, 1.95 mmol) and Pd(t-Bu₃P)₂ (74.88 mg, 0.15 mmol) in 1,4-dioxane (8 mL) and water (0.8 mL) under N₂ was stirred at 85° C. for 16 hours. The mixture was cooled to r.t., diluted with EtOAc (5 mL), filtered through silica gel, eluted with EtOAc (5 mL) and concentrated to give the crude product. The crude product was purified by silica gel column (PE:EtOAc=5:1 to 1:1) to give 5-[5-fluoro-6-(2,2,2-trifluoroethoxy)-3-pyridyl]pyrazolo[1,5-a]pyrimidine (350 mg) as a solid. ¹H NMR (400 MHz, CDCl₃) δ_{H} 8.75 (d, 1H), 8.59 (d, 1H), 8.29 (dd, 1H), 8.17 (d, 1H), 7.23 (d, 1H), 6.74 (d, 1H), 4.93 (q, 2H).

To a solution of 5-[5-fluoro-6-(2,2,2-trifluoroethoxy)-3-pyridyl]pyrazolo[1,5-a]pyrimidine (350 mg, 1.12 mmol) in DMF (3 mL) was added NIS (378.31 mg, 1.68 mmol). The resulting mixture was stirred at 15° C. for 16 hours to give a solution. Water (20 mL) was added to the reaction mixture. The solid was collected by filtration and dried in oven to give 5-[5-fluoro-6-(2,2,2-trifluoroethoxy)-3-pyridyl]-3-iodo-pyrazolo[1,5-a]pyrimidine (300 mg, 61% yield) as a solid. ¹H NMR (400 MHz, DMSO-d) δ_{H} 9.30 (d, 1H), 8.94 (d, 1H), 8.56 (dd, 1H), 8.37 (s, 1H), 7.80 (d, 1H), 5.22 (q, 2H).

To a mixture of fluorosilver (1.4 g, 11.03 mmol) in DMF (75 mL) was added trimethyl(trifluoromethyl)silane (2.04 g, 14.35 mmol) under N₂ at 20° C., then the mixture was stirred at 20° C. for 3 hours. To the mixture was added copper (1.12 g, 17.66 mmol), then the mixture was stirred at 20° C. for 16 hours under N₂. The mixture turned to green. trifluoromethylcopper (1460 mg) was obtained as a solution in DMF (˜0.145 M), which was used next step directly.

A mixture of 5-[5-fluoro-6-(2,2,2-trifluoroethoxy)-3-pyridyl]-3-iodo-pyrazolo[1,5-a]pyrimidine (100 mg, 0.23 mmol) in CuCF₃/DMF (12 mL, 1.74 mmol) was stirred in sealed tube at 20° C. for 2 hours and 90° C. for 16 hours to give a suspension. The reaction mixture was cooled to room temperature and filtered through Celite. EtOAc (30 mL) and saturated NH₄Cl (30 mL) was added to the filtrate, and after separated, the organic layer was washed with brine (10 m), dried over anhydrous Na₂SO₄, filtered and concentrated to give the crude product. The crude product was purified by Prep-HPLC (water (10 mM NH₄HCO₃)-ACN) to give 5-[5-fluoro-6-(2,2,2-trifluoroethoxy)-3-pyridyl]-3-(trifluoromethyl)pyrazolo[1,5-a]pyrimidine (4.19 mg, 5% yield) as a solid. ¹H NMR (400 MHz, DMSO-d₆) δ_{H} 9.46 (d, 1H), 8.96 (s, 1H), 8.70 (s, 1H), 8.55 (d, 1H), 8.01 (d, 1H), 5.22 (q, 2H). LCMS Rₜ=1.281 min in 2.0 min chromatography, MS ESI calcd. for C₁₄H₈F₇N₄O [M+H]⁺ 381.1, found 381.0.

### Example 40: Synthesis of Compound 40

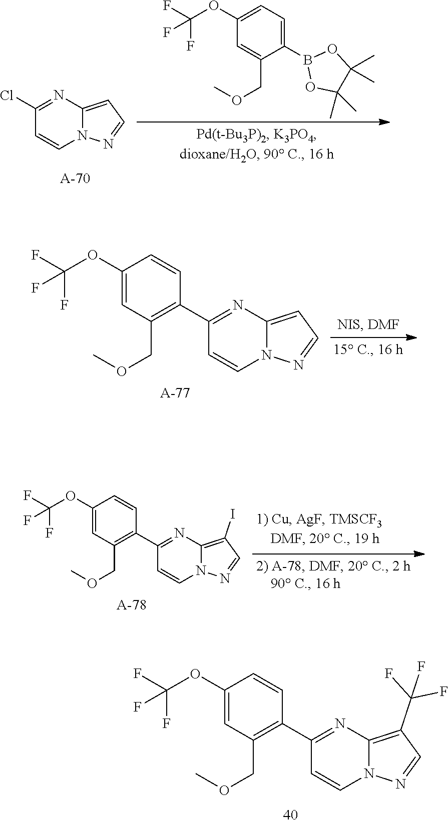

A mixture of 5-chloropyrazolo[1,5-a]pyrimidine (200 mg, 1.3 mmol), 2-[2-(methoxymethyl)-4-(trifluoromethoxy)phenyl]-4,4,5,5-tetramethyl-1,3,2-dioxaborolane (519.04 mg, 1.56 mmol), Pd(t-Bu₃P)₂ (99.83 mg, 0.2 mmol) and K₃PO₄ (552.19 mg, 2.6 mmol) in 1,4-dioxane (8 mL) and water (0.8 mL) under N₂ was stirred at 90° C. for 16 hours. The mixture was cooled to r.t., diluted with EtOAc (5 mL), filtered through silica gel, eluted with EtOAc (5 mL) and concentrated to give the crude product. The crude product was purified by silica gel column (PE:EtOAc=5:1 to 3:1) to give 5-[2-(methoxymethyl)-4-(trifluoromethoxy)phenyl]pyrazolo[1,5-a]pyrimidine (350 mg, 83% yield) as a solid. ¹H NMR (400 MHz, DMSO-d₆) δ_{H} 9.22 (d, 1H), 8.28 (d, 1H), 7.78 (d, 1H), 7.54 (s, 1H), 7.47 (d, 1H), 7.31 (d, 1H), 6.78 (d, 1H), 4.68 (s, 2H), 3.27 (s, 3H).

A mixture of 5-[2-(methoxymethyl)-4-(trifluoromethoxy)phenyl]pyrazolo[1,5-a]pyrimidine (350 mg, 1.08 mmol) and NIS (292.33 mg, 1.3 mmol) in DMF (3 mL) was stirred at 15° C. for 16 hours. The mixture was diluted with H₂O (10 m), and a solid was reformed. The solid was collected by filtration, washed with H₂O (10 mL) and dried to give 3-iodo-5-[2-(methoxymethyl)-4-(trifluoromethoxy)phenyl]pyrazolo[1,5-a]pyrimidine (380 mg, 78% yield) as a solid. ¹H NMR (400 MHz, CDCl₃) δ_{H} 8.71 (d, 1H), 8.19 (s, 1H), 7.67 (d, 1H), 7.57 (s, 1H), 7.33-7.27 (m, 1H), 7.14 (d, 1H), 4.81 (s, 2H), 3.43 (s, 3H).

To a mixture of fluorosilver (1.4 g, 11.03 mmol) in DMF (75 mL) was added trimethyl(trifluoromethyl)silane (2.04 g, 14.35 mmol) under N₂ at 20° C., then the mixture was stirred at 20° C. for 3 hours. To the mixture was added copper (1.12 g, 17.66 mmol), then the mixture was stirred at 20° C. for 16 hours under N₂. The mixture turned to green. trifluoromethylcopper (1460 mg) was obtained as a solution in DMF (˜0.145 M), which was used next step directly.

A mixture of 3-iodo-5-[2-(methoxymethyl)-4-(trifluoromethoxy)phenyl]pyrazolo[1,5-a]pyrimidine (100 mg, 0.22 mmol) in CuCF₃/DMF (12 mL, 1.74 mmol) was stirred in a sealed tube at 20° C. for 2 hours and 90° C. for 16 hours to give a suspension The reaction mixture was cooled to room temperature and filtered through Celite. EtOAc (30 mL) and saturated NH₄Cl (30 mL) were added to the filtrate, and after separated, the organic layer was washed with brine (10 m), dried over anhydrous Na₂SO₄, filtered and concentrated to give the crude product. The crude product was purified by Prep-HPLC (water (10 mM NH₄HCO₃)-ACN) to give 5-[2-(methoxymethyl)-4-(trifluoromethoxy)phenyl]-3-(trifluoromethyl)pyrazolo[1,5-a]pyrimidine (44.71 mg, 51% yield) as a solid. ¹H NMR (400 MHz, CDCl₃) δ_{H} 8.79 (d, 1H), 8.36 (s, 1H), 7.69 (d, 1H), 7.59 (s, 1H), 7.32-7.28 (m, 2H), 4.80 (s, 2H), 3.42 (s, 3H). LCMS Rₜ=1.310 min in 2.0 min chromatography, MS ESI calcd. for C₁₆H₁₂F₆N₃O₂ [M+H]⁺ 392.1, found 392.0.

### Example 41: Synthesis of Compound 41

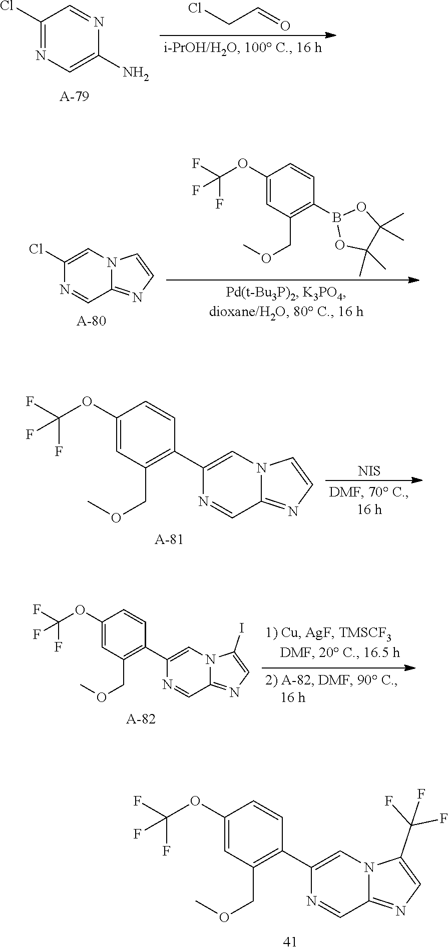

To a solution of 5-chloropyrazin-2-amine (2.5 g, 19.3 mmol) in IPA (25 mL) was added 2-chloroacetaldehyde (5.68 g, 28.95 mmol). The resulting mixture was stirred at 100° C. for 16 hours to give a black suspension. The reaction solution was cooled to room temperature and concentrated to give a residue. The residue was dissolved in EtOAc (150 mL) and K₂CO₃ aqueous (60 m). After separation, the organic layer was washed with brine (50 mL×2), dried over anhydrous Na₂SO₄, filtered and concentrated to give a crude product. The crude product was purified by silica gel column with EtOAc in PE=(10% to 50%) to give 6-chloroimidazo[1,2-a]pyrazine (1000 mg, 33% yield) as a solid. ¹H NMR (400 MHz, CDCl₃) δ_{H} 8.94 (s, 1H), 8.20 (s, 1H), 7.88 (s, 1H), 7.73 (s, 1H). LCMS Rₜ=0.174 min in 1.5 min chromatography, MS ESI calcd. for C₆H₅ClN₃ [M+H]⁺ 154.0, found 153.8.

To a solution of 6-chloroimidazo[1,2-a]pyrazine (110 mg, 0.72 mmol) in 1,4-dioxane (5 mL) and water (0.50 mL) was added 2-[2-(methoxymethyl)-4-(trifluoromethoxy)phenyl]-4,4,5,5-tetramethyl-1,3,2-dioxaborolane (285.47 mg, 0.86 mmol), Pd(t-Bu₃P)₂ (73.22 mg, 0.14 mmol) and K₃PO₄ (304.13 mg, 1.43 mmol). The resulting mixture was stirred at 80° C. under N₂ for 16 hours to give a suspension. The reaction mixture was cooled to room temperature and filtered through Celite. The filtrate was concentrated to give a crude product. The crude product was purified by silica gel column with EtOAc in PE (0% to 30% to 60% to 90%) to give 6-[2-(methoxymethyl)-4-(trifluoromethoxy)phenyl]imidazo[1,2-a]pyrazine (100 mg, 35% yield) as an oil. ¹H NMR (400 MHz, CDCl₃) δ_{H} 9.18 (br s, 1H), 8.39 (s, 1H), 7.88 (s, 1H), 7.79-7.72 (m, 1H), 7.60 (d, 1H), 7.47 (s, 1H), 7.29 (s, 1H), 4.55 (s, 2H), 3.41 (s, 3H). LCMS Rₜ=0.730 min in 1.5 min chromatography, MS ESI calcd. for C₁₅H₁₃F₃N₃O₂ [M+H]⁺ 324.1, found 323.9.

To a solution of 6-[2-(methoxymethyl)-4-(trifluoromethoxy)phenyl]imidazo[1,2-a]pyrazine (100 mg, 0.31 mmol) in DMF (2 mL) was added NIS (104.39 mg, 0.46 mmol). The resulting mixture was stirred at 70° C. for 16 hours to give a solution. The reaction mixture was cooled to room temperature. EtOAc (15 mL) and H₂O (10 mL) were added to the mixture. After separated, the organic layer was washed with brine (10 mL×2), dried over anhydrous Na₂SO₄, filtered and concentrated to give the crude product. The crude product was purified by silica gel column with EtOAc in PE=(0% to 30% to 60%) to give 3-iodo-6-[2-(methoxymethyl)-4-(trifluoromethoxy)phenyl]imidazo[1,2-a]pyrazine (66 mg, 48% yield) as a solid. ¹H NMR (400 MHz, CDCl₃) δ_{H} 9.07 (d, 1H), 8.46 (d, 1H), 7.93 (s, 1H), 7.69 (d, 1H), 7.48 (s, 1H), 7.32 (d, 1H), 4.52 (s, 2H), 3.48 (s, 3H).

To a mixture of fluorosilver (1.4 g, 11.03 mmol) in DMF (75 mL) was added trimethyl(trifluoromethyl)silane (2.04 g, 14.35 mmol) under N₂ at 20° C., then the mixture was stirred at 20° C. for 3 hours. To the mixture was added copper (1.12 g, 17.66 mmol), then the mixture was stirred at 20° C. for 16 hours under N₂. The mixture turned to green. trifluoromethylcopper (1460 mg) was obtained as a solution in DMF (0.145 M), which was used next step directly.

To a solution of 3-iodo-6-[2-(methoxymethyl)-4-(trifluoromethoxy)phenyl]imidazo[1,2-a]pyrazine (66 mg, 0.15 mmol) in DMF (1 mL) was added CuCF₃/DMF (12 mL, 1.74 mmol). The resulting mixture was stirred at sealed tube under N₂ for 16 hours to give a suspension. The reaction mixture was cooled to room temperature. EtOAc (50 mL) and Water (30 mL) was added to the mixture. After separation, the organic layer was washed with brine (20 mL×2), dried over anhydrous Na₂SO₄, filtered and concentrated to give a crude product. The crude product was purified by Prep-HPLC (water (0.05% ammonia hydroxide v/v)-ACN) to give 6-[2-(methoxymethyl)-4-(trifluoromethoxy)phenyl]-3-(trifluoromethyl)imidazo[1,2-a]pyrazine (27.03 mg, 47% yield) as a solid. ¹H NMR (400 MHz, CDCl₃) δ_{H} 9.31 (s, 1H), 8.63 (s, 1H), 8.16 (s, 1H), 7.71 (d, 1H), 7.46 (s, 1H), 7.34 (d, 1H), 4.49 (s, 2H), 3.45 (s, 3H). LCMS Rₜ=1.289 min in 2.0 min chromatography, MS ESI calcd. for C₁₆H₁₂F₆N₃O₂ [M+H]⁺ 392.1, found 392.0.

### Example 42: Synthesis of Compound 42

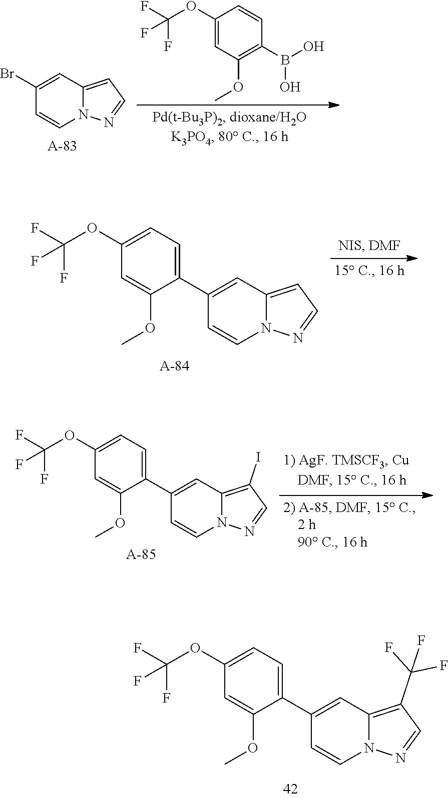

A mixture of 5-bromopyrazolo[1,5-a]pyridine (150.00 mg, 761.31 μmol), [2-methoxy-4-(trifluoromethoxy)phenyl]boronic acid (197.59 mg, 837.44 μmol), Pd(t-Bu₃P)₂ (58.36 mg, 114.20 umol) and K₃PO₄ (323.20 mg, 1.52 mmol) in dioxane (2 mL) and H₂O (0.2 mL) was stirred at 80° C. for 16 hours. The mixture was cooled to r.t., diluted with EtOAc (5 mL), filtered through silica gel, eluted with EtOAc (10 mL) and concentrated to give the crude product. The crude product was purified by silica gel column (PE:EtOAc=10:1 to 5:1) to give 5-[2-methoxy-4-(trifluoromethoxy)phenyl]pyrazolo[1,5-a]pyridine (200.00 mg, 85% yield) as an oil. ¹H NMR (400 MHz, DMSO-d₆) δ_{H} 8.68 (d, 1H), 8.01 (d, 1H), 7.78 (d, 1H), 7.54 (d, 1H), 7.16 (d, 1H), 7.06 (dd, 1H), 7.00 (dd, 1H), 6.64 (d, 1H), 3.85 (s, 3H).

To a solution of 5-[2-methoxy-4-(trifluoromethoxy)phenyl]pyrazolo[1,5-a]pyridine (200 mg, 648.82 μmol) in DMF (5 mL) was added NIS (175.17 mg, 778.58 μmol), and the mixture was stirred at 15° C. for 16 hours. The mixture was diluted with H₂O (50 mL). A solid was formed and collected by filtration. The solid was dried to 3-iodo-5-[2-methoxy-4-(trifluoromethoxy)phenyl]pyrazolo[1,5-a]pyridine (230.00 mg, 82% yield). ¹H NMR (400 MHz, CDCl₃) δ_{H} 8.44 (d, 1H), 7.96 (s, 1H), 7.53 (d, 1H), 7.41 (d, 1H), 7.00 (dd, 1H), 6.95 (d, 1H), 6.86 (s, 1H), 3.87 (s, 3H).

To a solution of AgF (30.39 mg, 239.55 μmol) in DMF (10 mL) was added TMSCF₃ (41.93 mg, 294.83 μmol) and the mixture was stirred at 15° C. for 1 hour. To the mixture added Cu (23.42 mg, 368.54 μmol) and the mixture was stirred at 15° C. for 16 hours. To the mixture added 3-iodo-5-[2-methoxy-4-(trifluoromethoxy)phenyl]pyrazolo[1,5-a]pyridine (80.00 mg, 184.27 μmol) and the mixture was stirred at 15° C. for 2 hours and 90° C. for 16 hours. The mixture was cooled to r.t., diluted with EtOAc (5 mL), filtered through silica gel, eluted with EtOAc (10 mL) and the filtrate was concentrated to give the crude product. The crude product was purified by Prep-HPLC (column: Waters Xbridge 150*25 5u; mobile phase: [water (10 mM NH₄HCO₃)-ACN]; B %: 54%-84%, 10 min) to give 5-[2-methoxy-4-(trifluoromethoxy)phenyl]-3-(trifluoromethyl)pyrazolo[1,5-a]pyridine (17.59 mg, 25% yield) as a solid. ¹H NMR (400 MHz, CDCl₃) δ_{H} 8.52 (d, 1H), 8.17 (s, 1H), 7.77 (s, 1H), 7.42 (d, 1H), 7.14 (dd, 1H), 6.97 (d, 1H), 6.88 (s, 1H), 3.88 (s, 3H). LCMS Rₜ=1.470 min in 2.0 min chromatography, MS ESI calcd. for C₁₆H₁₁F₆N₂O₂ [M+H]⁺ 377.1, found 377.0.

### Example 43: Synthesis of Compound 43

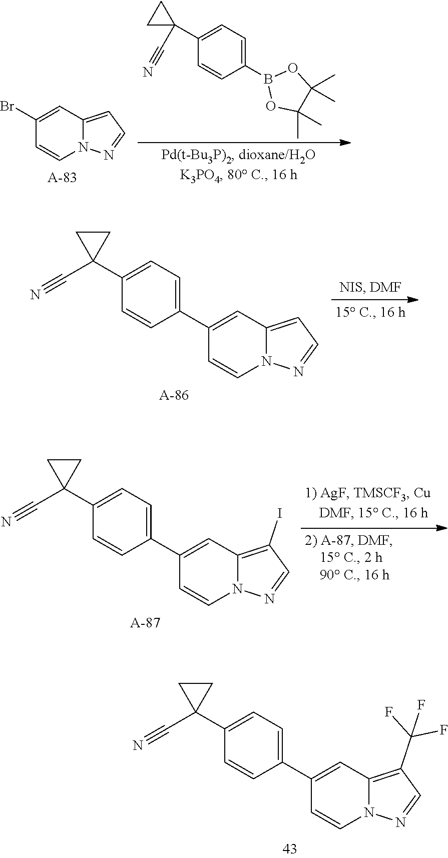

A mixture of 5-bromopyrazolo[1,5-a]pyridine (0.15 g, 761.30 μmol), 1-[4-(4,4,5,5-tetramethyl-1,3,2-dioxaborolan-2-yl)phenyl]cyclopropanecarbonitrile (245.88 mg, 913.56 μmol), Pd(t-Bu₃P)₂ (58.36 mg, 114.19 mol) and K₃PO₄ (323.20 mg, 1.52 mmol) in dioxane (2 mL) and H₂O (0.2 mL) was stirred at 80° C. for 16 hours. The crude product was cooled to r.t., diluted with H₂O (10 mL), extracted with EtOAc (30 mL×2). The combined organic phase was washed with brine (10 mL), dried over Na₂SO₄, filtered and concentrated to give the crude product. The crude product was purified by silica gel column (PE:EtOAc=10:1 to 5:1 to 1:1) to give 1-(4-pyrazolo[1,5-a]pyridin-5-ylphenyl)cyclopropanecarbonitrile (150 mg, 76% yield) as a solid. ¹H NMR (400 MHz, CDCl₃) δ_{H} 8.53 (d, 1H), 7.99 (d, 1H), 7.72 (d, 1H), 7.64 (d, 2H), 7.41 (d, 2H), 7.00 (dd, 1H), 6.58 (d, 1H), 1.83-1.78 (m, 2H), 1.50-1.46 (m, 2H).

To a solution of 1-(4-pyrazolo[1,5-a]pyridin-5-ylphenyl)cyclopropanecarbonitrile (150 mg, 578.47 μmol) in DMF (10 mL) was added NIS (156.18 mg, 694.16 μmol), and the mixture was stirred at 15° C. for 16 hours. The mixture was diluted with H₂O (50 mL). A solid was formed and collected by filtration. The solid was dried to give 1-[4-(3-iodopyrazolo[1,5-a]pyridin-5-yl)phenyl]cyclopropanecarbonitrile (140 mg, 63% yield) as a solid. ¹H NMR (400 MHz, DMSO-d₆) δ_{H} 8.82 (d, 1H), 8.15 (s, 1H), 7.88 (d, 2H), 7.70 (s, 1H), 7.47 (d, 2H), 7.31 (dd, 1H), 1.85-1.79 (m, 2H), 1.62-1.55 (m, 2H).

To a solution of AgF (30.39 mg, 239.55 μmol) in DMF (10 mL) was added TMSCF₃ (41.93 mg, 294.83 μmol) and the mixture was stirred at 15° C. for 1 hour. To the mixture added Cu (23.42 mg, 368.54 μmol) and the mixture was stirred at 15° C. for 6 hours. To the mixture was added 1-[4-(3-iodopyrazolo[1,5-a]pyridin-5-yl)phenyl]cyclopropanecarbonitrile (70.98 mg, 184.27 μmol) and the mixture was stirred at 15° C. for 2 hours then at 90° C. for 16 hours. The mixture was cooled to r.t., diluted with EtOAc (5 mL), filtered through silica gel, eluted with EtOAc (10 mL) and concentrated to give the crude product. The crude product was purified by Prep-HPLC (column: Waters Xbridge 150*25 5u; mobile phase: [water (10 mM NH₄HCO₃)-ACN]; B %: 42%-72%, 10 min) to 1-[4-[3-(trifluoromethyl)pyrazolo[1,5-a]pyridin-5-yl]phenyl]cyclopropanecarbonitrile (11.72 mg, 19% yield) as a solid. ¹H NMR (400 MHz, DMSO-d₆) δ_{H} 8.97 (dd, 1H), 8.50 (s, 1H), 7.95 (s, 1H), 7.90 (d, 2H), 7.51 (dd, 1H), 7.48 (d, 2H), 1.85-1.80 (m, 2H), 1.61-1.57 (m, 2H). LCMS Rₜ=1.228 min in 2.0 min chromatography, MS ESI calcd. for C₁₈H₁₃F₃N₃ [M+H]⁺ 328.1, found 327.9.

### Example 44: Synthesis of Compound 44

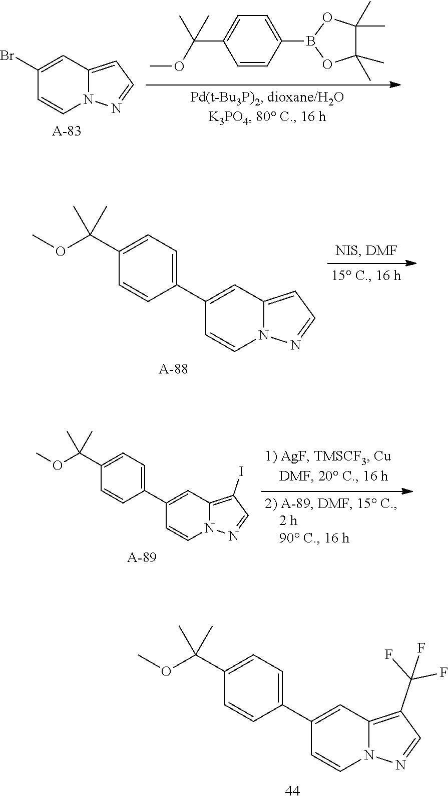

A mixture of 5-bromopyrazolo[1,5-a]pyridine (150 mg, 761.30 μmol), 2-[4-(1-methoxy-1-methyl-ethyl)phenyl]-4,4,5-trimethyl-1,3,2-dioxaborolane (598.73 mg, 2.28 mmol), Pd(t-Bu₃P)₂ (58.36 mg, 114.19 μmol) and K₃PO₄ (323.20 mg, 1.52 mmol) in dioxane (2 mL) and H₂O (0.2 mL) was stirred at 80° C. for 16 hours. The mixture was cooled to r.t., diluted with H₂O (10 mL), extracted with EtOAc (30 mL×2). The combined organic phase was washed with brine (10 mL), dried over Na₂SO₄, filtered and concentrated to give the crude product. The crude product was purified by silica gel column (PE:EtOAc=8:1 to 5:1) to give 5-[4-(1-methoxy-1-methyl-ethyl)phenyl]pyrazolo[1,5-a]pyridine (120 mg, 59% yield) as a solid. ¹H NMR (400 MHz, DMSO-d₆) δ_{H} 8.73 (d, 1H), 8.02-7.99 (m, 2H), 7.78 (d, 2H), 7.50 (d, 2H), 7.23 (dd, 1H), 6.65 (d, 1H), 3.01 (s, 3H), 1.48 (s, 6H).

To a solution of 5-[4-(1-methoxy-1-methyl-ethyl)phenyl]pyrazolo[1,5-a]pyridine (120 mg, 450.56 μmol) in DMF (10 mL) was added NIS (121.64 mg, 540.67 μmol), and the mixture was stirred at 15° C. for 16 hours. The mixture was diluted with H₂O (50 mL). A solid was formed and collected by filtration. The solid was dried to give 3-iodo-5-[4-(1-methoxy-1-methyl-ethyl)phenyl]pyrazolo[1,5-a]pyridine (150 mg, 85% yield) as a solid. ¹H NMR (400 MHz, DMSO-d₆) δ_{H} 8.81 (d, 1H), 8.14 (s, 1H), 7.82 (d, 2H), 7.68 (s, 1H), 7.52 (d, 2H), 7.31 (dd, 1H), 3.02 (s, 3H), 1.49 (s, 6H).

To a solution of AgF (62.96 mg, 496.26 μmol) in DMF (5 mL) was added TMSCF₃ (86.85 mg, 610.78 μmol) and the mixture was stirred at 15° C. for 1 hour. To the mixture added Cu (48.52 mg, 763.48 μmol) and the mixture was stirred at 15° C. for 16 hours. To the mixture added 3-iodo-5-[4-(1-methoxy-1-methyl-ethyl)phenyl]pyrazolo[1,5-a]pyridine (74.87 mg, 190.87 μmol) and the mixture was stirred at 15° C. for 2 hours and 90° C. for 16 hours. The mixture was cooled to r.t., diluted with EtOAc (5 mL), filtered through silica gel, eluted with EtOAc (10 mL) and concentrated to give the crude product. The crude product was purified by Prep-HPLC (column: Waters Xbridge 150*25 Su; mobile phase: [water (10 mM NH₄HCO₃)-ACN]; B %: 50%-80%, 10 min) to give 5-[4-(1-methoxy-1-methyl-ethyl)phenyl]-3-(trifluoromethyl)pyrazolo[1,5-a]pyridine (10.13 mg, 16% yield) as a solid. ¹H NMR (400 MHz, DMSO-d₆) δ_{H} 8.97 (dd, 1H), 8.50 (s, 1H), 7.94 (s, 1H), 7.85 (d, 2H), 7.54 (d, 2H), 7.51 (d, 1H), 3.02 (s, 3H), 1.49 (s, 6H). LCMS Rₜ=1.278 min in 2.0 min chromatography, MS ESI calcd. for C₁₈H₁₈F₃N₂O [M+H]⁺ 335.1, found 334.9.

### Example 45: Synthesis of Compound 45

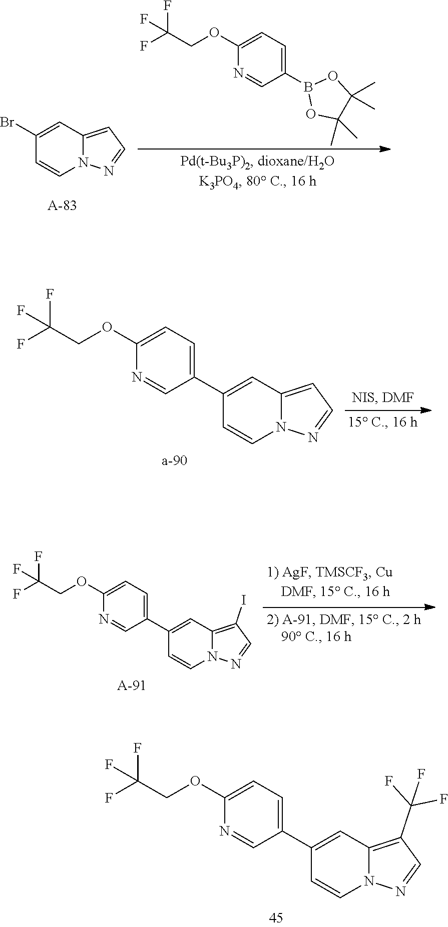

A mixture of 5-bromopyrazolo[1,5-a]pyridine (150.00 mg, 761.31 μmol), 5-(4,4,5,5-tetramethyl-1,3,2-dioxaborolan-2-yl)-2-(2,2,2-trifluoroethoxy)pyridine (253.81 mg, 837.44 mol), Pd(t-Bu₃P)₂ (58.36 mg, 114.20 mol) and K₃PO₄ (323.20 mg, 1.52 mmol) in dioxane (2 mL) and H₂O (0.2 mL) was stirred at 80° C. for 16 hours. The crude product was cooled to r.t., diluted with EtOAc (10 mL), filtered through silica gel, eluted with EtOAc (20 mL) and concentrated to give the crude product. The crude product was purified by silica gel column (PE:EtOAc=10:1 to 5:1) to give 5-[6-(2,2,2-trifluoroethoxy)-3-pyridyl]pyrazolo[1,5-a]pyridine (120.0 mg, 53% yield) as a solid. LCMS Rₜ=0.868 min in 1.5 min chromatography, MS ESI calcd. for C₁₄H₁₁F₃N₃O [M+H]⁺ 294.1, found 293.9.

To a solution of 5-[6-(2,2,2-trifluoroethoxy)-3-pyridyl]pyrazolo[1,5-a]pyridine (120.00 mg, 409.22 umol, 1.00 eq) in DMF (10.00 mL) was added NIS (110.48 mg, 491.06 μmol), and the mixture was stirred at 15° C. for 16 hours. The mixture was diluted with H₂O (50 mL). A solid was formed and collected by filtration. The solid was dried to give 3-iodo-5-[6-(2,2,2-trifluoroethoxy)-3-pyridyl]pyrazolo[1,5-a]pyridine (150.0 mg, 87% yield). ¹H NMR (400 MHz, CDCl₃) δ_{H} 8.52 (dd, 1H), 8.47 (d, 1H), 8.00 (s, 1H), 7.96 (dd, 1H), 7.59-7.57 (m, 1H), 7.05-6.97 (m, 2H), 4.85 (q, 2H).

To a solution of AgF (62.96 mg, 496.26 μmol) in DMF (5 mL) was added TMSCF₃ (86.85 mg, 610.78 μmol) and the mixture was stirred at 15° C. for 1 hour. To the mixture added Cu (48.52 mg, 763.48 μmol) and the mixture was stirred at 15° C. for 16 hours. To the mixture added 3-iodo-5-[6-(2,2,2-trifluoroethoxy)-3-pyridyl]pyrazolo[1,5-a]pyridine (80.00 mg, 190.87 mol) and the mixture was stirred at 15° C. for 2 hours and 90° C. for 16 hours. The mixture was cooled to r.t., diluted with EtOAc (5 mL), filtered through silica gel, eluted with EtOAc (10 mL) and concentrated to give the crude product. The crude product was purified by Prep-HPLC (column: Waters Xbridge 150*25 5u; mobile phase: [water (10 mM NH₄HCO₃)-ACN]; B %: 48%-78%, 10 min) to give 5-[6-(2,2,2-trifluoroethoxy)-3-pyridyl]-3-(trifluoromethyl)pyrazolo[1,5-a]pyridine (10.06 mg, 14% yield) as a solid. ¹H NMR (400 MHz, CDCl₃) δ_{H} 8.60 (d, 1H), 8.47 (d, 1H), 8.19 (s, 1H), 7.95 (dd, 1H), 7.80 (s, 1H), 7.14 (dd, 1H), 7.03 (d, 1H), 4.85 (q, 2H). LCMS Rₜ=1.391 min in 2.0 min chromatography, MS ESI calcd. for C₁₅H₁₀F₆N₃O [M+H]⁺ 362.1, found 362.0.

### Example 46: Synthesis of Compound 46

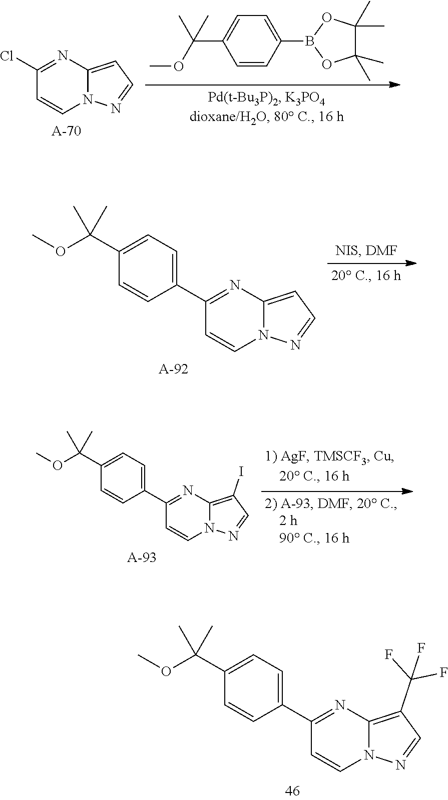

A mixture of 5-chloropyrazolo[1,5-a]pyrimidine (200.0 mg, 1.30 mmol), 2-[4-(1-methoxy-1-methyl-ethyl)phenyl]-4,4,5,5-tetramethyl-1,3,2-dioxaborolane (538.55 mg, 1.95 mmol), Pd(t-Bu₃P)₂ (99.65 mg, 195.00 μmol) and K₃PO₄ (551.90 mg, 2.60 mmol) in dioxane (10 mL) and H₂O (2.60 mL) was stirred at 80° C. for 16 hours under N₂. After cooling to r.t., the mixture was concentrated to give the crude product. The crude product was purified by silica gel column (PE in EtOAc=7:1 to 5:1 to 4:1 to 3:1) to give 5-[4-(1-methoxy-1-methyl-ethyl)phenyl]pyrazolo[1,5-a]pyrimidine (320.00 mg, 77% yield) as a solid. ¹H NMR (400 MHz, CDCl₃) δ_{H} 8.73 (br s, 1H), 8.14 (d, 1H), 8.08 (d, 2H), 7.59-7.55 (m, 2H), 7.29 (d, 1H), 6.72 (d, 1H), 3.13 (s, 3H), 1.59 (s, 6H). LCMS Rₜ=0.787 min in 1.5 min chromatography, MS ESI calcd. for C₁₆H₁₈N₃O [M+H]⁺ 268.1, found 267.9.

To a solution of 5-[4-(1-methoxy-1-methyl-ethyl)phenyl]pyrazolo[1,5-a]pyrimidine (220.0 mg, 822.95 μmol) in DMF (5 mL) was added NIS (222.18 mg, 987.54 μmol), and the mixture was stirred at 20° C. for 16 hours. To the mixture was added H₂O (15 mL) and the solid formed was collected by filtration, washed with water (10 mL×3) and dried in an oven to give 3-iodo-5-[4-(1-methoxy-1-methyl-ethyl)phenyl]pyrazolo[1,5-a]pyrimidine (190.00 mg, 49% yield) as a solid. ¹H NMR (400 MHz, DMSO-d) δ_{H} 9.18 (d, 1H), 8.32 (s, 1H), 8.22 (d, 2H), 7.69 (d, 1H), 7.59 (d, 2H), 3.03 (s, 3H), 1.50 (s, 6H). LCMS Rₜ=0.888 min in 1.5 min chromatography, MS ESI calcd. for C₁₆H₁₇IN₃O [M+H]⁺ 394.0, found 393.9.

To a mixture of AgF (41.94 mg, 330.60 μmol) was added TMSCF₃ (57.86 mg, 406.90 mol), then the mixture was stirred at 20° C. for 1 hour. To the mixture was added Cu (32.32 mg, 508.62 μmol), then the mixture was stirred at 20° C. 15 hours under N₂. To the mixture was added 3-iodo-5-[4-(1-methoxy-1-methyl-ethyl)phenyl]pyrazolo[1,5-a]pyrimidine (100.00 mg, 254.31 mol), then the mixture was stirred at 20° C. for 2 hours and 90° C. for 16 hours. After cooling to r.t., the mixture was diluted with H₂O (10 mL), and the mixture was extracted with EtOAc (20 mL×2). The combined organic phase was washed with brine (15 mL), dried over Na₂SO₄, filtered and concentrated to give the crude product. The crude product was purified by Prep-HPLC (column: Kromasil 150*25 mm*10 um; mobile phase: [water (0.05% ammonia hydroxide v/v)-ACN]; B %: 58%-68%, 8 min) to give 5-[4-(1-methoxy-1-methyl-ethyl)phenyl]-3-(trifluoromethyl)pyrazolo[1,5-a]pyrimidine (24.70 mg, 29% yield) as a solid. ¹H NMR (400 MHz, CDCl₃) δ_{H} 8.75 (d, 1H), 8.31 (s, 1H), 8.17 (d, 2H), 7.60 (d, 2H), 7.48 (d, 1H), 3.13 (s, 3H), 1.59 (s, 6H). LCMS Rₜ=1.235 min in 2.0 min chromatography, MS ESI calcd. for C₁₇H₁₇F₃N₃O [M+H]⁺ 336.1, found 335.9.

### Example 47: Synthesis of Compound 47

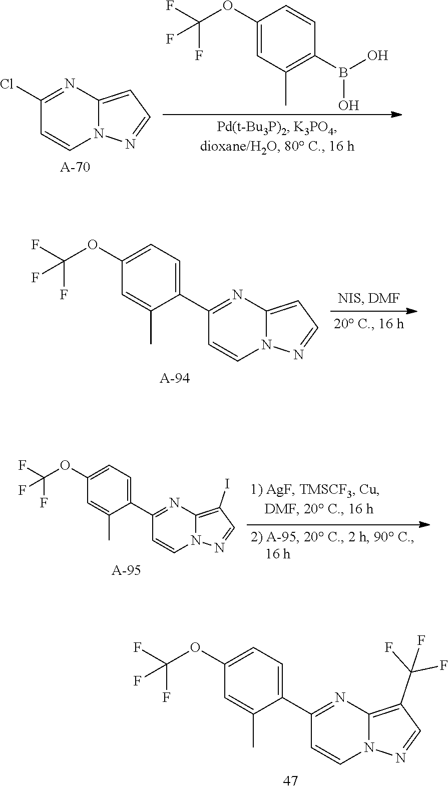

A mixture of 5-chloropyrazolo[1,5-a]pyrimidine (125.68 mg, 818.36 μmol), [2-methyl-4-(trifluoromethoxy)phenyl]boronic acid (150.00 mg, 681.97 μmol), Pd(t-Bu₃P)₂ (52.28 mg, 102.30 μmol) and K₃PO₄ (289.52 mg, 1.36 mmol) in dioxane (10 mL) and H₂O (1.50 mL) was stirred at 80° C. for 16 hours. After cooling to r.t., the mixture was concentrated to give the crude product. The crude product was purified by silica gel column (PE in EtOAc=1:5 to 1:3 to 1:1) to give 5-[2-methyl-4-(trifluoromethoxy)phenyl]pyrazolo[1,5-a]pyrimidine (220.00 mg, 93% yield) as a solid. ¹H NMR (400 MHz, CDCl₃) δ_{H} 8.75 (br s, 1H), 8.18 (d, 1H), 7.59-7.48 (m, 1H), 7.22-7.16 (m, 2H), 6.96 (d, 1H), 6.73 (d, 1H), 2.49 (s, 3H). LCMS Rₜ=0.846 min in 1.5 min chromatography, MS ESI calcd. for C₁₄H₁₁F₃N₃O [M+H]⁺ 294.1, found 293.9.

To a solution of 5-[2-methyl-4-(trifluoromethoxy)phenyl]pyrazolo[1,5-a]pyrimidine (170.00 mg, 579.73 μmol) in DMF (5 mL) was added NIS (156.51 mg, 695.68 μmol), and the mixture was stirred at 20° C. for 16 hours. To the mixture was added H₂O (20 mL) and the solid formed was collected by filtration, washed with water (10 mL×3) and dried in oven to give 3-iodo-5-[2-methyl-4-(trifluoromethoxy)phenyl]pyrazolo[1,5-a]pyrimidine (140.0 mg, 57% yield) as a solid. ¹H NMR (400 MHz, CDCl₃) δ_{H} 8.70 (d, 1H), 8.19 (s, 1H), 7.58 (d, 1H), 7.22-7.17 (m, 2H), 7.01 (d, 1H), 2.57 (s, 3H). LCMS Rₜ=0.932 min in 1.5 min chromatography, MS ESI calcd. for C₁₄H₁₀F₃IN₃O [M+H]⁺ 420.0, found 419.9.

To a mixture of AgF (31.48 mg, 248.13 μmol) was added TMSCF₃ (43.43 mg, 305.39 mol), then the mixture was stirred at 20° C. for 1 hour. To the mixture was added Cu (24.26 mg, 381.74 μmol), then the mixture was stirred at 20° C. 16 hours under N₂. To the mixture was added 3-iodo-5-[2-methyl-4-(trifluoromethoxy)phenyl]pyrazolo[1,5-a]pyrimidine (80.00 mg, 190.87 μmol), then the mixture was stirred at 20° C. for 2 hours and 90° C. for 16 hours. After cooling to r.t., the mixture was poured into water (20 mL), extracted with EtOAc (20 mL×2). The combined organic phase was washed with brine (15 mL), dried over Na₂SO₄, filtered and concentrated to give the crude product. The crude product was purified by Prep-HPLC (column: Kromasil 150*25 mm*10 um; mobile phase: [water (0.05% ammonia hydroxide v/v)-ACN]; B %: 52%-82%, 8 min) to give 5-[2-methyl-4-(trifluoromethoxy)phenyl]-3-(trifluoromethyl)pyrazolo[1,5-a]pyrimidine (34.16 mg, 50% yield) as a solid. ¹H NMR (400 MHz, CDCl₃) δ_{H} 8.79 (d, 1H), 8.36 (s, 1H), 7.60 (d, 1H), 7.23-7.16 (m, 3H), 2.57 (s, 3H). LCMS Rₜ=1.285 min in 2.0 min chromatography, MS ESI calcd. for C₁₅HOF₆N₃O [M+H]⁺ 362.1, found 361.8.

### Example 48: Synthesis of Compound 48

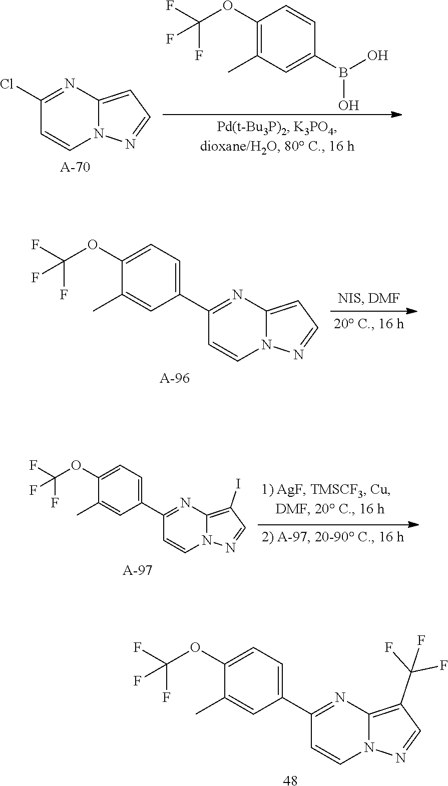

A mixture of 5-chloropyrazolo[1,5-a]pyrimidine (83.78 mg, 545.58 μmol), [3-methyl-4-(trifluoromethoxy)phenyl]boronic acid (100.00 mg, 454.65 μmol), Pd(t-Bu₃P)₂ (34.85 mg, 68.20 mol) and K₃PO₄ (193.02 mg, 909.30 μmol) in dioxane (5 mL) and H₂O (0.6 mL) was stirred at 80° C. for 16 hours. After cooling to r.t., the mixture was concentrated to give the crude product. The crude product was purified by Prep-TLC (PE:EtOAc=2:1) to give 5-[3-methyl-4-(trifluoromethoxy)phenyl]pyrazolo[1,5-a]pyrimidine (110.00 mg, 79% yield) as a solid. ¹H NMR (400 MHz, CDCl₃) δ_{H} 8.74 (br s, 1H), 8.16 (d, 1H), 8.04 (d, 1H), 7.92 (dd, 1H), 7.35 (dd, 1H), 7.26 (d, 1H), 6.74 (d, 1H), 2.44 (s, 3H). LCMS Rₜ=0.984 min in 1.5 min chromatography, MS ESI calcd. for C₁₄H₁₁F₃N₃O [M+H]⁺ 294.1, found 293.9.

To a solution of 5-[3-methyl-4-(trifluoromethoxy)phenyl]pyrazolo[1,5-a]pyrimidine (100.00 mg, 341.02 μmol) in DMF (3 mL) was added NIS (92.07 mg, 409.22 μmol), and the mixture was stirred at 20° C. for 16 hours. To the mixture was added H₂O (3 mL) and the solid formed was collected by filtration, washed with water (4 mL×3) and dried in oven to give 3-iodo-5-[3-methyl-4-(trifluoromethoxy)phenyl]pyrazolo[1,5-a]pyrimidine (120.00 mg, 79% yield) as a solid. ¹H NMR (400 MHz, CDCl₃) δ_{H} 8.69 (d, 1H), 8.16 (s, 1H), 8.09 (d, 1H), 8.01 (dd, 1H), 7.37 (dd, 1H), 7.29 (d, 1H), 2.45 (s, 3H). LCMS Rₜ=0.965 min in 1.5 min chromatography, MS ESI calcd. for C₁₄H₁₀F₃IN₃O [M+H]⁺ 420.0, found 419.8.

To a mixture of AgF (31.48 mg, 248.13 μmol) in DMF (10 mL) was added TMSCF₃ (43.43 mg, 305.39 μmol), then the mixture was stirred at 20° C. for 1 hour. To the mixture was added Cu (24.26 mg, 381.74 μmol), then the mixture was stirred at 20° C. for 15 hours under N₂. To the mixture was added 3-iodo-5-[3-methyl-4-(trifluoromethoxy)phenyl]pyrazolo[1,5-a]pyrimidine (80.00 mg, 190.87 mol), then the mixture was stirred at 20° C. for 2 hours and 90° C. for 16 hours. After cooling to r.t., the mixture was diluted with H₂O (20 mL), and the mixture was extracted with EtOAc (15 mL×2). The combined organic phase was washed with brine (10 mL), dried over Na₂SO₄, filtered and concentrated to give the crude product. The crude product was purified by Prep-HPLC (column: Kromasil 150*25 mm*10 um; mobile phase: [water (0.05% ammonia hydroxide v/v)-ACN]; B %: 55%-85%, 8 min) to give 5-[3-methyl-4-(trifluoromethoxy)phenyl]-3-(trifluoromethyl)pyrazolo[1,5-a]pyrimidine (16.70 mg, 24% yield) as a solid. ¹H NMR (400 MHz, CDCl₃) δ_{H} 8.76 (d, 1H), 8.33 (s, 1H), 8.09 (s, 1H), 8.02 (dd, 1H), 7.44 (d, 1H), 7.38 (dd, 1H), 2.45 (s, 3H). LCMS Rₜ=1.339 min in 2.0 min chromatography, MS ESI calcd. for C₁₅H₁₀F₆N₃O [M+H]⁺ 362.1, found 361.9.

### Example 49: Synthesis of Compound 49

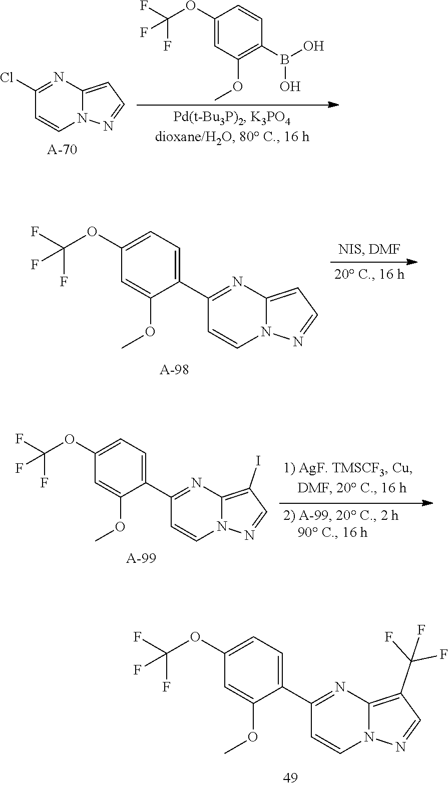

A mixture of 5-chloropyrazolo[1,5-a]pyrimidine (200.00 mg, 1.30 mmol), [2-methoxy-4-(trifluoromethoxy)phenyl]boronic acid (306.73 mg, 1.30 mmol), Pd(t-Bu₃P)₂ (99.65 mg, 195.00 mol) and K₃PO₄ (551.90 mg, 2.60 mmol) in dioxane (10 mL) and H₂O (3 mL) was stirred at 80° C. for 16 hours. After cooling to r.t., the mixture was concentrated to give the crude product. The crude product was purified by silica gel column (PE:EtOAc=7:1 to 5:1 to 2:1 to EtOAc) to give 5-[2-methoxy-4-(trifluoromethoxy)phenyl]pyrazolo[1,5-a]pyrimidine (400.00 mg, 87% yield) as an oil. ¹H NMR (400 MHz, CDCl₃) δ_{H} 8.66 (br s, 1H), 8.13 (d, 1H), 7.96 (d, 1H), 7.41 (d, 1H), 6.99 (d, 1H), 6.87 (s, 1H), 6.72 (d, 1H), 3.93 (s, 3H). LCMS Rₜ=0.908 min in 1.5 mins chromatography, MS ESI calcd. for C₁₄H₁₁F₃N₃O₂ [M+H]⁺ 310.1, found 309.9.

To the mixture of 5-[2-methoxy-4-(trifluoromethoxy)phenyl]pyrazolo[1,5-a]pyrimidine (400.00 mg, 1.29 mmol) in DMF (7 mL) was added NIS (348.27 mg, 1.55 mmol), and the mixture was stirred at 20° C. for 16 hours. To the mixture was added H₂O (6 mL) and the solid formed was collected by filtration, washed with water (4 mL×3) and dried in oven to give 3-iodo-5-[2-methoxy-4-(trifluoromethoxy)phenyl]pyrazolo[1,5-a]pyrimidine (450.00 mg, 76.% yield) as a solid. ¹H NMR (400 MHz, CDCl₃) δ_{H} 8.61 (d, 1H), 8.17-8.08 (m, 2H), 7.51 (d, 1H), 7.02 (dd, 1H), 6.87 (s, 1H), 3.94 (s, 3H). LCMS Rₜ=1.030 mins in 1.5 mins chromatography, MS ESI calcd. for C₁₄H₁₀F₃IN₃O₂ [M+H]⁺ 436.0, found 435.8.

To a mixture of AgF (56.86 mg, 448.14 μmol) in DMF (10 mL) was added TMSCF₃ (78.43 mg, 551.55 μmol), then the mixture was stirred at 20° C. for 1 hour. To the mixture was added Cu (43.81 mg, 689.44 μmol), then the mixture was stirred at 20° C. 15 hours under N₂. To the mixture was added 3-iodo-5-[2-methoxy-4-(trifluoromethoxy)phenyl]pyrazolo[1,5-a]pyrimidine (150.00 mg, 344.72 μmol), then the mixture was stirred at 20° C. for 2 hours and 90° C. for 16 hours. After cooling to r.t., the mixture was diluted with H₂O (20 mL), and the mixture was extracted with EtOAc (20 mL×2). The combined organic phase was washed with brine (15 mL), dried over Na₂SO₄, filtered and concentrated to give the crude product. The crude product was purified by Prep-HPLC (column: Kromasil 150*25 mm*10 um; mobile phase: [water (0.05% ammonia hydroxide v/v)-ACN]; B %: 55%-85%, 8 mins) to give 5-[2-methoxy-4-(trifluoromethoxy)phenyl]-3-(trifluoromethyl)pyrazolo[1,5-a]pyrimidine (65.28 mg, 50% yield) as a solid. ¹H NMR (400 MHz, CDCl₃) δ_{H} 8.68 (d, 1H), 8.31 (s, 1H), 8.15 (d, 1H), 7.70 (d, 1H), 7.03 (d, 1H), 6.88 (s, 1H), 3.96 (s, 3H). LCMS Rₜ=1.292 mins in 2.0 mins chromatography, MS ESI calcd. for C₁₅H₁₀F₆N₃O₂ [M+H]⁺ 378.1, found 377.9.

### Example 50: Synthesis of Compound 50

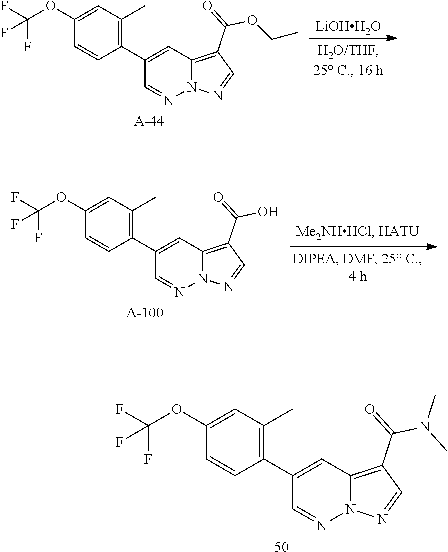

To a mixture of ethyl 5-[2-methyl-4-(trifluoromethoxy)phenyl]pyrazolo[1,5-b]pyridazine-3-carboxylate (60 mg, 0.16 mmol) in THF (3 mL) and Water (1.5 mL) was added LiOH.H₂O (20.46 mg, 0.49 mmol), then the mixture was stirred at 25° C. for 16 hours. The mixture was acidified with 1 N HCl to pH ˜5. The mixture was concentrated to give 5-[2-methyl-4-(trifluoromethoxy)phenyl]pyrazolo[1,5-b]pyridazine-3-carboxylic acid (55 mg, 98% yield) as a solid. The crude product was used next step without further purification. LCMS Rₜ=0.80 min in 1.5 min chromatography, MS ESI calcd. for C₅H₁₁F₃N₃O₃ [M+H]⁺ 338.1, found 337.9.

A mixture of 5-[2-methyl-4-(trifluoromethoxy)phenyl]pyrazolo[1,5-b]pyridazine-3-carboxylic acid (55 mg, 0.16 mmol), HATU (93.01 mg, 0.24 mmol), N-methylmethanamine hydrochloride (26.6 mg, 0.33 mmol) and DIPEA (0.1 mL, 0.57 mmol) in DMF (3 mL) was stirred at 25° C. for 4 hours. The mixture was diluted with H₂O (20 m), and the mixture was extracted with EtOAc (30 mL×2). The combined organic phase was washed with water (20 mL) and brine (20 mL), dried over Na₂SO₄, filtered and concentrated to give the crude product. The crude product was purified by Prep-TLC (silica gel, PE:EtOAc=1:1) to give N,N-dimethyl-5-[2-methyl-4-(trifluoromethoxy)phenyl]pyrazolo [1,5-b]pyridazine-3-carboxamide (33.9 mg, 57% yield) as a solid. ¹H NMR (400 MHz, DMSO-d₆) δ_{H} 8.61 (d, 1H), 8.42 (s, 1H), 8.37 (d, 1H), 7.53 (d, 1H), 7.38 (s, 1H), 7.32 (d, 1H), 3.15 (s, 6H), 2.38 (s, 3H). LCMS Rₜ=1.15 min in 2.0 min chromatography, MS ESI calcd. for C₁₇H₁₆F₃N₄O₂ [M+H]⁺ 365.1, found 365.0.

### Example 51: Efficacy of Exemplary Compounds in the Modulation of Late Sodium Current (INaL)

Functional characterization of exemplary compounds to modulate INaL expressed by the Nav1.6 voltage-gated sodium channel was accomplished using the PatchXpress' high throughput electrophysiology platform (Molecular Devices, Sunnyvale, Calif.). HEK-293 cells expressing recombinant, human Nav1.6 (hNav1.6) were grown in DMEM/high-glucose Dulbecco's modified, 10% FBS, 2 mM sodium pyruvate, 10 mM HEPES and 400 μg/mL G418. Cells were grown to 50%-80% confluency prior to harvesting. Trypsinized cells were washed, allowed to recover for 1 hour and then resuspended in extracellular recording solution at a concentration of 1×10⁶ cells/ml. The onboard liquid handling facility of the PatchXpress was used for dispensing cells and applying test compounds. Nav late currents were evoked by the application of 300 nM ATX-II. INaL was evoked by depolarizing pulses to 0 mV for 200 ms from a non-inactivating holding potential (e.g., −120 mV) at a frequency of 0.1 Hz. INaL amplitude and stability were determined by analyzing the mean current amplitude over the final 20 ms of thetest pulse. Following steady state block with exemplary compounds (e.g., as described herein), a Na⁺ free solution containing an impermeant cation (e.g., Choline or NDMG) was added to confirm the identify of the sodium current. Percent steady-state inhibition of INaL was calculated as: [(INaL_compound)/(INaL_control)]*100, where INaL_compound and INaL_control represent INaL recorded in the presence or absence of compound, respectively.

Results from this assay relating to percent inhibition of hNav1.6 at 1 μM are summarized in Table 1 below. In this table, “A” indicates inhibition between less than 0% to 50% and “B” indicates inhibition of greater than 50%.

TABLE 1

| | INaL hNaᵥ1.6 |
|---|---|
| Compound No. | (1 μM, % inhibition) |
| 1 | B |
| 2 | B |
| 3 | B |
| 4 | B |
| 5 | A |
| 6 | A |
| 7 | A |
| 8 | B |
| 9 | B |
| 10 | A |
| 11 | B |
| 12 | A |
| 13 | A |
| 14 | B |
| 15 | B |
| 16 | A |
| 17 | A |
| 18 | A |
| 19 | A |
| 20 | A |
| 21 | B |
| 22 | A |
| 23 | A |
| 24 | A |
| 25a | B |
| 25b | B |
| 26 | A |
| 27 | A |
| 28 | A |
| 29 | B |
| 30 | A |
| 31 | A |
| 32 | A |
| 33 | A |
| 34 | B |
| 35 | A |
| 36 | B |
| 37 | A |
| 38 | A |
| 39 | A |
| 40 | A |
| 41 | B |
| 42 | B |
| 43 | A |
| 44 | A |
| 45 | B |
| 46 | A |
| 47 | B |
| 48 | B |
| 49 | B |
| 50 | A |

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating a neurological disorder or a psychiatric disorder, wherein the method comprises administering to a subject in need thereof a compound of Formula (II): 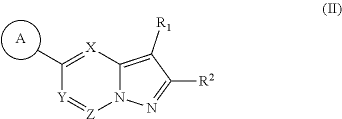
or a pharmaceutically acceptable salt thereof, wherein:
each of X, Y, and Z is independently N or CR′;
A is aryl or heteroaryl (e.g., a monocyclic 6-membered aryl or heteroaryl), wherein aryl and heteroaryl are optionally substituted with one or more R³;
R′ is hydrogen, alkyl, or —OR^{c};
each of R¹ and R² is hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴;
each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵;
each R⁴ and R⁵ is independently alkyl, halo, cyano, nitro, or —OR^{c};
each R^{c} is independently hydrogen, alkyl, aryl, or heteroaryl, wherein alkyl, aryl, or heteroaryl is optionally substituted by one or more R⁶;
each R^{d} is independently hydrogen or alkyl; and
each R⁶ is independently alkyl, carbocyclyl, heterocyclyl, halo, cyano, nitro, or —OH.

2. A method of treating a neurological disorder or a psychiatric disorder, wherein the method comprises administering to a subject in need thereof a compound of Formula (II-2): 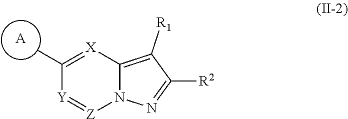
or a pharmaceutically acceptable salt thereof, wherein:
each of X, Y, and Z is independently N or CR′;
A is aryl or heteroaryl (e.g., a monocyclic 6-membered aryl or heteroaryl), wherein aryl and heteroaryl are optionally substituted with one or more R³;
R′ is hydrogen, alkyl, or —OR^{c};
each of R¹ and R² is hydrogen, —OR^{c}, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴;
each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵;
each R⁴ and R⁵ is independently alkyl, halo, cyano, nitro, or —OR^{c};
each R^{c} is independently hydrogen, alkyl, aryl, cycloalkyl, heterocyclyl, or heteroaryl, wherein alkyl, aryl, cycloalkyl, heterocyclyl, or heteroaryl is optionally substituted by one or more R⁶;
each R^{d} is independently hydrogen or alkyl; and
each R⁶ is independently alkyl, haloalkyl, carbocyclyl, heterocyclyl, halo, cyano, nitro, or —OH.

3. The method of claim 1, wherein the neurological disorder is epilepsy.

4. The method of claim 3, wherein the neurological disorder is an epileptic encephalopathy.

5. The method of claim 4, wherein the epileptic encephalopathy comprises Dravet syndrome, infantile spasms, or Lennox-Gastaut syndrome.

6. The method of claim 1, wherein each of X, Y, and Z is independently CR′.

7. The method of claim 2, wherein CR′ is CH.

8. The method of claim 1, wherein one of X, Y, and Z is independently N.

9. The method of claim 8, wherein X is N and each of Y and Z is independently CR′.

10. The method of claim 9, wherein CR′ is CH.

11. The method of any one of the preceding claims, wherein A is aryl (e.g., phenyl).

12. The method of any one of the preceding claims, wherein A is phenyl substituted by 1-3 R³.

13. The method of any one of claims 1-10, wherein A is heteroaryl (e.g., pyridyl).

14. The method of claim 13, wherein A is pyridyl substituted by 1-3 R³.

15. The method of any one of the preceding claims, wherein each R³ is independently alkyl, halo, cyano, carbocyclyl, or —OR^{c}.

16. The method of claim 15, wherein R³ is alkyl or —OR^{c}.

17. The method of any one of the preceding claims, wherein each of R¹ and R² is hydrogen.

18. The method of any one of claims 1-16, wherein R¹ is alkyl (e.g., substituted with one or more R⁴) and R² is hydrogen.

19. The method of claim 18, wherein R¹ is —CF₃.

20. The method of any one of the preceding claims, wherein the compound of Formula (II-2) is selected from the group consisting of: 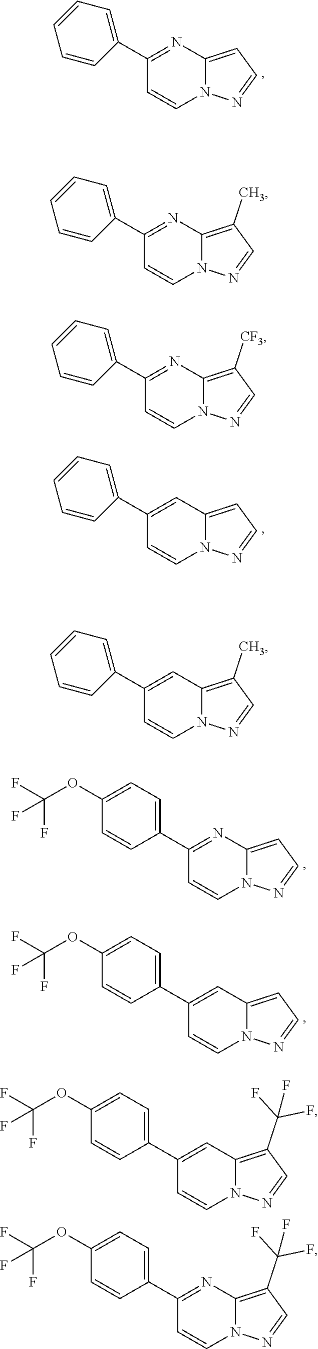
and a pharmaceutically acceptable salt thereof.

21. The method of any one of the preceding claims, wherein the compound of Formula (II-2) is selected from the group consisting of: 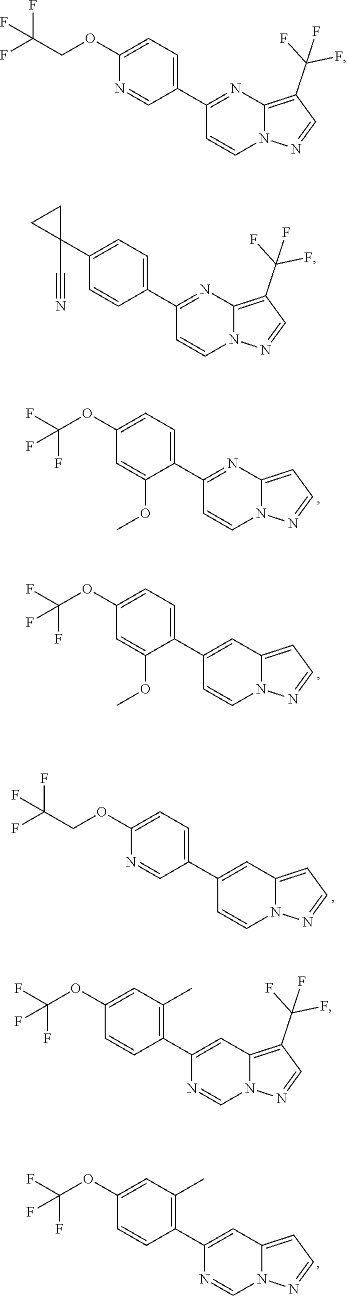 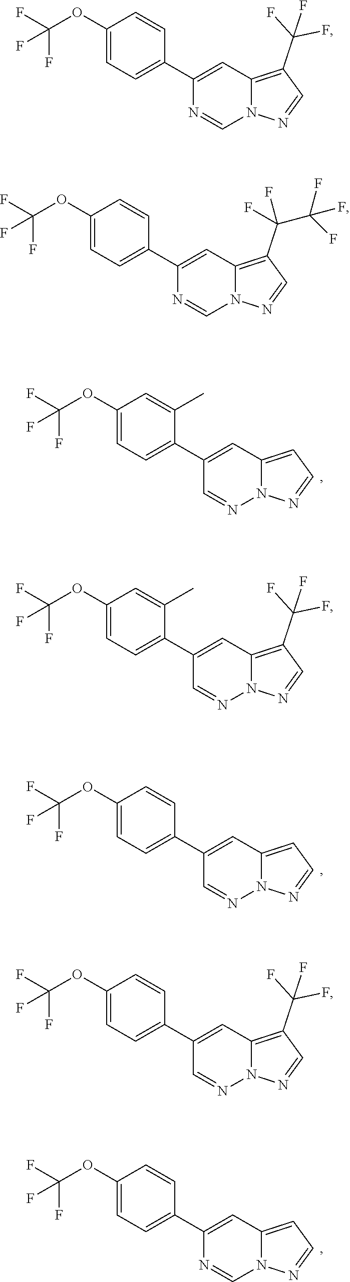 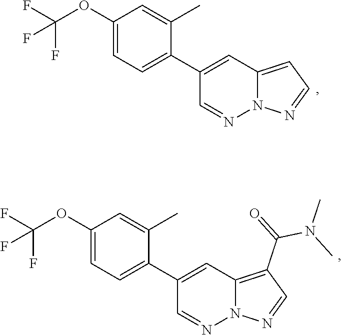 [SMILES: Cc1cc(OC(F)(F)F)ccc1-c1cnn2nccc2c1 ; Cc1cc(OC(F)(F)F)ccc1-c1cnn2ncc(C(=O)N(C)C)c2c1]
and a pharmaceutically acceptable salt thereof.

22. A method of treating a neurological disorder or a psychiatric disorder, wherein the method comprises administering to a subject in need thereof a compound of Formula (III): 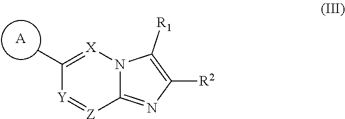
or a pharmaceutically acceptable salt thereof, wherein:
each of X, Y, and Z is independently N or CR′;
A is aryl or heteroaryl (e.g., a monocyclic 6-membered aryl or heteroaryl), wherein aryl and heteroaryl are optionally substituted with one or more R³;
R′ is hydrogen, alkyl, or —OR^{c};
each of R¹ and R² is hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴;
each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵;
each R⁴ and R⁵ is independently alkyl, halo, cyano, nitro, or —OR^{c};
each R^{c} is independently hydrogen, alkyl, aryl, cycloalkyl, heterocyclyl, or heteroaryl, wherein alkyl, aryl, cycloalkyl, heterocyclyl, or heteroaryl is optionally substituted by one or more R⁶;
each R^{d} is independently hydrogen or alkyl; and
each R⁶ is independently alkyl, haloalkyl, carbocyclyl, heterocyclyl, halo, cyano, nitro, or —OH.

23. The method of claim 22, wherein the neurological disorder is epilepsy.

24. The method of claim 23, wherein the neurological disorder is an epileptic encephalopathy.

25. The method of claim 24, wherein the epileptic encephalopathy comprises Dravet syndrome, infantile spasms, or Lennox-Gastaut syndrome.

26. The method of claim 22, wherein each of X, Y, and Z is independently CR′.

27. The method of claim 23, wherein CR′ is CH.

28. The method of claim 22, wherein one of X, Y, and Z is independently N.

29. The method of claim 28, wherein X is N and each of Y and Z is independently CR′.

30. The method of claim 29, wherein CR′ is CH.

31. The method of any one of claims 26-30, wherein A is aryl (e.g., phenyl).

32. The method of claim 31, wherein A is phenyl substituted by 1-3 R³.

33. The method of any one of claims 26-30, wherein A is heteroaryl (e.g., pyridyl).

34. The method of claim 33, wherein A is pyridyl substituted by 1-3 R³.

35. The method of any one of claims 26-34, wherein each R³ is independently alkyl, halo, cyano, carbocyclyl, or —OR^{c}.

36. The method of claim 35, wherein R³ is alkyl or —OR^{c}.

37. The method of any one of claims 26-36, wherein each of R and R² is hydrogen.

38. The method of any one of claims 25-35, wherein R¹ is alkyl (e.g., substituted with one or more R⁴) and R² is hydrogen.

39. The method of claim 38, wherein R¹ is —CF₃.

40. The method of any one of claims 26-39, wherein the compound of Formula (III) is selected from the group consisting of: 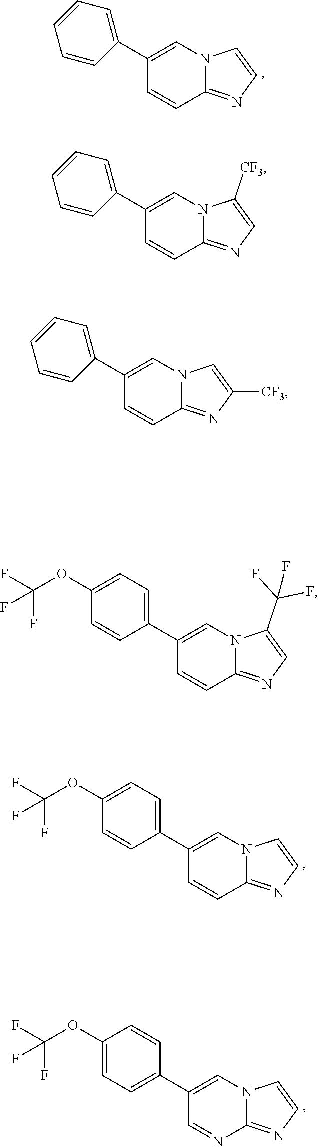 [SMILES: c1ccc(-c2ccc3nccn3c2)cc1 ; FC(F)(F)c1cnc2ccc(-c3ccccc3)cn12 ; FC(F)(F)c1cn2cc(-c3ccccc3)ccc2n1 ; FC(F)(F)Oc1ccc(-c2ccc3ncc(C(F)(F)F)n3c2)cc1 ; FC(F)(F)Oc1ccc(-c2ccc3nccn3c2)cc1 ; FC(F)(F)Oc1ccc(-c2cnc3nccn3c2)cc1]
and a pharmaceutically acceptable salt thereof.

41. The method of any one of claims 26-39, wherein the compound of Formula (III) is selected from the group consisting of: 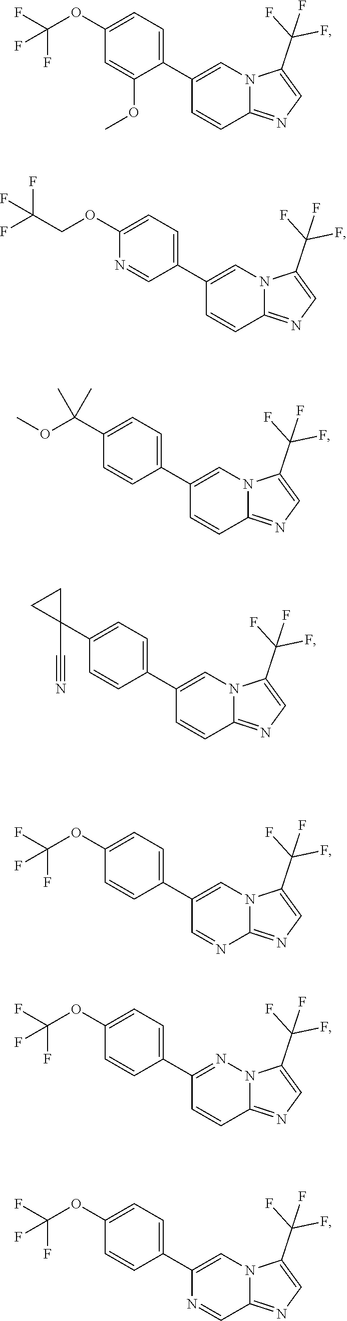 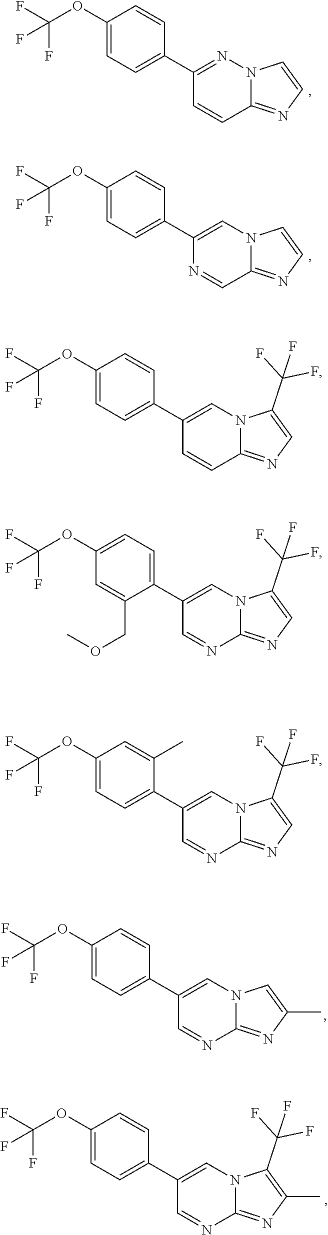 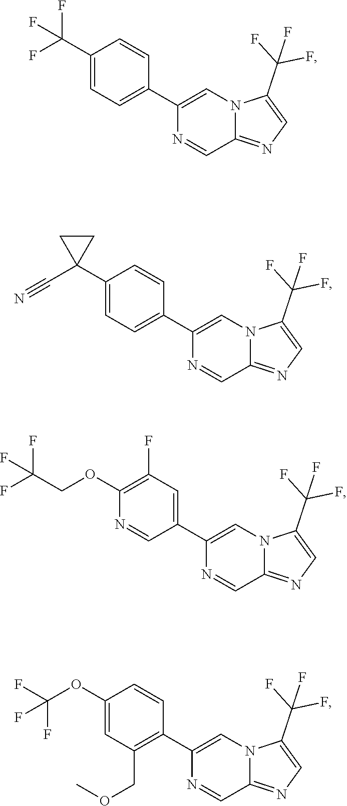 [SMILES: FC(F)(F)c1ccc(-c2cn3c(C(F)(F)F)cnc3cn2)cc1 ; N#CC1(c2ccc(-c3cn4c(C(F)(F)F)cnc4cn3)cc2)CC1 ; Fc1cc(-c2cn3c(C(F)(F)F)cnc3cn2)cnc1OCC(F)(F)F ; COCc1cc(OC(F)(F)F)ccc1-c1cn2c(C(F)(F)F)cnc2cn1]
and a pharmaceutically acceptable salt thereof.

42. A compound of Formula (IIa): 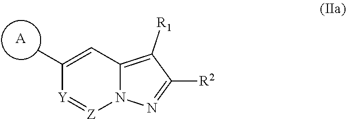
or a pharmaceutically acceptable salt thereof, wherein:
each of Y and Z is independently N or CR′, wherein at least one of Y and Z is N;
A is aryl or heteroaryl (e.g., a monocyclic 6-membered aryl or heteroaryl), wherein aryl and heteroaryl are substituted with one or more R³;
R′ is hydrogen, alkyl, or —OR^{c};
each of R¹ and R² is hydrogen, alkyl, alkenyl, alkynyl, carbocyclyl, heterocyclyl, aryl, or heteroaryl, wherein alkyl, alkenyl, alkynyl, carbocyclyl, heterocyclyl, aryl, and heteroaryl are optionally substituted with one or more R⁴;
each R³ is independently alkyl, halo, cyano, nitro, carbocyclyl, heterocyclyl, —OR^{c}, —N(R^{d})₂, —C(O)R^{c}, —C(O)OR^{c}, or —C(O)N(R^{d})₂, wherein alkyl, carbocyclyl, and heterocyclyl are optionally substituted with one or more R⁵;
each R⁴ and R⁵ is independently alkyl, halo, cyano, nitro, or —OR^{c};
each R^{c} is independently hydrogen, alkyl, aryl, cycloalkyl, heterocyclyl, or heteroaryl, wherein alkyl, aryl, cycloalkyl, or heteroaryl is optionally substituted by one or more R⁶;
each R^{d} is independently hydrogen or alkyl; and
each R⁶ is independently alkyl, haloalkyl, carbocyclyl, heterocyclyl, halo, cyano, nitro, or —OH.

43. The compound of claim 42, wherein Y is N and Z is CR′ (e.g., CH).

44. The compound of claim 42, wherein Z is N and Y is CR′ (e.g., CH).

45. The compound of any one of claims 42-44, wherein A is aryl (e.g., phenyl) substituted by 1-3 R³.

46. The compound of any one of claims 42-44, wherein A is heteroaryl (e.g., pyridyl) substituted by 1-3 R³.

47. The compound of any one of claims 42-44, wherein each R³ is independently alkyl, halo, cyano, carbocyclyl, or —OR^{c}.

48. The compound of claim 47, wherein at least one R³ is alkyl or —OR^{c}.

49. The compound of any one of claims 42-48, wherein R¹ is hydrogen or alkyl (e.g., substituted with one or more R⁴) and R² is hydrogen.

50. The compound of claim 49, wherein R¹ is —CF₃.

51. A compound of Formula (IIb): 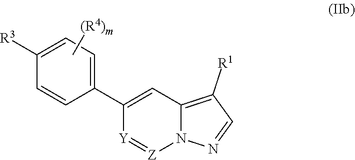
or a pharmaceutically acceptable salt thereof, wherein:
each of Y and Z is independently N or CH, wherein one of Y and Z is N and the other is CH;
R¹ is hydrogen or C₁₋₆alkyl, —C(O)N(R^{d})₂, —C(O)R^{c}, wherein C₁₋₆ alkyl is optionally substituted with one or more halo, cyano, nitro, C₃₋₁₀ carbocyclyl, C₃₋₁₀ heterocyclyl, —OR^{c}, —N(R^{d})₂, —C(O)R^{c}, —C(O)OR^{c}, or —C(O)N(R^{d})₂, wherein C₃₋₁₀ carbocyclyl and C₃₋₁₀ heterocyclyl are each optionally substituted with one or more R⁵;
each R³ is independently C₁₋₆alkyl, halo, cyano, nitro, C₃₋₁₀ carbocyclyl, C₃₋₁₀ heterocyclyl, —OR^{c}, —N(R^{d})₂, —C(O)R^{c}, —C(O)OR^{c}, or —C(O)N(R^{d})₂, wherein C₁₋₆ alkyl, C₃₋₁₀ carbocyclyl, C₃₋₁₀ heterocyclyl are optionally substituted with one or more R⁵;
R⁴ is C₁₋₆alkyl, halo, or OR^{c}, wherein C₁₋₆ alkyl is optionally substituted with one or more R⁵;
m is 0, 1, or 2;
R⁵ is independently C₁₋₆ alkyl, halo, cyano, nitro, or —OR^{c};
each R^{c} is independently hydrogen, C₁₋₆ alkyl, phenyl, C₃₋₈carbocyclyl, 5-8 membered heterocyclyl, or 5-8 membered heteroaryl, wherein C₁₋₆ alkyl, phenyl, C₃₋₈carbocyclyl, 5-8 membered heterocyclyl, or 5-8 membered heteroaryl is optionally substituted by one or more R⁶;
each R^{d} is independently hydrogen or C₁₋₆ alkyl; and
each R⁶ is independently C₁₋₆ alkyl, C₁₋₆haloalkyl, C₃₋₁₀ carbocyclyl, C₃₋₁₀ heterocyclyl, halo, cyano, nitro, or —OH.

52. The compound of claim 51, wherein the compound is a compound of formula (IIb-1): 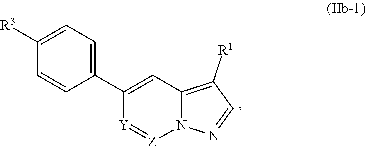
or a pharmaceutically acceptable salt thereof, wherein the variables are as defined in claim 42.

53. The compound of claim 51 or 52, wherein Y is N and Z is CH.

54. The compound of claim 51 or 52, wherein Y is CH and Z is N.

55. The compound of any one of claims 51-54, wherein R is selected from the group consisting of hydrogen or C₁₋₆alkyl, and —C(O)N(R^{d})₂, wherein C₁₋₆alkyl is optionally substituted with one or more halogen.

56. The compound of any one of claims 51-55, wherein R is selected from the group consisting of hydrogen, —CF₃, —CF₂—CF₃, or —C(O)N(CH₃)₂.

57. The compound of any one of claims 51-56, wherein R³ is independently selected from C₁₋₆alkyl or —OR^{c}, wherein R^{c} is C₁₋₆alkyl optionally substituted with one or more halogen or C₃₋₈ carbocyclyl optionally substituted with C₁₋₆haloalkyl or cyano.

58. The compound of any one of claims 51-57, wherein R³ is independently selected from the group consisting of methyl, —OCF₃ and O—CH₂—CF₃.

59. The compound of any one of claims 51-58, wherein R⁴ is selected from the group consisting of methyl, F, —OMe, and —CH₂—OMe.

60. The compound of any one of claims 51-59, wherein m is 0.

61. The compound of any one of claims 51-59, wherein the compound is selected from the group consisting of: 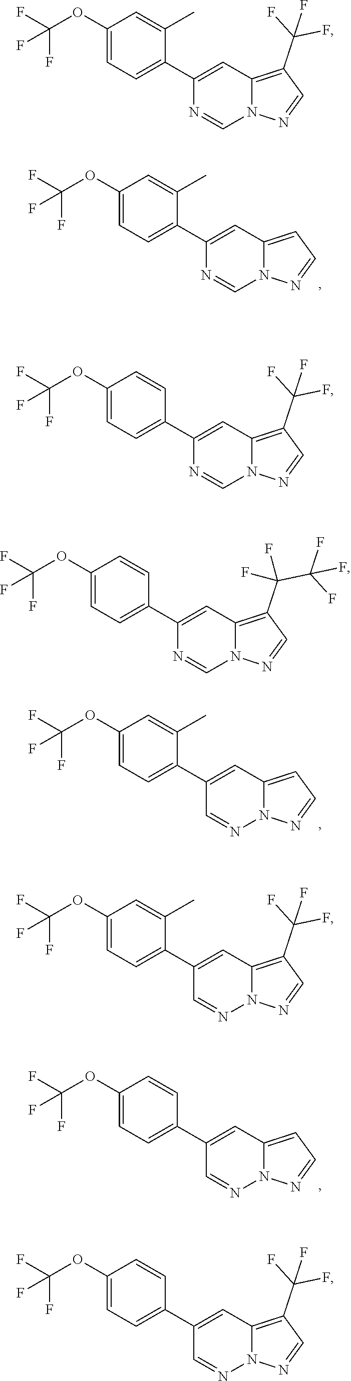 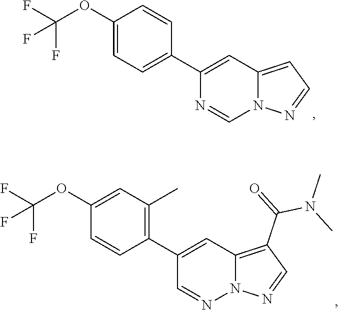 [SMILES: FC(F)(F)Oc1ccc(-c2cc3ccnn3cn2)cc1 ; Cc1cc(OC(F)(F)F)ccc1-c1cnn2ncc(C(=O)N(C)C)c2c1]
and a pharmaceutically acceptable salt thereof.

62. A compound of Formula (IIc): 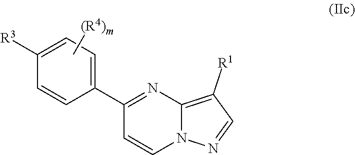
or a pharmaceutically acceptable salt thereof, wherein:
R¹ is hydrogen or C₁₋₆alkyl, —C(O)N(R^{d})₂, —C(O)R^{c}, wherein C₁₋₆ alkyl is optionally substituted with one or more halo, cyano, nitro, C₃₋₁₀ carbocyclyl, C₃₋₁₀ heterocyclyl, —OR^{c}, —N(R^{d})₂, —C(O)R^{c}, —C(O)OR^{c}, or —C(O)N(R^{d})₂, wherein C₃₋₁₀ carbocyclyl and C₃₋₁₀ heterocyclyl are each optionally substituted with one or more R⁵;
each R³ is independently C₁₋₆alkyl, halo, cyano, nitro, C₃₋₁₀ carbocyclyl, C₃₋₁₀ heterocyclyl, —OR^{c}, —N(R^{d})₂, —C(O)R^{c}, —C(O)OR^{c}, or —C(O)N(R^{d})₂, wherein C₁₋₆ alkyl, C₃₋₁₀ carbocyclyl, C₃₋₁₀ heterocyclyl are optionally substituted with one or more R⁵;
R⁴ is C₁₋₆alkyl, halo, or OR^{c}, wherein C₁₋₆ alkyl is optionally substituted with one or more R⁵;
m is 0, 1, or 2;
R⁵ is independently C₁₋₆ alkyl, halo, cyano, nitro, or —OR^{c};
each R^{c} is independently hydrogen, C₁₋₆ alkyl, phenyl, C₃₋₈carbocyclyl, 5-8 membered heterocyclyl, or 5-8 membered heteroaryl, wherein C₁₋₆ alkyl, phenyl, C₃₋₈carbocyclyl, 5-8 membered heterocyclyl, or 5-8 membered heteroaryl is optionally substituted by one or more R⁶;
each R^{d} is independently hydrogen or C₁₋₆ alkyl; and
each R⁶ is independently C₁₋₆ alkyl, C₁₋₆haloalkyl, C₃₋₁₀ carbocyclyl, C₃₋₁₀ heterocyclyl, halo, cyano, nitro, or —OH, wherein the compound is not: 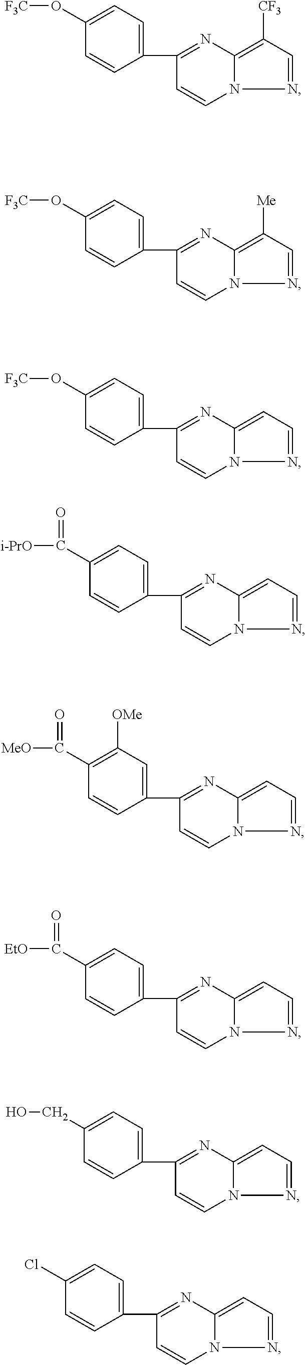
or a pharmaceutically acceptable salt thereof.

63. The compound of claim 62, wherein R¹ is selected from the group consisting of hydrogen or C₁₋₆alkyl, and —C(O)N(R^{d})₂, wherein C₁₋₆alkyl is optionally substituted with one or more halogen.

64. The compound of claim 62 or 63, wherein R¹ is selected from the group consisting of hydrogen, —CF₃, —CF₂—CF₃, or —C(O)N(CH₃)₂.

65. The compound of any one of claims 62-64, wherein R³ is independently selected from C₁₋₆alkyl or —OR^{c}, wherein R^{c} is C₁₋₆alkyl optionally substituted with one or more halogen or C₃₋₈ carbocyclyl optionally substituted with C₁₋₆haloalkyl or cyano.

66. The compound of any one of claims 62-64, wherein R³ is independently selected from the group consisting of methyl, —OCF₃ and O—CH₂—CF₃.

67. The compound of any one of claims 62-67, wherein R⁴ is selected from the group consisting of methyl, F, —OMe, and —CH₂—OMe.

68. The compound of any one of claims 62-67, wherein m is 0.

69. The compound of any one of claims 62-67, wherein the compound is selected from the group consisting of: 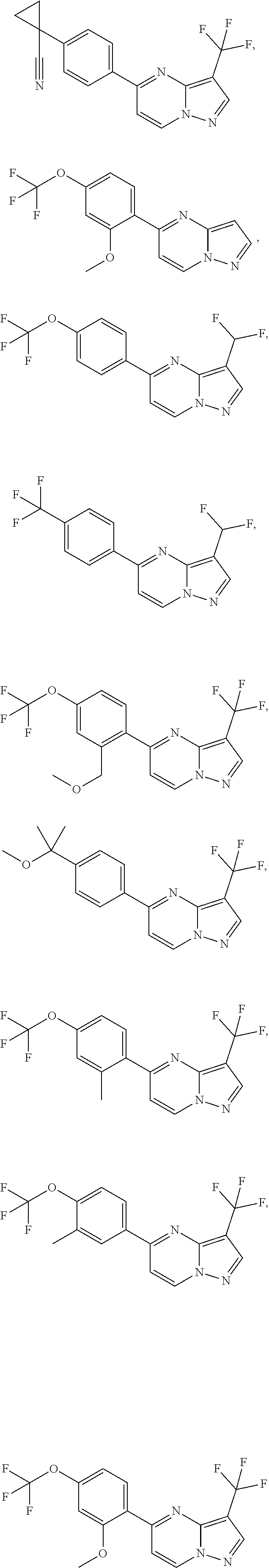
and a pharmaceutically acceptable salt thereof.

70. A compound of Formula (IId): 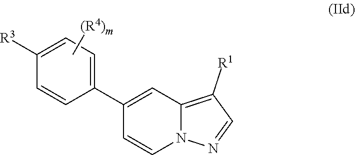
or a pharmaceutically acceptable salt thereof, wherein:
R¹ is hydrogen or C₁₋₆alkyl, —C(O)N(R^{d})₂, —C(O)R^{c}, wherein C₁₋₆ alkyl is optionally substituted with one or more halo, cyano, nitro, C₃₋₁₀ carbocyclyl, C₃₋₁₀ heterocyclyl, —OR^{c}, —N(R^{d})₂, —C(O)R^{c}, —C(O)OR^{c}, or —C(O)N(R^{d})₂, wherein C₃₋₁₀ carbocyclyl and C₃₋₁₀ heterocyclyl are each optionally substituted with one or more R⁵;
each R³ is independently C₁₋₆alkyl, halo, cyano, nitro, C₃₋₁₀ carbocyclyl, C₃₋₁₀ heterocyclyl, —OR^{c}, —N(R^{d})₂, —C(O)R^{c}, —C(O)OR^{c}, or —C(O)N(R^{d})₂, wherein C₁₋₆ alkyl, C₃₋₁₀ carbocyclyl, C₃₋₁₀ heterocyclyl are optionally substituted with one or more R⁵;
R⁴ is C₁₋₆alkyl, halo, or OR^{c}, wherein C₁₋₆ alkyl is optionally substituted with one or more R⁵;
m is 0, 1, or 2;
R⁵ is independently C₁₋₆ alkyl, halo, cyano, nitro, or —OR^{c};
each R^{c} is independently hydrogen, C₁₋₆ alkyl, phenyl, C₃₋₈carbocyclyl, 5-8 membered heterocyclyl, or 5-8 membered heteroaryl, wherein C₁₋₆ alkyl, phenyl, C₃₋₈carbocyclyl, 5-8 membered heterocyclyl, or 5-8 membered heteroaryl is optionally substituted by one or more R⁶;
each R^{d} is independently hydrogen or C₁₋₆ alkyl; and
each R⁶ is independently C₁₋₆ alkyl, C₁₋₆haloalkyl, C₃₋₁₀ carbocyclyl, C₃₋₁₀ heterocyclyl, halo, cyano, nitro, or —OH, wherein the compound is not: 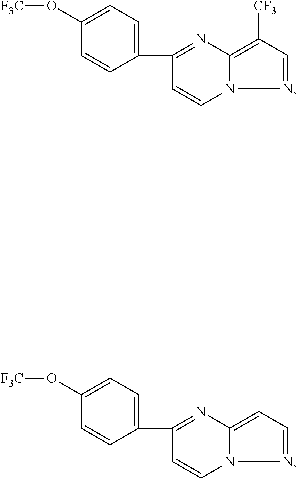 [SMILES: FC(F)(F)Oc1ccc(-c2ccn3ncc(C(F)(F)F)c3n2)cc1 ; FC(F)(F)Oc1ccc(-c2ccn3nccc3n2)cc1]
or a pharmaceutically acceptable salt thereof.

71. The compound of claim 70, wherein R¹ is selected from the group consisting of hydrogen or C₁₋₆alkyl, and —C(O)N(R^{d})₂, wherein C₁₋₆alkyl is optionally substituted with one or more halogen.

72. The compound of claim 70 or 71, wherein R¹ is selected from the group consisting of hydrogen, —CF₃, —CF₂—CF₃, or —C(O)N(CH₃)₂.

73. The compound of any one of claims 70-72, wherein R³ is independently selected from C₁₋₆alkyl or —OR^{c}, wherein R^{c} is C₁₋₆alkyl optionally substituted with one or more halogen or C₃₋₈ carbocyclyl optionally substituted with C₁₋₆haloalkyl or cyano.

74. The compound of any one of claims 70-72, wherein R³ is independently selected from the group consisting of methyl, —OCF₃ and O—CH₂—CF₃.

75. The compound of any one of claims 70-74, wherein R⁴ is selected from the group consisting of methyl, F, —OMe, and —CH₂—OMe.

76. The compound of any one of claims 70-75, wherein m is 0.

77. A compound selected from the group consisting of: 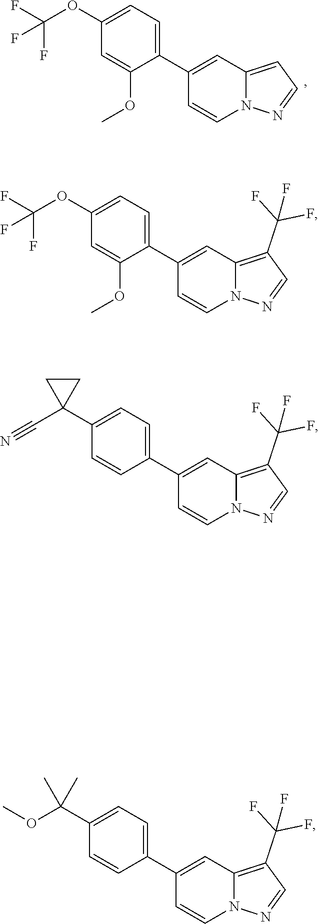 [SMILES: COc1cc(OC(F)(F)F)ccc1-c1ccn2nccc2c1 ; COc1cc(OC(F)(F)F)ccc1-c1ccn2ncc(C(F)(F)F)c2c1 ; N#CC1(c2ccc(-c3ccn4ncc(C(F)(F)F)c4c3)cc2)CC1 ; COC(C)(C)c1ccc(-c2ccn3ncc(C(F)(F)F)c3c2)cc1]
and a pharmaceutically acceptable salt thereof.

78. A compound of formula (IIe): 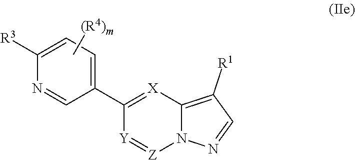
or a pharmaceutically acceptable salt thereof, wherein:
each of X, Y, and Z is independently N or CH;
R¹ is selected from the group consisting of: C₁₋₆ alkyl, C₁₋₆haloalkyl, and C₃₋₈carbocyclyl;
R³ is selected from the group consisting of: C₁₋₆alkyl, cyano, C₃₋₁₀ carbocyclyl, —OR^{c}, —C(O)R^{c}, —C(O)OR^{c}, or —C(O)N(R^{d})₂, wherein C₁₋₆ alkyl or C₃₋₁₀ carbocyclyl is optionally substituted with one or more R⁵;
R⁴ is C₁₋₆alkyl, halo, or OR^{c}, wherein C₁₋₆ alkyl is optionally substituted with one or more R⁵;
m is 0, 1, or 2;
R⁵ is independently C₁₋₆ alkyl, halo, cyano, nitro, or —OR^{c};
each R^{c} is independently hydrogen, C₁₋₆ alkyl, phenyl, C₃₋₈carbocyclyl, 5-8 membered heterocyclyl, or 5-8 membered heteroaryl, wherein C₁₋₆ alkyl, phenyl, C₃₋₈carbocyclyl, 5-8 membered heterocyclyl, or 5-8 membered heteroaryl is optionally substituted by one or more R⁶;
each R^{d} is independently hydrogen or C₁₋₆ alkyl; and
each R⁶ is independently C₁₋₆ alkyl, C₁₋₆haloalkyl, C₃₋₁₀ carbocyclyl, C₃₋₁₀ heterocyclyl, halo, cyano, nitro, or —OH.

79. The compound of claim 78, wherein the compound is a compound of formula (IIe-1): 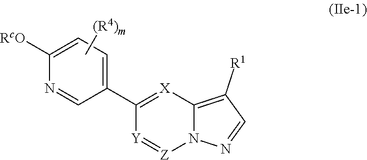
or a pharmaceutically acceptable salt thereof, wherein the variables are as defined in claim 54.

80. The compound of claim 78 or 79, wherein X is N, Y and Z are CH.

81. The compound of any one of claims 78-80, wherein Y is N, Y and Z are CH.

82. The compound of any one of claims 78-81, wherein Z is N, X and Y are CH.

83. The compound of any one of claims 78-82, wherein X, Y, and Z are CH.

84. The compound of any one of claims 78-83, wherein R is selected from the group consisting of: —CH₃, CF₃, CHF₂, and cyclopropyl.

85. The compound of any one of claims 78-84, wherein R³ is —OR^{c}, wherein R^{c} is selected from the group consisting of C₁₋₆alkyl substituted with 1, 2, or 3 halogens or C₃₋₈carbocyclyl optionally substituted with cyano or CF₃.

86. The compound of claim 85, wherein R^{c} is selected from the group consisting of: —CF₃, —CH₂CF₃, or

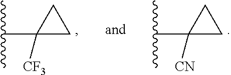 [SMILES: *C1(C(F)(F)F)CC1 ; *C1(C#N)CC1]

87. The compound of claim 86, wherein R^{c} is —CH₂CF₃.

88. The compound of any one of claims 78-87, wherein R⁴ is selected from the group consisting of methyl, F, —OMe, and —CH₂—OMe.

89. The compound of any one of claims 78-88, wherein m is 0.

90. The compound of any one of claims 78-87, wherein the compound is selected from the group consisting of: 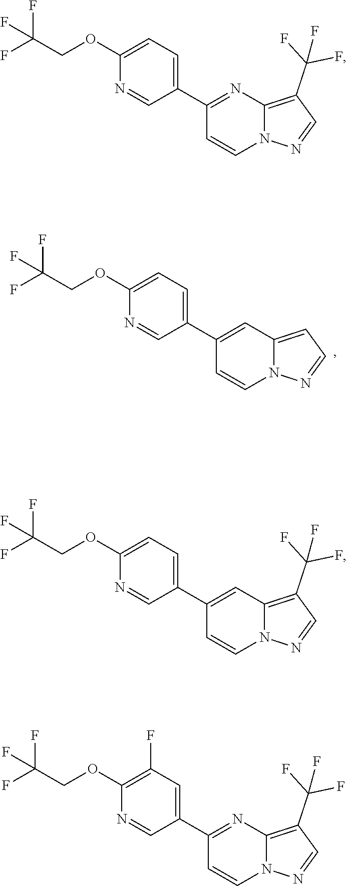 [SMILES: FC(F)(F)COc1ccc(-c2ccn3ncc(C(F)(F)F)c3n2)cn1 ; FC(F)(F)COc1ccc(-c2ccn3nccc3c2)cn1 ; FC(F)(F)COc1ccc(-c2ccn3ncc(C(F)(F)F)c3c2)cn1 ; Fc1cc(-c2ccn3ncc(C(F)(F)F)c3n2)cnc1OCC(F)(F)F]
and a pharmaceutically acceptable salt thereof.

91. A compound of Formula (IIIa): 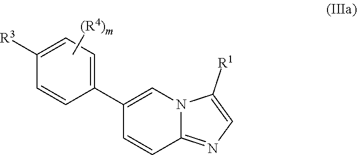
or a pharmaceutically acceptable salt thereof, wherein:
R¹ is hydrogen or C₁₋₆ haloalkyl;
R³ is —(C₁₋₆alkylene)-O—(C₁₋₆alkyl) or C₃₋₈ cycloalkyl optionally substituted with —CF₃ or —CN;
R⁴ is selected from the group consisting of methyl, F, —OMe, and —CH₂—OMe; and
m is 0, 1, or 2.

92. The compound of claim 91, wherein R is hydrogen or CF₃.

93. The compound of claim 91 or 92, wherein R³ is —(C₁₋₆alkylene)-O—(C₁₋₆alkyl).

94. The compound of any one of claims 91-93, wherein R³ is

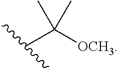 [SMILES: *C(C)(C)OC]

95. The compound of any one of claims 91-93, wherein R³ is C₃₋₈ cycloalkyl optionally substituted with —CF₃ or —CN.

96. The compound of any one of claims 91-95, wherein R³ is

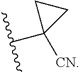 [SMILES: *C1(C#N)CC1]

97. The compound of any one of claims 91-96, wherein m is 0.

98. The compound of any one of claims 91-97, wherein the compound is selected from the group consisting of: 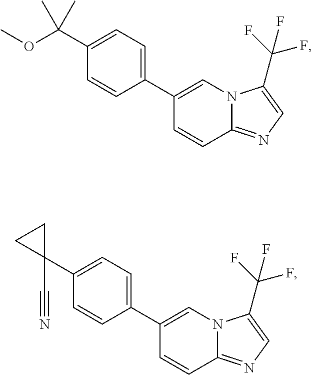 [SMILES: COC(C)(C)c1ccc(-c2ccc3ncc(C(F)(F)F)n3c2)cc1 ; N#CC1(c2ccc(-c3ccc4ncc(C(F)(F)F)n4c3)cc2)CC1]
and a pharmaceutically acceptable salt thereof.

99. A compound represented by: 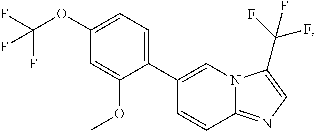 [SMILES: COc1cc(OC(F)(F)F)ccc1-c1ccc2ncc(C(F)(F)F)n2c1]
or a pharmaceutically acceptable salt thereof.

100. A compound of formula (IIb): 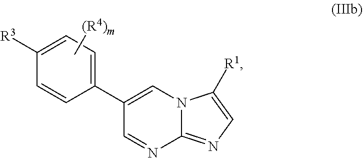
or a pharmaceutically acceptable alt thereof, wherein:
R¹ is C₁₋₆haloalkyl,
R³ is selected from the group consisting of —O—C₁₋₄haloalkyl, —(C₁₋₆alkylene)-O—(C₁₋₆alkyl), and C₃₋₇ cycloalkyl optionally substituted with —CF₃ or —CN;
R⁴ is selected from the group consisting of methyl, F, —OMe, and —CH₂—OMe; and
m is 0, 1, or 2.

101. The compound of claim 100, wherein R¹ is CF₃ or CHF₂.

102. The compound of claim 100 or 101, wherein R is CF₃.

103. The compound of any one of claims 100-102, wherein R³ is —O—C₁₋₄haloalkyl.

104. The compound of any one of claims 100-103, wherein R³ is —O—CH₂—CF₃ or —O—CF₃.

105. The compound of any one of claims 100-104, wherein R³ is —O—CF₃.

106. The compound of any one of claims 100-102, wherein R³ is —(C₁₋₆alkylene)-O—(C₁₋₆alkyl).

107. The compound of any one of claims 100-106, wherein R³ is

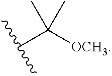 [SMILES: *C(C)(C)OC]

108. The compound of any one of claims 100-107, wherein R³ is C₃₋₇ cycloalkyl optionally substituted with —CF₃ or —CN.

109. The compound of any one of claims 100-108, wherein R³ is

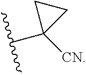 [SMILES: *C1(C#N)CC1]

110. The compound of any one of claims 100-109, wherein m is 0.

111. The compound of any one of claims 100-109, wherein the compound is selected from the group consisting of: 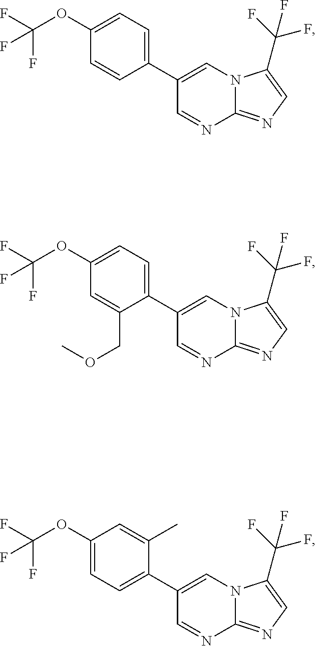 [SMILES: FC(F)(F)Oc1ccc(-c2cnc3ncc(C(F)(F)F)n3c2)cc1 ; COCc1cc(OC(F)(F)F)ccc1-c1cnc2ncc(C(F)(F)F)n2c1 ; Cc1cc(OC(F)(F)F)ccc1-c1cnc2ncc(C(F)(F)F)n2c1]
and a pharmaceutically acceptable salt thereof.

112. A compound selected from the group consisting of: 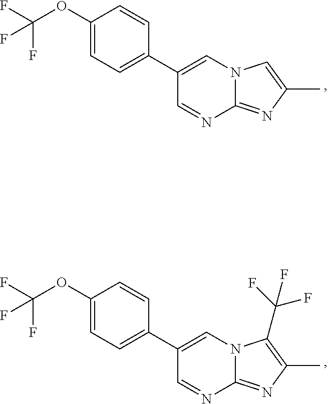 [SMILES: Cc1cn2cc(-c3ccc(OC(F)(F)F)cc3)cnc2n1 ; Cc1nc2ncc(-c3ccc(OC(F)(F)F)cc3)cn2c1C(F)(F)F]
and a pharmaceutically acceptable salt thereof.

113. A compound of formula (IIIc): 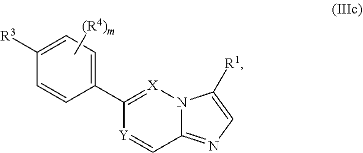
or a pharmaceutically acceptable salt thereof, wherein:
X is N and Y is CH, or X is CH and Y is N;
R¹ is hydrogen or C₁₋₄haloalkyl;
R³ is selected from the group consisting of —O—C₁₋₆haloalkyl, —(C₁₋₆alkylene)-O—(C₁₋₆alkyl), and C₃₋₇ cycloalkyl optionally substituted with —CF₃ or —CN;
R⁴ is selected from the group consisting of methyl, F, —OMe, and —CH₂—OMe; and
m is 0, 1, or 2.

114. The compound of claim 113, wherein X is N and Y is CH.

115. The compound of claim 113 or 114, wherein X is CH and Y is N.

116. The compound of any one of claims 113-115, wherein R¹ is hydrogen.

117. The compound of any one of claims 113-116, wherein R¹ is CF₃ or CHF₂.

118. The compound of any one of claims 113-117, wherein R¹ is CF₃.

119. The compound of any one of claims 113-118, wherein R³ is —O—C₁₋₄haloalkyl.

120. The compound of any one of claims 113-119, wherein R³ is —O—CH₂—CF³ or —O—CF₃.

121. The compound of any one of claims 113-120, wherein R³ is —O—CF₃.

122. The compound of any one of claims 113-118, wherein R³ is —(C₁₋₆alkylene)-O—(C₁₋₆alkyl).

123. The compound of any one of claims 113-121, wherein R³ is

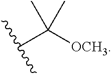 [SMILES: *C(C)(C)OC]

124. The compound of any one of claims 113-118, wherein R³ is C₃₋₇ cycloalkyl optionally substituted with —CF₃ or —CN.

125. The compound of any one of claim 124, wherein R³ is C₃₋₇ cycloalkyl substituted with —CN.

126. The compound of any one of claim 125, wherein R³ is

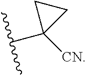 [SMILES: *C1(C#N)CC1]

127. The compound of any one of claims 113-125, wherein m is 0.

128. The compound of any one of claims 113-126, wherein the compound is selected from the group consisting of: 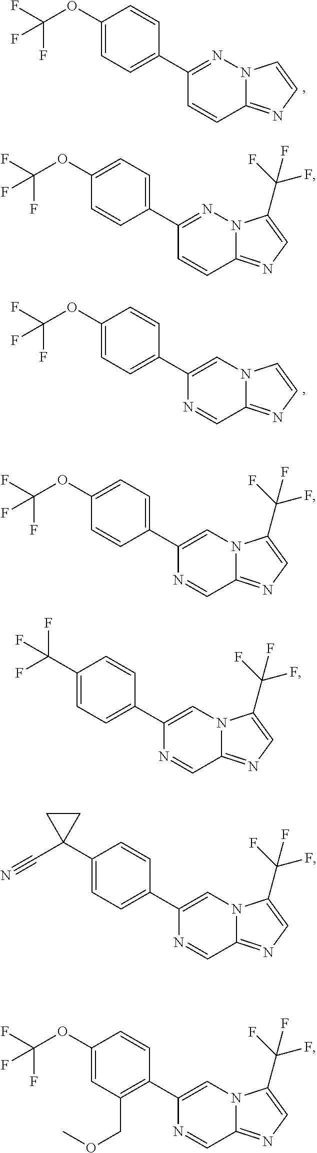
and a pharmaceutically acceptable salt thereof.

129. A compound of formula (IIId): 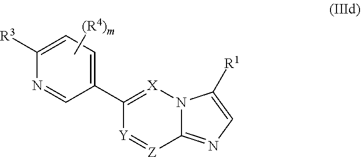
or a pharmaceutically acceptable salt thereof, wherein:
each of X, Y, and Z is independently N or CH;
R¹ is selected from the group consisting of: unsubstituted C₁₋₆ alkyl, C₁₋₆haloalkyl, and unsubstituted C₃₋₈cycloalkyl;
R³ is selected from the group consisting of: C₁₋₆alkyl, cyano, C₃₋₁₀ carbocyclyl, —OR^{c}, —C(O)R^{c}, —C(O)OR^{c}, and —C(O)N(R^{d})₂, wherein C₁₋₆ alkyl or C₃₋₁₀ carbocyclyl is optionally substituted with one or more R⁵;
R⁴ is C₁₋₆alkyl, halo, or OR^{c}, wherein C₁₋₆ alkyl is optionally substituted with one or more R⁵;
m is 0, 1, or 2;
R⁵ is independently C₁₋₆ alkyl, halo, cyano, nitro, or —OR^{c};
each R^{c} is independently hydrogen, C₁₋₆ alkyl, phenyl, C₃₋₈carbocyclyl, 5-8 membered heterocyclyl, or 5-8 membered heteroaryl, wherein C₁₋₆ alkyl, phenyl, C₃₋₈carbocyclyl, 5-8 membered heterocyclyl, or 5-8 membered heteroaryl is optionally substituted by one or more R⁶;
each R^{d} is independently hydrogen or C₁₋₆ alkyl; and
each R⁶ is independently C₁₋₆ alkyl, C₁₋₆haloalkyl, C₃₋₁₀ carbocyclyl, C₃₋₁₀ heterocyclyl, halo, cyano, nitro, or —OH.

130. The compound of claim 129, wherein the compound is a compound of formula (IIId-1): 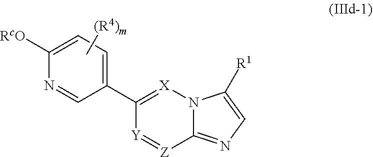
or a pharmaceutically acceptable salt thereof, wherein the variables are as defined in claim 116.

131. The compound of claim 129 or 130, wherein X is N, Y and Z are CH.

132. The compound of anyone of claims 129-131, wherein Y is N, Y and Z are CH.

133. The compound of any one of claims 129-132, wherein Z is N, X and Y are CH.

134. The compound of any one of claims 129-133, wherein X, Y, and Z are CH.

135. The compound of any one of claims 129-134, wherein R¹ is selected from the group consisting of: —CH₃, CF₃, CHF₂, and cyclopropyl.

136. The compound of any one of claims 129-135, wherein R³ is —OR^{c}, wherein R³ is selected from the group consisting of C₁₋₆alkyl substituted with 1, 2, or 3 halogens or C₃₋₈carbocyclyl optionally substituted with cyano or CF₃.

137. The compound of any one of claims 129-136, wherein R³ is selected from the group consisting of: —CF₃,

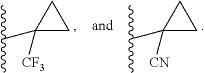 [SMILES: *C1(C(F)(F)F)CC1 ; *C1(C#N)CC1]

138. The compound of any one of claims 129-137, wherein R⁴ is selected from the group consisting of methyl, F, —OMe, and —CH₂—OMe.

139. The compound of any one of claims 129-138, wherein m is 0.

140. The compound of any one of claims 129-138, wherein the compound is selected from the group consisting of: 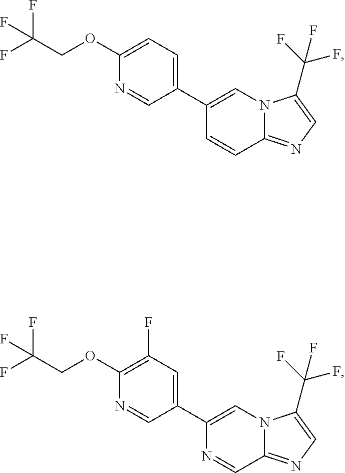 [SMILES: FC(F)(F)COc1ccc(-c2ccc3ncc(C(F)(F)F)n3c2)cn1 ; Fc1cc(-c2cn3c(C(F)(F)F)cnc3cn2)cnc1OCC(F)(F)F]
or a pharmaceutically acceptable salt thereof.

141. A pharmaceutical composition comprising a compound of any one of claims 42-140, or pharmaceutically acceptable salt thereof, and a pharmaceutically acceptable carrier.

142. A method of treating a neurological disorder or a psychiatric disorder, wherein the method comprises administering to a subject in need thereof a compound of any one of claims 42-140 or a pharmaceutical composition of claim 141.

143. The method of claim 142, wherein the neurological disorder is epilepsy.

144. The method of claim 143, wherein the neurological disorder is an epileptic encephalopathy.

145. The method of claim 144, wherein the epileptic encephalopathy comprises Dravet syndrome, infantile spasms, or Lennox-Gastaut syndrome.
